SEQUENCE LISTING (699 sequences)

SEQ ID NO: 1 - protein, 185 aa, synthetic construct
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 2 - skipped

SEQ ID NO: 3 - protein, 5 aa, synthetic construct
TYAMN

SEQ ID NO: 4 - skipped

SEQ ID NO: 5 - protein, 19 aa, synthetic construct
RIRSKYNNYATYYADSVKD

SEQ ID NO: 6 - protein, 14 aa, synthetic construct
  VARIANT 12: note = X is W or F
HGNFGNSYVSWXAY

SEQ ID NO: 7 - protein, 14 aa, synthetic construct
HGNFGNSYVSWWAY

SEQ ID NO: 8 - protein, 14 aa, synthetic construct
HGNFGNSYVSWFAY

SEQ ID NO: 9 - skipped

SEQ ID NO: 10 - protein, 14 aa, synthetic construct
  VARIANT 1: note = X is R or G
  VARIANT 13: note = X is P or V
XSSTGAVTTSNYXN

SEQ ID NO: 11 - protein, 14 aa, synthetic construct
RSSTGAVTTSNYPN

SEQ ID NO: 12 - protein, 14 aa, synthetic construct
RSSTGAVTTSNYVN

SEQ ID NO: 13 - protein, 14 aa, synthetic construct
GSSTGAVTTSNYVN

SEQ ID NO: 14 - protein, 7 aa, synthetic construct
GTNKRAP

SEQ ID NO: 15 - protein, 9 aa, synthetic construct
  VARIANT 1: note = X is V or I
  VARIANT 7: note = X is R or L
XLWYSNXWV

SEQ ID NO: 16 - protein, 9 aa, synthetic construct
VLWYSNRWV

SEQ ID NO: 17 - protein, 9 aa, synthetic construct
VLWYSNLWV

SEQ ID NO: 18 - protein, 9 aa, synthetic construct
ILWYSNRWV

SEQ ID NO: 19 - skipped

SEQ ID NO: 20 - protein, 125 aa, synthetic construct
  VARIANT 112: note = X is W or F
  VARIANT 117: note = X is Q or C
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWXAYWGXGTLVTVSS

SEQ ID NO: 21 - protein, 109 aa, synthetic construct
  VARIANT 23: note = X is R or G
  VARIANT 35: note = X is P or V
  VARIANT 44: note = X is Q or C
  VARIANT 80: note = X is V or A
  VARIANT 91: note = X is V or I
  VARIANT 97: note = X is R or L
QTVVTQEPSLTVSPGGTVTLTCXSSTGAVTTSNYXNWVQQKPGXAPRGLIGGTNKRAPGTPARFSGSLJGGKAALTLSGXQPEDEAEYYCXLWYSNXWVFGGGTKLTVL

SEQ ID NO: 22 - protein, 125 aa, synthetic construct
  VARIANT 112: note = X is W or F
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWXAYWGQGTLVTVSS

SEQ ID NO: 23 - protein, 109 aa, synthetic construct
  VARIANT 23: note = X is R or G
  VARIANT 35: note = X is P or V
  VARIANT 80: note = X is V or A
  VARIANT 91: note = X is V or I
  VARIANT 97: note = X is R or L
QTVVTQEPSLTVSPGGTVTLTCXSSTGAVTTSNYXNWVQQKPGQAPRGLIGGTNKRAPGTPARFSGSLJGGKAALTLSGXQPEDEAEYYCXLWYSNXWVFGGGTKLTVL

SEQ ID NO: 24 - protein, 125 aa, synthetic construct
  VARIANT 112: note = X is W or F
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWXAYWGCGTLVTVSS

SEQ ID NO: 25 - protein, 109 aa, synthetic construct
  VARIANT 23: note = X is R or G
  VARIANT 35: note = X is P or V
  VARIANT 80: note = X is V or A
  VARIANT 91: note = X is V or I
  VARIANT 97: note = X is R or L
QTVVTQEPSLTVSPGGTVTLTCXSSTGAVTTSNYXNWVQQKPGCAPRGLIGGTNKRAPGTPARFSGSLJGGKAALTLSGXQPEDEAEYYCXLWYSNXWVFGGGTKLTVL

SEQ ID NO: 26 - protein, 229 aa, synthetic construct
  VARIANT 229: note = X is K or absent
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGX

SEQ ID NO: 27 - protein, 230 aa, synthetic construct
  VARIANT 230: note = X is K or absent
GSSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGX

SEQ ID NO: 28 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 29 - protein, 292 aa, synthetic construct
QGQSGSGYLWGCEWNCGGITTGSSGGSGGSGGLSGRSDDHGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 30 - protein, 292 aa, synthetic construct
QGQSGSGYLWGCEWNCGGITTGSSGGSGGSGGLSGRSDDHGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGCGTLVTVSSGGGGSGGGGSGGGGSQTVVTQEPSLTVSPGGTVTLTCGSSTGAVTTSNYVNWVQQKPGCAPRGLIGGTNKRAPGTPARFSGSLIGGKAALTLSGAQPEDEAEYYCVLWYSNRWVFGGGTKLTVL

SEQ ID NO: 31 - protein, 292 aa, synthetic construct
QGQSGSGYLWGCEWNCGGITTGSSGGSGGSGGLSGRSDDHGGSEVQLVESGGGLVQPGGSLKLSCAASGFTFSTYAMNWVRQASGKGCEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVCGGGTKLTVL

SEQ ID NO: 32 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGCGTLVTVSSGGGGSGGGGSGGGGSQTVVTQEPSLTVSPGGTVTLTCGSSTGAVTTSNYVNWVQQKPGCAPRGLIGGTNKRAPGTPARFSGSLIGGKAALTLSGAQPEDEAEYYCVLWYSNRWVFGGGTKLTVL

SEQ ID NO: 33 - protein, 249 aa, synthetic construct
EVQLVESGGGLVQPGGSLKLSCAASGFTFSTYAMNWVRQASGKGCEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVCGGGTKLTVL

SEQ ID NO: 34 - protein, 330 aa, synthetic construct
  VARIANT 330: note = X is K or absent
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 35 - protein, 326 aa, synthetic construct
  VARIANT 326: note = X is K or absent
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGX

SEQ ID NO: 36 - protein, 377 aa, synthetic construct
  VARIANT 377: note = X is K or absent
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGX

SEQ ID NO: 37 - protein, 327 aa, synthetic construct
  VARIANT 327: note = X is K or absent
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 38 - protein, 327 aa, synthetic construct
  VARIANT 327: note = X is K or absent
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 39 - protein, 228 aa, synthetic construct
  VARIANT 228: note = X is K or absent
SKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGX

SEQ ID NO: 40 - protein, 225 aa, synthetic construct
  VARIANT 225: note = X is K or absent
GPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGX

SEQ ID NO: 41 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGX

SEQ ID NO: 42 - protein, 227 aa, synthetic construct
  VARIANT 227: note = X is K or absent
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 43 - protein, 125 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 44 - protein, 109 aa, synthetic construct
QTVVTQEPSLTVSPGGTVTLTCRSSTGAVTTSNYANWVQQKPGQAPRGLIGGTNKRAPGTPARFSGSLIGGKAALTLSGAQPEDEAEYYCALWYSNLWVFGGGTKLTVL

SEQ ID NO: 45 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLIGGKAALTLSGAQPEDEAEYYCALWYSNLWVFGGGTKLTVL

SEQ ID NO: 46 - protein, 125 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSS

SEQ ID NO: 47 - protein, 109 aa, synthetic construct
QTVVTQEPSLTVSPGGTVTLTCRSSTGAVTTSNYANWVQQKPGQAPRGLIGGTKFLAPGTPARFSGSLIGGKAALTLSGAQPEDEAEYYCALWYSNRWVFGGGTKLTVL

SEQ ID NO: 48 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYANWVQQKPGQAPRGLIGGTKFLAPGTPARFSGSLIGGKAALTLSGAQPEDEAEYYCALWYSNRWVFGGGTKLTVL

SEQ ID NO: 49 - protein, 109 aa, synthetic construct
QTVVTQEPSLTVSPGGTVTLTCRSSTGAVTTSNYPNWVQQKPGQAPRGLIGGTNKRAPGTPARFSGSLLGGKAALTLSGVQPEDEAEYYCVLWYSNRWVFGGGTKLTVL

SEQ ID NO: 50 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYPNWVQQKPGQAPRGLIGGTNKRAPGTPARFSGSLLGGKAALTLSGVQPEDEAEYYCVLWYSNRWVFGGGTKLTVL

SEQ ID NO: 51 - protein, 125 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTVHGNFGNSYVSWWAYWGQGTLVTVSS

SEQ ID NO: 52 - protein, 110 aa, synthetic construct
SQTVVTQEPSLTVSPGGTVTLTCRSSTGAVTTSNYPNWVQQKPGQAPRGLIGGTNKRAPGTPARFSGSLLGGKAALTLSGVQPEDEAEYYCVLWYSNRWVFGGGTKLTVL

SEQ ID NO: 53 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTVHGNFGNSYVSWWAYWGQGTLVTVSSGGGGSGGGGSGGGGSQTVVTQEPSLTVSPGGTVTLTCRSSTGAVTTSNYPNWVQQKPGQAPRGLIGGTNKRAPGTPARFSGSLLGGKAALTLSGVQPEDEAEYYCVLWYSNRWVFGGGTKLTVL

SEQ ID NO: 54 - protein, 125 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYISYWAYWGQGTLVTVSS

SEQ ID NO: 55 - protein, 109 aa, synthetic construct
QTVVTQEPSLTVSPGGTVTLTCRSSTGAVTTSNYPNWVQQKPGQAPRGLIGGTNKRAPGTPARFSGSLIGGKAALTLSGAQPEDEAEYYCVLWYSNRWVFGGGTKLTVL

SEQ ID NO: 56 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYISYWAYWGQGTLVTVSSGGGGSGGGGSGGGGSQTVVTQEPSLTVSPGGTVTLTCRSSTGAVTTSNYPNWVQQKPGQAPRGLIGGTNKRAPGTPARFSGSLIGGKAALTLSGAQPEDEAEYYCVLWYSNRWVFGGGTKLTVL

SEQ ID NO: 57 - protein, 125 aa, synthetic construct
EVQLVESGGGLVQPGGSLKLSCAASGFTFNKYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 58 - protein, 109 aa, synthetic construct
QTVVTQEPSLTVSPGGTVTLTCRSSTGAVTSGNYANWVQQKPGQAPRGLIGGTNKRAPGTPARFSGSLIGGKAALTLSGVQPEDEAEYYCALWYSNRWVFGGGTKLTVL

SEQ ID NO: 59 - protein, 249 aa, synthetic construct
EVQLVESGGGLVQPGGSLKLSCAASGFTFNKYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSGGGGSGGGGSQTVVTQEPSLTVSPGGTVTLTCRSSTGAVTSGNYANWVQQKPGQAPRGLIGGTNKRAPGTPARFSGSLIGGKAALTLSGVQPEDEAEYYCALWYSNRWVFGGGTKLTVL

SEQ ID NO: 60 - protein, 109 aa, synthetic construct
QTVVTQEPSLTVSPGGTVTLTCGSSTGAVTTSNYANWVQQKPGQAPRGLIGGTNKRAPGTPARFSGSLIGGKAALTLSGAQPEDEAEYYCVLWYSNLWVFGGGTKLTVL

SEQ ID NO: 61 - protein, 249 aa, synthetic construct
EVQLVESGGGLVQPGGSLKLSCAASGFTFNKYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSGGGGSGGGGSQTVVTQEPSLTVSPGGTVTLTCGSSTGAVTTSNYANWVQQKPGQAPRGLIGGTNKRAPGTPARFSGSLIGGKAALTLSGAQPEDEAEYYCVLWYSNLWVFGGGTKLTVL

SEQ ID NO: 62 - protein, 109 aa, synthetic construct
QTVVTQEPSLTVSPGGTVTLTCRSSTGAVTSGNYPNWVQQKPGQAPRGLIGGTKFLAPGTPARFSGSLLGGKAALTLSGVQPEDEAEYYCALWYSNLWVFGGGTKLTVL

SEQ ID NO: 63 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSGGGGSGGGGSQTVVTQEPSLTVSPGGTVTLTCRSSTGAVTSGNYPNWVQQKPGQAPRGLIGGTKFLAPGTPARFSGSLLGGKAALTLSGVQPEDEAEYYCALWYSNLWVFGGGTKLTVL

SEQ ID NO: 64 - protein, 109 aa, synthetic construct
QTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 65 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTVHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 66 - protein, 125 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYISYWAYWGQGTLVTVSS

SEQ ID NO: 67 - protein, 109 aa, synthetic construct
QTVVTQEPSLTVSPGGTVTLTCRSSTGAVTTSNYANWVQQKPGQAPRGLIGGTKFLAPGTPARFSGSLIGGKAALTLSGAQPEDEAEYYCALWYSNLWVFGGGTKLTVL

SEQ ID NO: 68 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYISYWAYWGQGTLVTVSSGGGGSGGGGSGGGGSQTVVTQEPSLTVSPGGTVTLTCRSSTGAVTTSNYANWVQQKPGQAPRGLIGGTKFLAPGTPARFSGSLIGGKAALTLSGAQPEDEAEYYCALWYSNLWVFGGGTKLTVL

SEQ ID NO: 69 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYISYWAYWGQGTLVTVSSGGGGSGGGGSGGGGSQTVVTQEPSLTVSPGGTVTLTCRSSTGAVTTSNYANWVQQKPGQAPRGLIGGTKFLAPGTPARFSGSLIGGKAALTLSGAQPEDEAEYYCALWYSNLWVFGGGTKLTVL

SEQ ID NO: 70 - protein, 125 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGQGTLVTVSS

SEQ ID NO: 71 - protein, 109 aa, synthetic construct
QTVVTQEPSLTVSPGGTVTLTCGSSTGAVTSGNYPNWVQQKPGQAPRGLIGGTNKRAPGTPARFSGSLIGGKAALTLSGAQPEDEAEYYCALWYSNLWVFGGGTKLTVL

SEQ ID NO: 72 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGQGTLVTVSSGGGGSGGGGSGGGGSQTVVTQEPSLTVSPGGTVTLTCGSSTGAVTSGNYPNWVQQKPGQAPRGLIGGTNKRAPGTPARFSGSLIGGKAALTLSGAQPEDEAEYYCALWYSNLWVFGGGTKLTVL

SEQ ID NO: 73 - protein, 125 aa, synthetic construct
EVQLVESGGGLVQPGGSLKLSCAASGFTFNKYAMNWVRQASGKGLEWVGRIRSKYNNYATYYADSVKDRFTISRDDSKNTAYLQMNSLKTEDTAVYYCVRHGNFGNSYVSWWAYWGQGTLVTVSS

SEQ ID NO: 74 - protein, 109 aa, synthetic construct
QTVVTQEPSLTVSPGGTVTLTCGSSTGAVTSGNYPNWVQQKPGQAPRGLIGGTKFLAPGTPARFSGSLIGGKAALTLSGAQPEDEAEYYCVLWYSNLWVFGGGTKLTVL

SEQ ID NO: 75 - protein, 249 aa, synthetic construct
EVQLVESGGGLVQPGGSLKLSCAASGFTFNKYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLIGGKAALTLSGAQPEDEAEYYCVLWYSNLWVFGGGTKLTVL

SEQ ID NO: 76 - protein, 125 aa, synthetic construct
EVQLVESGGGLVQPGGSLKLSCAASGFTFNKYAMNWVRQASGKGLEWVGRIRSKYNNYATYYADSVKDRFTISRDDSKNTAYLQMNSLKTEDTAVYYCVRHGNFGNSYISYWAYWGQGTLVTVSS

SEQ ID NO: 77 - protein, 249 aa, synthetic construct
EVQLVESGGGLVQPGGSLKLSCAASGFTFNKYAMNWVRQASGKGLEWVGRIRSKYNNYATYYADSVKDRFTISRDDSKNTAYLQMNSLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLIGGKAALTLSGAQPEDEAEYYCVLWYSNLWVFGGGTKLTVL

SEQ ID NO: 78 - protein, 125 aa, synthetic construct
EVQLVESGGGLVQPGGSLKLSCAASGFTFNTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSS

SEQ ID NO: 79 - protein, 249 aa, synthetic construct
EVQLVESGGGLVQPGGSLKLSCAASGFTFNTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLIGGKAALTLSGAQPEDEAEYYCVLWYSNLWVFGGGTKLTVL

SEQ ID NO: 80 - protein, 109 aa, synthetic construct
QTVVTQEPSLTVSPGGTVTLTCGSSTGVVTSSNYPNWVQQKPGQAPRGLIGGTKFLAPGTPARFSGSLIGGKAALTLSGAQPEDEAEYYCVLWYSNLWVFGGGTKLTVL

SEQ ID NO: 81 - protein, 249 aa, synthetic construct
EVQLVESGGGLVQPGGSLKLSCAASGFTFNTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGSGGSGGGGSGGGGSQTVVTQEPSLTVSPGGTVTLTCGSSTGVVTSSNYPNWVQQKPGQAPRGLIGGTKFLAPGTPARFSGSLIGGKAALTLSGAQPEDEAEYYCVLWYSNLWVFGGGTKLTVL

SEQ ID NO: 82 - protein, 125 aa, synthetic construct
EVQLVESGGGLVQPGGSLKLSCAASGFTFSTYAMNWVRQAPGKGLEWVARIRSKYNNYATYYADSVKDRFTISRDDSKNTAYLQMNSLKTEDTAVYYCVRHGNFGNSYVSWFAYWGQGTLVTVSS

SEQ ID NO: 83 - protein, 249 aa, synthetic construct
EVQLVESGGGLVQPGGSLKLSCAASGFTFSTYAMNWVRQAPGKGLEWVARIRSKYNNYATYYADSVKDRFTISRDDSKNTAYLQMNSLKTEDTAVYYCVRHGNFGNSYVSWFAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLIGGKAALTLSGAQPEDEAEYYCALWYSNLWVFGGGTKLTVL

SEQ ID NO: 84 - protein, 249 aa, synthetic construct
EVQLVESGGGLVQPGGSLKLSCAASGFTFSTYAMNWVRQAPGKGLEWVARIRSKYNNYATYYADSVKDRFTISRDDSKNTAYLQMNSLKTEDTAVYYCVRHGNFGNSYVSWFAYWGQGTLVTVSSGGGGSGGGGSGGGGSQTVVTQEPSLTVSPGGTVTLTCGSSTGAVTSGNYPNWVQQKPGQAPRGLIGGTNKRAPGTPARFSGSLIGGKAALTLSGAQPEDEAEYYCALWYSNLWVFGGGTKLTVL

SEQ ID NO: 85 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 86 - protein, 109 aa, synthetic construct
QTVVTQEPSLTVSPGGTVTLTCGSSTGAVTTSNYAVWVQQKPGQAPRGLIGGTNKRAPGTPARFSGSLIGGKAALTLSGAQPEDEAEYYCVLWYSNRWVFGGGTKLTVL

SEQ ID NO: 87 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGQGTLVTVSSGGGGSGGGGSGGGGSQTVVTQEPSLTVSPGGTVTLTCGSSTGAVTTSNYAVWVQQKPGQAPRGLIGGTNKRAPGTPARFSGSLIGGKAALTLSGAQPEDEAEYYCVLWYSNRWVFGGGTKLTVL

SEQ ID NO: 88 - protein, 109 aa, synthetic construct
QTVVTQEPSLTVSPGGTVTLTCGSSTGAVTTSNYAFWVQQKPGQAPRGLIGGTNKRAPGTPARFSGSLIGGKAALTLSGAQPEDEAEYYCVLWYSNRWVFGGGTKLTVL

SEQ ID NO: 89 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGQGTLVTVSSGGGGSGGGGSGGGGSQTVVTQEPSLTVSPGGTVTLTCGSSTGAVTTSNYAFWVQQKPGQAPRGLIGGTNKRAPGTPARFSGSLIGGKAALTLSGAQPEDEAEYYCVLWYSNRWVFGGGTKLTVL

SEQ ID NO: 90 - protein, 125 aa, synthetic construct
EVQLVESGGGLVQPGGSLKLSCAASGFTFSTYAMHWVRQASGKGLEWVGRIRSKYNNYATYYADSVKDRFTISRDDSKNTAYLQMNSLKTEDTAVYYCVRHGNFGNSYVSWWAYWGQGTLVTVSS

SEQ ID NO: 91 - protein, 249 aa, synthetic construct
EVQLVESGGGLVQPGGSLKLSCAASGFTFSTYAMHWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 92 - protein, 125 aa, synthetic construct
EVQLVESGGGLVQPGGSLKLSCAASGFTFSTYAMIWVRQASGKGLEWVGRIRSKYNNYATYYADSVKDRFTISRDDSKNTAYLQMNSLKTEDTAVYYCVRHGNFGNSYVSWWAYWGQGTLVTVSS

SEQ ID NO: 93 - protein, 249 aa, synthetic construct
EVQLVESGGGLVQPGGSLKLSCAASGFTFSTYAMIWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 94 - protein, 125 aa, synthetic construct
EVQLVESGGGLVQPGGSLKLSCAASGFTFSTYAMLWVRQASGKGLEWVGRIRSKYNNYATYYADSVKDRFTISRDDSKNTAYLQMNSLKTEDTAVYYCVRHGNFGNSYVSWWAYWGQGTLVTVSS

SEQ ID NO: 95 - protein, 249 aa, synthetic construct
EVQLVESGGGLVQPGGSLKLSCAASGFTFSTYAMLWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 96 - protein, 109 aa, synthetic construct
QTVVTQEPSLTVSPGGTVTLTCGSSTGAVTTSNYAYWVQQKPGQAPRGLIGGTNKRAPGTPARFSGSLIGGKAALTLSGAQPEDEAEYYCVLWYSNRWVFGGGTKLTVL

SEQ ID NO: 97 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGQGTLVTVSSGGGGSGGGGSGGGGSQTVVTQEPSLTVSPGGTVTLTCGSSTGAVTTSNYAYWVQQKPGQAPRGLIGGTNKRAPGTPARFSGSLIGGKAALTLSGAQPEDEAEYYCVLWYSNRWVFGGGTKLTVL

SEQ ID NO: 98 - protein, 109 aa, synthetic construct
QTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNLWVFGGGTKLTVL

SEQ ID NO: 99 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNLWVFGGGTKLTVL

SEQ ID NO: 100 - protein, 109 aa, synthetic construct
QTVVTQEPSLTVSPGGTVTLTCRSSTGAVTTSNYANWVQQKPGQAPRGLIGGTNKRAPGTPARFSGSLIGGKAALTLSGAQPEDEAEYYCVLWYSNLWVFGGGTKLTVL

SEQ ID NO: 101 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLIGGKAALTLSGAQPEDEAEYYCVLWYSNLWVFGGGTKLTVL

SEQ ID NO: 102 - protein, 125 aa, synthetic construct
EVQLVESGGGLVQPGGSLKLSCAASGFTFSTYAMIWVRQASGKGLEWVGRIRSKYNNYATYYADSVKDRFTISRDDSKNTAYLQMNSLKTEDTAVYYCVRHGNFGNSYVSWFAYWGQGTLVTVSS

SEQ ID NO: 103 - protein, 109 aa, synthetic construct
QTVVTQEPSLTVSPGGTVTLTCRSSTGAVTTSNYAYWVQQKPGQAPRGLIGGTNKRAPGTPARFSGSLIGGKAALTLSGAQPEDEAEYYCVLWYSNLWVFGGGTKLTVL

SEQ ID NO: 104 - protein, 249 aa, synthetic construct
EVQLVESGGGLVQPGGSLKLSCAASGFTFSTYAMIWVRQASGKGLEWVGRIRSKYNNYATYYADSVKDRFTISRDDSKNTAYLQMNSLKTEDTAVYYCVRHGNFGNSYVSWFAYWGQGTLVTVSSGGGGSGGGGSGGGGSQTVVTQEPSLTVSPGGTVTLTCRSSTGAVTTSNYAYWVQQKPGQAPRGLIGGTNKRAPGTPARFSGSLIGGKAALTLSGAQPEDEAEYYCVLWYSNLWVFGGGTKLTVL

SEQ ID NO: 105 - protein, 109 aa, synthetic construct
QTVVTQEPSLTVSPGGTVTLTCGSSTGAVTTSNYANWVQQKPGQAPRGLIGGTNKRAPGTPARFSGSLIGGKAALTLSGAQPEDEAEYYCVLWYSNRWVFGGGTKLTVL

SEQ ID NO: 106 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYANWVQQKPGQAPRGLIGGTNKRAPGTPARFSGSLIGGKAALTLSGAQPEDEAEYYCVLWYSNRWVFGGGTKLTVL

SEQ ID NO: 107 - protein, 109 aa, synthetic construct
QTVVTQEPSLTVSPGGTVTLTCRSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 108 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 109 - protein, 109 aa, synthetic construct
QTVVTQEPSLTVSPGGTVTLTCGSSTGAVTTSNYVNWVQQKPGQAPRGLIGGTNKRAPGTPARFSGSLIGGKAALTLSGAQPEDEAEYYCALWYSNRWVFGGGTKLTVL

SEQ ID NO: 110 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCALWYSNRWVFGGGTKLTVL

SEQ ID NO: 111 - protein, 125 aa, synthetic construct
EVQLVESGGGLVQPGGSLKLSCAASGFTFSTYAMNWVRQAPGKGLEWVARIRSKYNNYATYYADSVKDRFTISRDDSKNTAYLQMNSLKTEDTAVYYCVRHGNFGNSYVSWWAYWGQGTLVTVSS

SEQ ID NO: 112 - protein, 249 aa, synthetic construct
EVQLVESGGGLVQPGGSLKLSCAASGFTFSTYAMNWVRQAPGKGLEWVARIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 113 - protein, 109 aa, synthetic construct
QTVVTQEPSLTVSPGGTVTLTCRSSTGAVTTSNYVNWVQQKPGQAPRGLIGGTNKRAPGTPARFSGSLIGGKAALTLSGAQPEDEAEYYCILWYSNRWVFGGGTKLTVL

SEQ ID NO: 114 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYVNWVQQKPGQAPRGLIGGTNKRAPGTPARFSGSLIGGKAALTLSGAQPEDEAEYYCILWYSNRWVFGGGTKLTVL

SEQ ID NO: 115 - protein, 109 aa, synthetic construct
QTVVTQEPSLTVSPGGTVTLTCRSSTGAVTTSNYVNWVQQKPGQAPRGLIGGTNKRAPGTPARFSGSLIGGKAALTLSGAQPEDEAEYYCILWYSNLWVFGGGTKLTVL

SEQ ID NO: 116 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYVNWVQQKPGQAPRGLIGGTNKRAPGTPARFSGSLIGGKAALTLSGAQPEDEAEYYCILWYSNLWVFGGGTKLTVL

SEQ ID NO: 117 - protein, 109 aa, synthetic construct
QTVVTQEPSLTVSPGGTVTLTCPSSTGAVTTSNYGNWVQQKPGQAPRGLIGGTNKRAPGTPARFSGSLIGGKAALTLSGAQPEDEAEYYCVLWYSNRWVFGGGTKLTVL

SEQ ID NO: 118 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCPSSTGAVTTSNYGNWVQQKPGQAPRGLIGGTNKRAPGTPARFSGSLIGGKAALTLSGAQPEDEAEYYCVLWYSNRWVFGGGTKLTVL

SEQ ID NO: 119 - protein, 109 aa, synthetic construct
QTVVTQEPSLTVSPGGTVTLTCPSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 120 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCPSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 121 - protein, 125 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWCQGTLVTVSS

SEQ ID NO: 122 - protein, 109 aa, synthetic construct
QTVVTQEPSLTVSPGGTVTLTCGSSTGAVTTSNYVNWVQQKPGCAPRGLIGGTNKRAPGTPARFSGSLIGGKAALTLSGAQPEDEAEYYCVLWYSNRWVFGGGTKLTVL

SEQ ID NO: 123 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWCQGTLVTVSSGGGGSGGGGSGGGGSQTVVTQEPSLTVSPGGTVTLTCGSSTGAVTTSNYVNWVQQKPGCAPRGLIGGTNKRAPGTPARFSGSLIGGKAALTLSGAQPEDEAEYYCVLWYSNRWVFGGGTKLTVL

SEQ ID NO: 124 - protein, 109 aa, synthetic construct
QTVVTQEPSLTVSPGGTVTLTCGSSTGAVTTSNYVNWVQQKPGQCPRGLIGGTNKRAPGTPARFSGSLIGGKAALTLSGAQPEDEAEYYCVLWYSNRWVFGGGTKLTVL

SEQ ID NO: 125 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWCQGTLVTVSSGGGGSGGGGSGGGGSQTVVTQEPSLTVSPGGTVTLTCGSSTGAVTTSNYVNWVQQKPGQCPRGLIGGTNKRAPGTPARFSGSLIGGKAALTLSGAQPEDEAEYYCVLWYSNRWVFGGGTKLTVL

SEQ ID NO: 126 - protein, 109 aa, synthetic construct
QTVVTQEPSLTVSPGGTVTLTCGSSTGAVTTSNYVNWVQQKPGQACRGLIGGTNKRAPGTPARFSGSLIGGKAALTLSGAQPEDEAEYYCVLWYSNRWVFGGGTKLTVL

SEQ ID NO: 127 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWCQGTLVTVSSGGGGSGGGGSGGGGSQTVVTQEPSLTVSPGGTVTLTCGSSTGAVTTSNYVNWVQQKPGQACRGLIGGTNKRAPGTPARFSGSLIGGKAALTLSGAQPEDEAEYYCVLWYSNRWVFGGGTKLTVL

SEQ ID NO: 128 - protein, 125 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGCGTLVTVSS

SEQ ID NO: 129 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGCGTLVTVSSGGGGSGGGGSGGGGSQTVVTQEPSLTVSPGGTVTLTCGSSTGAVTTSNYVNWVQQKPGQCPRGLIGGTNKRAPGTPARFSGSLIGGKAALTLSGAQPEDEAEYYCVLWYSNRWVFGGGTKLTVL

SEQ ID NO: 130 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGCGTLVTVSSGGGGSGGGGSGGGGSQTVVTQEPSLTVSPGGTVTLTCGSSTGAVTTSNYVNWVQQKPGQACRGLIGGTNKRAPGTPARFSGSLIGGKAALTLSGAQPEDEAEYYCVLWYSNRWVFGGGTKLTVL

SEQ ID NO: 131 - protein, 124 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGQCTLVTVS

SEQ ID NO: 132 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGQCTLVTVSSGGGGSGGGGSGGGGSQTVVTQEPSLTVSPGGTVTLTCGSSTGAVTTSNYVNWVQQKPGCAPRGLIGGTNKRAPGTPARFSGSLIGGKAALTLSGAQPEDEAEYYCVLWYSNRWVFGGGTKLTVL

SEQ ID NO: 133 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGQCTLVTVSSGGGGSGGGGSGGGGSQTVVTQEPSLTVSPGGTVTLTCGSSTGAVTTSNYVNWVQQKPGQCPRGLIGGTNKRAPGTPARFSGSLIGGKAALTLSGAQPEDEAEYYCVLWYSNRWVFGGGTKLTVL

SEQ ID NO: 134 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGQCTLVTVSSGGGGSGGGGSGGGGSQTVVTQEPSLTVSPGGTVTLTCGSSTGAVTTSNYVNWVQQKPGQACRGLIGGTNKRAPGTPARFSGSLIGGKAALTLSGAQPEDEAEYYCVLWYSNRWVFGGGTKLTVL

SEQ ID NO: 135 - protein, 125 aa, synthetic construct
EVQLVESGGGLVQPGGSLKLSCAASGFTFSTYAMNWVRQASGKCLEWVGRIRSKYNNYATYYADSVKDRFTISRDDSKNTAYLQMNSLKTEDTAVYYCVRHGNFGNSYVSWWAYWGQGTLVTVSS

SEQ ID NO: 136 - protein, 109 aa, synthetic construct
QTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGCGTKLTVL

SEQ ID NO: 137 - protein, 249 aa, synthetic construct
EVQLVESGGGLVQPGGSLKLSCAASGFTFSTYAMNWVRQASGKCLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGCGTKLTVL

SEQ ID NO: 138 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTVHGNFGNSYVSWWAYWGQGTLVTVSSGGGGSGGGGSGGGGSQTVVTQEPSLTVSPGGTVTLTCRSSTGAVTTSNYANWVQQKPGQAPRGLIGGTKFLAPGTPARFSGSLIGGKAALTLSGAQPEDEAEYYCALWYSNRWVFGGGTKLTVL

SEQ ID NO: 139 - protein, 125 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTVHGNFGNSYISYWAYWGQGTLVTVSS

SEQ ID NO: 140 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTVHGNFGNSYISYWAYWGQGTLVTVSSGGGGSGGGGSGGGGSQTVVTQEPSLTVSPGGTVTLTCRSSTGAVTTSNYPNWVQQKPGQAPRGLIGGTNKRAPGTPARFSGSLIGGKAALTLSGAQPEDEAEYYCVLWYSNRWVFGGGTKLTVL

SEQ ID NO: 141 - protein, 109 aa, synthetic construct
QTVVTQEPSLTVSPGGTVTLTCGSSTGAVTSGNYPNWVQQKPGQAPRGLIGGTNKRAPGTPARFSGSLIGGKAALTLSGAQPEDEAEYYCVLWYSNLWVFGGGTKLTVL

SEQ ID NO: 142 - protein, 249 aa, synthetic construct
EVQLVESGGGLVQPGGSLKLSCAASGFTFNKYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSGGGGSGGGGSQTVVTQEPSLTVSPGGTVTLTCGSSTGAVTSGNYPNWVQQKPGQAPRGLIGGTNKRAPGTPARFSGSLIGGKAALTLSGAQPEDEAEYYCVLWYSNLWVFGGGTKLTVL

SEQ ID NO: 143 - protein, 109 aa, synthetic construct
QTVVTQEPSLTVSPGGTVTLTCRSSTGAVTTSNYPNWVQQKPGQAPRGLIGGTKFLAPGTPARFSGSLIGGKAALTLSGAQPEDEAEYYCALWYSNLWVFGGGTKLTVL

SEQ ID NO: 144 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYISYWAYWGQGTLVTVSSGGGGSGGGGSGGGGSQTVVTQEPSLTVSPGGTVTLTCRSSTGAVTTSNYPNWVQQKPGQAPRGLIGGTKFLAPGTPARFSGSLIGGKAALTLSGAQPEDEAEYYCALWYSNLWVFGGGTKLTVL

SEQ ID NO: 145 - protein, 125 aa, synthetic construct
EVQLVESGGGLVQPGGSLKLSCAASGFTFSTYAMNWVRQASGKGLEWVGRIRSKYNNYATYYARSVKDRFTISRDDSKNTAYLQMNSLKTEDTAVYYCVRHGNFGNSYVSWWAYWGQGTLVTVSS

SEQ ID NO: 146 - protein, 109 aa, synthetic construct
QTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNDWVFGGGTKLTVL

SEQ ID NO: 147 - protein, 249 aa, synthetic construct
EVQLVESGGGLVQPGGSLKLSCAASGFTFSTYAMNWVRQASGKGLEWVGRIRSKYNNYATYYARSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNDWVFGGGTKLTVL

SEQ ID NO: 148 - protein, 109 aa, synthetic construct
QTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNKWVFGGGTKLTVL

SEQ ID NO: 149 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNKWVFGGGTKLTVL

SEQ ID NO: 150 - protein, 125 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGCGTLVTVSS

SEQ ID NO: 151 - protein, 109 aa, synthetic construct
QTVVTQEPSLTVSPGGTVTLTCRSSTGAVTTSNYANWVQQKPGCAPRGLIGGTNKRAPGTPARFSGSLIGGKAALTLSGAQPEDEAEYYCVLWYSNLWVFGGGTKLTVL

SEQ ID NO: 152 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGCGTLVTVSSGGGGSGGGGSGGGGSQTVVTQEPSLTVSPGGTVTLTCRSSTGAVTTSNYANWVQQKPGCAPRGLIGGTNKRAPGTPARFSGSLIGGKAALTLSGAQPEDEAEYYCVLWYSNLWVFGGGTKLTVL

SEQ ID NO: 153 - protein, 125 aa, synthetic construct
EVQLVESGGGLVQPGGSLKLSCAASGFTFSTYAMNWVRQAPGKGLEWVARIRSKYNNYATYYADSVKDRFTISRDDSKNTAYLQMNSLKTEDTAVYYCVRHGNFGNSYVSWWAYWGCGTLVTVSS

SEQ ID NO: 154 - protein, 249 aa, synthetic construct
EVQLVESGGGLVQPGGSLKLSCAASGFTFSTYAMNWVRQAPGKGLEWVARIRSKYNNYATYYADSVKDRFTISRDDSKNTAYLQMNSLKTEDTAVYYCVRHGNFGNSYVSWWAYWGCGTLVTVSSGGGGSGGGGSGGGGSQTVVTQEPSLTVSPGGTVTLTCRSSTGAVTTSNYANWVQQKPGCAPRGLIGGTNKRAPGTPARFSGSLIGGKAALTLSGAQPEDEAEYYCVLWYSNLWVFGGGTKLTVL

SEQ ID NO: 155 - protein, 125 aa, synthetic construct
EVQLVESGGGLVQPGGSLKLSCAASGFTFSTYAMNWVRQAPGKGLEWVARIRSKYNNYATYYADSVKDRFTISRDDSKNTAYLQMNSLKTEDTAVYYCVRHGNFGNSYVSWFAYWGCGTLVTVSS

SEQ ID NO: 156 - protein, 249 aa, synthetic construct
EVQLVESGGGLVQPGGSLKLSCAASGFTFSTYAMNWVRQAPGKGLEWVARIRSKYNNYATYYADSVKDRFTISRDDSKNTAYLQMNSLKTEDTAVYYCVRHGNFGNSYVSWFAYWGCGTLVTVSSGGGGSGGGGSGGGGSQTVVTQEPSLTVSPGGTVTLTCRSSTGAVTTSNYANWVQQKPGCAPRGLIGGTNKRAPGTPARFSGSLIGGKAALTLSGAQPEDEAEYYCVLWYSNLWVFGGGTKLTVL

SEQ ID NO: 157 - protein, 109 aa, synthetic construct
QTVVTQEPSLTVSPGGTVTLTCRSSTGAVTTSNYVNWVQQKPGCAPRGLIGGTNKRAPGTPARFSGSLIGGKAALTLSGAQPEDEAEYYCVLWYSNLWVFGGGTKLTVL

SEQ ID NO: 158 - protein, 249 aa, synthetic construct
EVQLVESGGGLVQPGGSLKLSCAASGFTFSTYAMNWVRQAPGKGLEWVARIRSKYNNYATYYADSVKDRFTISRDDSKNTAYLQMNSLKTEDTAVYYCVRHGNFGNSYVSWWAYWGCGTLVTVSSGGGGSGGGGSGGGGSQTVVTQEPSLTVSPGGTVTLTCRSSTGAVTTSNYVNWVQQKPGCAPRGLIGGTNKRAPGTPARFSGSLIGGKAALTLSGAQPEDEAEYYCVLWYSNLWVFGGGTKLTVL

SEQ ID NO: 159 - protein, 249 aa, synthetic construct
EVQLVESGGGLVQPGGSLKLSCAASGFTFSTYAMNWVRQAPGKGLEWVARIRSKYNNYATYYADSVKDRFTISRDDSKNTAYLQMNSLKTEDTAVYYCVRHGNFGNSYVSWFAYWGCGTLVTVSSGGGGSGGGGSGGGGSQTVVTQEPSLTVSPGGTVTLTCRSSTGAVTTSNYVNWVQQKPGCAPRGLIGGTNKRAPGTPARFSGSLIGGKAALTLSGAQPEDEAEYYCVLWYSNLWVFGGGTKLTVL

SEQ ID NO: 160 - protein, 109 aa, synthetic construct
QTVVTQEPSLTVSPGGTVTLTCRSSTGAVTTSNYVNWVQQKPGQAPRGLIGGTNKRAPGTPARFSGSLIGGKAALTLSGAQPEDEAEYYCVLWYSNLWVFGGGTKLTVL

SEQ ID NO: 161 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYVNWVQQKPGQAPRGLIGGTNKRAPGTPARFSGSLIGGKAALTLSGAQPEDEAEYYCVLWYSNLWVFGGGTKLTVL

SEQ ID NO: 162 - protein, 125 aa, synthetic construct
EVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 163 - protein, 109 aa, synthetic construct
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVL

SEQ ID NO: 164 - protein, 249 aa, synthetic construct
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 165 - protein, 109 aa, synthetic construct
QTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNEWVFGGGTKLTVL

SEQ ID NO: 166 - protein, 249 aa, synthetic construct
EVQLVESGGGLVQPGGSLKLSCAASGFTFSTYAMNWVRQASGKGLEWVGRIRSKYNNYATYYARSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNEWVFGGGTKLTVL

SEQ ID NO: 167 - protein, 10 aa, synthetic construct
AANALAHGLF

SEQ ID NO: 168 - protein, 4 aa, synthetic construct
AANL

SEQ ID NO: 169 - protein, 10 aa, synthetic construct
AANLGSGGSS

SEQ ID NO: 170 - protein, 5 aa, synthetic construct
AAPRS

SEQ ID NO: 171 - protein, 6 aa, synthetic construct
AAPRSF

SEQ ID NO: 172 - protein, 8 aa, synthetic construct
AARGPAIH

SEQ ID NO: 173 - protein, 8 aa, synthetic construct
AAYHLVSQ

SEQ ID NO: 174 - protein, 10 aa, synthetic construct
AFPDMRSVRS

SEQ ID NO: 175 - protein, 7 aa, synthetic construct
AFQALRM

SEQ ID NO: 176 - protein, 6 aa, synthetic construct
AFRHLR

SEQ ID NO: 177 - protein, 8 aa, synthetic construct
AGLGISST

SEQ ID NO: 178 - protein, 8 aa, synthetic construct
AGLGVVER

SEQ ID NO: 179 - protein, 4 aa, synthetic construct
AGPR

SEQ ID NO: 180 - protein, 4 aa, synthetic construct
AHGL

SEQ ID NO: 181 - protein, 5 aa, synthetic construct
AHGLF

SEQ ID NO: 182 - protein, 7 aa, synthetic construct
AHQALRM

SEQ ID NO: 183 - protein, 10 aa, synthetic construct
AIPRVRLFDV

SEQ ID NO: 184 - protein, 5 aa, synthetic construct
ALAHG

SEQ ID NO: 185 - protein, 6 aa, synthetic construct
ALAHGL

SEQ ID NO: 186 - protein, 7 aa, synthetic construct
ALAHGLF

SEQ ID NO: 187 - protein, 12 aa, synthetic construct
ALAHGLFAPRSF

SEQ ID NO: 188 - protein, 13 aa, synthetic construct
ALAHGLFSGRSAN

SEQ ID NO: 189 - protein, 12 aa, synthetic construct
ALAHGLPTFVHL

SEQ ID NO: 190 - protein, 8 aa, synthetic construct
ALGLLRLP

SEQ ID NO: 191 - protein, 10 aa, synthetic construct
ALPSVKMVSE

SEQ ID NO: 192 - protein, 5 aa, synthetic construct
ALRAP

SEQ ID NO: 193 - protein, 7 aa, synthetic construct
ANQALRM

SEQ ID NO: 194 - protein, 8 aa, synthetic construct
ANQALRMA

SEQ ID NO: 195 - protein, 10 aa, synthetic construct
APPLVKSMVV

SEQ ID NO: 196 - protein, 10 aa, synthetic construct
APPSFKLVNA

SEQ ID NO: 197 - protein, 4 aa, synthetic construct
APRS

SEQ ID NO: 198 - protein, 11 aa, synthetic construct
APRSALAHGLF

SEQ ID NO: 199 - protein, 5 aa, synthetic construct
APRSF

SEQ ID NO: 200 - protein, 8 aa, synthetic construct
AQFVLTEG

SEQ ID NO: 201 - protein, 8 aa, synthetic construct
AQNLLGMV

SEQ ID NO: 202 - protein, 4 aa, synthetic construct
ARGP

SEQ ID NO: 203 - protein, 5 aa, synthetic construct
ARGPS

SEQ ID NO: 204 - protein, 6 aa, synthetic construct
ARGPSF

SEQ ID NO: 205 - protein, 7 aa, synthetic construct
ARGPSFK

SEQ ID NO: 206 - protein, 8 aa, synthetic construct
ASGLLRFP

SEQ ID NO: 207 - protein, 10 aa, synthetic construct
ASPTMKTVGL

SEQ ID NO: 208 - protein, 8 aa, synthetic construct
AVGLLAPP

SEQ ID NO: 209 - protein, 18 aa, synthetic construct
AVGLLAPPGGLSGRSANI

SEQ ID NO: 210 - protein, 18 aa, synthetic construct
AVGLLAPPGGLSGRSANP

SEQ ID NO: 211 - protein, 18 aa, synthetic construct
AVGLLAPPGGLSGRSDDH

SEQ ID NO: 212 - protein, 18 aa, synthetic construct
AVGLLAPPGGLSGRSDIH

SEQ ID NO: 213 - protein, 18 aa, synthetic construct
AVGLLAPPGGLSGRSDNH

SEQ ID NO: 214 - protein, 18 aa, synthetic construct
AVGLLAPPGGLSGRSDNI

SEQ ID NO: 215 - protein, 18 aa, synthetic construct
AVGLLAPPGGLSGRSDNP

SEQ ID NO: 216 - protein, 18 aa, synthetic construct
AVGLLAPPGGLSGRSDQH

SEQ ID NO: 217 - protein, 18 aa, synthetic construct
AVGLLAPPGGLSGRSDTH

SEQ ID NO: 218 - protein, 18 aa, synthetic construct
AVGLLAPPGGLSGRSDYH

SEQ ID NO: 219 - protein, 17 aa, synthetic construct
AVGLLAPPGGLSGRSNI

SEQ ID NO: 220 - protein, 18 aa, synthetic construct
AVGLLAPPGGLSGRSNIG

SEQ ID NO: 221 - protein, 19 aa, synthetic construct
AVGLLAPPGGLSGRSNIGS

SEQ ID NO: 222 - protein, 22 aa, synthetic construct
AVGLLAPPGGTSTSGRSANPRG

SEQ ID NO: 223 - protein, 17 aa, synthetic construct
AVGLLAPPSGRSANPRG

SEQ ID NO: 224 - protein, 18 aa, synthetic construct
AVGLLAPPTSGRSANPRG

SEQ ID NO: 225 - protein, 10 aa, synthetic construct
AVPKVRVVPE

SEQ ID NO: 226 - protein, 7 aa, synthetic construct
CGPPLGR

SEQ ID NO: 227 - protein, 7 aa, synthetic construct
CSPPLGR

SEQ ID NO: 228 - protein, 10 aa, synthetic construct
DEVDGSGGSS

SEQ ID NO: 229 - protein, 8 aa, synthetic construct
DISHWRRS

SEQ ID NO: 230 - protein, 7 aa, synthetic construct
DLAHPLL

SEQ ID NO: 231 - protein, 10 aa, synthetic construct
DLPLVKSLPS

SEQ ID NO: 232 - protein, 12 aa, synthetic construct
DRLSGRSANHKK

SEQ ID NO: 233 - protein, 12 aa, synthetic construct
DRLSGRSDNHKK

SEQ ID NO: 234 - protein, 10 aa, synthetic construct
DRPEMKSLSG

SEQ ID NO: 235 - protein, 10 aa, synthetic construct
DRPKVKTMDF

SEQ ID NO: 236 - protein, 8 aa, synthetic construct
DVAQFVLT

SEQ ID NO: 237 - protein, 10 aa, synthetic construct
DVPPMKTLRP

SEQ ID NO: 238 - protein, 8 aa, synthetic construct
DWLYWMGI

SEQ ID NO: 239 - protein, 8 aa, synthetic construct
DWLYWMSI

SEQ ID NO: 240 - protein, 8 aa, synthetic construct
DWLYWPGI

SEQ ID NO: 241 - protein, 8 aa, synthetic construct
DWLYWPSI

SEQ ID NO: 242 - protein, 10 aa, synthetic construct
EAPKVKALPK

SEQ ID NO: 243 - protein, 10 aa, synthetic construct
EHPRVKVVSE

SEQ ID NO: 244 - protein, 10 aa, synthetic construct
EKPRMKLFQG

SEQ ID NO: 245 - protein, 8 aa, synthetic construct
EPQALAMS

SEQ ID NO: 246 - protein, 10 aa, synthetic construct
EQPEVKMVKG

SEQ ID NO: 247 - protein, 10 aa, synthetic construct
ERPGVKSLVL

SEQ ID NO: 248 - protein, 8 aa, synthetic construct
ESLPVVAV

SEQ ID NO: 249 - protein, 10 aa, synthetic construct
ESPVMKSMAL

SEQ ID NO: 250 - protein, 5 aa, synthetic construct
ESRRW

SEQ ID NO: 251 - protein, 6 aa, synthetic construct
ESRRWM

SEQ ID NO: 252 - protein, 7 aa, synthetic construct
ESRRWMP

SEQ ID NO: 253 - protein, 10 aa, synthetic construct
ETPSVKTMGR

SEQ ID NO: 254 - protein, 12 aa, synthetic construct
ETPSVKTMGRSS

SEQ ID NO: 255 - protein, 10 aa, synthetic construct
FPRPLGITGL

SEQ ID NO: 256 - protein, 8 aa, synthetic construct
FRLLDWQW

SEQ ID NO: 257 - protein, 8 aa, synthetic construct
GCGPPLGR

SEQ ID NO: 258 - protein, 8 aa, synthetic construct
GCSPPLGR

SEQ ID NO: 259 - protein, 10 aa, synthetic construct
GFPHMKTFQH

SEQ ID NO: 260 - protein, 12 aa, synthetic construct
GFPHMKTFQHSS

SEQ ID NO: 261 - protein, 8 aa, synthetic construct
GGGPPLGR

SEQ ID NO: 262 - protein, 7 aa, synthetic construct
GGPPLGR

SEQ ID NO: 263 - protein, 10 aa, synthetic construct
GGQPSGMWGW

SEQ ID NO: 264 - protein, 7 aa, synthetic construct
GGSIDGR

SEQ ID NO: 265 - protein, 8 aa, synthetic construct
GGSPPLGR

SEQ ID NO: 266 - protein, 8 aa, synthetic construct
GGWHTGRN

SEQ ID NO: 267 - protein, 5 aa, synthetic construct
GIAGQ

SEQ ID NO: 268 - protein, 10 aa, synthetic construct
GLGTPRGLFA

SEQ ID NO: 269 - protein, 6 aa, synthetic construct
GLPTFV

SEQ ID NO: 270 - protein, 7 aa, synthetic construct
GLPTFVH

SEQ ID NO: 271 - protein, 8 aa, synthetic construct
GLPTFVHL

SEQ ID NO: 272 - protein, 12 aa, synthetic construct
GLPTFVHLPRQV

SEQ ID NO: 273 - protein, 12 aa, synthetic construct
GLSGRSDNHGSS

SEQ ID NO: 274 - protein, 8 aa, synthetic construct
GPEGLRVG

SEQ ID NO: 275 - protein, 8 aa, synthetic construct
GPLGIAGI

SEQ ID NO: 276 - protein, 12 aa, synthetic construct
GPLNGRSDNHKA

SEQ ID NO: 277 - protein, 12 aa, synthetic construct
GPLNGRSDNHKK

SEQ ID NO: 278 - protein, 12 aa, synthetic construct
GPLNGRSDNHKR

SEQ ID NO: 279 - protein, 12 aa, synthetic construct
GPLNGRSDNHQA

SEQ ID NO: 280 - protein, 12 aa, synthetic construct
GPLNGRSDNHQK

SEQ ID NO: 281 - protein, 12 aa, synthetic construct
GPLNGRSDNHQR

SEQ ID NO: 282 - protein, 12 aa, synthetic construct
GPLNGRSDNHRA

SEQ ID NO: 283 - protein, 12 aa, synthetic construct
GPLNGRSDNHRK

SEQ ID NO: 284 - protein, 12 aa, synthetic construct
GPLNGRSDNHRR

SEQ ID NO: 285 - protein, 12 aa, synthetic construct
GPLSGRSDNHKA

SEQ ID NO: 286 - protein, 12 aa, synthetic construct
GPLSGRSDNHKK

SEQ ID NO: 287 - protein, 12 aa, synthetic construct
GPLSGRSDNHKR

SEQ ID NO: 288 - protein, 12 aa, synthetic construct
GPLSGRSDNHQA

SEQ ID NO: 289 - protein, 12 aa, synthetic construct
GPLSGRSDNHQK

SEQ ID NO: 290 - protein, 12 aa, synthetic construct
GPLSGRSDNHQR

SEQ ID NO: 291 - protein, 12 aa, synthetic construct
GPLSGRSDNHRA

SEQ ID NO: 292 - protein, 12 aa, synthetic construct
GPLSGRSDNHRK

SEQ ID NO: 293 - protein, 12 aa, synthetic construct
GPLSGRSDNHRR

SEQ ID NO: 294 - protein, 6 aa, synthetic construct
GPPLGR

SEQ ID NO: 295 - protein, 8 aa, synthetic construct
GPQGIAGQ

SEQ ID NO: 296 - protein, 8 aa, synthetic construct
GPQGLLGA

SEQ ID NO: 297 - protein, 6 aa, synthetic construct
GPRSFG

SEQ ID NO: 298 - protein, 7 aa, synthetic construct
GPRSFGL

SEQ ID NO: 299 - protein, 8 aa, synthetic construct
GPSHLVLT

SEQ ID NO: 300 - protein, 4 aa, synthetic construct
GPTN

SEQ ID NO: 301 - protein, 11 aa, synthetic construct
GPTNALAHGLF

SEQ ID NO: 302 - protein, 5 aa, synthetic construct
GRSML

SEQ ID NO: 303 - protein, 6 aa, synthetic construct
GRSMLL

SEQ ID NO: 304 - protein, 7 aa, synthetic construct
GRSMLLG

SEQ ID NO: 305 - protein, 8 aa, synthetic construct
GRSMLLGG

SEQ ID NO: 306 - protein, 8 aa, synthetic construct
GRSMLLGP

SEQ ID NO: 307 - protein, 8 aa, synthetic construct
GRSMLLGS

SEQ ID NO: 308 - protein, 7 aa, synthetic construct
GRSMLLP

SEQ ID NO: 309 - protein, 8 aa, synthetic construct
GRSMLLPG

SEQ ID NO: 310 - protein, 8 aa, synthetic construct
GRSMLLPP

SEQ ID NO: 311 - protein, 8 aa, synthetic construct
GRSMLLPS

SEQ ID NO: 312 - protein, 7 aa, synthetic construct
GRSMLLS

SEQ ID NO: 313 - protein, 8 aa, synthetic construct
GRSMLLSG

SEQ ID NO: 314 - protein, 8 aa, synthetic construct
GRSMLLSP

SEQ ID NO: 315 - protein, 8 aa, synthetic construct
GRSMLLSS

SEQ ID NO: 316 - protein, 6 aa, synthetic construct
GRSMLM

SEQ ID NO: 317 - protein, 7 aa, synthetic construct
GRSMLMG

SEQ ID NO: 318 - protein, 8 aa, synthetic construct
GRSMLMGG

SEQ ID NO: 319 - protein, 8 aa, synthetic construct
GRSMLMGP

SEQ ID NO: 320 - protein, 8 aa, synthetic construct
GRSMLMGS

SEQ ID NO: 321 - protein, 7 aa, synthetic construct
GRSMLMP

SEQ ID NO: 322 - protein, 8 aa, synthetic construct
GRSMLMPG

SEQ ID NO: 323 - protein, 8 aa, synthetic construct
GRSMLMPP

SEQ ID NO: 324 - protein, 8 aa, synthetic construct
GRSMLMPS

SEQ ID NO: 325 - protein, 7 aa, synthetic construct
GRSMLMS

SEQ ID NO: 326 - protein, 8 aa, synthetic construct
GRSMLMSG

SEQ ID NO: 327 - protein, 8 aa, synthetic construct
GRSMLMSP

SEQ ID NO: 328 - protein, 8 aa, synthetic construct
GRSMLMSS

SEQ ID NO: 329 - protein, 8 aa, synthetic construct
GSGPPLGR

SEQ ID NO: 330 - protein, 7 aa, synthetic construct
GSPPLGR

SEQ ID NO: 331 - protein, 8 aa, synthetic construct
GSSPPLGR

SEQ ID NO: 332 - protein, 12 aa, synthetic construct
GTGRGPSWVGSS

SEQ ID NO: 333 - protein, 4 aa, synthetic construct
PTNL

SEQ ID NO: 334 - protein, 10 aa, synthetic construct
PTNLGSGGSS

SEQ ID NO: 335 - protein, 8 aa, synthetic construct
PVGYTSSL

SEQ ID NO: 336 - protein, 10 aa, synthetic construct
PVPRLKLIKD

SEQ ID NO: 337 - protein, 8 aa, synthetic construct
PVQPIGPQ

SEQ ID NO: 338 - protein, 8 aa, synthetic construct
QALAMSAI

SEQ ID NO: 339 - protein, 7 aa, synthetic construct
QFQALRM

SEQ ID NO: 340 - protein, 10 aa, synthetic construct
QGPMFKSLWD

SEQ ID NO: 341 - protein, 8 aa, synthetic construct
QGRAITFI

SEQ ID NO: 342 - protein, 7 aa, synthetic construct
QHQALRM

SEQ ID NO: 343 - protein, 8 aa, synthetic construct
QNQALRIA

SEQ ID NO: 344 - protein, 7 aa, synthetic construct
QNQALRM

SEQ ID NO: 345 - protein, 8 aa, synthetic construct
QNQALRMA

SEQ ID NO: 346 - protein, 22 aa, synthetic construct
QNQALRMAGGSGGSLSGRSDNH

SEQ ID NO: 347 - protein, 22 aa, synthetic construct
QNQALRMAGGSGGSLSGRSGNH

SEQ ID NO: 348 - protein, 6 aa, synthetic construct
QSRRVP

SEQ ID NO: 349 - protein, 7 aa, synthetic construct
QSRRVPL

SEQ ID NO: 350 - protein, 7 aa, synthetic construct
QSRRVPV

SEQ ID NO: 351 - protein, 6 aa, synthetic construct
QTRRVP

SEQ ID NO: 352 - protein, 7 aa, synthetic construct
QTRRVPL

SEQ ID NO: 353 - protein, 7 aa, synthetic construct
QTRRVPV

SEQ ID NO: 354 - protein, 8 aa, synthetic construct
QYIVSRSA

SEQ ID NO: 355 - protein, 6 aa, synthetic construct
RALRAP

SEQ ID NO: 356 - protein, 10 aa, synthetic construct
REPFMKSLPW

SEQ ID NO: 357 - protein, 6 aa, synthetic construct
RFPLKV

SEQ ID NO: 358 - protein, 10 aa, synthetic construct
RFPSLKSFPL

SEQ ID NO: 359 - protein, 7 aa, synthetic construct
RFPYGVW

SEQ ID NO: 360 - protein, 8 aa, synthetic construct
RFYRNQFF

SEQ ID NO: 361 - protein, 4 aa, synthetic construct
RGPA

SEQ ID NO: 362 - protein, 8 aa, synthetic construct
RGPAFNPM

SEQ ID NO: 363 - protein, 8 aa, synthetic construct
RGPATPIM

SEQ ID NO: 364 - protein, 7 aa, synthetic construct
RGPKLYW

SEQ ID NO: 365 - protein, 6 aa, synthetic construct
RHLAKL

SEQ ID NO: 366 - protein, 8 aa, synthetic construct
RIGRSDNH

SEQ ID NO: 367 - protein, 8 aa, synthetic construct
RKMPNITV

SEQ ID NO: 368 - protein, 14 aa, synthetic construct
RKSSIIIRMRDVVL

SEQ ID NO: 369 - protein, 8 aa, synthetic construct
RKTVQHWW

SEQ ID NO: 370 - protein, 8 aa, synthetic construct
RLGRSDNN

SEQ ID NO: 371 - protein, 8 aa, synthetic construct
RMHLRSLG

SEQ ID NO: 372 - protein, 8 aa, synthetic construct
RPLARAGI

SEQ ID NO: 373 - protein, 8 aa, synthetic construct
RPLARAGL

SEQ ID NO: 374 - protein, 12 aa, synthetic construct
RPLNGRSDNHKA

SEQ ID NO: 375 - protein, 12 aa, synthetic construct
RPLNGRSDNHKK

SEQ ID NO: 376 - protein, 12 aa, synthetic construct
RPLNGRSDNHKR

SEQ ID NO: 377 - protein, 12 aa, synthetic construct
RPLNGRSDNHQA

SEQ ID NO: 378 - protein, 12 aa, synthetic construct
RPLNGRSDNHQK

SEQ ID NO: 379 - protein, 12 aa, synthetic construct
RPLNGRSDNHQR

SEQ ID NO: 380 - protein, 12 aa, synthetic construct
RPLNGRSDNHRA

SEQ ID NO: 381 - protein, 12 aa, synthetic construct
RPLNGRSDNHRK

SEQ ID NO: 382 - protein, 12 aa, synthetic construct
RPLNGRSDNHRR

SEQ ID NO: 383 - protein, 12 aa, synthetic construct
RPLSGRSDNHKA

SEQ ID NO: 384 - protein, 12 aa, synthetic construct
RPLSGRSDNHKK

SEQ ID NO: 385 - protein, 12 aa, synthetic construct
RPLSGRSDNHKR

SEQ ID NO: 386 - protein, 12 aa, synthetic construct
RPLSGRSDNHQA

SEQ ID NO: 387 - protein, 12 aa, synthetic construct
RPLSGRSDNHQK

SEQ ID NO: 388 - protein, 12 aa, synthetic construct
RPLSGRSDNHQR

SEQ ID NO: 389 - protein, 12 aa, synthetic construct
RPLSGRSDNHRA

SEQ ID NO: 390 - protein, 12 aa, synthetic construct
RPLSGRSDNHRK

SEQ ID NO: 391 - protein, 12 aa, synthetic construct
RPLSGRSDNHRR

SEQ ID NO: 392 - protein, 8 aa, synthetic construct
RPSPMWAY

SEQ ID NO: 393 - protein, 8 aa, synthetic construct
RRHDGLRA

SEQ ID NO: 394 - protein, 8 aa, synthetic construct
RRHDGLRS

SEQ ID NO: 395 - protein, 8 aa, synthetic construct
RSLVFAPI

SEQ ID NO: 396 - protein, 8 aa, synthetic construct
RSPSRLKC

SEQ ID NO: 397 - protein, 8 aa, synthetic construct
VAGRSMRP

SEQ ID NO: 398 - protein, 10 aa, synthetic construct
VAPQLKSLVP

SEQ ID NO: 399 - protein, 8 aa, synthetic construct
VAQFVLTE

SEQ ID NO: 400 - protein, 10 aa, synthetic construct
VHMPLGFLGP

SEQ ID NO: 401 - protein, 20 aa, synthetic construct
VHMPLGFLGPGGLSGRSDNH

SEQ ID NO: 402 - protein, 24 aa, synthetic construct
VHMPLGFLGPGGTSTSGRSANPRG

SEQ ID NO: 403 - protein, 10 aa, synthetic construct
VLPELRSVFS

SEQ ID NO: 404 - protein, 8 aa, synthetic construct
VLSKQMSF

SEQ ID NO: 405 - protein, 7 aa, synthetic construct
VPAGRRS

SEQ ID NO: 406 - protein, 8 aa, synthetic construct
VPAGRRSL

SEQ ID NO: 407 - protein, 4 aa, synthetic construct
VPRQ

SEQ ID NO: 408 - protein, 5 aa, synthetic construct
VPRQV

SEQ ID NO: 409 - protein, 5 aa, synthetic construct
VSRSA

SEQ ID NO: 410 - protein, 8 aa, synthetic construct
VVPEGRRS

SEQ ID NO: 411 - protein, 8 aa, synthetic construct
WATPRPMR

SEQ ID NO: 412 - protein, 8 aa, synthetic construct
WDHPISLL

SEQ ID NO: 413 - protein, 10 aa, synthetic construct
YDPZVKVVLA

SEQ ID NO: 414 - protein, 8 aa, synthetic construct
YGAGLGVV

SEQ ID NO: 415 - protein, 7 aa, synthetic construct
YIVSRSA

SEQ ID NO: 416 - protein, 8 aa, synthetic construct
YKKFVGSL

SEQ ID NO: 417 - protein, 10 aa, synthetic construct
YVPRVKALEM

SEQ ID NO: 418 - protein, 8 aa, synthetic construct
HMMQYARH

SEQ ID NO: 419 - protein, 8 aa, synthetic construct
HTGRSGAL

SEQ ID NO: 420 - protein, 5 aa, synthetic construct
HVPRQ

SEQ ID NO: 421 - protein, 6 aa, Synthecium elegans
HVPRQV

SEQ ID NO: 422 - protein, 11 aa, synthetic construct
HVPRQVAPRSF

SEQ ID NO: 423 - protein, 11 aa, synthetic construct
HVPRQVLSGRS

SEQ ID NO: 424 - protein, 13 aa, synthetic construct
HVPRQVLSGRSAN

SEQ ID NO: 425 - protein, 8 aa, synthetic construct
HWHLGPPT

SEQ ID NO: 426 - protein, 8 aa, synthetic construct
IANLLSMV

SEQ ID NO: 427 - protein, 4 aa, synthetic construct
IDGR

SEQ ID NO: 428 - protein, 4 aa, synthetic construct
IEGR

SEQ ID NO: 429 - protein, 8 aa, synthetic construct
ILNLLSMV

SEQ ID NO: 430 - protein, 8 aa, synthetic construct
ILPRSPAF

SEQ ID NO: 431 - protein, 8 aa, synthetic construct
IPFSWSRF

SEQ ID NO: 432 - protein, 8 aa, synthetic construct
IQNLLSMV

SEQ ID NO: 433 - protein, 5 aa, synthetic construct
ISSGL

SEQ ID NO: 434 - protein, 6 aa, synthetic construct
ISSGLL

SEQ ID NO: 435 - protein, 7 aa, synthetic construct
ISSGLLS

SEQ ID NO: 436 - protein, 13 aa, synthetic construct
ISSGLLSGRSANI

SEQ ID NO: 437 - protein, 13 aa, synthetic construct
ISSGLLSGRSANP

SEQ ID NO: 438 - protein, 15 aa, synthetic construct
ISSGLLSGRSANPRG

SEQ ID NO: 439 - protein, 13 aa, synthetic construct
ISSGLLSGRSDDH

SEQ ID NO: 440 - protein, 13 aa, synthetic construct
ISSGLLSGRSDIH

SEQ ID NO: 441 - protein, 13 aa, synthetic construct
ISSGLLSGRSDNI

SEQ ID NO: 442 - protein, 13 aa, synthetic construct
ISSGLLSGRSDNP

SEQ ID NO: 443 - protein, 13 aa, synthetic construct
ISSGLLSGRSDQH

SEQ ID NO: 444 - protein, 13 aa, synthetic construct
ISSGLLSGRSDTH

SEQ ID NO: 445 - protein, 13 aa, synthetic construct
ISSGLLSGRSDYH

SEQ ID NO: 446 - protein, 13 aa, synthetic construct
ISSGLLSGRSGNH

SEQ ID NO: 447 - protein, 12 aa, synthetic construct
ISSGLLSGRSNI

SEQ ID NO: 448 - protein, 13 aa, synthetic construct
ISSGLLSGRSNIG

SEQ ID NO: 449 - protein, 14 aa, synthetic construct
ISSGLLSGRSNIGS

SEQ ID NO: 450 - protein, 8 aa, synthetic construct
ISSGLLSS

SEQ ID NO: 451 - protein, 22 aa, synthetic construct
ISSGLLSSGGSGGSLSGRSDNH

SEQ ID NO: 452 - protein, 22 aa, synthetic construct
ISSGLLSSGGSGGSLSGRSGNH

SEQ ID NO: 453 - protein, 7 aa, synthetic construct
ISSGLSS

SEQ ID NO: 454 - protein, 6 aa, synthetic construct
IVSRSA

SEQ ID NO: 455 - protein, 12 aa, synthetic construct
KGLTGRSDRHQA

SEQ ID NO: 456 - protein, 10 aa, synthetic construct
KGPKVKVVTL

SEQ ID NO: 457 - protein, 12 aa, synthetic construct
KNLYGRSENNGN

SEQ ID NO: 458 - protein, 8 aa, synthetic construct
KRMPVQFL

SEQ ID NO: 459 - protein, 8 aa, synthetic construct
LAAPLGLL

SEQ ID NO: 460 - protein, 4 aa, synthetic construct
LAHG

SEQ ID NO: 461 - protein, 5 aa, synthetic construct
LAHGL

SEQ ID NO: 462 - protein, 6 aa, synthetic construct
LAHGLF

SEQ ID NO: 463 - protein, 9 aa, synthetic construct
LAPLGLQRR

SEQ ID NO: 464 - protein, 5 aa, synthetic construct
LARAG

SEQ ID NO: 465 - protein, 6 aa, synthetic construct
LARAGI

SEQ ID NO: 466 - protein, 6 aa, synthetic construct
LARAGL

SEQ ID NO: 467 - protein, 8 aa, synthetic construct
LKAAPRWA

SEQ ID NO: 468 - protein, 8 aa, synthetic construct
LKAAPRWF

SEQ ID NO: 469 - protein, 8 aa, synthetic construct
LKAAPVWA

SEQ ID NO: 470 - protein, 8 aa, synthetic construct
LKAAPVWF

SEQ ID NO: 471 - protein, 8 aa, synthetic construct
LKGRSYYY

SEQ ID NO: 472 - protein, 8 aa, synthetic construct
LLAPSHRA

SEQ ID NO: 473 - protein, 8 aa, synthetic construct
LLEALRAL

SEQ ID NO: 474 - protein, 8 aa, synthetic construct
LLESLRAL

SEQ ID NO: 475 - protein, 8 aa, synthetic construct
LLLPAHGG

SEQ ID NO: 476 - protein, 8 aa, synthetic construct
LLLPLLGS

SEQ ID NO: 477 - protein, 8 aa, synthetic construct
LLNALRAL

SEQ ID NO: 478 - protein, 8 aa, synthetic construct
LLNSLRAL

SEQ ID NO: 479 - protein, 8 aa, synthetic construct
LLQALRAL

SEQ ID NO: 480 - protein, 8 aa, synthetic construct
LLQSLRAL

SEQ ID NO: 481 - protein, 8 aa, synthetic construct
LLSALRAL

SEQ ID NO: 482 - protein, 8 aa, synthetic construct
LLSSLRAL

SEQ ID NO: 483 - protein, 8 aa, synthetic construct
LNGRSDNH

SEQ ID NO: 484 - protein, 7 aa, synthetic construct
LPAGLLL

SEQ ID NO: 485 - protein, 8 aa, synthetic construct
LPAGLLLR

SEQ ID NO: 486 - protein, 8 aa, synthetic construct
LPAHLVLL

SEQ ID NO: 487 - protein, 8 aa, synthetic construct
LPAHLVLV

SEQ ID NO: 488 - protein, 8 aa, synthetic construct
LPGGLSPW

SEQ ID NO: 489 - protein, 8 aa, synthetic construct
LPSHLVLL

SEQ ID NO: 490 - protein, 8 aa, synthetic construct
LPSHLVLV

SEQ ID NO: 491 - protein, 5 aa, synthetic construct
LPTFV

SEQ ID NO: 492 - protein, 6 aa, synthetic construct
LPTFVH

SEQ ID NO: 493 - protein, 7 aa, synthetic construct
LPTFVHL

SEQ ID NO: 494 - protein, 5 aa, synthetic construct
LRSGW

SEQ ID NO: 495 - protein, 4 aa, synthetic construct
LSGR

SEQ ID NO: 496 - protein, 5 aa, synthetic construct
LSGRS

SEQ ID NO: 497 - protein, 6 aa, synthetic construct
LSGRSA

SEQ ID NO: 498 - protein, 12 aa, synthetic construct
LSGRSALAHGLF

SEQ ID NO: 499 - protein, 7 aa, synthetic construct
LSGRSAN

SEQ ID NO: 500 - protein, 8 aa, synthetic construct
LSGRSANI

SEQ ID NO: 501 - protein, 8 aa, synthetic construct
LSGRSANP

SEQ ID NO: 502 - protein, 6 aa, synthetic construct
LSGRSD

SEQ ID NO: 503 - protein, 7 aa, synthetic construct
LSGRSDD

SEQ ID NO: 504 - protein, 7 aa, synthetic construct
LSGRSDI

SEQ ID NO: 505 - protein, 8 aa, synthetic construct
LSGRSDIH

SEQ ID NO: 506 - protein, 7 aa, synthetic construct
LSGRSDN

SEQ ID NO: 507 - protein, 8 aa, synthetic construct
LSGRSDNH

SEQ ID NO: 508 - protein, 18 aa, synthetic construct
LSGRSDNHGGAVGLLAPP

SEQ ID NO: 509 - protein, 22 aa, synthetic construct
LSGRSDNHGGSGGSISSGLLSS

SEQ ID NO: 510 - protein, 22 aa, synthetic construct
LSGRSDNHGGSGGSQNQALRMA

SEQ ID NO: 511 - protein, 20 aa, synthetic construct
LSGRSDNHGGVHMPLGFLGP

SEQ ID NO: 512 - protein, 8 aa, synthetic construct
LSGRSDNI

SEQ ID NO: 513 - protein, 8 aa, synthetic construct
LSGRSDNP

SEQ ID NO: 514 - protein, 7 aa, synthetic construct
LSGRSDQ

SEQ ID NO: 515 - protein, 8 aa, synthetic construct
LSGRSDQH

SEQ ID NO: 516 - protein, 7 aa, synthetic construct
LSGRSDT

SEQ ID NO: 517 - protein, 8 aa, synthetic construct
LSGRSDTH

SEQ ID NO: 518 - protein, 7 aa, synthetic construct
LSGRSDY

SEQ ID NO: 519 - protein, 8 aa, synthetic construct
LSGRSDYH

SEQ ID NO: 520 - protein, 8 aa, synthetic construct
LSGRSENH

SEQ ID NO: 521 - protein, 6 aa, synthetic construct
LSGRSG

SEQ ID NO: 522 - protein, 7 aa, synthetic construct
LSGRSGN

SEQ ID NO: 523 - protein, 8 aa, synthetic construct
LSGRSGNH

SEQ ID NO: 524 - protein, 22 aa, synthetic construct
LSGRSGNHGGSGGSISSGLLSS

SEQ ID NO: 525 - protein, 22 aa, synthetic construct
LSGRSGNHGGSGGSQNQALRMA

SEQ ID NO: 526 - protein, 8 aa, synthetic construct
LSGRSGNP

SEQ ID NO: 527 - protein, 8 aa, synthetic construct
LSGRSVTQ

SEQ ID NO: 528 - protein, 8 aa, synthetic construct
LSQARWRK

SEQ ID NO: 529 - protein, 8 aa, synthetic construct
LTFPTYIF

SEQ ID NO: 530 - protein, 8 aa, synthetic construct
LTFPTYWF

SEQ ID NO: 531 - protein, 8 aa, synthetic construct
LTGRSDRH

SEQ ID NO: 532 - protein, 7 aa, synthetic construct
LTGRSGA

SEQ ID NO: 533 - protein, 7 aa, synthetic construct
LTGRSGA

SEQ ID NO: 534 - protein, 8 aa, synthetic construct
LYAAPRWA

SEQ ID NO: 535 - protein, 8 aa, synthetic construct
LYAAPRWF

SEQ ID NO: 536 - protein, 8 aa, synthetic construct
LYAAPVWA

SEQ ID NO: 537 - protein, 8 aa, synthetic construct
LYAAPVWF

SEQ ID NO: 538 - protein, 8 aa, synthetic construct
LYGRSENN

SEQ ID NO: 539 - protein, 8 aa, synthetic construct
MDAFLESS

SEQ ID NO: 540 - protein, 8 aa, synthetic construct
MGLFSEAG

SEQ ID NO: 541 - protein, 6 aa, synthetic construct
MGPWFM

SEQ ID NO: 542 - protein, 8 aa, synthetic construct
MIAPVAYR

SEQ ID NO: 543 - protein, 6 aa, synthetic construct
MLRSGW

SEQ ID NO: 544 - protein, 7 aa, synthetic construct
MLRSGWR

SEQ ID NO: 545 - protein, 8 aa, synthetic construct
MLRSGWRG

SEQ ID NO: 546 - protein, 8 aa, synthetic construct
MLRSGWRL

SEQ ID NO: 547 - protein, 8 aa, synthetic construct
MLRSGWRS

SEQ ID NO: 548 - protein, 8 aa, synthetic construct
MTFPTYIF

SEQ ID NO: 549 - protein, 8 aa, synthetic construct
MTFPTYWF

SEQ ID NO: 550 - protein, 12 aa, synthetic construct
NHRIGRSDNHRR

SEQ ID NO: 551 - protein, 10 aa, synthetic construct
NMPSFKLVTG

SEQ ID NO: 552 - protein, 12 aa, synthetic construct
NTLSGRSENHSG

SEQ ID NO: 553 - protein, 12 aa, synthetic construct
NTLSGRSGNHGS

SEQ ID NO: 554 - protein, 10 aa, synthetic construct
NZPRVRLVLP

SEQ ID NO: 555 - protein, 8 aa, synthetic construct
PAGLWLDP

SEQ ID NO: 556 - protein, 5 aa, synthetic construct
PAGRR

SEQ ID NO: 557 - protein, 6 aa, synthetic construct
PAGRRS

SEQ ID NO: 558 - protein, 7 aa, synthetic construct
PAGRRSL

SEQ ID NO: 559 - protein, 8 aa, synthetic construct
PASLWYTQ

SEQ ID NO: 560 - protein, 8 aa, synthetic construct
PESRRWMP

SEQ ID NO: 561 - protein, 6 aa, synthetic construct
PFHLSR

SEQ ID NO: 562 - protein, 6 aa, synthetic construct
PHGFFQ

SEQ ID NO: 563 - protein, 7 aa, synthetic construct
PLARAGI

SEQ ID NO: 564 - protein, 7 aa, synthetic construct
PLARAGL

SEQ ID NO: 565 - protein, 4 aa, synthetic construct
PLGL

SEQ ID NO: 566 - protein, 6 aa, synthetic construct
PLGLAG

SEQ ID NO: 567 - protein, 6 aa, synthetic construct
PLGLWA

SEQ ID NO: 568 - protein, 7 aa, synthetic construct
PLGVRGK

SEQ ID NO: 569 - protein, 8 aa, synthetic construct
PLTGRSGG

SEQ ID NO: 570 - protein, 12 aa, synthetic construct
PLTGRSGGGGSS

SEQ ID NO: 571 - protein, 5 aa, synthetic construct
PPLGR

SEQ ID NO: 572 - protein, 10 aa, synthetic construct
PPPDMKLFPG

SEQ ID NO: 573 - protein, 10 aa, synthetic construct
PPPEVRSFSV

SEQ ID NO: 574 - protein, 10 aa, synthetic construct
PPPVLKLLEW

SEQ ID NO: 575 - protein, 12 aa, synthetic construct
PPSIARSDNLAN

SEQ ID NO: 576 - protein, 8 aa, synthetic construct
PQHRIVSF

SEQ ID NO: 577 - protein, 8 aa, synthetic construct
PRFKIIGG

SEQ ID NO: 578 - protein, 8 aa, synthetic construct
PRFRIIGG

SEQ ID NO: 579 - protein, 10 aa, synthetic construct
PRPFVKSVDQ

SEQ ID NO: 580 - protein, 4 aa, synthetic construct
PRQV

SEQ ID NO: 581 - protein, 4 aa, synthetic construct
PRSF

SEQ ID NO: 582 - protein, 10 aa, synthetic construct
PSPPVKMMPE

SEQ ID NO: 583 - protein, 10 aa, synthetic construct
PTNGGSGGSS

SEQ ID NO: 584 - protein, 10 aa, synthetic construct
RVPKVKVMLD

SEQ ID NO: 585 - protein, 8 aa, synthetic construct
SAGFSLPA

SEQ ID NO: 586 - protein, 8 aa, synthetic construct
SAPAVESE

SEQ ID NO: 587 - protein, 10 aa, synthetic construct
SAPYFRMMDM

SEQ ID NO: 588 - protein, 8 aa, synthetic construct
SARGPSRW

SEQ ID NO: 589 - protein, 8 aa, synthetic construct
SCGPPLGR

SEQ ID NO: 590 - protein, 8 aa, synthetic construct
SCSPPLGR

SEQ ID NO: 591 - protein, 8 aa, synthetic construct
SGGPLGVR

SEQ ID NO: 592 - protein, 8 aa, synthetic construct
SGGPPLGR

SEQ ID NO: 593 - protein, 7 aa, synthetic construct
SGPPLGR

SEQ ID NO: 594 - protein, 4 aa, synthetic construct
SGRS

SEQ ID NO: 595 - protein, 5 aa, synthetic construct
SGRSA

SEQ ID NO: 596 - protein, 6 aa, synthetic construct
SGRSAN

SEQ ID NO: 597 - protein, 7 aa, synthetic construct
SGRSANI

SEQ ID NO: 598 - protein, 7 aa, synthetic construct
SGRSANP

SEQ ID NO: 599 - protein, 9 aa, synthetic construct
SGRSANPRG

SEQ ID NO: 600 - protein, 5 aa, synthetic construct
SGRSD

SEQ ID NO: 601 - protein, 6 aa, synthetic construct
SGRSDD

SEQ ID NO: 602 - protein, 7 aa, synthetic construct
SGRSDDH

SEQ ID NO: 603 - protein, 6 aa, synthetic construct
SGRSDI

SEQ ID NO: 604 - protein, 7 aa, synthetic construct
SGRSDIH

SEQ ID NO: 605 - protein, 6 aa, synthetic construct
SGRSDN

SEQ ID NO: 606 - protein, 7 aa, synthetic construct
SGRSDNI

SEQ ID NO: 607 - protein, 7 aa, synthetic construct
SGRSDNP

SEQ ID NO: 608 - protein, 6 aa, synthetic construct
SGRSDQ

SEQ ID NO: 609 - protein, 7 aa, synthetic construct
SGRSDQH

SEQ ID NO: 610 - protein, 6 aa, synthetic construct
SGRSDT

SEQ ID NO: 611 - protein, 7 aa, synthetic construct
SGRSDTH

SEQ ID NO: 612 - protein, 6 aa, synthetic construct
SGRSDY

SEQ ID NO: 613 - protein, 7 aa, synthetic construct
SGRSDYH

SEQ ID NO: 614 - protein, 5 aa, synthetic construct
SGRSG

SEQ ID NO: 615 - protein, 6 aa, synthetic construct
SGRSGN

SEQ ID NO: 616 - protein, 7 aa, synthetic construct
SGRSGNH

SEQ ID NO: 617 - protein, 8 aa, synthetic construct
SGSPPLGR

SEQ ID NO: 618 - protein, 8 aa, synthetic construct
SIARSDNL

SEQ ID NO: 619 - protein, 14 aa, synthetic construct
SISSGLLSGRSDNI

SEQ ID NO: 620 - protein, 6 aa, synthetic construct
SNPFKY

SEQ ID NO: 621 - protein, 8 aa, synthetic construct
SPLPLRVP

SEQ ID NO: 622 - protein, 8 aa, synthetic construct
SPLTGRSG

SEQ ID NO: 623 - protein, 6 aa, synthetic construct
SPPLGR

SEQ ID NO: 624 - protein, 5 aa, synthetic construct
SRRVP

SEQ ID NO: 625 - protein, 6 aa, synthetic construct
SRRVPL

SEQ ID NO: 626 - protein, 6 aa, synthetic construct
SRRVPV

SEQ ID NO: 627 - protein, 8 aa, synthetic construct
SSGPPLGR

SEQ ID NO: 628 - protein, 7 aa, synthetic construct
SSPPLGR

SEQ ID NO: 629 - protein, 8 aa, synthetic construct
SSRGPAYL

SEQ ID NO: 630 - protein, 9 aa, synthetic construct
SSRHRRALD

SEQ ID NO: 631 - protein, 15 aa, synthetic construct
SSSFDKGKYKKGDDA

SEQ ID NO: 632 - protein, 15 aa, synthetic construct
SSSFDKGKYKRGDDA

SEQ ID NO: 633 - protein, 8 aa, synthetic construct
SSSPPLGR

SEQ ID NO: 634 - protein, 8 aa, synthetic construct
STFPFGMF

SEQ ID NO: 635 - protein, 6 aa, synthetic construct
STVFHM

SEQ ID NO: 636 - protein, 6 aa, synthetic construct
SVHHLI

SEQ ID NO: 637 - protein, 8 aa, synthetic construct
SVSGLLSH

SEQ ID NO: 638 - protein, 8 aa, synthetic construct
SVSGLLSS

SEQ ID NO: 639 - protein, 8 aa, synthetic construct
SVSGLRSH

SEQ ID NO: 640 - protein, 8 aa, synthetic construct
SVSGLRSS

SEQ ID NO: 641 - protein, 4 aa, synthetic construct
TARG

SEQ ID NO: 642 - protein, 5 aa, synthetic construct
TARGP

SEQ ID NO: 643 - protein, 12 aa, synthetic construct
TARGPALAHGLF

SEQ ID NO: 644 - protein, 7 aa, synthetic construct
TARGPSF

SEQ ID NO: 645 - protein, 6 aa, synthetic construct
TARGPS

SEQ ID NO: 646 - protein, 8 aa, synthetic construct
TARGPSFK

SEQ ID NO: 647 - protein, 7 aa, synthetic construct
TARGPSW

SEQ ID NO: 648 - protein, 7 aa, synthetic construct
TARGPSW

SEQ ID NO: 649 - protein, 10 aa, synthetic construct
TARGPVPRQV

SEQ ID NO: 650 - protein, 4 aa, synthetic construct
TFVH

SEQ ID NO: 651 - protein, 12 aa, synthetic construct
TGLSGRSVTQTS

SEQ ID NO: 652 - protein, 8 aa, synthetic construct
TGRGPSWV

SEQ ID NO: 653 - protein, 12 aa, synthetic construct
TLRLGRSDNNKN

SEQ ID NO: 654 - protein, 8 aa, synthetic construct
TLSGLRSP

SEQ ID NO: 655 - protein, 8 aa, synthetic construct
TSGRSANP

SEQ ID NO: 656 - protein, 8 aa, synthetic construct
TSGRSGNP

SEQ ID NO: 657 - protein, 12 aa, synthetic construct
TSLSGRSANPRG

SEQ ID NO: 658 - protein, 12 aa, synthetic construct
TSLSGRSGNPRG

SEQ ID NO: 659 - protein, 8 aa, synthetic construct
TSSGLRSP

SEQ ID NO: 660 - protein, 12 aa, synthetic construct
TSTSGRSANPRG

SEQ ID NO: 661 - protein, 22 aa, synthetic construct
TSTSGRSANPRGGGAVGLLAPP

SEQ ID NO: 662 - protein, 24 aa, synthetic construct
TSTSGRSANPRGGGVHMPLGFLGP

SEQ ID NO: 663 - protein, 12 aa, synthetic construct
TSTSGRSGNPRG

SEQ ID NO: 664 - protein, 8 aa, synthetic construct
TVSGLRSP

SEQ ID NO: 665 - protein, 8 aa, synthetic construct
LSGRSDDH

SEQ ID NO: 666 - protein, 13 aa, synthetic construct
ISSGLLSGRSDNH

SEQ ID NO: 667 - protein, 5 aa, synthetic construct
GGSGG

SEQ ID NO: 668 - protein, 5 aa, synthetic construct
GSGSG

SEQ ID NO: 669 - protein, 5 aa, synthetic construct
GSGGG

SEQ ID NO: 670 - protein, 5 aa, synthetic construct
GGGSG

SEQ ID NO: 671 - protein, 5 aa, synthetic construct
GSSSG

SEQ ID NO: 672 - protein, 11 aa, synthetic construct
GSSGGSGGSGG

SEQ ID NO: 673 - protein, 4 aa, synthetic construct
GGGS

SEQ ID NO: 674 - protein, 8 aa, synthetic construct
GGGSGGGS

SEQ ID NO: 675 - protein, 12 aa, synthetic construct
GGGSGGGSGGGS

SEQ ID NO: 676 - protein, 15 aa, synthetic construct
GGGGSGGGGSGGGGS

SEQ ID NO: 677 - protein, 25 aa, synthetic construct
GGGGSGGGGSGGGGSGGGGSGGGGS

SEQ ID NO: 678 - protein, 10 aa, synthetic construct
GGGGSGGGGS

SEQ ID NO: 679 - protein, 5 aa, synthetic construct
GGGGS

SEQ ID NO: 680 - protein, 7 aa, synthetic construct
GGGGSGS

SEQ ID NO: 681 - protein, 17 aa, synthetic construct
GGGGSGGGGSGGGGSGS

SEQ ID NO: 682 - protein, 12 aa, synthetic construct
GGSLDPKGGGGS

SEQ ID NO: 683 - protein, 20 aa, synthetic construct
PKSCDKTHTCPPCPAPELLG

SEQ ID NO: 684 - protein, 17 aa, synthetic construct
SKYGPPCPPCPAPEFLG

SEQ ID NO: 685 - protein, 12 aa, synthetic construct
GKSSGSGSESKS

SEQ ID NO: 686 - protein, 14 aa, synthetic construct
GSTSGSGKSSEGKG

SEQ ID NO: 687 - protein, 18 aa, synthetic construct
GSTSGSGKSSEGSGSTKG

SEQ ID NO: 688 - protein, 18 aa, synthetic construct
GSTSGSGKPGSGEGSTKG

SEQ ID NO: 689 - protein, 16 aa, synthetic construct
GSTSGSGKPGSSEGST

SEQ ID NO: 690 - protein, 16 aa, synthetic construct
GSTSGSGKPGSSEGST

SEQ ID NO: 691 - protein, 15 aa, synthetic construct
GYLWGCEWNCGGITT

SEQ ID NO: 692 - protein, 15 aa, synthetic construct
NAFRCWWDPPCQPMT

SEQ ID NO: 693 - protein, 15 aa, synthetic construct
ARGLCWWDPPCTHDL

SEQ ID NO: 694 - protein, 15 aa, synthetic construct
NHSLCYWDPPCEPST

SEQ ID NO: 695 - protein, 15 aa, synthetic construct
MMYCGGNEVLCGPRV

SEQ ID NO: 696 - protein, 15 aa, synthetic construct
GYRWGCEWNCGGITT

SEQ ID NO: 697 - protein, 15 aa, synthetic construct
MMYCGGNEIFCEPRG

SEQ ID NO: 698 - protein, 15 aa, synthetic construct
GYGWGCEWNCGGSSP

SEQ ID NO: 699 - protein, 15 aa, synthetic construct
MMYCGGNEIFCGPRG

(19) United States
(12) Patent Application Publication
(10) Pub. No.: US 2025/0230239 A1
(43) Pub. Date: Jul. 17, 2025

(54) CD3-BINDING PROTEINS AND METHODS OF USE THEREOF

(71) Applicant: CytomX Therapeutics, Inc., South San Francisco, CA (US)

(72) Inventors: Madan M. PAIDHUNGAT, San Francisco, CA (US); Sayantan MITRA, Mountain View, CA (US); Leila M. BOUSTANY, Redwood City, CA (US); Ellaine Anne Mariano FOX, San Carlos, CA (US); Trang T.T. VU, San Mateo, CA (US); Laurie WONG, San Mateo, CA (US); Veena VINOD, Daly City, CA (US); Nicole G. LAPUYADE, San Francisco, CA (US); Melissa TRIEU, San Mateo, CA (US); Vijit DALAL, Pleasant Hill, CA (US)

(21) Appl. No.: 18/852,947

(22) PCT Filed: Mar. 31, 2023
(86) PCT No.: PCT/US2023/017130

§ 371 Date: Sep. 30, 2024

Related U.S. Application Data
(60) Provisional application No. 63/326,668, filed on Apr. 1, 2022.

(51) Int. Cl. C07K 16/28; C07K 16/32

(52) U.S. Cl. CPC C07K 16/2809; C07K 16/32; C07K 2317/31; C07K 2317/35; C07K 2317/569; C07K 2317/92

(57) ABSTRACT

The present disclosure includes target-binding proteins having heavy chain variable domains and light chain variable domains with specific sequences that bind to CD3 epsilon and wherein the heavy chain variable domains and the light chain variable domains are disposed within one or more polypeptides. The present disclosure also includes compositions, nucleic acids, vectors, cells, and methods of making and using the proteins, compositions, and nucleic acids.

### CROSS-REFERENCE TO RELATED APPLICATION

This application is a 35 U.S.C. 371 National Phase Entry application of PCT/US2023/017130, filed Mar. 31, 2023, which claims the benefit of U.S. Provisional Application No. U.S. 63/326,668, filed Apr. 1, 2022, which is incorporated herein by reference in its entirety.

### SEQUENCE LISTING

The Sequence Listing filed with this application by EFS, which is entitled “4862-124PCT.xml,” was created on Mar. 31, 2023 and is 643,441 bytes in size, is hereby incorporated by reference in its entirety.

### TECHNICAL FIELD

The present disclosure relates to the field of biotechnology, and more specifically, to target-binding molecules, including molecules that bind CD3.

### BACKGROUND

Anti-CD3 epsilon (CD3ε) antibodies have been in use for several years and have been designed to function in a variety of roles and combat a number of diseases. Many of these anti-CD3ε antibodies are derived from only a few variants, including SP-34. In order to further develop the efficacy, safety, and manufacturability of anti-CD3ε molecules, additional anti-CD3ε binding proteins are desired. There is a need for anti-CD3ε binding proteins with different binding affinities compared to the SP-34 antibody, improved manufacturability, improved stability, and related therapeutics with tailored CD3ε binding affinities to treat specific diseases. There also is a need for anti-CD3ε binding proteins that display improved binding affinity, stability, and manufacturability in the context of molecular structures other than full-length antibodies, including in the context of both multivalent and monovalent binding proteins.

### SUMMARY

The present disclosure provides target-binding proteins with CD3ε-binding domains, and related compositions and methods.

In an aspect, the present disclosure provides a target-binding protein comprising: a heavy chain variable domain comprising a variable heavy chain complementarity determining region 1 (VH CDR1) comprising a sequence of TYAMN (SEQ ID NO: 3), a variable heavy chain complementarity determining region 2 (VH CDR2) comprising a sequence of RIRSKYNNYATYYADSVKD (SEQ ID NO: 5), and a variable heavy chain complementarity determining region 3 (VH CDR3) comprising a sequence of HGNFGNSYVSWX₁AY (SEQ ID NO: 6), wherein X₁ is W or F; and a light chain variable domain comprising a variable light chain complementarity determining region 1 (VL CDR1) comprising a sequence of X₂SSTGAVTTSNYX₃N (SEQ ID NO: 10), wherein X₂ is R or G; and X₃ is P or V, a variable light chain complementarity determining region 2 (VL CDR2) comprising a sequence of GTNKRAP (SEQ ID NO: 14), and a variable light chain complementarity determining region 3 (VL CDR3) comprising a sequence of X₄LWYSNX₅WV (SEQ ID NO: 15), wherein X₄ is V or I; and X₅ is R or L, wherein the heavy chain variable domain and the light chain variable domain are disposed within one or more polypeptides, wherein the target-binding protein specifically binds to CD3 epsilon.

In some embodiments, the VH CDR1 comprises a sequence of TYAMN (SEQ ID NO: 3); the VH CDR2 comprises a sequence of RIRSKYNNYATYYADSVKD (SEQ ID NO: 5); the VH CDR3 comprises a sequence of HGNFGNSYVSWWAY (SEQ ID NO: 7) or HGNFGNSYVSWFAY (SEQ ID NO: 8); the VL CDR1 comprises a sequence of RSSTGAVTTSNYPN (SEQ ID NO: 11), RSSTGAVTTSNYVN (SEQ ID NO: 12), or GSSTGAVTTSNYVN (SEQ ID NO: 13); the VL CDR2 comprises a sequence of GTNKRAP (SEQ ID NO: 14); and the VL CDR3 comprises a sequence of VLWYSNRWV (SEQ ID NO: 16), VLWYSNLWV (SEQ ID NO: 17), or ILWYSNRWV (SEQ ID NO: 18).

In some embodiments, the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWWAY (SEQ ID NO: 7), the VL CDR1 comprises RSSTGAVTTSNYPN (SEQ ID NO: 11), the VL CDR2 comprises GTNKRAP (SEQ ID NO: 14), and the VL CDR3 comprises VLWYSNRWV (SEQ ID NO: 16); the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWWAY (SEQ ID NO: 7), the VL CDR1 comprises GSSTGAVTTSNYVN (SEQ ID NO: 13), the VL CDR2 comprises GTNKRAP (SEQ ID NO: 14), and the VL CDR3 comprises VLWYSNRWV (SEQ ID NO: 16); the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWFAY (SEQ ID NO: 8), the VL CDR1 comprises GSSTGAVTTSNYVN (SEQ ID NO: 13), the VL CDR2 comprises GTNKRAP (SEQ ID NO: 14), and the VL CDR3 comprises VLWYSNRWV (SEQ ID NO: 16); the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWWAY (SEQ ID NO: 7), the VL CDR1 comprises RSSTGAVTTSNYVN (SEQ ID NO: 12), the VL CDR2 comprises GTNKRAP, and the VL CDR3 comprises ILWYSNRWV (SEQ ID NO: 18); the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWWAY (SEQ ID NO: 7), the VL CDR1 comprises GSSTGAVTTSNYVN (SEQ ID NO: 13), the VL CDR2 comprises GTNKRAP (SEQ ID NO: 14), and the VL CDR3 comprises VLWYSNLWV (SEQ ID NO: 17); or the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWWAY (SEQ ID NO: 7), the VL CDR1 comprises RSSTGAVTTSNYVN (SEQ ID NO: 12), the VL CDR2 comprises GTNKRAP (SEQ ID NO: 14), and the VL CDR3 comprises VLWYSNRWV (SEQ ID NO: 16).

In some embodiments, the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWWAY (SEQ ID NO: 7), and the light chain variable domain comprises the VL CDR1 comprising RSSTGAVTTSNYPN (SEQ ID NO: 11), the VL CDR2 comprising GTNKRAP (SEQ ID NO: 14), and the VL CDR3 comprising VLWYSNRWV (SEQ ID NO: 16). In some embodiments, the heavy chain variable domain comprises the foregoing VH CDRs and comprises a sequence that is at least 90%, or at least 91%, 92%, 93%, 94, 95%, 96%, 97%, 98%, or 99% identical to SEQ ID NO: 46; and the light chain variable domain comprises the foregoing VL CDRs and comprises a sequence that is at least 90%, or at least 91%, 92%, 93%, 94, 95%, 96%, 97%, 98%, or 99% identical to SEQ ID NO: 49. In some embodiments, the heavy chain variable domain comprises the amino acid sequence of SEQ ID NO: 46 and the light chain variable domain comprises the amino acid sequence of SEQ ID NO: 49.

In some embodiments, the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWWAY (SEQ ID NO: 7), and the light chain variable domain comprises the VL CDR1 comprising GSSTGAVTTSNYVN (SEQ ID NO: 13), the VL CDR2 comprising GTNKRAP (SEQ ID NO: 14), and the VL CDR3 comprising VLWYSNRWV (SEQ ID NO: 16). In some embodiments, the heavy chain variable domain comprises the foregoing VH CDRs and comprises a sequence that is at least 90%, or at least 91%, 92%, 93%, 94, 95%, 96%, 97%, 98%, or 99% identical to SEQ ID NO: 46; and the light chain variable domain comprises the foregoing VL CDRs and comprises a sequence that is at least 90%, or at least 91%, 92%, 93%, 94, 95%, 96%, 97%, 98%, or 99% identical to SEQ ID NO: 64. In some embodiments, the heavy chain variable domain comprises a sequence of SEQ ID NO: 46, and the light chain variable domain comprises a sequence of SEQ ID NO: 64.

In some embodiments, the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWFAY (SEQ ID NO: 8), and the light chain variable domain comprises the VL CDR1 comprising GSSTGAVTTSNYVN (SEQ ID NO: 13), the VL CDR2 comprising GTNKRAP (SEQ ID NO: 14), and the VL CDR3 comprising VLWYSNRWV (SEQ ID NO: 16). In some embodiments, the heavy chain variable domain comprises the foregoing VH CDRs and comprises a sequence that is at least 90%, or at least 91%, 92%, 93%, 94, 95%, 96%, 97%, 98%, or 99% identical to SEQ ID NO: 128; and the light chain variable domain comprises the foregoing VL CDRs and comprises a sequence that is at least 90%, or at least 91%, 92%, 93%, 94, 95%, 96%, 97%, 98%, or 99% identical to SEQ ID NO: 122. In some embodiments, the heavy chain variable domain comprises a sequence of SEQ ID NO: 128, and the light chain variable domain comprises a sequence of SEQ ID NO: 122.

In some embodiments, the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWWAY (SEQ ID NO: 7), and the light chain variable domain comprises the VL CDR1 comprising RSSTGAVTTSNYVN (SEQ ID NO: 12), the VL CDR2 comprising GTNKRAP (SEQ ID NO: 14), and the VL CDR3 comprising ILWYSNRWV (SEQ ID NO: 18).

In some embodiments, the heavy chain variable domain comprises the foregoing VH CDRs and comprises a sequence that is at least 90%, or at least 91%, 92%, 93%, 94, 95%, 96%, 97%, 98%, or 99% identical to SEQ ID NO: 46; and the light chain variable domain comprises the foregoing VL CDRs and comprises a sequence that is at least 90%, or at least 91%, 92%, 93%, 94, 95%, 96%, 97%, 98%, or 99% identical to SEQ ID NO: 113. In some embodiments, the heavy chain variable domain comprises a sequence of SEQ ID NO: 46, and the light chain variable domain comprises a sequence of SEQ ID NO: 113.

In some embodiments, the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWWAY (SEQ ID NO: 7), and the light chain variable domain comprises the VL CDR1 comprising GSSTGAVTTSNYVN (SEQ ID NO: 13), the VL CDR2 comprising GTNKRAP (SEQ ID NO: 14), and the VL CDR3 comprising VLWYSNLWV (SEQ ID NO: 17). In some embodiments, the heavy chain variable domain comprises the foregoing VH CDRs and comprises a sequence that is at least 90%, or at least 91%, 92%, 93%, 94, 95%, 96%, 97%, 98%, or 99% identical to SEQ ID NO: 46; and the light chain variable domain comprises the foregoing VL CDRs and comprises a sequence that is at least 90%, or at least 91%, 92%, 93%, 94, 95%, 96%, 97%, 98%, or 99% identical to SEQ ID NO: 98. In some embodiments, the heavy chain variable domain comprises a sequence of SEQ ID NO: 46, and the light chain variable domain comprises a sequence of SEQ ID NO: 98.

In some embodiments, the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWWAY (SEQ ID NO: 7), and the light chain variable domain comprises the VL CDR1 comprising RSSTGAVTTSNYVN (SEQ ID NO: 12), the VL CDR2 comprising GTNKRAP (SEQ ID NO: 14), and the VL CDR3 comprising VLWYSNRWV (SEQ ID NO: 16). In some embodiments, the heavy chain variable domain comprises the foregoing VH CDRs and comprises a sequence that is at least 90%, or at least 91%, 92%, 93%, 94, 95%, 96%, 97%, 98%, or 99% identical to SEQ ID NO: 46; and the light chain variable domain comprises the foregoing VL CDRs and comprises a sequence that is at least 90%, or at least 91%, 92%, 93%, 94, 95%, 96%, 97%, 98%, or 99% identical to SEQ ID NO: 107. In some embodiments, the heavy chain variable domain comprises a sequence of SEQ ID NO: 46, and the light chain variable domain comprises a sequence of SEQ ID NO: 107.

In another aspect, the present disclosure provides a target-binding protein comprising: a heavy chain variable domain comprising a sequence of EVQLVESGGGLVQPGGSLKLSCAASGFTFSTYAMNWVRQASGKGLEWVGRIRSKYNN YATYYADSVKDRFTISRDDSKNTAYLQMNSLKTEDTAVYYCVRHGNFGNSYVSWX₁A YWGX₆GTLVTVSS (SEQ ID NO: 20); and a light chain variable domain comprising a sequence of QTVVTQEPSLTVSPGGTVTLTCX₂SSTGAVTTSNYX₃NWVQQKPGX₇APRGLIGGTNKRA PGTPARFSGSLJGGKAALTLSGX₉QPEDEAEYYCX₄LWYSNX₅WVFGGGTKLTVL (SEQ ID NO: 21), wherein: X₁ is a) W or F; X₂ is R or G; X₃ is P or V; X₄ is V or I; X₅ is R or L, b) X₆ is Q and X₇ is Q; or X₆ is C and X₇ is C, c) J is L when X₉ is V; or J is I when X₉ is A, and d) the target-binding protein specifically binds to CD3 epsilon.

In some embodiments, X₆ is Q and X₇ is Q. In some embodiments, X₆ is C and X₇ is C.

In some embodiments, the heavy chain variable domain comprises a sequence of SEQ ID NO: 46 and the light chain variable domain comprises a sequence of SEQ ID NO: 49; the heavy chain variable domain comprises a sequence of SEQ ID NO: 46 and the light chain variable domain comprises a sequence of SEQ ID NO: 64; the heavy chain variable domain comprises a sequence of SEQ ID NO: 46 and the light chain variable domain comprises a sequence of SEQ ID NO: 113; the heavy chain variable domain comprises a sequence of SEQ ID NO: 46 and the light chain variable domain comprises a sequence of SEQ ID NO: 98; the heavy chain variable domain comprises a sequence of SEQ ID NO: 46 and the light chain variable domain comprises a sequence of SEQ ID NO: 107; or the heavy chain variable domain comprises a sequence of SEQ ID NO: 128 and the light chain variable domain comprises a sequence of SEQ ID NO: 122.

In some embodiments, the heavy chain variable domain and the light chain variable domain are disposed within the same polypeptide. In some embodiments, the heavy chain variable domain and the light chain variable domain are coupled via a linker. In some embodiments, the linker has a length of 5 to 30, 6 to 29, 7 to 28, 8 to 27, 9 to 26, 10 to 25, 11, 12, 13, 14, 15, 16, 17, 18, 19, 20, 21, 22, 23, or 24 amino acids.

In some embodiments, the target-binding protein comprises a sequence of SEQ ID NO: 50, 28, 114, 99, 108, or 32. In some embodiments, the target-binding protein comprises a single chain variable fragment (scFv). In some embodiments, the target-binding protein comprises a BiTE, a (scFv)₂, a NANOBODY®, a nanobody-HSA VHH-scAb, a VHH-Fab, a Dual scFab, a F(ab′)2, a diabody, a CROSSMAB®, a DAF (two-in-one), a DAE (four-in-one), a DUTAMAB®, a DT-IgG, a knobs-in-holes common light chain, a knobs-in-holes assembly, a charge pair, a Fab-arm exchange, a SEEDbody, a LUZ-Y, a FcAb, a kl-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, ZYBODY™, DVI-IgG, Diabody-CH3, a triple body, a miniantibody, a minibody, a TriBi minibody, scFv-CH3 KIH, Fab-scFv, a F(ab′)2-scFv2, a scFv-KrH, a Fab-scFv-Fc, a tetravalent HCAb, a scDiabody-Fc, a Diabody-Fc, a tandem scFv-Fc, a VHH-Fc, a tandem VHH-Fc, a L′HH-Fc KiH, a Fab-VHH-Fc, an Intrabody, a dock and lock, an ImmTAC® (immune-mobilizing monoclonal TCRs (T cell receptors) against cancer), an IgG-IgG conjugate, a Cov-X-Body, a scFv1-PEG-scFv2, an Adnectin, a DARPin, or a fibronectin, an IgG, an IgM, an IgA, an IgE, an IgD, or a DEP conjugate, TMEAbody™, SAFEbody®, TRITAC®, or SHIELD antibody.

In some embodiments, the target-binding protein is or comprises an IgG, IgM, IgA, IgE, or IgD antibody or fragment thereof. In some embodiments, the target-binding protein is an IgG1, IgG2, IgG3, or IgG4 antibody. In some embodiments, the target-binding protein is humanized.

In some embodiments, the target-binding protein further comprises a masking moiety that inhibits binding of the target-binding protein to CD3 in an inactive state. In some embodiments, the masking moiety is coupled to the target-binding protein via a cleavable moiety (either directly or indirectly, e.g., via one or more linkers), and the cleavable moiety is a substrate for a protease. In some embodiments, the protease is ADAMS, ADAMTS, ADAM8, ADAM9, ADAM10, ADAM12, ADAM15, ADAM17/TACE, ADAMDEC1, ADAMTS1, ADAMTS4, ADAMTS5, Aspartate proteases, BACE, Renin, Aspartic cathepsins, Cathepsin D, Cathepsin E, Caspases, Caspase 1, Caspase 2, Caspase 3, Caspase 4, Caspase 5, Caspase 6, Caspase 7, Caspase 8, Caspase 9, Caspase 10, Caspase 14, Cysteine cathepsins, Cathepsin B, Cathepsin C, Cathepsin K, Cathepsin L, Cathepsin S, Cathepsin V/L2, Cathepsin X/Z/P, Cysteine proteinases, Cruzipain, Legumain, Otubain-2, KLKs, KLK4, KLK5, KLK6, KLK7, KLK8, KLK10, KLK11, KLK13, KLK14, Metallo proteinases, Meprin, Neprilysin, PSMA, BMP-1, MMPs, MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27, Serine proteases, activated protein C, Cathepsin A, Cathepsin G, Chymase, coagulation factor proteases, FVIIa, FIXa, FXa, FXIa, FXIIa, Elastase, Granzyme B, Guanidinobenzoatase, HtrA1, Human Neutrophil Elastase, Lactoferrin, Marapsin, NS3/4A, PACE4, Plasmin, PSA, tPA, Thrombin, Tryptase, uPA, Type II Transmembrane, Serine Proteases, TTSPs, DESC1, DPP-4, FAP, Hepsin, Matriptase-2, MT-SP1/Matriptase, TMPRSS2, TMPRSS3, or TMPRSS4.

In some embodiments, the target-binding protein further comprises a second target-binding domain specifically binding to a second target.

In some embodiments, the heavy chain variable domain and/or the light chain variable domain is conjugated to a toxin, radioisotope, small molecule, diagnostic agent, therapeutic macromolecule, targeting moiety, or detectable moiety, via a conjugating moiety. In some embodiments, the conjugating moiety is cleavable by a protease. In some embodiments, the conjugating moiety is non-cleavable by a protease.

In another aspect, the present disclosure provides a composition comprising the target-binding protein herein and a carrier. In some embodiments, the composition is a pharmaceutical composition, wherein the carrier is a pharmaceutically acceptable carrier.

In another aspect, the present disclosure provides a container, vial, syringe, injector pen, or kit comprising at least one dose of the composition herein.

In another aspect, the present disclosure provides a nucleic acid comprising a sequence encoding the target-binding protein herein.

In another aspect, the present disclosure provides a vector comprising the nucleic acid herein.

In another aspect, the present disclosure provides a cell comprising the nucleic acid herein or the vector herein.

In another aspect, the present disclosure provides a method of treating a subject in need thereof comprising administering to the subject a therapeutically effective amount of the target-binding protein herein or the composition herein. In some embodiments, the subject has been identified or diagnosed as having a cancer.

In another aspect, the present disclosure provides a method of producing a target-binding protein, comprising: culturing the cell herein in a culture medium under a condition sufficient to produce the target-binding protein; and recovering the target-binding protein from the cell or the culture medium. In some embodiments, the method further comprises isolating the target-binding protein recovered from the cell or the culture medium. In some embodiments, the method further comprises formulating the target-binding protein into a pharmaceutical composition.

An understanding of the features and advantages of the present invention will be obtained by reference to the following detailed description that sets forth illustrative embodiments, in which the principles of the invention may be utilized, and the accompanying drawings.

### DETAILED DESCRIPTION

Provided herein are target-binding proteins that specifically bind to cluster of differentiation 3 (CD3) (e.g., CD3 epsilon). In one aspect, the target-binding protein may include a heavy chain variable domain and a light chain variable domain, which form a target-binding domain that specifically binds to CD3 (e.g., CD3 epsilon). In some embodiments, compared to known anti-CD3 proteins generated from a non-human species (e.g., the sp34 mouse anti-human CD3 antibody), the CD3-binding molecules herein comprise one or more mutations in their amino acid sequences that makes them more humanized, e.g., increases their similarities to a CD3-binding molecule produced naturally in humans.

In some embodiments, the target-binding proteins may be single chain proteins, such as single chain antibodies. For example, the single chain antibodies may be single chain fragment variable (scFv) antibodies. In some embodiments, the target-binding proteins may be multi-chain proteins (e.g., multi-chain antibodies) that include a protein complex formed by multiple polypeptides.

In some embodiments, the target-binding proteins may be activatable molecules, such as activatable CD3-binding molecules that include a masking moiety coupled to the CD3-binding domain via a cleavable moiety (either directly or indirectly, e.g., via one or more linkers). The cleavable moiety may be cleaved under certain conditions (e.g., when exposed to a protease in a tumor microenvironment) to thereby release the masking moiety from the CD3-binding domain.

In some embodiments, the target-binding proteins may be multispecific (e.g., bispecific) binding proteins that bind to one or more additional targets other than CD3. For example, the multispecific proteins may specifically bind to CD3 and a tumor associated antigen, e.g., HER2, Jagged, EGFR, and the like.

Also provided herein are related compositions, kits, nucleic acids, vectors, and recombinant cells, as well as related methods, including methods of using and methods of producing any of the target-binding proteins described herein.

### Definitions

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The term “a” and “an” refers to one or more (i.e., at least one) of the grammatical object of the article. By way of example, “a cell” encompasses one or more cells.

As used herein, the terms “about” and “approximately,” when used to modify an amount specified in a numeric value or range, indicate that the numeric value as well as reasonable deviations from the value known to the skilled person in the art. For example ±20%, ±10%, or ±5%, are within the intended meaning of the recited value where appropriate.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” or “consisting of” would find direct support due to this definition for any elements disclosed throughout this disclosure. Based on this definition, any element disclosed herein or incorporated by reference may be included in or excluded from the claimed invention.

As used herein, a plurality of compounds, elements, or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a more concrete fashion.

Furthermore, certain molecules, constructs, compositions, elements, moieties, excipients, disorders, conditions, properties, steps, or the like may be discussed in the context of one specific embodiment or aspect or in a separate paragraph or section of this disclosure. It is understood that this is merely for convenience and brevity, and any such disclosure is equally applicable to and intended to be combined with any other embodiments or aspects found anywhere in the present disclosure and claims, which all form the application and claimed invention at the filing date. For example, a list of constructs, molecules, method steps, kits, or compositions described with respect to a construct, composition, or method is intended to and does find direct support for embodiments related to constructs, compositions, formulations, and methods described in any other part of this disclosure, even if those method steps, active agents, kits, or compositions are not re-listed in the context or section of that embodiment or aspect.

### Target-Binding Proteins

A target-binding protein herein may comprise a heavy chain variable domain and a light chain variable domain, which form a target-binding domain that specifically bind to CD3 (e.g., CD3 epsilon). An exemplary CD3 epsilon molecule that can be bound by the proteins herein is wild type human CD3 epsilon (SEQ ID NO: 1).

In some embodiments, the heavy chain variable domain and the light chain variable domain are disposed within the same polypeptide. In certain embodiments, the heavy chain variable domain and the light chain variable domain are coupled together by one or more linkers. The linker may be a peptide linker described in the Linkers section below. In some examples, the target-binding domain in the single chain polypeptide may be an scFv. Alternatively, the heavy chain variable domain and the light chain variable domain are disposed within two different polypeptides.

In some embodiments, the target-binding protein may be a protein complex that comprises multiple polypeptides (e.g., two, three, four, five, six, seven, eight, nine, ten, or more polypeptides). In some examples, some or all (e.g., two, three, four, five, six, seven, eight, nine, ten, or more polypeptides) of the multiple polypeptides may be identical. In some examples, each of the multiple polypeptides in the target-binding complex is different from the other.

The target-binding domain may reside within any of a variety of different constructs, including an antibody or a fragment thereof, a VH domain, a VHH domain, a VNAR domain, and a single chain fragment variable (scFv), BiTE or a component thereof, a (scFv)₂, a NANOBODY®, a nanobody-HSA, VHH-scAb, a VHH-Fab, a Dual scFab, a F(ab′)2, a diabody, a CROSSMAB®, a DAF (two-in-one), a DAE (four-in-one), a DUTAMAB®, a DT-IgG, a knobs-in-holes common light chain, a knobs-in-holes assembly, a charge pair, a Fab-arm exchange, a SEEDbody, a LUZ-Y, a FcAb, a kl-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, ZYBODY™, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a L′HH-Fc KiH, a Fab-VHH-Fc, an Intrabody, a dock and lock, an ImmTAC® (immune-mobilizing monoclonal TCRs (T cell receptors) against cancer), an IgG-IgG conjugate, a Cov-X-Body, a scFv1-PEG-scFv2, an Adnectin, a DARPin®, a fibronectin, an IgG, an IgM, an IgA, an IgE, an IgD, a DEP conjugate, TMEAbody™, SAFEbody®, TRITAC®, or SHIELD antibody. A VHH domain is a single monomeric variable antibody domain that can be found in camelids. A VNAR domain is a single monomeric variable antibody domain that can be found in cartilaginous fish. Non-limiting aspects of VHH domains and VNAR domains are described in, e.g., Cromie et al., Curr. Top. Med. Chem. 15:2543-2557, 2016; De Genst et al. Dev. Comp. Immunol. 30:187-198, 2006; De Meyer et al, Trends Biotechnol 32:263-270, 2014; Kijanka et al., Nanomedicine 10:161-174, 2015; Kovaleva et al., Expert. Opin. Biol. Ther. 14: 1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001, Muyldermans et al., Trends Biocheni. Sci. 26:230-235, 2001; Muyldermans, Ann. Rev. Biochem. 82:775-797, 2013; Rahbarizadeh et al., Immunol, invest. 40:299-338, 2011; Van Audenhove et al., EBioMedicine 8:40-48, 2016; Van Bockstaele et al., Curr. Opin. Investig. Drugs 10:1212-1224, 2009; Vincke et al. Methods Mol, Biol, 911:15-26, 2012; and Wesolowski et al. Med. Microbiol. Immunol. 198:157-174, 2009, each of which is incorporated by reference herein in its entirety.

The term “antibody” is used herein in its broadest sense and includes certain types of immunoglobulin molecules that include one or more target-binding domains that specifically bind to an antigen or epitope. Examples of antibodies include intact antibodies (e.g., intact immunoglobulins), antibody fragments, bispecific, and multi-specific antibodies. One example of a target-binding domain is formed by a V_{H}-V_{L} dimer. Additional examples of an antibody are described herein. Additional examples of an antibody are known in the art.

A “light chain” includes one variable domain (VL) and one constant domain (CL). There are two different light chains termed kappa or lambda. A “heavy chain” consists of one variable domain (VH) and three constant region domains (CH1, CH2, CH3). There are five main heavy-chain classes or isotypes, some of which have several subtypes, and these determine the functional activity of an antibody molecule. The five major classes of immunoglobulin are immunoglobulin M (IgM), immunoglobulin D (IgD), immunoglobulin G (IgG), immunoglobulin A (IgA), and immunoglobulin E (IgE). IgG is by far the most abundant immunoglobulin and has several subclasses (IgG1, IgG2, IgG3, and IgG4 in humans).

A “fragment antigen binding” (Fab) includes a complete light chain paired with the VH domain and the CH1 domain of a heavy chain. A F(ab′)₂ fragment is formed when an antibody is cleaved by pepsin (or otherwise truncated) below the hinge region, in which case the two fragment target-binding domains (Fabs) of the antibody molecule remain linked. A F(ab′)₂ fragment contains two complete light chains paired with the two VH and CH1 domains of the heavy chains joined together by the hinge region. A “fragment crystallizable” (Fc) fragment (also referred to herein as Fc domain) corresponds to the paired CH2 and CH3 domains and is the part of the antibody molecule that interacts with effector molecules and cells. The functional differences between heavy-chain isotypes lie mainly in the Fc fragment. A “single chain fragment variable” (scFv) contains only the variable domain of a light chain (VL) linked by a stretch of peptide to a variable domain of a heavy chain (VH). The name single-chain Fv is derived from Fragment variable. A “hinge region” or “interdomain” is flexible amino acid stretch that joins or links the Fab fragment to the Fc domain. A “synthetic hinge region” is an amino acid sequence that joins or links a Fab fragment to an Fc domain.

An “Fv” fragment includes a non-covalently-linked dimer of one heavy chain variable domain and one light chain variable domain. A “dual variable domain immunoglobulin G” or “DVD-IgG” refers to multivalent and multispecific target-binding proteins as described, e.g., in DiGiammarino et al., Methods Mol. Biol. 899:145-156, 2012, Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety. Examples of DARTs are described in, e.g., Garber, Nature Reviews Drug Discovery 13:799-801, 2014.

In some embodiments, the target-binding protein may be a mouse, rat, rabbit, goat, camel, donkey, primate, chimeric, human, or humanized protein. In one example, the target-binding protein may be a human protein. In one example, the target-binding protein may be a humanized (e.g., fully humanized) protein.

The term “humanized” refer to an target-binding protein having an amino acid sequence that includes VH and VL region sequences from a reference protein raised in a non-human species (e.g., a mouse), but also includes modifications in those sequences relative to the reference protein intended to render them more “human-like,” i.e., more similar to human germline variable sequences. In some embodiments, a “humanized” target-binding protein is one that immunospecifically binds to an antigen of interest and that has a framework (FR) region having substantially the amino acid sequence as that of a human protein, and a complementary determining region (CDR) having substantially the amino acid sequence as that of a non-human protein contains humanized VH and VL regions.

The term “human protein” is intended to include target-binding proteins having variable and constant regions generated, assembled, or derived from human immunoglobulin sequences. In some embodiments, target-binding proteins may be considered to be “human” even though their amino acid sequences include residues or elements not encoded by human germline immunoglobulin sequences (e.g., include sequence variations, for example that may (originally) have been introduced by random or site-specific mutagenesis in vitro or by somatic mutation in vivo), e.g., in one or more CDRs.

In some embodiments, in the target-binding protein, the heavy chain variable domain may comprise three complementarity determining regions (CDRs) (VH CDR1, VH CDR2, and VH CDR3) and the light chain variable domain may comprise three CDRs (VL CDR1, VL CDR2, and VL CDR3). The VH CDR1 may comprise a sequence of SEQ ID NO: 3. The VH CDR2 may comprise a sequence of SEQ ID NO: 5. The VH CDR3 may comprise a sequence of SEQ ID NO: 6. For example, the VH CDR3 may comprise a sequence of SEQ ID NO: 7 or 8. The VL CDR1 may comprise a sequence of SEQ ID NO: 10. For example, the VL CDR1 may comprise a sequence of SEQ ID NO: 11, 12, or 13. The VL CDR2 may comprise a sequence of SEQ ID NO: 14. The VL CDR3 may comprise a sequence of SEQ ID NO: 15. For example, the VL CDR3 may comprise a sequence of SEQ ID NOs: 16, 17, or 18.

The target-binding protein may comprise a heavy chain variable domain comprising VH CDR1, VH CDR2, and VH CDR3, and a light chain variable domain comprising VL CDR1, VL CDR2, and VL CDR3. In some examples, in the target-binding protein, the VH CDR1 may comprise a sequences of TYAMN (SEQ ID NO: 3); the VH CDR2 may comprise a sequence of RIRSKYNNYATYYADSVKD (SEQ ID NO: 5); the VH CDR3 may comprise a sequence of HGNFGNSYVSWWAY (SEQ ID NO: 7) or HGNFGNSYVSWFAY (SEQ ID NO: 8); the VL CDR1 may comprise a sequence of RSSTGAVTTSNYPN (SEQ ID NO: 11), RSSTGAVTTSNYVN (SEQ ID NO: 12), or GSSTGAVTTSNYVN (SEQ ID NO: 13); the VL CDR2 may comprise a sequence of GTNKRAP (SEQ ID NO: 14); and the VL CDR3 may comprise a sequence of VLWYSNRWV (SEQ ID NO: 16), VLWYSNLWV (SEQ ID NO: 17), or ILWYSNRWV (SEQ ID NO: 18).

In one example, in the target-binding protein, the VH CDR1 may comprise TYAMN (SEQ ID NO: 3), the VH CDR2 may comprise RIRSKYNNYATYYADSVKD (SEQ ID NO: 5), the VH CDR3 may comprise HGNFGNSYVSWWAY (SEQ ID NO: 7), the VL CDR1 may comprise RSSTGAVTTSNYPN (SEQ ID NO: 11), the VL CDR2 may comprise GTNKRAP (SEQ ID NO: 14), and the VL CDR3 may comprise VLWYSNRWV (SEQ ID NO: 16). In another example, the VH CDR1 may comprise TYAMN (SEQ ID NO: 3), the VH CDR2 may comprise RIRSKYNNYATYYADSVKD (SEQ ID NO: 5), the VH CDR3 may comprise HGNFGNSYVSWWAY (SEQ ID NO: 7), the VL CDR1 may comprise GSSTGAVTTSNYVN (SEQ ID NO: 13), the VL CDR2 may comprise GTNKRAP (SEQ ID NO: 14), and the VL CDR3 may comprise VLWYSNRWV (SEQ ID NO: 16). In another example, the VH CDR1 may comprise TYAMN (SEQ ID NO: 3), the VH CDR2 may comprise RIRSKYNNYATYYADSVKD (SEQ ID NO: 5), the VH CDR3 may comprise HGNFGNSYVSWFAY (SEQ ID NO: 8), the VL CDR1 may comprise GSSTGAVTTSNYVN (SEQ ID NO: 13), VL CDR2 may comprise GTNKRAP (SEQ ID NO: 14), and the VL CDR3 may comprise VLWYSNRWV (SEQ ID NO: 16). In another example, the VH CDR1 may comprise TYAMN (SEQ ID NO: 3), the VH CDR2 may comprise RIRSKYNNYATYYADSVKD (SEQ ID NO: 5), the VH CDR3 may comprise HGNFGNSYVSWWAY (SEQ ID NO: 7), the VL CDR1 may comprise RSSTGAVTTSNYVN (SEQ ID NO: 12), the VL CDR2 may comprise GTNKRAP (SEQ ID NO: 14), and the VL CDR3 may comprise ILWYSNRWV (SEQ ID NO: 18). In another example, the VH CDR1 may comprise TYAMN (SEQ ID NO: 3), the VH CDR2 may comprise RIRSKYNNYATYYADSVKD (SEQ ID NO: 5), the VH CDR3 may comprise HGNFGNSYVSWWAY (SEQ ID NO: 7), the VL CDR1 may comprise GSSTGAVTTSNYVN (SEQ ID NO: 13), the VL CDR2 may comprise GTNKRAP (SEQ ID NO: 14), and the VL CDR3 may comprise VLWYSNLWV (SEQ ID NO: 17). In another example, the VH CDR1 may comprise TYAMN (SEQ ID NO: 3), the VH CDR2 may comprise RIRSKYNNYATYYADSVKD (SEQ ID NO: 5), the VH CDR3 may comprise HGNFGNSYVSWWAY (SEQ ID NO: 7), the VL CDR1 may comprise RSSTGAVTTSNYVN (SEQ ID NO: 12), the VL CDR2 may comprise GTNKRAP (SEQ ID NO: 14), and the VL CDR3 may comprise VLWYSNRWV (SEQ ID NO: 16).

Additional examples of VH CDR1s include the sequences of amino acids at positions 31-35 of the heavy chain variable domains in Table 9B. Additional examples of VH CDR2s include the sequences of amino acids at positions 50-68 of the heavy chain variable domains in Table 9B. Additional examples of VH CDR3s include the sequences of amino acids at positions 101-114 of the heavy chain variable domains in Table 9B. Additional examples of VL CDR1s include the sequences of amino acids at positions 23-36 of the light chain variable domains in Table 9B. Additional examples of VH CDR2s include the sequences of amino acids at positions 52-58 of the light chain variable domains in Table 9B. Additional examples of VH CDR3s include the sequences of amino acids at positions 91-99 of the light chain variable domains in Table 9B.

In some embodiments, in the target-binding protein, the heavy chain variable domain may comprise a sequence of SEQ ID NO: 20 and the light chain variable domain may comprise a sequence of SEQ ID NO: 21.

In some examples, the heavy chain variable domain and the light chain variable domain may comprise one or more cysteines that form disulfide bonds. In some examples, the disulfide bond may improve the stability while does not interfere with the target binding of the molecules. For example, the heavy chain domain may comprise a sequence of SEQ ID NO: 24 and the light chain domain may comprise a sequence of SEQ ID NO: 25. In some examples, the heavy chain and light chain variable domains do not have such cysteine mutations. For example, the heavy chain domain may comprise a sequence of SEQ ID NO: 22 and the light chain domain may comprise a sequence of SEQ ID NO: 23.

For example, the heavy chain variable domain may comprise a sequence of SEQ ID NOs: 46, or 128, and the light chain variable domain in the light chain variable domain may comprise a sequence of SEQ ID NOs: 49, 64, 113, 98, 107, or 122. In some examples, the heavy chain variable domain comprises a VH CDR1 comprising a sequence of SEQ ID NO: 3, a VH CDR2 comprising a sequence of SEQ ID NO: 5, and a VH CDR3 comprising a sequence of SEQ ID NO: 6, 7, or 8, and the heavy chain variable domain comprises a sequence that is at least 80% identical (e.g., at least 82%, at least 84%, at least 86%, at least 88%, at least 90%, at least 92%, at least 94%, at least 96%, at least 98%, at least 99%, or 100% identical) to any one of SEQ ID NOs: 46, or 128; and the light chain variable domain comprises a LH CDR1 comprising a sequence of SEQ ID NO: 10, 11, 12, or 13, a LH CDR2 comprising a sequence of SEQ ID NO: 14, and a LH CDR3 comprising a sequence of SEQ ID NO: 15, 16, 17, or 18, and the light chain variable domain comprises a sequence that is at least 80% identical (e.g., at least 82%, at least 84%, at least 86%, at least 88%, at least 90%, at least 92%, at least 94%, at least 96%, at least 98%, at least 99%, or 100% identical) to any one of SEQ ID NOs: 49, 64, 113, 98, 107, or 122. In one example, the heavy chain variable domain comprises a VH CDR1 comprising a sequence of SEQ ID NO: 3, a VH CDR2 comprising a sequence of SEQ ID NO: 5, and a VH CDR3 comprising a sequence of SEQ ID NO: 6, 7, or 8, and the heavy chain variable domain comprises a sequence that is at least 95% identical to any one of SEQ ID NOs: 46 or 128; and the light chain variable domain comprises a LH CDR1 comprising a sequence of SEQ ID NO: 10, 11, 12, or 13, a LH CDR2 comprising a sequence of SEQ ID NO: 14, and a LH CDR3 comprising a sequence of SEQ ID NO: 15, 16, 17, or 18, and the light chain variable domain comprises a sequence that is at least 95% identical to any one of SEQ ID NOs: 49, 64, 113, 98, 107, or 122. In one example, the heavy chain variable domain comprises a sequence of SEQ ID NO: 46; and the light chain variable domain comprises a sequence of SEQ ID NO: 49. In some examples, the heavy chain variable domain comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), and the heavy chain variable domain comprises a sequence that is at least 80% identical (e.g., at least 82%, at least 84%, at least 86%, at least 88%, at least 90%, at least 92%, at least 94%, at least 96%, at least 98%, at least 99%, or 100% identical) to SEQ ID NO: 46; and the light chain variable domain comprises a VL CDR1 comprising RSSTGAVTTSNYPN (SEQ ID NO: 11), a VL CDR2 comprising GTNKRAP (SEQ ID NO: 14), and a VL CDR3 comprising VLWYSNRWV (SEQ ID NO: 16), and the light chain variable domain comprises a sequence that is at least 80% identical (e.g., at least 82%, at least 84%, at least 86%, at least 88%, at least 90%, at least 92%, at least 94%, at least 96%, at least 98%, at least 99%, or 100% identical) to SEQ ID NO: 49.

In one example, the heavy chain variable domain in the heavy chain variable domain comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), and the heavy chain variable domain comprises a sequence that is at least 95% identical to SEQ ID NO: 46; and the light chain variable domain comprises a VL CDR1 comprising RSSTGAVTTSNYPN (SEQ ID NO: 11), a VL CDR2 comprising GTNKRAP (SEQ ID NO: 14), and a VL CDR3 comprising VLWYSNRWV (SEQ ID NO: 16), and the light chain variable domain comprises a sequence of that is at least 95% identical to SEQ ID NO: 49.

In another example, the heavy chain variable domain comprises a sequence of SEQ ID NO: 46; and the light chain variable domain comprises a sequence of SEQ ID NO: 64. In some examples, the heavy chain variable domain comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), and the heavy chain variable domain comprises a sequence that is at least 80% identical (e.g., at least 82%, at least 84%, at least 86%, at least 88%, at least 90%, at least 92%, at least 94%, at least 96%, at least 98%, at least 99%, or 100% identical) to SEQ ID NO: 46; and the light chain variable domain comprises a VL CDR1 comprising GSSTGAVTTSNYVN (SEQ ID NO: 13), a VL CDR2 comprising GTNKRAP (SEQ ID NO: 14), a the VL CDR3 comprising VLWYSNRWV (SEQ ID NO: 16), and the light chain variable domain comprises a sequence that is at least 80% identical (e.g., at least 82%, at least 84%, at least 86%, at least 88%, at least 90%, at least 92%, at least 94%, at least 96%, at least 98%, at least 99%, or 100% identical) to SEQ ID NO: 64. In one example, the heavy chain variable domain comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), and the heavy chain variable domain comprises a sequence that is at least 95% identical to SEQ ID NO: 46; and the light chain variable domain comprises a VL CDR1 comprising GSSTGAVTTSNYVN (SEQ ID NO: 13), a VL CDR2 comprising GTNKRAP (SEQ ID NO: 14), a VL CDR3 comprising VLWYSNRWV (SEQ ID NO: 16), and the light chain variable domain comprises a sequence of that is at least 95% identical to SEQ ID NO: 64.

In another example, the heavy chain variable domain comprises a sequence of SEQ ID NO: 46 and the light chain variable domain comprises a sequence of SEQ ID NO: 113. In some examples, the heavy chain variable domain comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), and the heavy chain variable domain comprises a sequence that is at least 80% identical (e.g., at least 82%, at least 84%, at least 86%, at least 88%, at least 90%, at least 92%, at least 94%, at least 96%, at least 98%, at least 99%, or 100% identical) to SEQ ID NO: 46; and the light chain variable domain comprises a VL CDR1 comprising RSSTGAVTTSNYVN (SEQ ID NO: 12), a VL CDR2 comprising GTNKRAP (SEQ ID NO: 14), and a VL CDR3 comprising ILWYSNRWV (SEQ ID NO: 18), and the light chain variable domain comprises a sequence that is at least 80% identical (e.g., at least 82%, at least 84%, at least 86%, at least 88%, at least 90%, at least 92%, at least 94%, at least 96%, at least 98%, at least 99%, or 100% identical) to SEQ ID NO: 113. In one example, the heavy chain variable domain comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), and the heavy chain variable domain comprises a sequence that is at least 95% identical to SEQ ID NO: 46; and the light chain variable domain comprises a VL CDR1 comprising RSSTGAVTTSNYVN (SEQ ID NO: 12), a VL CDR2 comprising GTNKRAP (SEQ ID NO: 14), and a VL CDR3 comprising ILWYSNRWV (SEQ ID NO: 18), and the light chain variable domain comprises a sequence of that is at least 95% identical to SEQ ID NO: 113.

In another example, the heavy chain variable domain comprises a sequence of SEQ ID NO: 46 and the light chain variable domain comprises a sequence of SEQ ID NO: 98. In some examples, the heavy chain variable domain comprises a VH CDR1 comprising TYAMN (SEQ ID NO: 3), a VH CDR2 comprising RIRSKYNNYATYYADSVKD (SEQ ID NO: 5), and a VH CDR3 comprising HGNFGNSYVSWWAY (SEQ ID NO: 7), and the heavy chain variable domain comprises a sequence that is at least 80% identical (e.g., at least 82%, at least 84%, at least 86%, at least 88%, at least 90%, at least 92%, at least 94%, at least 96%, at least 98%, at least 99%, or 100% identical) to SEQ ID NO: 46 and the light chain variable domain comprises a VL CDR1 comprising GSSTGAVTTSNYVN (SEQ ID NO: 13), a VL CDR2 comprising GTNKRAP (SEQ ID NO: 14), and a VL CDR3 comprising VLWYSNLWV (SEQ ID NO: 17), and the light chain variable domain comprises a sequence that is at least 80% identical (e.g., at least 82%, at least 84%, at least 86%, at least 88%, at least 90%, at least 92%, at least 94%, at least 96%, at least 98%, at least 99%, or 100% identical) to SEQ ID NO: 98. In one example, the heavy chain variable domain comprises a VH CDR1 comprising TYAMN (SEQ ID NO: 3), a VH CDR2 comprising RIRSKYNNYATYYADSVKD (SEQ ID NO: 5), and a VH CDR3 comprising HGNFGNSYVSWWAY (SEQ ID NO: 7), and the heavy chain variable domain comprises a sequence that is at least 95% identical to SEQ ID NO: 46; and the light chain variable domain comprises a VL CDR1 comprising GSSTGAVTTSNYVN (SEQ ID NO: 13), a VL CDR2 comprising GTNKRAP (SEQ ID NO: 14), and a VL CDR3 comprising VLWYSNLWV (SEQ ID NO: 17), and the light chain variable domain comprises a sequence of that is at least 95% identical to SEQ ID NO: 98.

In another example, the heavy chain variable domain comprises a sequence of SEQ ID NO: 46 and the light chain variable domain comprises a sequence of SEQ ID NO: 107. In some examples, the heavy chain variable domain comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), and the heavy chain variable domain comprises a sequence that is at least 80% identical (e.g., at least 82%, at least 84%, at least 86%, at least 88%, at least 90%, at least 92%, at least 94%, at least 96%, at least 98%, at least 99%, or 100% identical) to SEQ ID NO: 46; and the light chain variable domain comprises a VL CDR1 comprising RSSTGAVTTSNYVN (SEQ ID NO: 12), a VL CDR2 comprising GTNKRAP (SEQ ID NO: 14), and a VL CDR3 comprising VLWYSNRWV (SEQ ID NO: 16), and the light chain variable domain comprises a sequence that is at least 80% identical (e.g., at least 82%, at least 84%, at least 86%, at least 88%, at least 90%, at least 92%, at least 94%, at least 96%, at least 98%, at least 99%, or 100% identical) to SEQ ID NO: 107. In one example, the heavy chain variable domain comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), and the heavy chain variable domain comprises a sequence that is at least 95% identical to SEQ ID NO: 46; and the light chain variable domain comprises a VL CDR1 comprising RSSTGAVTTSNYVN (SEQ ID NO: 12), a VL CDR2 comprising GTNKRAP (SEQ ID NO: 14), and a VL CDR3 comprising VLWYSNRWV (SEQ ID NO: 16), and the light chain variable domain comprises a sequence of that is at least 95% identical to SEQ ID NO: 107.

In another example, the heavy chain variable domain in the heavy chain variable domain may comprise a sequence of SEQ ID NO: 128 and the light chain variable domain in the light chain variable domain may comprise a sequence of SEQ ID NO: 122. In some examples, the heavy chain variable domain comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWFAY (SEQ ID NO: 8), the heavy chain variable domain comprises a sequence that is at least 80% identical (e.g., at least 82%, at least 84%, at least 86%, at least 88%, at least 90%, at least 92%, at least 94%, at least 96%, at least 98%, at least 99%, or 100% identical) to SEQ ID NO: 128; and the light chain variable domain comprises a VL CDR1 comprising GSSTGAVTTSNYVN (SEQ ID NO: 13), a VL CDR2 comprising GTNKRAP (SEQ ID NO: 14), and a VL CDR3 comprising VLWYSNRWV (SEQ ID NO: 16), and the light chain variable domain comprises a sequence that is at least 80% identical (e.g., at least 82%, at least 84%, at least 86%, at least 88%, at least 90%, at least 92%, at least 94%, at least 96%, at least 98%, or at least 99%, or 100% identical) to SEQ ID NO: 122. In one example, the heavy chain variable domain comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWFAY (SEQ ID NO: 8), the heavy chain variable domain comprises a sequence that is at least 95% identical to SEQ ID NO: 128; and the light chain variable domain i comprises a VL CDR1 comprising GSSTGAVTTSNYVN (SEQ ID NO: 13), a VL CDR2 comprising GTNKRAP (SEQ ID NO: 14), and a VL CDR3 comprising VLWYSNRWV (SEQ ID NO: 16), and the light chain variable domain comprises a sequence of that is at least 95% identical to SEQ ID NO: 122.

Additional examples of the heavy chain variable domains and light chain variable domains include those in Table 9B.

In the cases where the target-binding proteins are single chain proteins (e.g., scFv), the single chain target-binding proteins may comprise a sequence of SEQ ID NOs: 50, 28, 114, 99, 108, or 32. In some examples, the single chain target-binding protein comprises a VH CDR1 comprising a sequence of SEQ ID NO: 3, a VH CDR2 comprising a sequence of SEQ ID NO: 5, a VH CDR3 comprising a sequence of SEQ ID NO: 6, 7, or 8, a LH CDR1 comprising a sequence of SEQ ID NO: 10, 11, 12, or 13, a LH CDR2 comprising a sequence of SEQ ID NO: 14, and a LH CDR3 comprising a sequence of SEQ ID NO: 15, 16, 17, or 18, and the single chain target-binding protein comprises a sequence that is at least 80% identical (e.g., at least 82%, at least 84%, at least 86%, at least 88%, at least 90%, at least 92%, at least 94%, at least 96%, at least 98%, at least 99%, or 100% identical) to any one of SEQ ID NOs: 50, 28, 114, 99, 188, or 32. For example, the single chain target-binding protein comprises a VH CDR1 comprising a sequence of SEQ ID NO: 3, a VH CDR2 comprising a sequence of SEQ ID NO: 5, a VH CDR3 comprising a sequence of SEQ ID NO: 6, 7, or 8, a LH CDR1 comprising a sequence of SEQ ID NO: 10, 11, 12, or 13, a LH CDR2 comprising a sequence of SEQ ID NO: 14, and a LH CDR3 comprising a sequence of SEQ ID NO: 15, 16, 17, or 18, and the single chain target-binding protein comprises a sequence that is at least 95% identical to any one of SEQ ID NOs: 50, 28, 114, 99, 188, or 32.

In some examples, the single chain target-binding proteins may comprise a sequence of SEQ ID NO: 50. In some examples, the single chain target-binding protein comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), a VL CDR1 comprising RSSTGAVTTSNYPN (SEQ ID NO: 11), a VL CDR2 comprising GTNKRAP (SEQ ID NO: 14), and a VL CDR3 comprising VLWYSNRWV (SEQ ID NO: 16), and the single chain target-binding protein comprises a sequence that is at least 80% identical (e.g., at least 82%, at least 84%, at least 86%, at least 88%, at least 90%, at least 92%, at least 94%, at least 96%, at least 98%, or at least 99% identical) to SEQ ID NO: 50. For example, the single chain target-binding protein comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), a VL CDR1 comprising RSSTGAVTTSNYPN (SEQ ID NO: 11), a VL CDR2 comprising GTNKRAP (SEQ ID NO: 14), and a VL CDR3 comprising VLWYSNRWV (SEQ ID NO: 16), and the single chain target-binding protein comprises a sequence that is at least 95% identical to SEQ ID NO: 50.

In some examples, the single chain target-binding proteins may comprise a sequence of SEQ ID NO: 28. In some examples, the single chain target-binding protein comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), a VL CDR1 comprising GSSTGAVTTSNYVN (SEQ ID NO: 13), a VL CDR2 comprising GTNKRAP (SEQ ID NO: 14), a VL CDR3 comprising VLWYSNRWV (SEQ ID NO: 16), and the single chain target-binding protein comprises a sequence that is at least 80% identical (e.g., at least 82%, at least 84%, at least 86%, at least 88%, at least 90%, at least 92%, at least 94%, at least 96%, at least 98%, or at least 99% identical) to SEQ ID NO: 28. For example, the single chain target-binding protein comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), a VL CDR1 comprising GSSTGAVTTSNYVN (SEQ ID NO: 13), a VL CDR2 comprising GTNKRAP (SEQ ID NO: 14), a VL CDR3 comprising VLWYSNRWV (SEQ ID NO: 16), and the single chain target-binding protein comprises a sequence that is at least 95% identical to SEQ ID NO: 28.

In some examples, the single chain target-binding proteins may comprise a sequence of SEQ ID NO: 114. In some examples, the single chain target-binding protein comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), a VL CDR1 comprising RSSTGAVTTSNYVN (SEQ ID NO: 12), a VL CDR2 comprising GTNKRAP (SEQ ID NO: 14), and a VL CDR3 comprising ILWYSNRWV (SEQ ID NO: 18), and the single chain target-binding protein comprises a sequence that is at least 80% identical (e.g., at least 82%, at least 84%, at least 86%, at least 88%, at least 90%, at least 92%, at least 94%, at least 96%, at least 98%, or at least 99% identical) to SEQ ID NO: 114. For example, the single chain target-binding protein comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), a VL CDR1 comprising RSSTGAVTTSNYVN (SEQ ID NO: 12), a VL CDR2 comprising GTNKRAP (SEQ ID NO: 14), and a VL CDR3 comprising ILWYSNRWV (SEQ ID NO: 18), and the single chain target-binding protein comprises a sequence that is at least 95% identical to SEQ ID NO: 114.

In some examples, the single chain target-binding proteins may comprise a sequence of SEQ ID NO: 99. In some examples, the single chain target-binding protein comprises a VH CDR1 comprising TYAMN (SEQ ID NO: 3), a VH CDR2 comprising RIRSKYNNYATYYADSVKD (SEQ ID NO: 5), and a VH CDR3 comprising HGNFGNSYVSWWAY (SEQ ID NO: 7), a VL CDR1 comprising GSSTGAVTTSNYVN (SEQ ID NO: 13), a VL CDR2 comprising GTNKRAP (SEQ ID NO: 14), and a VL CDR3 comprising VLWYSNLWV (SEQ ID NO: 17), and the single chain target-binding protein comprises a sequence that is at least 80% identical (e.g., at least 82%, at least 84%, at least 86%, at least 88%, at least 90%, at least 92%, at least 94%, at least 96%, at least 98%, or at least 99% identical) to SEQ ID NO: 99. For example, the single chain target-binding protein comprises a VH CDR1 comprising TYAMN (SEQ ID NO: 3), a VH CDR2 comprising RIRSKYNNYATYYADSVKD (SEQ ID NO: 5), and a VH CDR3 comprising HGNFGNSYVSWWAY (SEQ ID NO: 7), a VL CDR1 comprising GSSTGAVTTSNYVN (SEQ ID NO: 13), a VL CDR2 comprising GTNKRAP (SEQ ID NO: 14), and a VL CDR3 comprising VLWYSNLWV (SEQ ID NO: 17), and the single chain target-binding protein comprises a sequence that is at least 95% identical to SEQ ID NO: 99.

In some examples, the single chain target-binding a sequence of SEQ ID NO: 108. In some examples, the single chain target-binding protein comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), a VL CDR1 comprising RSSTGAVTTSNYVN (SEQ ID NO: 12), a VL CDR2 comprising GTNKRAP (SEQ ID NO: 14), and a VL CDR3 comprising VLWYSNRWV (SEQ ID NO: 16), and the single chain target-binding protein comprises a sequence that is at least 80% identical (e.g., at least 82%, at least 84%, at least 86%, at least 88%, at least 90%, at least 92%, at least 94%, at least 96%, at least 98%, or at least 99% identical) to SEQ ID NO: 108. For example, the single chain target-binding protein comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWWAY (SEQ ID NO: 7), a VL CDR1 comprising RSSTGAVTTSNYVN (SEQ ID NO: 12), a VL CDR2 comprising GTNKRAP (SEQ ID NO: 14), and a VL CDR3 comprising VLWYSNRWV (SEQ ID NO: 16), and the single chain target-binding protein comprises a sequence that is at least 95% identical to SEQ ID NO: 108.

In some examples, the single chain target-binding proteins may comprise a sequence of SEQ ID NO: 32. In some examples, the single chain target-binding protein comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWFAY (SEQ ID NO: 8), a VL CDR1 comprising GSSTGAVTTSNYVN (SEQ ID NO: 13), a VL CDR2 comprising GTNKRAP (SEQ ID NO: 14), and a VL CDR3 comprising VLWYSNRWV (SEQ ID NO: 16), and the single chain target-binding protein comprises a sequence that is at least 80% identical (e.g., at least 82%, at least 84%, at least 86%, at least 88%, at least 90%, at least 92%, at least 94%, at least 96%, at least 98%, or at least 99% identical) to SEQ ID NO: 32. For example, the single chain target-binding protein comprises a VH CDR1 comprising TYAMN (SEQ ID NO: 3), a VH CDR2 comprising RIRSKYNNYATYYADSVKD (SEQ ID NO: 5), a VH CDR3 comprising HGNFGNSYVSWFAY (SEQ ID NO: 8), a VL CDR1 comprising GSSTGAVTTSNYVN (SEQ ID NO: 13), a VL CDR2 comprising GTNKRAP (SEQ ID NO: 14), and a VL CDR3 comprising VLWYSNRWV (SEQ ID NO: 16), and the single chain target-binding protein comprises a sequence that is at least 95% identical to SEQ ID NO: 32.

Additional examples of single chain target-binding proteins include those disclosed in Table 9B.

The target-binding proteins may specifically bind to CD3 (e.g., CD3 epsilon). As used herein, the terms “specific binding” and “specifically binds” refer to the non-covalent interactions of the type that occur between an immunoglobulin molecule and an antigen for which the immunoglobulin is specific. The strength or affinity of binding interactions can be expressed in terms of the dissociation constant (K_{d} or K_{D}) of the interaction, wherein a smaller K_{d} represents a greater affinity. The strength or affinity of binding interactions can be expressed in terms of the rate of association (Kon) or the rate of dissociation (Koff). The strength or affinity of binding interaction refers to the strength of the sum total of non-covalent interactions between a target-binding domain and its binding partner (e.g., an antigen or epitope). Unless indicated otherwise, as used herein, “affinity” refers to intrinsic binding affinity, which reflects a 1:1 interaction between members of a target-binding domain and an antigen or epitope. Affinity can be measured by common methods known in the art, including those described herein. Affinity can be determined, for example, using surface plasmon resonance (SPR) technology (e.g., BIACORE®) or biolayer interferometry (e.g., FORTEBIO®). Additional methods for determining the affinity for a target-binding domain and its corresponding antigen or epitope are known in the art.

As used herein, a statement that a CD3-binding protein “specifically binds” to CD3 refers to a protein that binds to CD3 with a dissociation constant (K_{d}) of less than 100 μM (e.g., less than 5 μM or 10 μM). The target-binding proteins may specifically bind to CD3 with a K_{d} of about 0.01 nM to about 500 nM (e.g., about 0.01 nM to about 450 nM, about 0.01 nM to about 400 nM, about 0.01 nM to about 350 nM, about 0.01 nM to about 300, about 0.01 nM to about 250 nM, about 0.01 nM to about 200 nM, about 0.01 nM to about 150 nM, about 0.01 nM to about 100 nM, about 0.01 nM to about 80 nM, about 0.01 nM to about 60 nM, about 0.01 nM to about 50 nM, about 0.01 nM to about 40 nM, about 0.01 nM to about 25 nm, about 0.01 nM to about 20 nM, about 0.01 nM to about 15 nM, about 0.01 nM to about 10 nM, about 0.01 nM to about 8 nM, about 0.01 nM to about 6 nM, about 0.01 nM to about 5 nM, about 0.01 nM to about 4 nM, about 0.01 nM to about 3 nM, about 0.01 nM to about 2 nM, about 0.01 nM to about 1 nM, about 0.01 nM to about 0.8 nM, about 0.01 nM to about 0.6 nM, about 0.01 nM to about 0.4 nM, about 0.01 nM to about 0.2 nM, about 0.01 nM to about 0.1 nM, about 0.01 nM to about 0.05 nM, about 0.05 nM to about 500 nM, about 0.05 nM to about 450 nM, about 0.05 nM to about 400 nM, about 0.05 nM to about 350 nM, about 0.05 nM to about 300, about 0.05 nM to about 250 nM, about 0.05 nM to about 200 nM, about 0.05 nM to about 150 nM, about 0.05 nM to about 100 nM, about 0.05 nM to about 80 nM, about 0.05 nM to about 60 nM, about 0.05 nM to about 50 nM, about 0.05 nM to about 40 nM, about 0.05 nM to about 25 nm, about 0.05 nM to about 20 nM, about 0.05 nM to about 15 nM, about 0.05 nM to about 10 nM, about 0.05 nM to about 8 nM, about 0.05 nM to about 6 nM, about 0.05 nM to about 5 nM, about 0.05 nM to about 4 nM, about 0.05 nM to about 3 nM, about 0.05 nM to about 2 nM about 0.05 nM to about 1 nM, about 0.05 nM to about 0.8 nM, about 0.05 nM to about 0.6 nM, about 0.05 nM to about 0.4 nM, about 0.05 nM to about 0.2 nM, about 0.05 nM to about 0.1 nM, about 0.1 nM to about 500 nM, about 0.1 nM to about 450 nM, about 0.1 nM to about 400 nM, about 0.1 nM to about 350 nM, about 0.1 nM to about 300, about 0.1 nM to about 250 nM, about 0.1 nM to about 200 nM, about 0.1 nM to about 150 nM, about 0.1 nM to about 100 nM, about 0.1 nM to about 80 nM, about 0.1 nM to about 60 nM, about 0.1 nM to about 50 nM, about 0.1 nM to about 40 nM, about 0.1 nM to about 25 nm, about 0.1 nM to about 20 nM, about 0.1 nM to about 15 nM, about 0.1 nM to about 10 nM, about 0.1 nM to about 8 nM, about 0.1 nM to about 6 nM, about 0.1 nM to about 5 nM, about 0.1 nM to about 4 nM, about 0.1 nM to about 3 nM, about 0.1 nM to about 2 nM, about 0.1 nM to about 1 nM, about 0.1 nM to about 0.8 nM, about 0.1 nM to about 0.6 nM, about 0.1 nM to about 0.4 nM, about 0.1 nM to about 0.2 nM, about 0.2 nM to about 500 nM, about 0.2 nM to about 450 nM, about 0.2 nM to about 400 nM, about 0.2 nM to about 350 nM, about 0.2 nM to about 300, about 0.2 nM to about 250 nM, about 0.2 nM to about 200 nM, about 0.2 nM to about 150 nM, about 0.2 nM to about 100 nM, about 0.2 nM to about 80 nM, about 0.2 nM to about 60 nM, about 0.2 nM to about 50 nM, about 0.2 nM to about 40 nM, about 0.2 nM to about 25 nM, about 0.2 nM to about 20 nM, about 0.2 nM to about 15 nM, about 0.2 nM to about 10 nM, about 0.2 nM to about 8 nM, about 0.2 nM to about 6 nM, about 0.2 nM to about 5 nM, about 0.2 nM to about 4 nM, about 0.2 nM to about 3 nM, about 0.2 nM to about. 2 nM, about 0.2 nM to about 1 nM, about 0.2 nM to about 0.8 nM, about 0.2 nM to about 0.6 nM, about 0.2 nM to about 0.4 nM, about 0.4 nM to about 500 nM, about 0.4 nM to about 450 nM, about 0.4 nM to about 400 nM, about 0.4 nM to about 350 nM, about 0.4 nM to about 300, about 0.4 nM to about 250 nM, about 0.4 nM to about 200 nM, about 0.4 nM to about 150 nM, about 0.4 nM to about 100 nM, about 0.4 nM to about 80 nM, about 0.4 nM to about 60 nM, about 0.4 nM to about 50 nM, about 0.4 nM to about 40 nM, about 0.4 nM to about 25 nm, about 0.4 nM to about 20 nM, about 0.4 nM to about 15 nM, about 0.4 nM to about 10 nM, about 0.4 nM to about 8 nM, about 0.4 nM to about 6 nM, about 0.4 nM to about 5 nM, about 0.4 nM to about 4 nM, about 0.4 nM to about 3 nM, about 0.4 nM to about 2 nM, about 0.4 nM to about 1 nM, about 0.4 nM to about 0.8 nM, about 0.4 nM to about 0.6 nM, about 0.5 nM to about 500 nM, about 0.5 nM to about 450 nM, about 0.5 nM to about 400 nM, about 0.5 nM to about 350 nM, about 0.5 nM to about 300, about 0.5 nM to about 250 nM, about 0.5 nM to about 200 nM, about 0.5 nM to about 150 nM, about 0.5 nM to about 100 nM, about 0.5 nM to about 80 nM, about 0.5 nM to about 60 nM, about 0.5 nM to about 50 nM, about 0.5 nM to about 40 nM, about 0.5 nM to about 25 nm, about 0.5 nM to about 20 nM, about 0.5 nM to about 15 nM, about 0.5 nM to about 10 nM, about 0.5 nM to about 8 nM, about 0.5 nM to about 6 nM, about 0.5 nM to about 5 nM, about 0.5 nM to about 4 nM, about 0.5 nM to about. 3 nM, about 0.5 nM to about 2 nM, about 0.5 nM to about 1 nM, about 0.5 nM to about 0.8 nM, about 0.5 nM to about 0.6 nM, about 0.6 nM to about 500 nM, about 0.6 nM to about 450 nM, about 0.6 nM to about 400 nM, about 0.6 nM to about 350 nM, about 0.6 nM to about 300, about 0.6 nM to about 250 nM, about 0.6 nM to about 200 nM, about 0.6 nM to about 150 nM, about 0.6 nM to about 100 nM, about 0.6 nM to about 80 nM, about 0.6 nM to about 60 nM, about 0.6 nM to about 50 nM, about 0.6 nM to about 40 nM, about 0.6 nM to about 25 nM, about 0.6 nM to about 20 nM, about 0.6 nM to about 15 nM, about 0.6 nM to about 10 nM, about 0.6 nM to about 8 nM, about 0.6 nM to about 6 nM, about 0.6 nM to about 5 nM, about 0.6 nM to about 4 nM, about 0.6 nM to about 3 nM, about 0.6 nM to about 2 nM, about. 0.6 nM to about 1 nM, about 0.6 nM to about 0.8 nM, about 1 nM to about 500 nM, about 1 nM to about 450 nM, about 1 nM to about 400 nM, about 1 nM to about 350 nM, about 1 nM to about 300, about 1 nM to about 250 nM, about 1 nM to about 200 nM, about 1 nM to about 150 nM, about 1 nM to about 100 nM, about 1 nM to about 80 nM, about 1 nM to about 60 nM, about 1 nM to about 50 nM, about 1 nM to about 40 nM, about 1 nM to about 25 nm, about 1 nM to about 20 nM, about 1 nM to about 15 nM, about 1 nM to about 10 nM, about 1 nM to about 8 nM, about 1 nM to about 6 nM, about 1 nM to about 5 nM, about 1 nM to about 4 nM, about 1 nM to about 3 nM, about 1 nM to about 2 nM, about 2 nM to about 500 nM, about 2 nM to about 450 nM, about 2 nM to about 400 nM, about 2 nM to about 350 nM, about 2 nM to about 300, about 2 nM to about 250 nM, about 2 nM to about 200 nM, about 2 nM to about 150 nM, about 2 nM to about 100 nM, about 2 nM to about 80 nM, about 2 nM to about 60 nM, about 2 nM to about 50 nM, about 2 nM to about 40 nM, about 2 nM to about 25 nm, about 2 nM to about 20 nM, about 2 nM to about 15 nM, about 2 nM to about 10 nM, about 2 nM to about 8 nM, about 2 nM to about 6 nM, about 2 nM to about 5 nM, about 2 nM to about 4 nM, about 2 nM to about 3 nM, about 5 nM to about 500 nM, about 5 nM to about 450 nM, about 5 nM to about 400 nM, about 5 nM to about 350 nM, about 5 nM to about 300, about 5 nM to about 250 nM, about 5 nM to about 200 nM, about 5 nM to about 150 nM, about 5 nM to about 100 nM, about 5 nM to about 80 nM, about 5 nM to about 60 nM, about 5 nM to about 50 nM, about 5 nM to about 40 nM, about 5 nM to about 25 nm, about 5 nM to about 20 nM, about 5 nM to about 15 nM, about 5 nM to about 10 nM, about 5 nM to about 8 nM, about 5 nM to about 6 nM, about 10 nM to about 500 nM, about 10 nM to about 450 nM, about 10 nM to about 400 nM, about 10 nM to about 350 nM, about 10 nM to about 300, about 10 nM to about 250 nM, about 10 nM to about 200 nM, about 10 nM to about 150 nM, about 10 nM to about 100 nM, about 10 nM to about 80 nM, about 10 nM to about 60 nM, about 10 nM to about 50 nM, about 10 nM to about 40 nM, about 10 nM to about 25 nm, about 10 nM to about 20 nM, about 10 nM to about 15 nM, about 20 nM to about 500 nM, about 20 nM to about 450 nM, about 20 nM to about 400 nM, about 20 nM to about 350 nM, about 20 nM to about 300, about 20 nM to about 250 nM, about 20 nM to about 200 nM, about 20 nM to about 150 nM, about 20 nM to about 100 nM, about 20 nM to about 80 nM, about 20 nM to about 60 nM, about 20 nM to about 50 nM, about 20 nM to about 40 nM, about 50 nM to about 500 nM, about 50 nM to about 450 nM, about 50 nM to about 400 nM, about 50 nM to about 350 nM, about 50 nM to about 300, about. 50 nM to about. 250 nM, about 50 nM to about 200 nM, about 50 nM to about 150 nM, about 50 nM to about 100 nM, about 50 nM to about 80 nM, about 100 nM to about 500 nM, about 100 nM to about 450 nM, about 100 nM to about 400 nM, about 100 nM to about 350 nM, about 100 nM to about 300, about 100 nM to about 250 nM, about 100 nM to about 200 nM, about 100 nM to about 150 nM, about 250 nM to about 500 nM, about 250 nM to about 450 nM, about 250 nM to about 400 nM, about 250 nM to about 350 nM, about 250 nM to about 300, about 400 nM to about 500 nM, or about 400 nM to about 450 nM).

### Linkers

The target-binding proteins may comprise one or more linkers. The linkers may comprise a stretch of amino acid sequence that link two components (e.g., between the heavy chain variable domain and the light chain variable domains, between the target-binding domain and a cleavable moiety, between the target-binding domain and a masking moiety, between a masking moiety and a cleavable moiety, or between a half-life extending moiety and another component in the target-binding proteins. The linkers may be non-cleavable by any protease, or non-cleavable by any protease naturally occurring in humans. In some embodiments, the linker(s) may be flexible linkers, which may be introduced into the target-binding proteins to provide flexibility at one or more of the junctions between domains, between moieties, between moieties and domains, or at any other junctions where a linker would be beneficial. In some embodiments, where the target-binding protein is provided as a conformationally constrained construct, a linker may be inserted to facilitate the formation and maintenance of a structure.

Any of the linkers described herein may provide the desired flexibility to facilitate the inhibition of the binding of a target, or to facilitate cleavage of a cleavable moiety by a protease. In some embodiments, the linkers may be all or partially flexible, such that the linker can include a flexible linker as well as one or more portions that confer less flexible structure to provide for a desired target-binding protein. Some linkers may include cysteine residues, which may form disulfide bonds and reduce flexibility of the construct.

In some embodiments, when an target-binding protein comprises a masking moiety, a linker coupled to the masking moiety may have a length that allows the masking moiety to be in a position in the tertiary or quaternary structure of to effectively mask the target-binding domain in the protein that allows the masking moiety to mask the target-binding domain in the target-binding protein. For example, in the tertiary or quaternary structure, the masking moiety may be proximal to the target-binding domain to be masked.

In most instances, the length of a linker may be determined by counting, in a N- to C-direction, the number of amino acids from the N-terminus of the linker adjacent to the C-terminal amino acid of the preceding component, to the C-terminus of the linker adjacent to the N-terminal amino acid of the following component (i.e., where the linker length does not include either the C-terminal amino acid of the preceding component or the N-terminal amino acid of the following component).

In some embodiments, a linker may comprise a total of 1 to 50, 1 to 40, 1 to 30, 1 to 25 (e.g., 1 to 24, 1 to 22, 1 to 20, 1 to 18, 1 to 16, 1 to 15, 1 to 14, 1 to 12, 1 to 10, 1 to 8, 1 to 6, 1 to 5, 1 to 4, 1 to 3, 1 to 2, 2 to 25, 2 to 24, 2 to 22, 2 to 20, 2 to 18, 2 to 16, 2 to 15, 2 to 14, 2 to 12, 2 to 10, 2 to 8, 2 to 6, 2 to 5, 2 to 4, 2 to 3, 4 to 25, 4 to 24, 4 to 22, 4 to 20, 4 to 18, 4 to 16, 4 to 15, 4 to 14, 4 to 12, 4 to 10, 4 to 8, 4 to 6, 4 to 5, 5 to 25, 5 to 24, 5 to 22, 5 to 20, 5 to 18, 5 to 16, 5 to 15, 5 to 14, 5 to 12, 5 to 10, 5 to 8, 5 to 6, 6 to 25, 6 to 24, 6 to 22, 6 to 20, 6 to 18, 6 to 16, 6 to 15, 6 to 14, 6 to 12, 6 to 10, 6 to 8, 8 to 25, 8 to 24, 8 to 22, 8 to 20, 8 to 18, 8 to 16, 8 to 15, 8 to 14, 8 to 12, 8 to 10, 10 to 25, 10 to 24, 10 to 22, 10 to 20, 10 to 18, 10 to 16, 10 to 15, 10 to 14, 10 to 12, 12 to 25, 12 to 24, 12 to 22, 12 to 20, 12 to 18, 12 to 16, 12 to 15, 12 to 14, 14 to 25, 14 to 24, 14 to 22, 14 to 20, 14 to 18, 14 to 16, 14 to 15, 15 to 25, 15 to 24, 15 to 22, 15 to 20, 15 to 18, 15 to 16, 16 to 25, 16 to 24, 16 to 22, 16 to 20, 16 to 18, 18 to 25, 18 to 24, 18 to 22, 18 to 20, 20 to 25, 20 to 24, 20 to 22, 22 to 25, 22 to 24, or 24 to 25 amino acids). In some embodiments, the linker may comprise a total of 1, 2, 3, 4, 5, 6, 7, 8, 9, 10, 11, 12, 13, 14, 15, 16, 17, 18, 19, 20, 21, 22, 23, 24, or 25 amino acids.

In some embodiments, a linker may be rich in glycine (Gly or G) residues. In some embodiments, the linker may be rich in serine (Ser or S) residues. In some embodiments, the linker may be rich in glycine and serine residues. In some embodiments, the linker may have one or more glycine-serine residue pairs (GS) (e.g., 1, 2, 3, 4, 5, 6, 7, 8, 9, or 10 or more GS pairs).

In some embodiments, the linker may have one or more Gly-Gly-Gly-Ser (GGGS) sequences (e.g., 1, 2, 3, 4, 5, 6, 7, 8, 9, or 10 or more GGGS sequences). In some embodiments, the linker may have one or more Gly-Gly-Gly-Gly-Ser (GGGGS) sequences (e.g., 1, 2, 3, 4, 5, 6, 7, 8, 9, or 10 or more GGGGS sequences). In some embodiments, the linker may have one or more Gly-Gly-Ser-Gly (GGSG) sequences (e.g., 1, 2, 3, 4, 5, 6, 7, 8, 9, or 10 or more GGSG sequences). Examples of the linkers may include glycine polymers (G)n, glycine-serine polymers (including, for example, (GS)n, (GGS)n, (GSGGS)n and (GGGS)n, where n is an integer of at least one), glycine-alanine polymers, alanine-serine polymers, and other flexible linkers known in the art. Glycine and glycine-serine polymers may be relatively unstructured, and therefore may be able to serve as a neutral link between components. Glycine accesses significantly more phi-psi space than even alanine, and is much less restricted than residues with longer side chains (see Scheraga, Rev. Computational Chem. 11173-142 (1992)). Example flexible linkers include one of or a combination of one or more of: GGSG, GGSGG (SEQ ID NO: 667), GSGSG (SEQ ID NO: 668), GSGGG (SEQ ID NO: 669), GGGSG (SEQ ID NO: 670), GSSSG (SEQ ID NO: 671), GSSGGSGGSGG (SEQ ID NO: 672), GGGS (SEQ ID NO: 673), GGGSGGGS (SEQ ID NO: 674), GGGSGGGSGGGS (SEQ ID NO: 675), GGGGSGGGGSGGGGS (SEQ ID NO: 676), GGGGSGGGGSGGGGSGGGGSGGGGS (SEQ ID NO: 677), GGGGSGGGGS (SEQ ID NO: 678), GGGGS (SEQ ID NO: 679), GS, GGGGSGS (SEQ ID NO: 680), GGGGSGGGGSGGGGSGS (SEQ ID NO: 681), GGSLDPKGGGGS (SEQ ID NO: 682), PKSCDKTHTCPPCPAPELLG (SEQ ID NO: 683), SKYGPPCPPCPAPEFLG (SEQ ID NO: 684), GKSSGSGSESKS (SEQ ID NO: 685), GSTSGSGKSSEGKG (SEQ ID NO: 686), GSTSGSGKSSEGSGSTKG (SEQ ID NO: 687), GSTSGSGKPGSGEGSTKG (SEQ ID NO: 688), and GSTSGSGKPGSSEGST (SEQ ID NO: 689), or GSTSGSGKPGSSEGST (SEQ ID NO: 690).

Examples of linkers may further include a sequence that is at least 70% identical (e.g., at least 72%, at least 74%, at least 75%, at least 76%, at least 78%, at least 80%, at least 82%, at least 84%, at least 85%, at least 86%, at least 88%, at least 90%, at least 92%, at least 94%, at least 95%, at least 96%, at least 97%, at least 98%, at least 99%, or 100% identical) to the example linkers described herein. An ordinarily skilled artisan will recognize that design can include linkers that are all or partially flexible, such that the linker can include a flexible linker as well as one or more portions that confer less flexible structure to provide for a desired target-binding protein structure.

In some embodiments, a target-binding protein may include one, two, three, four, five, six, seven, eight, nine, or ten linker(s) (e.g., the same or different linker sequences of any of the exemplary linker sequences described herein or known in the art). In some embodiments, a linker may comprise non-amino acid-based linkers including but not limited to sulfo-SIAB (sulfosuccinimidyl (4-iodoacetyl)aminobenzoate), SMPB (succinimidyl 4-(N-maleimidophenyl)butyrate), and sulfo-SMPB (sulfosuccinimidyl 4-(N-maleimidophenyl)butyrate), wherein the linkers react with primary amines sulfhydryls.

### Half-Life Extending Moieties (EMs)

The target-binding protein may further comprise a half-life extending moiety (EM). In some examples, the half-life extending moiety may be a serum half-life extending moiety, i.e., capable of extending the serum half-life of the molecule attached to the EM.

In some examples, the EM may comprise a fragment crystallizable region (Fc domain) of an antibody. For example, the EM may be the Fc domain of an IgG (e.g., IgG1, IgG2, IgG3, or IgG4). In some examples, the EM may comprise a dimer formed by two Fc domains. The Fc domain may be a wild type peptide or a mutant. For example, the EM may comprise a dimer formed by two Fc domain mutants. In such cases, the two Fc domain mutants may be a Fc domain hole mutant and a Fc domain knob mutant. The knob and hole mutants may interact with each other to facilitate the dimerization of the two Fc domains. In some embodiments, the knob and hole mutants may comprise one or more amino acid modifications within the interface between two Fc domains (e.g., in the CH3 domain). In one example, the modifications comprise amino acid substitution T366W and optionally the amino acid substitution S354C in one IgG Fc domain and the amino acid substitutions T366S, L368A, Y407V and optionally Y349C in the other IgG Fc domain (numbering according to EU numbering system). Example of Fc mutants also include SEQ ID NOs: 26-27.

Examples of the Fc domain mutants also include those described in U.S. Pat. Nos. 7,695,936, which is incorporated herein by reference in its entirety. In one example, the modifications comprise amino acid substitution T366Y in one IgG Fc domain, and the amino acid substitutions Y407T in the other IgG Fc domain. In one example, the modifications comprise amino acid substitution T366W in one IgG Fc domain, and the amino acid substitutions Y407A in the other IgG Fc domain. In one example, the modifications comprise amino acid substitution F405A in one IgG Fc domain, and the amino acid substitutions T394W in the other IgG Fc domain. In one example, the modifications comprise amino acid substitution T366Y and F405A in one IgG Fc domain, and the amino acid substitutions T394W and Y407T in the other IgG Fc domain. In one example, the modifications comprise amino acid substitution T366W and F405W in one IgG Fc domain, and the amino acid substitutions T394S and Y407A in the other IgG Fc domain. In one example, the modifications comprise amino acid substitution F405W and Y407A in one IgG Fc domain, and the amino acid substitutions T366W and T394S in the other IgG Fc domain. In one example, the modifications comprise amino acid substitution F405W in one IgG Fc domain, and the amino acid substitutions T394S in the other IgG Fc domain. The mutation positions in the Fe domains are numbered according to EU numbering system. The IgG Fc domain may be comprise a sequence of SEQ ID NOs: 34-37 (IgG1, IgG2, IgG3 or IgG4). In these sequences, amino acids 1-107 correspond to EU numbering 341-447.

In some examples, the Fe domains mutants may have reduced effector function. Examples of such Fe domains include those disclosed in in US20190135943, which incorporated herein by reference in its entirety. Further examples of Fe domains include SEQ ID NOs: 38-42.

Further examples of EMs include immunoglobulin (e.g., IgG), serum albumin (e.g., human serum albumin (HSA), hexa-hat GST (glutathione S-transferase) glutathione affinity, Calmodulin-binding peptide (CBP), Strep-tag, Cellulose Binding Domain, Maltose Binding Protein, S-Peptide Tag, Chitin Binding Tag, Immuno-reactive Epitopes, Epitope Tags, E2Tag, HA Epitope Tag, Myc Epitope, FLAG Epitope, AU1 and AU5 Epitopes, Glu-Glu Epitope, KT3 Epitope, IRS Epitope, Btag Epitope, Protein Kinase-C Epitope, and VSV Epitope.

In some embodiments, the serum half-life of the target-binding protein may be longer than that of a reference protein (e.g., a substantially the same target-binding protein that does not have the half-life extending moiety), e.g., the pK of the target-binding protein is longer than that of the reference protein. In some examples, the target-binding protein with an EM may have a serum half-life that is at least 10%, 20%, 30%, 40%, 50%, 60%, 70%, 80%, 90%, 2-fold, 4-fold, 6-fold, 8-fold, 10-fold, 20-fold, 30-fold, 40-fold, 50-fold, 60-fold, 70-fold, 80-fold, 90-fold, 100-fold longer than the serum half-life of a reference target-binding protein that is the same but does not have the EM. In some embodiments, the serum half-life of the target-binding protein may be at least 15 days, 12 days, 11 days, 10 days, 9 days, 8 days, 7 days, 6 days, 5 days, 4 days, 3 days, 2 days, 1 day, 20 hours, 18 hours, 16 hours, 14 hours, 12 hours, 10 hours, 8 hours, 6 hours, 4 hours, 3 hours, 2 hours, or 1 hour when administered to an organism.

### Additional Target-Binding Domains

The target-binding proteins may comprise one or more target-binding domains in addition to the CD3-binding domain described herein. In some embodiments, the target-binding protein may comprise an additional light chain variable domain and an additional heavy chain variable domain. The additional light chain variable domain and the additional heavy chain variable domain may form an additional target-binding domain. In some examples, the CD3-binding domain and the additional target-binding domain may be identical. In some examples, the CD3-binding domain and the additional target-binding domain may be different from each other (e.g., may specifically bind to the same or different antigens or epitopes).

In some embodiments, the CD3-binding domain and the additional target-binding domain may be both Fv fragments, or at least one may be a Fv fragment. In some embodiments, the CD3-binding domain and the additional target-binding domain may be both Fab fragments, or at least one may be a Fab fragment. In some embodiment, the CD3-binding domain and an additional target-binding domain may be a Fab′ fragment, or at least one can be a Fab′ fragment.

In some embodiments, the target-binding protein may be multispecific (e.g., bispecific, trispecific, tetraspecific, and other multispecific target-binding proteins), e.g., binding to CD3 and one or more additional targets. In some embodiments, the multispecific target-binding protein may be multivalent, e.g., comprising multiple target-binding sites regardless of whether the binding sites recognize the same or different targets. In some embodiment, the target-binding protein may be bispecific. The term “bispecific” means that target-binding protein is able to specifically bind to two distinct targets. Typically, a bispecific target-binding protein comprises two target-binding domains, each of which is capable of specifically binding to a different target. In some embodiments, the bispecific target-binding protein may be capable of simultaneously binding two targets, e.g., two target proteins expressed on two distinct cells.

In some embodiments, the target-binding protein may comprise the CD3-binding domain and an additional target-binding domain capable of binding to a molecule on the surface of a cell associated with a disease (e.g., a tumor cell). Such target-binding protein may simultaneously bind to an immune cell (e.g., T cell) and a cell associated with a disease (e.g., a tumor cell), thus activating the immune cell and crosslinking the activated immune cell to the cell associated with the disease. In some embodiments, the target-binding protein may be formulated as part of chimeric antigen receptor (CAR), a T cell engaging antibody (e.g., T cell engaging bispecific antibody), a pro-Bispecific T Cell Engager (pro-BITE) molecule, pro-Chimeric Antigen Receptor (pro-CAR) modified T cell, or other engineered receptor or other immune effector cell, such as a CAR modified NK cell.

In some examples, the target-binding protein may be a monovalent bispecific antibody comprising the CD3-binding domain and an additional target-binding domain described herein. As used herein, the term “monovalent bispecific antibody” refers to a bispecific antibody, in which only one antigen-binding domain is directed against a given target. The inventors have surprisingly discovered that certain CD3-binding domains described herein display improved stability, manufacturability, and/or CD3 binding affinity in the context of a monovalent CD3 binding protein, including monovalent bispecific antibodies that specifically bind CD3 (e.g., an anti-CD3 scFv).

The target of the additional target-binding domain may be a protein or other type of molecules, e.g., cell surface receptors and secreted binding proteins (e.g., growth factors), soluble enzymes, structural proteins (e.g. collagen, fibronectin) and the like.

In some examples, the additional target-binding domain may bind to a target that is a molecule on or inside a cell that is associated with a disease. For example, the additional target-binding domain may bind to a tumor cell. In such cases, the additional target-binding domain may bind to a tumor associated antigen. As used herein, the term “tumor associated antigen” refers to any antigen including a protein, glycoprotein, ganglioside, carbohydrate, lipid that is associated with cancer. Such antigen may be expressed on tumor cells (e.g., malignant cells) or in the tumor microenvironment such as on tumor-associated blood vessels, extracellular matrix, mesenchymal stroma, or immune infiltrates. In some embodiments, the tumor associated antigen may be human epidermal growth factor receptor 2 (HER2). For example, the additional target-binding domain may be trastuzumab or a fragment thereof. In another example, the additional target-binding domain may be pertuzumab or a fragment thereof.

### Activatable Target-Binding Proteins

In one aspect, the target-binding proteins herein include activatable target-binding proteins. In general, an activatable target-binding protein may comprise a prodomain, which refers to a polypeptide that, when linked to a target-binding protein, functions to inhibit target binding by the target-binding protein and includes an amino acid sequence that form a protease cleavable substrate. The portion of the prodomain that inhibits target binding is referred to as a masking moiety (MM) and the amino acid sequence that is a protease cleavable substrate is referred to as a cleavable moiety (CM). The prodomain may include a linker (L) between the MM and the CM and/or at the prodomain's terminus (e.g., carboxyl and/or amino terminus to facilitate the linkage of the prodomain to the target-binding protein). In certain embodiments, a prodomain may comprise one of the following formulae (representing an amino acid sequence in an N- to C-terminal direction): MM-CM, MM-L-CM, MM-CM-L, MM-L-CM-L, CM-MM, CM-L-MM, L-CM-MM, or L-CM-L-MM, wherein each “-” represents a direct or indirect (e.g., via a linker) linkage.

As used herein, the term “activatable target-binding protein” refers to a target-binding protein in its inactive (uncleaved or native) state. It will be apparent to the ordinarily skilled artisan that in some embodiments a cleaved activatable target-binding protein may be connected to a MM that is not reducing, inhibiting, or interfering with binding between the target-binding domain and its target. In some embodiments, a cleaved activatable target-binding protein may lack a MM due to cleavage of the CM (e.g., by a protease), resulting in release of the MM. As used herein, the term “cleaved state” or “active state” refers to the condition of the activatable target-binding proteins following cleavage of the CM by at least one protease. The term “uncleaved state” or “inactive state” refers to the condition of the activatable target-binding proteins in the absence of cleavage of the CM by a protease.

By activatable is meant that the activatable target-binding protein exhibits a first level of binding to a target when the activatable target-binding protein is in an inhibited, masked or uncleaved state (i.e., a first conformation), and a second level of binding to the target in the uninhibited, unmasked and/or cleaved state (i.e., a second conformation), where the second level of target binding is greater than the first level of binding. In general, the access of target to the target-binding domain of the activatable target-binding protein is greater in the presence of a cleaving agent capable of cleaving the CM, i.e., a protease, than in the absence of such a cleaving agent. Thus, when the activatable target-binding protein is in the uncleaved state, the target-binding domain is inhibited from target binding and can be masked from target binding (i.e., the first conformation is such that the target-binding domain cannot bind the target or is inhibited in binding the target), and in the cleaved state the target-binding domain is not inhibited or is unmasked to target binding.

In some embodiments, an activatable target-binding protein may be designed by selecting a target-binding domain of interest and constructing the remainder of the activatable target-binding protein so that, when conformationally constrained, the MM provides for masking of the target-binding domain or reduction of binding of the target-binding domain to its target. Structural design criteria can be to be taken into account to provide for this functional feature.

Activatable target-binding proteins herein may exhibit an activatable phenotype of a desired dynamic range for target binding in an inhibited versus an uninhibited conformation. Dynamic range generally refers to a ratio of (a) a maximum detected level of a parameter under a first set of conditions to (b) a minimum detected value of that parameter under a second set of conditions. For example, in the context of an activatable target-binding protein, the dynamic range refers to the ratio of (a) a maximum detected level of target protein binding to an activatable target-binding protein in the presence of a protease capable of cleaving a CM in the activatable target-binding proteins to (b) a minimum detected level of target protein binding to an activatable target-binding protein in the absence of the protease. The dynamic range of an activatable target-binding protein can be calculated as the ratio of the dissociation constant of an activatable target-binding protein cleaving agent (e.g., enzyme) treatment to the dissociation constant of the activatable target-binding proteins cleaving agent treatment. The greater the dynamic range of an activatable target-binding protein, the better the activatable phenotype of the activatable target-binding protein. Activatable target-binding proteins having relatively higher dynamic range values (e.g., greater than 1) exhibit more desirable activatable phenotypes such that target protein binding by the activatable target-binding proteins occurs to a greater extent (e.g., predominantly occurs) in the presence of a cleaving agent (e.g., enzyme) capable of cleaving the CM of the activatable target-binding proteins than in the absence of a cleaving agent.

The activatable target-binding protein herein may comprise a target-binding domain (TB), one or more masking moieties (MMs) reducing, inhibiting, or interfering with the binding of the target-binding domain to its target(s), one or more cleavable moieties (CMs) that couple the one or more MMs to the TB, and optionally one or more half-life extending moieties (EMs). In some embodiments, the activatable target-binding protein may comprise the TB that specifically binds to CD3, and a masking moiety (MM) inhibiting the binding of the TB and CD3, wherein the MM is coupled to the TB via a cleavable moiety (CM) (either directly or indirectly, e.g., via one or more linkers). As used herein and unless otherwise stated, components of the activatable target-binding protein that are “coupled” may be coupled either via a direct covalent linkage or indirect covalent linkage, e.g., via one or more linking peptides (also referred to as “linkers”), cleavable moieties, or other components of the activatable protein.

In some embodiments, the activatable target-binding protein may comprise more than one target-binding domains (TBs). For example, the activatable target-binding protein may comprise the first TB that specifically binds to CD3, a first MM (MM1) inhibiting the binding of the TB1 and CD3, wherein the MM1 is coupled to the TB1 via a first cleavable moiety (CM1) (either directly or indirectly, e.g., via one or more linkers), a second target-binding domain (TB2) that specifically binds to a second target, a second masking moiety (MM2) inhibiting the binding of the TB2 and the second target, wherein the MM2 is coupled to the TB2 via a second cleavable moiety (CM2) (either directly or indirectly, e.g., via one or more linkers). The activatable target binding protein may further comprise a half-life extending moiety (EM). In one example, the activatable target-binding protein comprise a scFv comprising the TB1.

### Masking Moieties (MMs)

The activatable target-binding proteins herein may comprise one or more masking moieties (MMs) capable of interfering with the binding of the TBs to the targets. A masking moiety in an activatable molecule (that is not yet activated) “masks” or reduces or otherwise inhibits the binding of the target-binding domain to its target. In some embodiments, the coupling or modifying of target-binding protein with a MM may inhibit the ability of the protein to specifically bind its target by means of inhibition known in the art (e.g., structural change and competition for antigen-binding domain). In some embodiments, the coupling or modifying of a target-binding protein with a MM may effect a structural change that reduces or inhibits the ability of the protein to specifically bind its target. In some embodiments, the coupling or modifying of a target-binding protein with a MM sterically blocks, reduces or inhibits the ability of the antigen-binding domain to specifically bind its target.

A MM may be coupled to a TB by a CM and optionally one or more linkers described herein. In some embodiments, when an activatable target-binding protein is not activated, the MM prevents the TB from target binding; but when the activatable target-binding protein is activated (when the CM is cleaved by a protease), the MMs does not substantially or significantly interfere with the TB's binding to the target.

In the activatable target-binding protein, a MM interfering with the target binding of a TB may be coupled to the TB (either directly or indirectly, e.g., via one or more linkers). Alternatively, a MM interfering with the target binding of a TB may be coupled, either directly or indirectly, to a component of the activatable target-binding protein that is not the TB. For example, the MM may be coupled, either directly or indirectly, to a different TB. In another example, the MM may be coupled, either directly or indirectly, with an EM. In either case, in the tertiary or quaternary structure of the activatable structure, the MM may be in a position (e.g., proximal to the TB to be masked) that allows the MM to mask the TB.

In some embodiments, a MM may interact with the TB, thus reducing or inhibiting the interaction between the TB and its binding partner. In some embodiments, the MM may comprise at least a partial or complete amino acid sequence of a naturally occurring binding partner of the TB. For example, the MM may be a fragment of a naturally occurring binding partner. The fragment may retain no more than 95%, 90%, 80%, 75%, 70%, 60%, 50%, 40%, 30%, 25%, or 20% nucleic acid or amino acid sequence homology to the naturally occurring binding partner. In some embodiments, the MM may be a cognate peptide of the TB. For example, the MM may comprise a sequence of the TB's epitope or a fragment thereof. The term “naturally occurring” as used herein as applied to an object refers to the fact that an object can be found in nature. For example, a polypeptide or polynucleotide sequence that is present in an organism (including viruses) that can be isolated from a source in nature and that has not been intentionally modified by man in the laboratory or otherwise is naturally occurring.

In some embodiments, the MM may comprise an amino acid sequence that is not naturally occurring or does not contain the amino acid sequence of a naturally occurring binding partner or target protein. In certain embodiments, the MM is not a natural binding partner of the TB. The MM may be a modified binding partner for the TB which contains amino acid changes that decrease affinity and/or avidity of binding to the TB. In some embodiments the MM may contain no or substantially no nucleic acid or amino acid homology to the TB's natural binding partner. In other embodiments the MM is no more than 5%, 10%, 15%, 20%, 25%, 30%, 35%, 40%, 45%, 50%, 55%, 60%, 65%, 70%, 75%, or 80% similar to the natural binding partner of the TB.

In some embodiments, the MM may not specifically bind to the TB, but still interfere with TB's binding to its binding partner through non-specific interactions such as steric hindrance. For example, the MM may be positioned in the activatable target-binding protein such that the tertiary or quaternary structure of the activatable target-binding protein allows the MM to mask the TB through charge-based interaction, thereby holding the MM in place to interfere with binding partner access to the TB.

In some embodiments, the MM may have a dissociation constant for binding to the TB that is no more than the dissociation constant of the TB to the target. In some embodiments, the MM may not interfere or compete with the TB for binding to the target in a cleaved state.

The structural properties of the MMs may be selected according to factors such as the minimum amino acid sequence required for interference with protein binding to target, the target protein-protein binding pair of interest, the size of the TB, the presence or absence of linkers, and the like.

In some embodiments, the MM may be unique for the coupled TB. Examples of MMs include MMs that were specifically screened to bind a binding domain of the TB or fragment thereof (e.g., affinity masks). Methods for screening MMs to obtain MMs unique for the TB and those that specifically and/or selectively bind a binding domain of a binding partner/target are provided herein and can include protein display methods.

As used herein, the term “masking efficiency” refers to the activity (e.g., EC₅₀) of the activatable target-binding protein in the inactivated state divided by the activity of a control antibody, wherein the control antibody may be either cleavage product of the activatable target-binding protein or the antibody or fragment thereof used as the TB of the activatable target-binding protein. An activatable target-binding protein having a reduced level of a TB activity may have a masking efficiency that is greater than 10. In some embodiments, the activatable target-binding proteins described herein may have a masking efficiency that is greater than 10, 100, 1000, or 5000.

In some embodiments, the MM may be a polypeptide of about 2 to 50 amino acids in length. For example, the MM may be a polypeptide of from 2 to 40, from 2 to 30, from 2 to 20, from 2 to 10, from 5 to 15, from 10 to 20, from 15 to 25, from 20 to 30, from 25 to 35, from 30 to 40, from 35 to 45, from 40 to 50 amino acids in length. For example, the MM may be a polypeptide with 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some examples, the MM may be a polypeptide of more than 50 amino acids in length, e.g., 100, 200, 300, 400, 500, 600, 700, 800, or more amino acids.

In some embodiments, in an inactive state of the activatable target-binding protein with an TB and an interfering MM, in the presence of the target of an TB, there is no binding or substantially no binding of the TB to the target, or no more than 0.001%, 0.01%, 0.1%, 1%, 2%, 3%, 4%, 5%, 6%, 7%, 8%, 9%, 10%, 15%, 20%, 25%, 30%, 35%, 40%, or 50% binding of the TB to its target, as compared to the binding of an counterpart antibody without the interfering MM, for at least 0.1, 0.5, 1, 2, 4, 6, 8, 12, 28, 24, 30, 36, 48, 60, 72, 84, 96 hours, or 5, 10, 15, 30, 45, 60, 90, 120, 150, 180 days, or 1, 2, 3, 4, 5, 6, 7, 8, 9, 10, 11, or 12 months when measured in vitro immunoabsorbant assay, e.g., as described in US20200308243A1.

The binding affinity of the TB towards the target or binding partner with an interfering MM may be at least 5, 10, 25, 50, 100, 250, 500, 1,000, 2,500, 5,000, 10,000, 50,000, 100,000, 500,000, 1,000,000, 5,000,000, 10,000,000, 50,000,000 times lower than the binding affinity of the TB towards its binding partner without an interfering MM, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times lower than the binding affinity of the TB towards its binding partner when there is no interfering MM.

The dissociation constant of the MM towards the TB it masks, may be greater than the dissociation constant of the TB towards the target. The dissociation constant of the MM towards the masked TB may be at least 5, 10, 25, 50, 100, 250, 500, 1,000, 2,500, 5,000, 10,000, 100,000, 1,000,000 or even 10,000,000 times greater than the dissociation constant of the TB towards the target. Conversely, the binding affinity of the MM towards the masked TB may be lower than the binding affinity of the TB towards the target. The binding affinity of MM towards the TB may be at least 5, 10, 25, 50, 100, 250, 500, 1,000, 2,500, 5,000, 10,000, 100,000, 1,000,000 or even 10,000,000 times lower than the binding affinity of the TB towards the target.

In some embodiments, the MMs may contain genetically encoded or genetically non-encoded amino acids. Examples of genetically non-encoded amino acids include but are not limited to D-amino acids, 3-amino acids, and 7-amino acids. In specific embodiments, the MMs contain no more than 50%, 40%, 30%, 20%, 15%, 10%, 5% or 1% of genetically non-encoded amino acids.

In some embodiments, once released from the activatable target-binding protein and in a free state, the MM may have a biological activity or a therapeutic effect, such as binding capability. For example, the free peptide may bind with the same or a different binding partner. In certain embodiments, the free MM may exert a therapeutic effect, providing a secondary function to the compositions disclosed herein. In some embodiments, once uncoupled from the activatable target-binding protein and in a free state, the MM may advantageously not exhibit biological activity. For example, in some embodiments the MM in a free state does not elicit an immune response in the subject.

Suitable MMs may be identified and/or further optimized through a screening procedure from a library of candidate activatable target-binding proteins having variable MMs. For example, a TB and a CM may be selected to provide for a desired enzyme/target combination, and the amino acid sequence of the MM can be identified by the screening procedure described below to identify a MM that provides for a switchable phenotype. For example, a random peptide library (e.g., of peptides comprising 2 to 40 amino acids or more) may be used in the screening methods disclosed herein to identify a suitable MM.

In some embodiments, MMs with specific binding affinity for a TB may be identified through a screening procedure that includes providing a library of peptide scaffolds comprising candidate MMs wherein each scaffold is made up of a transmembrane protein and the candidate MM. The library may then be contacted with an entire or portion of a protein such as a full length protein, a naturally occurring protein fragment, or a non-naturally occurring fragment containing a protein (also capable of binding the binding partner of interest), and identifying one or more candidate MMs having detectably bound protein. The screening may be performed by one more rounds of magnetic-activated sorting (MACS) or fluorescence-activated sorting (FACS), as well as determination of the binding affinity of MM towards the TB and subsequent determination of the masking efficiency, e.g., as described in WO2009025846 and US20200308243A1, which are incorporated herein by reference in their entireties.

In some embodiments, a MM may be selected for use with a specific antibody or antibody fragment. For example, suitable MM for use with a TB that binds to an epitope may comprise the sequence of the epitope. In some examples, suitable MM for masking the anti-CD3 binding proteins disclosed herein include MMs comprising the sequences of GYLWGCEWNCGGITT (SEQ ID NO: 691), NAFRCWWDPPCQPMT (SEQ ID NO: 692), ARGLCWWDPPCTHDL (SEQ ID NO: 693), or NHSLCYWDPPCEPST (SEQ ID NO: 694). Additional masking moieties for anti-CD3 binding proteins include the sequences of MMYCGGNEVLCGPRV (SEQ ID NO: 695), GYRWGCEWNCGGITT (SEQ ID NO: 696), MMYCGGNEIFCEPRG (SEQ ID NO: 697), GYGWGCEWNCGGSSP (SEQ ID NO: 698), and MMYCGGNEIFCGPRG (SEQ ID NO: 699).

Examples of suitable MMs are disclosed in WO2021207657, WO2021142029, WO2021061867, WO2020252349, WO2020252358, WO2020236679, WO2020176672, WO2020118109, WO2020092881, WO2020086665, WO2019213444, WO2019183218, WO2019173771, WO2019165143, WO2019075405, WO2019046652, WO2019018828, WO2019014586, WO2018222949, WO2018165619, WO2018085555, WO2017011580, WO2016179335, WO2016179285, WO2016179257, WO2016149201, and WO2016014974, which are incorporated herein by reference in their entireties.

### Cleavable Moieties (CMs)

The activatable target-binding protein may comprise one or more cleavable moieties (CMs). The terms “cleavable moiety” and “CM” are used interchangeably herein to refer to a peptide, the amino acid sequence of which comprises a substrate for a sequence-specific protease. In some embodiments, the CM may be positioned between a TB and a MM.

The CM and the TB of the activatable target-binding proteins may be selected so that the TB represents a binding moiety for a given target, and the CM represents a substrate for one or more proteases, where the protease is co-localized with the target in a tissue (e.g., at a treatment site or diagnostic site in a subject). The protease may cleave the CM in the activatable target-binding protein when the activatable target-binding protein is exposed to the protease. In some embodiments, the activatable target-binding proteins may find particular use where, for example, one or more proteases capable of cleaving a site in the CM, is present at relatively higher levels in target-containing tissue of a treatment site or diagnostic site than in tissue of non-treatment sites (for example in healthy tissue).

In some embodiments, the CMs herein may comprise substrates for proteases that have known substrates have been reported in a number of cancers. See, e.g., La Roca et al., British J. Cancer 90(7):1414-1421, 2004. Substrates suitable for use in the CM components employed herein include those which are more prevalently found in cancerous cells and tissue. Thus, in certain embodiments, the CM may comprise a substrate for a protease that is more prevalently found in diseased tissue associated with a cancer. Examples of the cancers include gastric cancer, breast cancer, osteosarcoma, esophageal cancer, breast cancer, a HER2-positive cancer, Kaposi sarcoma, hairy cell leukemia, chronic myeloid leukemia (CML), follicular lymphoma, renal cell cancer (RCC), melanoma, neuroblastoma, basal cell carcinoma, cutaneous T-cell lymphoma, nasopharyngeal adenocarcinoma, ovarian cancer, bladder cancer, BCG-resistant non-muscle invasive bladder cancer (NMIBC), endometrial cancer, pancreatic cancer, non-small cell lung cancer (NSCLC), colorectal cancer, esophageal cancer, gallbladder cancer, glioma, head and neck carcinoma, uterine cancer, cervical cancer, or testicular cancer, and the like. In some embodiments, the CM components comprise substrates for protease(s) that is/are more prevalent in tumor tissue. For example, the protease(s) may be produced by a tumor in a subject.

In some embodiments, the activatable target-binding protein may comprise two CMs (e.g., for coupling MMs to multiple TBs). In some examples, the first and the second CMs may comprise the substrates of the same protease. In some examples, the first and the second CMs may comprise the substrates of different proteases. In some examples, the first and the second CMs may comprise or consist of the same sequence. In some examples, the first and the second CMs may comprise or consist of different sequences.

Suitable CMs for use in the activatable target-binding protein herein include any of the protease substrates that are known the art. In some examples, the CM may comprise a substrate of a serine protease (e.g., u-type plasminogen activator (uPA, also referred to as urokinase), matriptase (also referred to herein as MT-SP1 or MTSP1). In some examples, the CM may comprise a substrate of a matrix metalloprotease (MMP). In some examples, the CM may comprise a substrate of cysteine protease (CP) (e.g., legumain).

In some embodiments, the CM may comprise a substrate for a disintegrin and metalloproteinase (ADAM) or disintegrin and metalloproteinase with thrombospondin motifs (ADAMTS)(e.g., ADAM8, ADAM9, ADAM10, ADAM12, ADAM15, ADAM17/TACE, ADEMDEC1, ADAMTS1, ADAMTS4, ADAMTS5), aspartate protease (e.g. BACE, Renin), aspartic cathepsin (e.g., Cathepsin D, Cathepsin E), Caspase (e.g., Caspase 1, Caspase 2, Caspase 3, Caspase 4, Caspase 5, Caspase 6, Caspase 7, Caspase 8, Caspase 9, Caspase 10, Caspase 14), cysteine cathepsin (e.g., Cathepsin A, Cathepsin B, Cathepsin C, Cathepsin G, Cathepsin K, Cathepsin L, Cathepsin S, Cathepsin V/L2, Cathepsin X/Z/P), cysteine proteinase (e.g., Cruzipain, Legumain, Otubain-2), Chymase, DESC1, DPP-4, FAP, Elastase, FVIIa, FIXA, FXa, FXIa, FXIIa, Granzyme B, Guanidinobenzoatase, Hepsin, HtrA1, Human Neutrophil Elastase, KLK (e.g., KLK4, KLK5, KLK6, KLK7, KLK8, KLK10, KLK11, KLK13, KLK14), metallo proteinase (e.g., Meprin, Neprilysin, PSMA, BMP-1), Lactoferrin, Marapsin, Matriptase-2, MT-SP1/Matriptase, NS3/4A, PACE4, Plasmin, PSA, a MMP (e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27), TMPRSS2, TMPRSS3, TMPRSS4, tPA, Thrombin, Tryptase, and uPA.

In some embodiments, the protease substrate in the CM may comprise a peptide sequence that is not substantially identical (e.g., no more than 90%, 80%, 70%, 60%, or 50% identical) to any polypeptide sequence that is naturally cleaved by the same protease.

In some embodiments, the CM may be or comprise a sequence of encompassed by the consensus of sequence of any one of the sequences in Table 6 below.

TABLE 6
Exemplary CM sequences

| | SEQ |
|---|---|
| | ID |
| Sequences | NO |
| AANALAHGLF | 167 |
| AANL | 168 |
| AANLGSGGSS | 169 |
| AAPRS | 170 |
| AAPRSF | 171 |
| AARGPAIH | 172 |
| AAYHLVSQ | 173 |
| AFPDMRSVRS | 174 |
| AFQALRM | 175 |
| AFRHLR | 176 |
| AGLGISST | 177 |
| AGLGVVER | 178 |
| AGPR | 179 |
| AHGL | 180 |
| AHGLF | 181 |
| AHQALRM | 182 |
| AIPRVRLFDV | 183 |
| ALAHG | 184 |
| ALAHGL | 185 |
| ALAHGLF | 186 |
| ALAHGLFAPRSF | 187 |
| ALAHGLFSGRSAN | 188 |
| ALAHGLPTFVHL | 189 |
| ALGLLRLP | 190 |
| ALPSVKMVSE | 191 |
| ALRAP | 192 |
| ANQALRM | 193 |
| ANQALRMA | 194 |
| APPLVKSMVV | 195 |
| APPSFKLVNA | 196 |
| APRS | 197 |
| APRSALAHGLF | 198 |
| APRSF | 199 |
| AQFVLTEG | 200 |
| AQNLLGMV | 201 |
| ARGP | 202 |
| ARGPS | 203 |
| ARGPSF | 204 |
| ARGPSFK | 205 |
| ASGLLRFP | 206 |
| ASPTMKTVGL | 207 |
| AVGLLAPP | 208 |
| AVGLLAPPGGLSGRSANI | 209 |
| AVGLLAPPGGLSGRSANP | 210 |
| AVGLLAPPGGLSGRSDDH | 211 |
| AVGLLAPPGGLSGRSDIH | 212 |
| AVGLLAPPGGLSGRSDNH | 213 |
| AVGLLAPPGGLSGRSDNI | 214 |
| AVGLLAPPGGLSGRSDNP | 215 |
| AVGLLAPPGGLSGRSDQH | 216 |
| AVGLLAPPGGLSGRSDTH | 217 |
| AVGLLAPPGGLSGRSDYH | 218 |
| AVGLLAPPGGLSGRSNI | 219 |
| AVGLLAPPGGLSGRSNIG | 220 |
| AVGLLAPPGGLSGRSNIGS | 221 |
| AVGLLAPPGGTSTSGRSANPRG | 222 |
| AVGLLAPPSGRSANPRG | 223 |
| AVGLLAPPTSGRSANPRG | 224 |
| AVPKVRVVPE | 225 |
| CGPPLGR | 226 |
| CSPPLGR | 227 |
| DEVDGSGGSS | 228 |
| DEXXXC(A/S) | |
| DISHWRRS | 229 |
| DLAHPLL | 230 |
| DLPLVKSLPS | 231 |
| DLXXT(A/S) | |
| DRLSGRSANHKK | 232 |
| DRLSGRSDNHKK | 233 |
| DRPEMKSLSG | 234 |
| DRPKVKTMDF | 235 |
| DVAQFVLT | 236 |
| DVPPMKTLRP | 237 |
| DWLYWMGI | 238 |
| DWLYWMSI | 239 |
| DWLYWPGI | 240 |
| DWLYWPSI | 241 |
| EAPKVKALPK | 242 |
| EHPRVKVVSE | 243 |
| EKPRMKLFQG | 244 |
| EPQALAMS | 245 |
| EQPEVKMVKG | 246 |
| ERPGVKSLVL | 247 |
| ESLPVVAV | 248 |
| ESPVMKSMAL | 249 |
| ESRRW | 250 |
| ESRRWM | 251 |
| ESRRWMP | 252 |
| ETPSVKTMGR | 253 |
| ETPSVKTMGRSS | 254 |
| FPRPLGITGL | 255 |
| FRLLDWQW | 256 |
| GCGPPLGR | 257 |
| GCSPPLGR | 258 |
| GFPHMKTFQH | 259 |
| GFPHMKTFQHSS | 260 |
| GGGPPLGR | 261 |
| GGPPLGR | 262 |
| GGQPSGMWGW | 263 |
| GGSIDGR | 264 |
| GGSPPLGR | 265 |
| GGWHTGRN | 266 |
| GIAGQ | 267 |
| GLGTPRGLFA | 268 |
| GLPTFV | 269 |
| GLPTFVH | 270 |
| GLPTFVHL | 271 |
| GLPTFVHLPRQV | 272 |
| GLSGRSDNHGSS | 273 |
| GPEGLRVG | 274 |
| GPLGIAGI | 275 |
| GPLNGRSDNHKA | 276 |
| GPLNGRSDNHKK | 277 |
| GPLNGRSDNHKR | 278 |
| GPLNGRSDNHQA | 279 |
| GPLNGRSDNHQK | 280 |
| GPLNGRSDNHQR | 281 |
| GPLNGRSDNHRA | 282 |
| GPLNGRSDNHRK | 283 |
| GPLNGRSDNHRR | 284 |
| GPLSGRSDNHKA | 285 |
| GPLSGRSDNHKK | 286 |
| GPLSGRSDNHKR | 287 |
| GPLSGRSDNHQA | 288 |
| GPLSGRSDNHQK | 289 |
| GPLSGRSDNHQR | 290 |
| GPLSGRSDNHRA | 291 |
| GPLSGRSDNHRK | 292 |
| GPLSGRSDNHRR | 293 |
| GPPLGR | 294 |
| GPQGIAGQ | 295 |
| GPQGLLGA | 296 |
| GPRSFG | 297 |
| GPRSFGL | 298 |
| GPSHLVLT | 299 |
| GPTN | 300 |
| GPTNALAHGLF | 301 |
| GRSML | 302 |
| GRSMLL | 303 |
| GRSMLLG | 304 |
| GRSMLLGG | 305 |
| GRSMLLGP | 306 |
| GRSMLLGS | 307 |
| GRSMLLP | 308 |
| GRSMLLPG | 309 |
| GRSMLLPP | 310 |
| GRSMLLPS | 311 |
| GRSMLLS | 312 |
| GRSMLLSG | 313 |
| GRSMLLSP | 314 |
| GRSMLLSS | 315 |
| GRSMLM | 316 |
| GRSMLMG | 317 |
| GRSMLMGG | 318 |
| GRSMLMGP | 319 |
| GRSMLMGS | 320 |
| GRSMLMP | 321 |
| GRSMLMPG | 322 |
| GRSMLMPP | 323 |
| GRSMLMPS | 324 |
| GRSMLMS | 325 |
| GRSMLMSG | 326 |
| GRSMLMSP | 327 |
| GRSMLMSS | 328 |
| GSGPPLGR | 329 |
| GSPPLGR | 330 |
| GSSPPLGR | 331 |
| GTGRGPSWVGSS | 332 |
| PTNL | 333 |
| PTNLGSGGSS | 334 |
| PVGYTSSL | 335 |
| PVPRLKLIKD | 336 |
| PVQPIGPQ | 337 |
| QALAMSAI | 338 |
| QFQALRM | 339 |
| QGPMFKSLWD | 340 |
| QGRAITFI | 341 |
| QHQALRM | 342 |
| QNQALRIA | 343 |
| QNQALRM | 344 |
| QNQALRMA | 345 |
| QNQALRMAGGSGGSLSGRSDNH | 346 |
| QNQALRMAGGSGGSLSGRSGNH | 347 |
| QSRRVP | 348 |
| QSRRVPL | 349 |
| QSRRVPV | 350 |
| QTRRVP | 351 |
| QTRRVPL | 352 |
| QTRRVPV | 353 |
| QYIVSRSA | 354 |
| RALRAP | 355 |
| REPFMKSLPW | 356 |
| RFPLKV | 357 |
| RFPSLKSFPL | 358 |
| RFPYGVW | 359 |
| RFYRNQFF | 360 |
| RGPA | 361 |
| RGPAFNPM | 362 |
| RGPATPIM | 363 |
| RGPKLYW | 364 |
| RHLAKL | 365 |
| RIGRSDNH | 366 |
| RKMPNITV | 367 |
| RKSSIIIRMRDVVL | 368 |
| RKTVQHWW | 369 |
| RLGRSDNN | 370 |
| RMHLRSLG | 371 |
| RPLARAGI | 372 |
| RPLARAGL | 373 |
| RPLNGRSDNHKA | 374 |
| RPLNGRSDNHKK | 375 |
| RPLNGRSDNHKR | 376 |
| RPLNGRSDNHQA | 377 |
| RPLNGRSDNHQK | 378 |
| RPLNGRSDNHQR | 379 |
| RPLNGRSDNHRA | 380 |
| RPLNGRSDNHRK | 381 |
| RPLNGRSDNHRR | 382 |
| RPLSGRSDNHKA | 383 |
| RPLSGRSDNHKK | 384 |
| RPLSGRSDNHKR | 385 |
| RPLSGRSDNHQA | 386 |
| RPLSGRSDNHQK | 387 |
| RPLSGRSDNHQR | 388 |
| RPLSGRSDNHRA | 389 |
| RPLSGRSDNHRK | 390 |
| RPLSGRSDNHRR | 391 |
| RPSPMWAY | 392 |
| RRHDGLRA | 393 |
| RRHDGLRS | 394 |
| RSLVFAPI | 395 |
| RSPSRLKC | 396 |
| VAGRSMRP | 397 |
| VAPQLKSLVP | 398 |
| VAQFVLTE | 399 |
| VHMPLGFLGP | 400 |
| VHMPLGFLGPGGLSGRSDNH | 401 |
| VHMPLGFLGPGGTSTSGRSANP | 402 |
| RG | |
| VLPELRSVFS | 403 |
| VLSKQMSF | 404 |
| VPAGRRS | 405 |
| VPAGRRSL | 406 |
| VPRQ | 407 |
| VPRQV | 408 |
| VSRSA | 40 |
| VVPEGRRS | 410 |
| WATPRPMR | 411 |
| WDHPISLL | 412 |
| XXQAR(A/V)X | |
| YDPZVKVVLA | 413 |
| YGAGLGVV | 414 |
| YIVSRSA | 415 |
| HMMQYARH | 418 |
| HTGRSGAL | 419 |
| HVPRQ | 420 |
| HVPRQV | 421 |
| HVPRQVAPRSF | 422 |
| HVPRQVLSGRS | 423 |
| HVPRQVLSGRSAN | 424 |
| HWHLGPPT | 425 |
| IANLLSMV | 426 |
| IDGR | 427 |
| IEGR | 428 |
| ILNLLSMV | 429 |
| ILPRSPAF | 430 |
| IPFSWSRF | 431 |
| IQNLLSMV | 432 |
| ISSGL | 433 |
| ISSGLL | 434 |
| ISSGLLS | 435 |
| ISSGLLSGRSANI | 436 |
| ISSGLLSGRSANP | 437 |
| ISSGLLSGRSANPRG | 438 |
| ISSGLLSGRSDDH | 439 |
| ISSGLLSGRSDIH | 440 |
| ISSGLLSGRSDNH | 666 |
| ISSGLLSGRSDNI | 441 |
| ISSGLLSGRSDNP | 442 |
| ISSGLLSGRSDQH | 443 |
| ISSGLLSGRSDTH | 444 |
| ISSGLLSGRSDYH | 445 |
| ISSGLLSGRSGNH | 446 |
| ISSGLLSGRSNI | 447 |
| ISSGLLSGRSNIG | 448 |
| ISSGLLSGRSNIGS | 449 |
| ISSGLLSS | 450 |
| ISSGLLSSGGSGGSLSGRSDNH | 451 |
| ISSGLLSSGGSGGSLSGRSGNH | 452 |
| ISSGLSS | 453 |
| IVSRSA | 454 |
| KGLTGRSDRHQA | 455 |
| KGPKVKVVTL | 456 |
| KNLYGRSENNGN | 457 |
| KRMPVQFL | 458 |
| LAAPLGLL | 459 |
| LAHG | 460 |
| LAHGL | 461 |
| LAHGLF | 462 |
| LAPLGLQRR | 463 |
| LARAG | 464 |
| LARAGI | 465 |
| LARAGL | 466 |
| LKAAPRWA | 467 |
| LKAAPRWF | 468 |
| LKAAPVWA | 469 |
| LKAAPVWF | 470 |
| LKGRSYYY | 471 |
| LLAPSHRA | 472 |
| LLEALRAL | 473 |
| LLESLRAL | 474 |
| LLLPAHGG | 475 |
| LLLPLLGS | 476 |
| LLNALRAL | 477 |
| LLNSLRAL | 478 |
| LLQALRAL | 479 |
| LLQSLRAL | 480 |
| LLSALRAL | 481 |
| LLSSLRAL | 482 |
| LNGRSDNH | 483 |
| LPAGLLL | 484 |
| LPAGLLLR | 485 |
| LPAHLVLL | 486 |
| LPAHLVLV | 487 |
| LPGGLSPW | 488 |
| LPSHLVLL | 489 |
| LPSHLVLV | 490 |
| LPTFV | 491 |
| LPTFVH | 492 |
| LPTFVHL | 493 |
| LRSGW | 494 |
| LSGR | 495 |
| LSGRS | 496 |
| LSGRSA | 497 |
| LSGRSALAHGLF | 498 |
| LSGRSAN | 499 |
| LSGRSANI | 500 |
| LSGRSANP | 501 |
| LSGRSD | 502 |
| LSGRSDD | 503 |
| LSGRSDDH | 665 |
| LSGRSDI | 504 |
| LSGRSDIH | 505 |
| LSGRSDN | 506 |
| LSGRSDNH | 507 |
| LSGRSDNHGGAVGLLAPP | 508 |
| LSGRSDNHGGSGGSISSGLLSS | 509 |
| LSGRSDNHGGSGGSQNQALRMA | 510 |
| LSGRSDNHGGVHMPLGFLGP | 511 |
| LSGRSDNI | 512 |
| LSGRSDNP | 513 |
| LSGRSDQ | 514 |
| LSGRSDQH | 515 |
| LSGRSDT | 516 |
| LSGRSDTH | 517 |
| LSGRSDY | 518 |
| LSGRSDYH | 519 |
| LSGRSENH | 520 |
| LSGRSG | 52 |
| LSGRSGN | 522 |
| LSGRSGNH | 523 |
| LSGRSGNHGGSGGSISSGLLSS | 524 |
| LSGRSGNHGGSGGSQNQALRMA | 525 |
| LSGRSGNP | 526 |
| LSGRSVTQ | 527 |
| LSQARWRK | 528 |
| LTFPTYIF | 529 |
| LTFPTYWF | 530 |
| LTGRSDRH | 531 |
| LTGRSGA | 532 |
| LTGRSGA | 533 |
| LYAAPRWA | 534 |
| LYAAPRWF | 535 |
| LYAAPVWA | 536 |
| LYAAPVWF | 537 |
| LYGRSENN | 538 |
| MDAFLESS | 539 |
| MGLFSEAG | 540 |
| MGPWFM | 541 |
| MIAPVAYR | 542 |
| MLRSGW | 543 |
| MLRSGWR | 544 |
| MLRSGWRG | 545 |
| MLRSGWRL | 546 |
| MLRSGWRS | 547 |
| MTFPTYIF | 548 |
| MTFPTYWF | 549 |
| NHRIGRSDNHRR | 550 |
| NMPSFKLVTG | 551 |
| NTLSGRSENHSG | 552 |
| NTLSGRSGNHGS | 553 |
| NZPRVRLVLP | 554 |
| PAGLWLDP | 555 |
| PAGRR | 556 |
| PAGRRS | 557 |
| PAGRRSL | 558 |
| PASLWYTQ | 559 |
| PESRRWMP | 560 |
| PFHLSR | 561 |
| PHGFFQ | 562 |
| PLARAGI | 563 |
| PLARAGL | 564 |
| PLGL | 565 |
| PLGLAG | 566 |
| PLGLWA | 567 |
| PLGVRGK | 568 |
| PLTGRSGG | 569 |
| PLTGRSGGGGSS | 570 |
| PPLGR | 571 |
| PPPDMKLFPG | 572 |
| PPPEVRSFSV | 573 |
| PPPVLKLLEW | 574 |
| PPSIARSDNLAN | 575 |
| PQHRIVSF | 576 |
| PRFKIIGG | 577 |
| PRFRIIGG | 578 |
| PRPFVKSVDQ | 579 |
| PRQV | 580 |
| PRSF | 581 |
| PSPPVKMMPE | 582 |
| PTNGGSGGSS | 583 |
| RVPKVKVMLD | 584 |
| SAGFSLPA | 585 |
| SAPAVESE | 586 |
| SAPYFRMMDM | 587 |
| SARGPSRW | 588 |
| SCGPPLGR | 589 |
| SCSPPLGR | 590 |
| SGGPLGVR | 591 |
| SGGPPLGR | 592 |
| SGPPLGR | 593 |
| SGRS | 594 |
| SGRSA | 595 |
| SGRSAN | 596 |
| SGRSANI | 597 |
| SGRSANP | 598 |
| SGRSANPRG | 599 |
| SGRSD | 600 |
| SGRSDD | 601 |
| SGRSDDH | 602 |
| SGRSDI | 603 |
| SGRSDIH | 604 |
| SGRSDN | 605 |
| SGRSDNI | 606 |
| SGRSDNP | 607 |
| SGRSDQ | 608 |
| SGRSDQH | 609 |
| SGRSDT | 610 |
| SGRSDTH | 611 |
| SGRSDY | 612 |
| SGRSDYH | 613 |
| SGRSG | 614 |
| SGRSGN | 615 |
| SGRSGNH | 616 |
| SGSPPLGR | 617 |
| SIARSDNL | 618 |
| SISSGLLSGRSDNI | 619 |
| SNPFKY | 620 |
| SPLPLRVP | 621 |
| SPLTGRSG | 622 |
| SPPLGR | 623 |
| SRRVP | 624 |
| SRRVPL | 625 |
| SRRVPV | 626 |
| SSGPPLGR | 627 |
| SSPPLGR | 628 |
| SSRGPAYL | 629 |
| SSRHRRALD | 630 |
| SSSFDKGKYKKGDDA | 631 |
| SSSFDKGKYKRGDDA | 632 |
| SSSPPLGR | 633 |
| STFPFGMF | 634 |
| STVFHM | 635 |
| SVHHLI | 636 |
| SVSGLLSH | 637 |
| SVSGLLSS | 638 |
| SVSGLRSH | 639 |
| SVSGLRSS | 640 |
| TARG | 641 |
| TARGP | 642 |
| TARGPALAHGLF | 643 |
| TARGPS | 644 |
| TARGPSF | 645 |
| TARGPSFK | 646 |
| TARGPSW | 647 |
| TARGPSW | 648 |
| TARGPVPRQV | 649 |
| TFVH | 650 |
| TGLSGRSVTQTS | 651 |
| TGRGPSWV | 652 |
| TLRLGRSDNNKN | 653 |
| TLSGLRSP | 654 |
| TSGRSANP | 655 |
| TSGRSGNP | 656 |
| TSLSGRSANPRG | 657 |
| TSLSGRSGNPRG | 658 |
| TSSGLRSP | 659 |
| TSTSGRSANPRG | 660 |
| TSTSGRSANPRGGGAVGLLAPP | 661 |
| TSTSGRSANPRGGGVHMPLGFLGP | 662 |
| TSTSGRSGNPRG | 663 |
| TVSGLRSP | 664 |
| YVPRVKALEM | 417 |
| YKKFVGSL | 416 |

Examples of CMs also include those described in WO 2010/081173, WO2021207669, WO2021207657, WO2021142029, WO2021061867, WO2020252349, WO2020252358, WO2020236679, WO2020176672, WO2020118109, WO2020092881, WO2020086665, WO2019213444, WO2019183218, WO2019173771, WO2019165143, WO2019075405, WO2019046652, WO2019018828, WO2019014586, WO2018222949, WO2018165619, WO2018085555, WO2017011580, WO2016179335, WO2016179285, WO2016179257, WO2016149201, WO2016014974, which are incorporated herein by reference in their entireties for all purposes.

In some embodiments, the CM may be or comprise a combination, a C-terminal truncation variant, or an N-terminal truncation variant of the example sequences discussed above. Truncation variants of the aforementioned amino acid sequences that are suitable for use in a CM may be any that retain the recognition site for the corresponding protease. These include C-terminal and/or N-terminal truncation variants comprising at least 3 contiguous amino acids of the above-described amino acid sequences, or at least 4, 5, 6, 7, 8, 9, or 10 amino acids of the foregoing amino acid sequences that retain a recognition site for a protease. In certain embodiments, the truncation variant of the above-described amino acid sequences may be an amino acid sequence corresponding to any of the above, but that is C- and/or N-terminally truncated by 1 to 10 amino acids, 1 to 9 amino acids, 1 to 8 amino acids, 1 to 7 amino acids, 1 to 6 amino acids, 1 to 5 amino acids, 1 to 4 amino acids, or 1 to 3 amino acids, and which: (1) has at least three amino acid residues; and (2) retains a recognition site for a protease. In some of the foregoing embodiments, the truncated CM is an N-terminally truncated CM. In some embodiments, the truncated CM is a C-terminally truncated CM. In some embodiments, the truncated C is a C- and an N-terminally truncated CM.

In some embodiments, the CM may comprise a total of 3 amino acids to 25 amino acids. In some embodiments, the CM may comprise a total of 3 to 25, 3 to 20, 3 to 15, 3 to 10, 3 to 5, 5 to 25, 5 to 20, 5 to 15, 5 to 10, 10 to 25, 10 to 20, 10 to 15, 15 to 25, 15 to 20, or 20 to 25 amino acids.

In some embodiments, the CM may be specifically cleaved by at least a protease at a rate of about 0.001-1500×10⁴ M⁻¹S⁻¹ or at least 0.001, 0.005, 0.01, 0.05, 0.1, 0.5, 1, 2.5, 5, 7.5, 10, 15, 20, 25, 50, 75, 100, 125, 150, 200, 250, 500, 750, 1000, 1250, or 1500×10⁴ M⁻¹S⁻¹. The rate may be measured as substrate cleavage kinetics (k_{cat}/Kₘ) as disclosed in WO2016118629.

### Conjugation Agents

In some aspects, the target-binding proteins (including the activatable target-binding proteins) may further comprise one or more additional agents, e.g., a targeting moiety to facilitate delivery to a cell or tissue of interest, a therapeutic agent (e.g., an antineoplastic agent such as chemotherapeutic or anti-neoplastic agent), a toxin, a radioisotope, a small molecule, a diagnostic agent, a targeting moiety, or a detectable moiety, or a fragment thereof. The additional agents may be conjugated to the target-binding proteins. The term “agent” is used herein to denote a chemical compound, a mixture of chemical compounds, a biological macromolecule, or an extract made from biological materials.

In some embodiments, the target-binding protein may be conjugated to a cytotoxic agent, e.g., a toxin (e.g., an enzymatically active toxin of bacterial, fungal, plant, or animal origin, or fragments thereof) or a radioactive isotope. In some embodiments, the target-binding protein may be conjugated to a T-cell activator such as, for example, small molecule agonists of toll-like receptors (TLRs), including TLR3, TLR7, TLR8 and TLR9. Non-limiting examples of such activators include: TLR3 agonists, such as (Riboxxol), RGC100, ARNAX, and poly-IC; TLR7/8 agonists, such as Resiquimod (R848) and motolimod (VTX-2337) (second-generation experimental derivatives of imiquimod, an imidazoquinoline), PF-4878691, BDC-1001, LHC165, NKTR-262, TQ-A3334, R07119929, DSP-0509, BNT411, and NJH395; TLR9 agonists, such as Bacillus Calmette-Gudrin (BCG), Cavrotolimod/AST-008 (Exicure), CMP-001 (Checkmate), CpG-28 (University of Paris), EnanDIM (Mologen AG), IMO-2055 (Idera), IMO-2125/tilsotolimod) (Idera), MGN1703/Lefitolimod (Mologen AG), NZ-TLR9 (LIDDS), PF-3512676 (Pfizer), SD-101 (Dynavax), and S-540956 (Shionogi).

Examples of cytotoxic agents that can be conjugated to the target-binding proteins include: dolastatins and derivatives thereof (e.g., auristatin E, AFP, monomethyl auristatin D (MMAD), monomethyl auristatin F (MMAF), monomethyl auristatin E (MMAE), desmethyl auristatin E (DMAE), auristatin F, desmethyl auristatin F (DMAF), dolastatin 16 (DmJ), dolastatin 16 (Dpv), auristatin derivatives (e.g., auristatin tyramine, auristatin quinolone), maytansinoids (e.g., DM-1, DM-4), maytansinoid derivatives, duocarmycin, alpha-amanitin, turbostatin, phenstatin, hydroxyphenstatin, spongistatin 5, spongistatin 7, halistatin 1, halistatin 2, halistatin 3, halocomstatin, pyrrolobenzimidazoles (PBI), cibrostatin6, doxaliform, cemadotin analogue (CemCH2-SH), Pseudomonas toxin A (PES8) variant, Pseudomonase toxin A (ZZ-PE38) variant, ZJ-101, anthracycline, doxorubicin, daunorubicin, bryostatin, camptothecin, 7-substituted campothecin, 10, 11-difluoromethylenedioxycamptothecin, combretastatins, debromoaplysiatoxin, KahaMide-F, discodermolide, and Ecteinascidins.

Examples of enzymatically active toxins that can be conjugated to the target-binding proteins include: diphtheria toxin, exotoxin A chain from Pseudomonas aeruginosa, ricin A chain, abrin A chain, modeccin A chain, alpha-sarcin, Aleuriies fordii proteins, dianfhin proteins, Phytoiaca Americana proteins (e.g., PAPI, PAPII, and PAP-8), Momordica charantia inhibitor, curcin, crotirs, Sapaonaria officinalis inhibitor, geionin, mitogeliin, restrictocin, phenomycin, neomycin, and tricothecenes.

Examples of anti-neoplastics that can be conjugated to the target-binding proteins include: adriamycin, cerubidine, bleomycin, alkeran, velban, oncovin, fluorouracil, methotrexate, thiotepa, bisantrene, novantrone, thioguanine, procarabizine, and cytarabine.

Examples of antivirals that can be conjugated to the target-binding proteins include: acyclovir, vira A, and symmetrel. Examples of antifungals that can be conjugated to the target-binding proteins include: nystatin. Examples of detection reagents that can be conjugated to the target-binding proteins include: fluorescein and derivatives thereof, fluorescein isothiocyanate (FITC). Examples of antibacterials that can be conjugated to the target-binding proteins include: aminoglycosides, streptomycin, neomycin, kanamycin, amikacin, gentamicin, and tobramycin. Examples of 3beta,16beta,17alpha-trihydroxycholest-5-en-22-one 16-O-(2-O-4-methoxybenzoyl-beta-D-xylopyranosyl)-(1-->3)-(2-O-acetyl-alpha-L-arabinopyranoside) (OSW-1) that can be conjugated to the target-binding proteins include: s-nitrobenzyloxycarbonyl derivatives of O6-benzylguanine, toposisomerase inhibitors, hemiasterlin, cephalotaxine, homoharringionine, pyrrol obenzodiazepine dimers (PBDs), functionalized pyrrolobenzodiazepenes, calcicheamicins, podophyiitoxins, taxanes, and vinca alkoids. Examples of radiopharmaceuticals that can be conjugated to the target-binding proteins include: ¹²³I, ⁸⁹Zr, ¹²⁵I, ¹³¹I, ⁹⁹mTc, ²⁰¹Tl, ⁶²Cu, ¹⁸F, ⁶⁸Ga, ¹³N, ¹⁵O, ³⁸K, ⁸²Rb, ¹¹¹In, ¹³³Xe, ¹¹C, and ⁹⁹mTc (Technetium). Examples of heavy metals that can be conjugated to the target-binding proteins include: barium, gold, and platinum. Examples of anti-mycoplasmals that can be conjugated to the target-binding proteins include: tylosine, spectinomycin, streptomycin B, ampicillin, sulfanilamide, polymyxin, and chloramphenicol.

In some embodiments, the target-binding protein may comprise a signal peptide. If comprising multiple peptides, the target-binding protein may comprise multiple signal peptides, e.g., one signal peptide for each of the multiple peptides. A signal peptide may be a peptide (e.g., 10-30 amino acids long) present at a terminus (e.g., the N-terminus or C-terminus) of a newly synthesized proteins that are destined toward the secretory pathway. In some embodiments, the signal peptide may be conjugated to the target-binding protein via a spacer. In some embodiments, the spacer may be conjugated to the target-binding protein in the absence of a signal peptide.

Those of ordinary skill in the art will recognize that a large variety of possible agents may be conjugated to any of the target-binding proteins described herein. The agents may be conjugated to another component of the target-binding protein by a conjugating moiety, which may be a linker, a CM, or other molecule or fragment thereof capable linking two molecules. In some examples, the conjugating moiety may be cleavable by an enzyme (e.g., a protease). In some examples, the conjugating moiety may be uncleavable by an enzyme *(e.g., a protease).

Conjugation may include any chemical reaction that binds the two molecules so long as the target-binding protein and the other moiety retain their respective activities. Conjugation may include many chemical mechanisms, e.g., covalent binding, affinity binding, intercalation, coordinate binding, and complexation. In some embodiments, the binding may be covalent binding. Covalent binding may be achieved either by direct condensation of existing side chains or by the incorporation of external bridging molecules. Many bivalent or polyvalent linking agents may be useful in conjugating any of the target-binding proteins described herein. For example, conjugation may include organic compounds, such as thioesters, carbodiimides, succinimide esters, glutaraldehyde, diazobenzenes, and hexamethylene diamines. In some embodiments, the target-binding proteins may include, or otherwise introduce, one or more non-natural amino acid residues to provide suitable sites for conjugation.

In some embodiments, an agent and/or conjugate may be attached by disulfide bonds (e.g., disulfide bonds on a cysteine molecule) to the antigen-binding domain. Since many cancers naturally release high levels of glutathione, a reducing agent, glutathione present in the cancerous tissue microenvironment can reduce the disulfide bonds, and subsequently release the agent and/or the conjugate at the site of delivery.

In some embodiments, when the conjugate binds to its target in the presence of complement within the target site (e.g., diseased tissue (e.g., cancerous tissue)), the amide or ester bond attaching the conjugate and/or agent to the linker is cleaved, resulting in the release of the conjugate and/or agent in its active form. These conjugates and/or agents when administered to a subject, may accomplish delivery and release of the conjugate and/or the agent at the target site (e.g., diseased tissue (e.g., cancerous tissue)). These conjugates and/or agents may be effective for the in vivo delivery of any of the conjugates and/or agents described herein.

In some embodiments, the conjugating moiety may be uncleavable by enzymes of the complement system. For example, the conjugate and/or agent is released without complement activation since complement activation ultimately lyses the target cell. In such embodiments, the conjugate and/or agent is to be delivered to the target cell (e.g., hormones, enzymes, corticosteroids, neurotransmitters, or genes). Furthermore, the conjugating moiety may be mildly susceptible to cleavage by serum proteases, and the conjugate and/or agent is released slowly at the target site.

In some embodiments, the conjugate and/or agent may be designed such that the conjugate and/or agent is delivered to the target site (e.g., disease tissue (e.g., cancerous tissue)) but the conjugate and/or agent is not released.

In some embodiments, the conjugate and/or agent may be attached to an antigen-binding domain either directly or via amino acids (e.g., D-amino acids), peptides, thiol-containing moieties, or other organic compounds that may be modified to include functional groups that can subsequently be utilized in attachment to antigen-binding domains by methods described herein.

In some embodiments, a target-binding protein may include at least one point of conjugation for an agent. In some embodiments, all possible points of conjugation are available for conjugation to an agent. In some embodiments, the one or more points of conjugation may include sulfur atoms involved in disulfide bonds, sulfur atoms involved in interchain disulfide bonds, sulfur atoms involved in interchain sulfide bonds but not sulfur atoms involved in intrachain disulfide bonds, and/or sulfur atoms of cysteine or other amino acid residues containing a sulfur atom. In such cases, residues may occur naturally in the protein construct structure or may be incorporated into the protein construct using methods including site-directed mutagenesis, chemical conversion, or mis-incorporation of non-natural amino acids.

The present disclosure also provides methods and materials for preparing a target-binding protein with one or more conjugated agents. In some embodiments, a target-binding protein may be modified to include one or more interchain disulfide bonds. For example, disulfide bonds may undergo reduction following exposure to a reducing agent such as, without limitation, TCEP, DTT, or β-mercaptoethanol. In some cases, the reduction of the disulfide bonds may be only partial. As used herein, the term partial reduction refers to situations where an target-binding protein is contacted with a reducing agent and a fraction of all possible sites of conjugation undergo reduction (e.g., not all disulfide bonds are reduced). In some embodiments, an target-binding protein may be partially reduced following contact with a reducing agent if less than 99%, (e.g., less than 98%, 97%, 96%, 95%, 90%, 85%, 80%, 75%, 70%, 65%, 60%, 55%, 50%, 45%, 40%, 35%, 30%, 25%, 20%, 15%, 10% or 5%) of all possible sites of conjugation are reduced. In some embodiments, the target-binding protein having a reduction in one or more interchain disulfide bonds may be conjugated to a drug reactive with free thiols.

The present disclosure also provides methods and materials for conjugating a therapeutic agent to a particular location on a target-binding protein. In some embodiments, a target-binding protein may be modified so that the therapeutic agents can be conjugated to the target-binding protein at particular locations on the target-binding protein. For example, a target-binding protein may be partially reduced in a manner that facilitates conjugation to the target-binding protein. In such cases, partial reduction of the target-binding protein may occur in a manner that conjugation sites in the target-binding protein are not reduced. In some embodiments, the conjugation site(s) on the target-binding protein may be selected to facilitate conjugation of an agent at a particular location on the protein construct. Various factors can influence the “level of reduction” of the target-binding protein upon treatment with a reducing agent. For example, without limitation, the ratio of reducing agent to the target-binding protein, length of incubation, incubation temperature, and/or pH of the reducing reaction solution can require optimization in order to achieve partial reduction of the target-binding protein with the methods and materials described herein. Any appropriate combination of factors (e.g., ratio of reducing agent to target-binding protein, the length and temperature of incubation with reducing agent, and/or pH of reducing agent) may be used to achieve partial reduction of the target-binding protein (e.g., general reduction of possible conjugation sites or reduction at specific conjugation sites).

An effective ratio of reducing agent to target-binding protein can be any ratio that at least partially reduces the target-binding protein in a manner that allows conjugation to an agent (e.g., general reduction of possible conjugation sites or reduction at specific conjugation sites). In some embodiments, the ratio of reducing agent to target-binding protein may be in a range from about 20:1 to 1:1, from 10:1 to 1:1, from 9:1 to 1:1, from 8:1 to 1:1, from 7:1 to 1:1, from 6:1 to 1:1, from 5:1 to 1:1, from 4:1 to 1:1, from 3:1 to 1:1, from 2:1 to 1:1, from 20:1 to 1:1.5, from 10:1 to 1:1.5, from 9:1 to 1:1.5, from 8:1 to 1:1.5, from 7:1 to 1:1.5, from 6:1 to 1:1.5, from 5:1 to 1:1.5, from 4:1 to 1:1.5, from 3:1 to 1:1.5, from 2:1 to 1:1.5, from 1.5:1 to 1:1.5, or from 1:1 to 1:1.5.

An effective incubation time and temperature for treating a target-binding protein with a reducing agent may be any time and temperature that at least partially reduces the target-binding protein in a manner that allows conjugation of an agent to a target-binding protein (e.g., general reduction of possible conjugation sites or reduction at specific conjugation sites). In some embodiments, the incubation time and temperature for treating an target-binding protein may be in a range from about 1 hour at 37° C. to about 12 hours at 37° C. (or any subranges therein).

An effective pH for a reduction reaction for treating a target-binding protein with a reducing agent can be any pH that at least partially reduces the target-binding protein in a manner that allows conjugation of the target-binding protein to an agent (e.g., general reduction of possible conjugation sites or reduction at specific conjugation sites).

When a partially-reduced target-binding protein is contacted with an agent containing thiols, the agent may conjugate to the interchain thiols in the target-binding protein. An agent can be modified in a manner to include thiols using a thiol-containing reagent (e.g., cysteine or N-acetyl cysteine). For example, the target-binding protein can be partially reduced following incubation with reducing agent (e.g., TEPC) for about 1 hour at about 37° C. at a desired ratio of reducing agent to target-binding protein. An effective ratio of reducing agent to target-binding protein may be any ratio that partially reduces at least two interchain disulfide bonds located in the target-binding protein in a manner that allows conjugation of a thiol-containing agent (e.g., general reduction of possible conjugation sites or reduction at specific conjugation sites).

In some embodiments, a target-binding protein may be reduced by a reducing agent in a manner that avoids reducing any intrachain disulfide bonds. In some embodiments of, a target-binding protein may be reduced by a reducing agent in a manner that avoids reducing any intrachain disulfide bonds and reduces at least one interchain disulfide bond.

In some embodiments, the agent (e.g., agent conjugated to a target-binding protein) may be a detectable moiety such as, for example, a label or other marker. For example, the agent may be or include a radiolabeled amino acid, one or more biotinyl moieties that can be detected by marked avidin (e.g., streptavidin containing a fluorescent marker or enzymatic activity that can be detected by optical or calorimetric methods), one or more radioisotopes or radionuclides, one or more fluorescent labels, one or more enzymatic labels, and/or one or more chemiluminescent agents. In some embodiments, detectable moieties may be attached by spacer molecules. In some embodiments, the detectable label may include an imaging agent, a contrasting agent, an enzyme, a fluorescent label, a chromophore, a dye, one or more metal ions, or a ligand-based label. In some embodiments, the imaging agent may comprise a radioisotope. In some embodiments, the radioisotope may be indium or technetium. In some embodiments, the contrasting agent may comprise iodine, gadolinium or iron oxide. In some embodiments, the enzyme may comprise horseradish peroxidase, alkaline phosphatase, or β-galactosidase. In some embodiments, the fluorescent label may comprise yellow fluorescent protein (YFP), cyan fluorescent protein (CFP), green fluorescent protein (GFP), modified red fluorescent protein (mRFP), red fluorescent protein tdimer2 (RFP tdimer2), HCRED, or a europium derivative. In some embodiments, the luminescent label may comprise an N-methylacrydium derivative. In some embodiments, the label may comprise an Alexa Fluor® label, such as Alex Fluor® 680 or Alexa Fluor® 750. In some embodiments, the ligand-based label may comprise biotin, avidin, streptavidin or one or more haptens.

Further examples of detectable labels also include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In some embodiments, the agent may be conjugated to the target-binding protein using a carbohydrate moiety, sulfhydryl group, amino group, or carboxylate group. In some embodiments, the agent may be conjugated to the target-binding protein via a linker and/or a CM described herein. In some embodiments, the agent may be conjugated to a cysteine or a lysine in the target-binding protein. In some embodiments, the agent may be conjugated to another residue of the target-binding protein, such as those residues disclosed herein.

In some embodiments, a variety of bifunctional protein-coupling agents may be used to conjugate the agent to the target-binding protein including N-succinimidyl-3-(2-pyridyldithiol) propionate (SPDP), iminothiolane (IT), bifunctional derivatives of imidoesters (e.g., dimethyl adipimidate HCL), active esters (e.g., disuccinimidyl suberate), aldehydes (e.g., glutareldehyde), bis-azido compounds (e.g., bis (p-azidobenzoyl) hexanediamine), bis-diazonium derivatives (e.g., bis-(p-diazoniumbenzoyl)-ethylenediamine), diisocyanates (e.g., tolyene 2,6-diisocyanate), and bis-active fluorine compounds (e.g., 1,5-difluoro-2,4-dinitrobenzene). For example, a ricin immunotoxin can be prepared as described in Vitetta et al., Science 238: 1098 (1987). In some embodiments, a carbon-14-labeled 1-isothiocyanatobenzyl-3-methyldiethylene triaminepentaacetic acid (MX-DTPA) chelating agent can be used to conjugate a radionucleotide to the target-binding protein. (See, e.g., WO94/11026).

Suitable conjugation moieties include those described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to a target-binding protein by way of an oligopeptide. In some embodiments, suitable conjugation moieties include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS (N-hydroxysulfo-succinimide: Pierce Chem. Co., Cat. #24510) conjugated to EDC. Additional example conjugation moieties include SMCC, sulfo-SMCC, SPDB, and sulfo-SPDB.

The conjugation moieties described above may contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the SMPT contains a sterically-hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodiimide couplings. Carbodiimide couplings (such as EDC) when used in conjunction with sulfo-NHS, forms esters that are more resistant to hydrolysis than the carbodiimide coupling reaction alone.

Those of ordinary skill in the art will recognize that a large variety of possible moieties can be coupled to the target-binding protein of the disclosure. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference). In general, an effective conjugation of an agent (e.g., cytotoxic agent) to a target-binding protein can be accomplished by any chemical reaction that will bind the agent to the target-binding protein while also allowing the agent and the target-binding protein to retain functionality.

### Nucleic Acids and Vectors

In some aspects, the present disclosure further provides nucleic acids comprising sequences that encode the target-binding proteins, or components or fragment thereof. The nucleic acids may comprise coding sequences for the heavy chain variable domains, light chain variable domains, TBs, the CMs, the MMs, the EM and the linker(s) in a target-binding protein. In cases where the target-binding protein comprises multiple peptides (e.g., multiple TBs on different peptides, or a TB comprises multiple peptides), the nucleic acid may comprise coding sequences for the multiple peptides. In some examples, the coding sequences for one of the peptides are disposed within one nucleic acid, and the coding sequences for another one of the peptides are disposed within another nucleic acid. In some examples, the coding sequences for two or more of the multiple peptides are disposed within the same nucleic acid. The present disclosure includes a polynucleotide encoding a protein as described herein or a portion thereof, and use of such polynucleotides to produce the proteins and/or for therapeutic purposes. Such polynucleotides may include DNA and RNA molecules (e.g., mRNA, self-replicating RNA, self-amplifying mRNA, etc.) that encode a protein as defined herein. The present disclosure includes compositions comprising such polynucleotides. In some aspects, such compositions may be used therapeutically or prophylactically.

Unless otherwise specified, a “nucleic acid sequence encoding a protein” includes all nucleotide sequences that are degenerate versions of each other and thus encode the same amino acid sequence. The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments, the nucleic acid is DNA. In some embodiments, the nucleic acid is RNA. In some embodiments, the nucleic acid comprise a mixture or hybrid of DNA and RNA.

The term “N-terminally positioned” when referring to a position of a first domain or sequence relative to a second domain or sequence in a polypeptide primary amino acid sequence means that the first domain is located closer to the N-terminus of the polypeptide primary amino acid sequence. In some embodiments, there may be additional sequences and/or domains between the first domain or sequence and the second domain or sequence. The term “C-terminally positioned” when referring to a position of a first domain or sequence relative to a second domain or sequence in a polypeptide primary amino acid sequence means that the first domain is located closer to the C-terminus of the polypeptide primary amino acid sequence. In some embodiments, there may be additional sequences and/or domains between the first domain or sequence and the second domain or sequence.

Modifications may be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include: amino acids with acidic side chains (e.g., aspartate and glutamate), amino acids with basic side chains (e.g., lysine, arginine, and histidine), non-polar amino acids (e.g., alanine, valine, leucine, isoleucine, proline, phenylalanine, methionine, and tryptophan), uncharged polar amino acids (e.g., glycine, asparagine, glutamine, cysteine, serine, threonine and tyrosine), hydrophilic amino acids (e.g., arginine, asparagine, aspartate, glutamine, glutamate, histidine, lysine, serine, and threonine), hydrophobic amino acids (e.g., alanine, cysteine, isoleucine, leucine, methionine, phenylalanine, proline, tryptophan, tyrosine, and valine). Other families of amino acids include: aliphatic-hydroxy amino acids (e.g., serine and threonine), amide family (e.g., asparagine and glutamine), aliphatic family (e.g., alanine, valine, leucine and isoleucine), and aromatic family (e.g., phenylalanine, tryptophan, and tyrosine).

The present disclosure further provides vectors and sets of vectors comprising any of the nucleic acids described herein. One skilled in the art will be capable of selecting suitable vectors or sets of vectors (e.g., expression vectors) for making any of the target-binding proteins described herein, and using the vectors or sets of vectors to express any of the target-binding proteins described herein. For example, in selecting a vector or a set of vectors, the type of cell may be selected such that the vector(s) may need to be able to integrate into a chromosome of the cell and/or replicate in it. Example vectors that can be used to produce a target-binding protein are also described herein. As used herein, the term “vector” refers to a polynucleotide capable of inducing the expression of a recombinant protein (e.g., a first or second monomer) in a cell (e.g., any of the cells described herein). A “vector” is able to deliver nucleic acids and fragments thereof into a host cell, and includes regulatory sequences (e.g., promoter, enhancer, poly(A) signal). Exogenous polynucleotides may be inserted into the expression vector in order to be expressed. The term “vector” also includes artificial chromosomes, plasmids, retroviruses, and baculovirus vectors.

Methods for constructing suitable vectors that comprise any of the nucleic acids described herein, and suitable for transforming cells (e.g., mammalian cells) are well-known in the art. See, e.g., Sambrook et al., Eds. “Molecular Cloning: A Laboratory Manual,” 2^{nd} Ed., Cold Spring Harbor Press, 1989 and Ausubel et al., Eds. “Current Protocols in Molecular Biology,” Current Protocols, 1993.

Examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. A vector may, for example, include sufficient cis-acting elements for expression; other elements for expression may be supplied by the host mammalian cell or in an in vitro expression system. Skilled practitioners will be capable of selecting suitable vectors and mammalian cells for making any target-binding protein described herein.

In some embodiments, the target-binding protein may be made biosynthetically using recombinant DNA technology and expression in eukaryotic or prokaryotic species.

### Cells

In some aspects, the present disclosure provides host cells comprising any of the vectors or nucleic acids described herein. The cells may be used to produce the target-binding proteins described herein. In some embodiments, the cell may be an animal cell, a mammalian cell (e.g., a human cell), a rodent cell (e.g., a mouse cell, a rat cell, a hamster cell, or a guinea pig cell), a non-human primate cell, an insect cell, a bacterial cell, a fungal cell, or a plant cell. In some embodiments, the cell may be a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Non-limiting examples of mammalian cells include Chinese hamster ovary (CHO) cells and human embryonic kidney cells (e.g., HEK293 cells). In some embodiments, the cell may be a prokaryotic cell.

Methods of introducing nucleic acids and vectors (e.g., any of the vectors or any of the sets of vectors described herein) into a cell are known in the art. Examples of methods that can be used to introducing a nucleic acid into a cell include: lipofection, transfection, calcium phosphate transfection, cationic polymer transfection, viral transduction (e.g., adenoviral transduction, lentiviral transduction), nanoparticle transfection, and electroporation.

In some embodiments, the introducing step includes introducing into a cell a vector (e.g., any of the vectors or sets of vectors described herein) including a nucleic acid encoding the monomers that make up any target-binding protein described herein.

### Compositions and Kits

The present disclosure also provides compositions and kits comprising the target-binding proteins described herein. The compositions and kits may further comprise one or more excipients, carriers, reagents, instructions needed for the use of the target-binding proteins.

In some embodiments, the compositions may be pharmaceutical compositions, which comprise the target-binding proteins, derivatives, fragments, analogs and homologs thereof. The pharmaceutical compositions may comprise the target-binding protein and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Suitable examples of such carriers or diluents include water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition may be formulated to be compatible with its intended route of administration. Examples of routes of administration include parenteral, e.g., intravenous, intradermal, subcutaneous, oral (e.g., inhalation), transdermal (e.g., topical), transmucosal, and rectal administration. Solutions or suspensions used for parenteral, intradermal, or subcutaneous application may include one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH may be adjusted with acids or bases, such as hydrochloric acid or sodium hydroxide. The parenteral preparation can be enclosed in ampoules, disposable syringes or multiple dose vials made of glass or plastic. In some, any of the target-binding proteins described herein are prepared with carriers that protect against rapid elimination from the body, e.g., sustained and controlled release formulations, including implants and microencapsulated delivery systems. Biodegradable, biocompatible polymers can be used, e.g., ethylene vinyl acetate, polyanhydrides, polyglycolic acid, collage, polyorthoesters, and polylactic acid. Methods for preparation of such pharmaceutical compositions and formulations are apparent to those skilled in the art. For example, the target-binding proteins may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and y ethyl-L-glutamate, non-degradable ethylene-vinyl acetate, degradable lactic acid-glycolic acid copolymers (e.g., injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

In some embodiments, pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL^{□} (BASF, Parsippany, N.J.) or phosphate buffered saline (PBS). The composition may sterile and should be fluid to the extent that easy syringeability exists. It may be stable under the conditions of manufacture and storage and preserved against the contaminating action of microorganisms such as bacteria and fungi. The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In some embodiments, isotonic agents, for example, sugars, polyalcohols such as mannitol, sorbitol, and sodium chloride may be included in the composition. Prolonged absorption of the injectable compositions may be brought about by including in the composition an agent that delays absorption, for example, aluminum monostearate and gelatin.

In some embodiments, the pharmaceutical composition may comprise a sterile injectable solution. Sterile injectable solutions may be prepared by incorporating the active compound in the required amount in an appropriate solvent with one or a combination of ingredients enumerated above, as required, followed by filtered sterilization. Generally, dispersions may be prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In some embodiments, the pharmaceutical composition may comprise an oral composition. Oral compositions may include an inert diluent or an edible carrier. They may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents, and/or adjuvant materials may be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In some embodiments, the pharmaceutical composition may be formulized for administration by inhalation. For example, the compounds may be delivered in the form of an aerosol spray from pressured container or dispenser that contains a suitable propellant, e.g., a gas such as carbon dioxide, or a nebulizer.

In some embodiments, the pharmaceutical composition may be formulized for systemic administration. For example, systemic administration may be by intravenous, as well by transmucosal or transdermal means. For transmucosal or transdermal administration, penetrants appropriate to the barrier to be permeated may be used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, the active compounds may be formulated into ointments, salves, gels, or creams as generally known in the art.

In some embodiments, the pharmaceutical composition may be prepared in the form of suppositories (e.g., with conventional suppository bases such as cocoa butter and other glycerides) or retention enemas for rectal delivery.

In one embodiment, the pharmaceutical composition may be prepared with carriers that protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art.

It may be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure may be dictated by and directly dependent on the unique characteristics of the active compound and the particular therapeutic effect to be achieved, and the limitations inherent in the art of compounding such an active compound for the treatment of individuals.

In some embodiments, the compositions (e.g., pharmaceutical compositions) may be included in a container, vial, syringe, injector pen, pack, or dispenser, optionally together with instructions for administration.

Also provided herein are kits that include any of the target-binding proteins described herein, any of the compositions that include any of the target-binding proteins described herein, or any of the pharmaceutical compositions that include any of the target-binding proteins described herein. Also provided are kits that include one or more second therapeutic agent(s) in addition to a target-binding protein described herein. The second therapeutic agent(s) may be provided in a dosage administration form that is separate from the target-binding proteins. Alternatively, the second therapeutic agent(s) may be formulated together with the target-binding proteins.

Any of the kits described herein can include instructions for using any of the compositions (e.g., pharmaceutical compositions) and/or any of the target-binding proteins described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein.

Also provided herein are target-binding proteins produced by any of the methods described herein. Also provided are compositions (e.g., pharmaceutical compositions) that comprise any of the target-binding proteins produced by any of the methods described herein. Also provided herein are kits that include at least one dose of any of the compositions (e.g., pharmaceutical compositions) described herein.

### Methods of Producing Target-Binding Proteins

Provided herein are methods of producing any target-binding protein described herein that include: (a) culturing any of the recombinant host cells described herein in a liquid culture medium under conditions sufficient to produce the target-binding protein; and (b) recovering the target-binding protein from the host cell and/or the liquid culture medium.

Methods of culturing cells are well known in the art. In some embodiments, cells may be maintained in vitro under conditions that favor cell proliferation, cell differentiation and cell growth. For example, cells may be cultured by contacting a cell (e.g., any of the cells described herein) with a cell culture medium that includes the necessary growth factors and supplements sufficient to support cell viability and growth.

In some embodiments, the method may further includes isolating the recovered target-binding protein. The isolation of the target-binding protein may be performed using any protein separation or purification techniques, e.g., Examples of methods of isolation include: isolation using a protein purification tag (e.g., His tag), ammonium sulfate precipitation, polyethylene glycol precipitation, size exclusion chromatography, ligand-affinity chromatography, ion-exchange chromatography (e.g., anion or cation), and hydrophobic interaction chromatography.

Compositions and methods described herein may involve use of non-reducing or partially-reducing conditions that allow disulfide bonds to form between the MM and the TB of the target-binding proteins.

In some embodiments, the method further includes formulating the isolated target-binding protein into a pharmaceutical composition. Various formulations are known in the art and are described herein. Any isolated target-binding protein described herein can be formulated for any route of administration (e.g., intravenous, intratumoral, subcutaneous, intradermal, oral (e.g., inhalation), transdermal (e.g., topical), transmucosal, or intramuscular).

### Methods of Using Target-Binding Proteins

In some aspects, the present disclosure further provides methods of using the target-binding proteins herein. In some embodiments, the present disclosure provides methods of the treating a disease (e.g., a cancer (e.g., any of the cancers described herein)) in a subject including administering a therapeutically effective amount of any of the target-binding proteins described herein to the subject. In some embodiments, the disclosure provides methods of preventing, delaying the progression of, treating, alleviating a symptom of, or otherwise ameliorating disease in a subject by administering a therapeutically effective amount of an target-binding protein described herein to a subject in need thereof. The term “treatment” refers to ameliorating at least one symptom of a disorder. The disorder may be a cancer, autoimmune disease, infectious disease, chronic inflammation, or transplant rejection (e.g., in kidney, liver, or heart transplantation). In some embodiments, the disorder being treated is a cancer, autoimmune diseases (e.g., Type 1 diabetes, Rheumatoid arthritis (RA), Psoriasis/psoriatic arthritis, Multiple sclerosis, Systemic lupus erythematosus, Inflammatory bowel disease (e.g., Crohn's disease, ulcerative colitis), Addison's disease, Graves' disease, Sjögren's syndrome, Hashimoto's thyroiditis, Myasthenia gravis, Autoimmune vasculitis, Pernicious anemia, Celiac disease), or infectious disease (e.g., Chickenpox, Common cold, Diphtheria, E. coli, Giardiasis, HIV/AIDS, Infectious mononucleosis, Influenza (flu), Lyme disease, Malaria, Measles, Meningitis, Mumps, Poliomyelitis (polio), Pneumonia, Rocky mountain spotted fever, Rubella (German measles), Salmonella infections, Severe acute respiratory syndrome (SARS), Sexually transmitted diseases, Shingles (herpes zoster), Tetanus, Toxic shock syndrome, Tuberculosis, Viral hepatitis, West Nile virus, Whooping cough (pertussis)) and the treatment is to ameliorate at least one symptom of a cancer, autoimmune diseases, or infectious disease.

As used herein, the term “subject” refers to any mammal. In some embodiments, the subject is a feline (e.g., a cat), a canine (e.g., a dog), an equine (e.g., a horse), a rabbit, a pig, a rodent (e.g., a mouse, a rat, a hamster or a guinea pig), a non-human primate (e.g., a simian (e.g., a monkey (e.g., a baboon, a marmoset), or an ape (e.g., a chimpanzee, a gorilla, an orangutan, or a gibbon)), or a human. In some embodiments, the subject is a human. The terms subject and patient are used interchangeably herein. In some embodiments, the subject has been previously identified or diagnosed as having the disease (e.g., cancer (e.g., any of the cancers described herein)).

In some embodiments, a subject can be identified as having a mutation in a HER2 gene that increase the expression and/or activity of HER2 in a mammalian cell (e.g., any of the mammalian cells described herein). For example, a mutation in a HER2 gene that increases the expression and/or activity of HER2 in a mammalian cell can be a gene duplication, a mutation that results in the expression of a HER2 having one or more amino acid substitutions (E.g., one or more amino acid substitutions selected from the group consisting of: G309A, G309E, S310F, R678Q, L755S, L755W, I767M, D769H, D769Y, V777L, Y835F, V842I, R896C, and G1201V) (as compared to the wild type protein). See, e.g., Weigelt and Reis-Filho, Cancer Discov. 2013, 3(2): 145-147.

Non-limiting examples of methods of detecting a HER2 associated disease in a subject include: immunohistochemistry, fluorescent in situ hybridization (FISH), chromogenic in situ hybridization (CISH). See, e.g., Yan et al., Cancer Metastasis Rev. 2015, 34: 157-164.

A therapeutically effective amount of a target-binding protein of the disclosure relates generally to the amount needed to achieve a therapeutic objective. As noted above, this may be a binding interaction between the antibody and its target antigens that, in certain cases, interferes with the functioning of the targets. The amount required to be administered will furthermore depend on the binding affinity of the target-binding protein for its specific target, and will also depend on the rate at which an administered target-binding protein is depleted from the free volume other subject to which it is administered. Common ranges for therapeutically effective dosing of an target-binding protein of the disclosure may be, by way of nonlimiting example, from about 0.001, 0.01, 0.1, 0.3, 0.5, 0.7, 0.8, 0.9, 1, 2, 3, 4, 5, 6, 7, 8, 9, 10, 15, 20, 25, 30, 35, 40, 45, 50 mg/kg body weight or higher. The structure of the target-binding protein of the present disclosure makes it possible to reduce the dosage of the target-binding protein that is administered to a subject compared to conventional target-binding proteins and compared to conventional antibodies. For example, the administered dose on a unit dosage basis or total dosage over a dosage regimen period may be reduced by 10, 20, 30, 40, or 50% compared to the corresponding dose of a corresponding conventional target-binding protein or a corresponding conventional antibody.

Common dosing frequencies may range, for example, from once or twice daily, weekly, biweekly, or monthly.

Efficaciousness of treatment is determined in association with any known method for diagnosing or treating the particular disorder. Methods for the screening of target-binding proteins that possess the desired specificity include, but are not limited to, enzyme linked immunosorbent assay (ELISA) and other immunologically mediated techniques known within the art.

In another embodiment, a target-binding protein directed to two or more targets are used in methods known within the art relating to the localization and/or quantitation of the targets (e.g., for use in measuring levels of one or more of the targets within appropriate physiological samples, for use in diagnostic methods, for use in imaging the protein, and the like). In a given embodiment, a target-binding protein directed to two or more targets, or a derivative, fragment, analog or homolog thereof, that contain the antibody derived antigen binding domain, are utilized as pharmacologically active compounds (referred to hereinafter as “Therapeutics”).

The target-binding protein used in any of the embodiments of these methods and uses may be administered at any stage of the disease. For example, such a target-binding protein may be administered to a patient suffering cancer of any stage, from early to metastatic. In some embodiments, the target-binding protein and formulations thereof may be administered to a subject suffering from or susceptible to a disease or disorder associated with aberrant target expression and/or activity.

A subject suffering from or susceptible to a disease or disorder associated with aberrant target expression and/or activity may be identified using any of a variety of methods known in the art. For example, subjects suffering from cancer or other neoplastic condition may be identified using any of a variety of clinical and/or laboratory tests such as, physical examination and blood, urine and/or stool analysis to evaluate health status. For example, subjects suffering from inflammation and/or an inflammatory disorder may be identified using any of a variety of clinical and/or laboratory tests such as physical examination and/or bodily fluid analysis, e.g., blood, urine and/or stool analysis, to evaluate health status.

In some embodiments, administration of a target-binding protein to a patient suffering from a disease or disorder associated with aberrant target expression and/or activity may be considered successful if any of a variety of laboratory or clinical objectives is achieved. For example, administration of a target-binding protein to a patient suffering from a disease or disorder associated with aberrant target expression and/or activity may be considered successful if one or more of the symptoms associated with the disease or disorder is alleviated, reduced, inhibited or does not progress to a further, i.e., worse, state. Administration of a target-binding protein to a patient suffering from a disease or disorder associated with aberrant target expression and/or activity may be considered successful if the disease or disorder enters remission or does not progress to a further, i.e., worse, state.

As used herein, the term “treat” includes reducing the severity, frequency or the number of one or more (e.g., 1, 2, 3, 4, or 5) symptoms or signs of a disease (e.g., a cancer (e.g., any of the cancers described herein)) in the subject (e.g., any of the subjects described herein). In some embodiments where the disease is cancer, treating results in reducing cancer growth, inhibiting cancer progression, inhibiting cancer metastasis, or reducing the risk of cancer recurrence in a subject having cancer.

In some embodiments, the disease may be a cancer. In some embodiments, the subject may have been identified or diagnosed as having a cancer. Examples of cancer include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, a lymphoma (e.g., B-cell lymphoma, B-cell non-Hodgkin's lymphoma, Hodgkin's lymphoma, cutaneous T-cell lymphoma), a leukemia (e.g., hairy cell leukemia, chronic lymphocytic leukemia (CLL), acute myeloid leukemia (AML), chronic myeloid leukemia (CML), acute lymphocytic leukemia (ALL)), myelodysplastic syndromes (MDS), Kaposi sarcoma, retinoblastoma, stomach cancer, urothelial carcinoma, lung cancer, renal cell carcinoma, gastric and esophageal cancer, pancreatic cancer, prostate cancer, brain cancer, colon cancer, bone cancer, lung cancer, breast cancer, colorectal cancer, ovarian cancer, nasopharyngeal adenocarcinoma, non-small cell lung carcinoma (NSCLC), squamous cell head and neck carcinoma, endometrial cancer, bladder cancer, cervical cancer, liver cancer, and hepatocellular carcinoma. In some embodiments, the cancer is a lymphoma. In some embodiments, the lymphoma is Burkitt's lymphoma. In some aspects, the subject has been identified or diagnosed as having familial cancer syndromes such as Li Fraumeni Syndrome, Familial Breast-Ovarian Cancer (BRCA1 or BRAC2 mutations) Syndromes, and others. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. Further examples of cancers that may be treated by the compositions and methods herein include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; Wilms' Tumor; diffuse large B-cell lymphoma (DLBCL); and mantle cell lymphoma (MCL). Metastases of the aforementioned cancers may also be treated or prevented in accordance with the methods described herein.

In some embodiments, the methods herein may result in a reduction in the number, severity, or frequency of one or more symptoms of the cancer in the subject (e.g., as compared to the number, severity, or frequency of the one or more symptoms of the cancer in the subject prior to treatment).

The methods may further comprise administering to a subject one or more additional agents. In some embodiments, the additional agent(s) may be a chemotherapeutic agent, such as a chemotherapeutic agent selected from the group consisting of docetaxel, paclitaxel, abraxane (i.e., albumin-conjugated paclitaxel), doxorubicin, oxaliplatin, carboplatin, cisplatin, irinotecan, and gemcitabine. In some embodiments, the additional agent(s) may be a checkpoint inhibitor, a kinase inhibitor, an agent targeting inhibitors in the tumor microenvironment, and/or a T cell or NK agonist. In some embodiments, the additional agent(s) may be radiation therapy, alone or in combination with another additional agent(s) such as a chemotherapeutic or anti-neoplastic agent. In some embodiments, the additional agent(s) may be a vaccine, an oncovirus, and/or a DC-activating agent such as, by way of non-limiting example, a toll-like receptor (TLR) agonist and/or α-CD40. In some embodiments, the additional agent(s) may be a tumor-targeted antibody designed to kill the tumor via ADCC or via direct conjugation to a toxin (e.g., an antibody drug conjugate (ADC).

In some embodiments, the checkpoint inhibitor may be an inhibitor of a protein such as CTLA-4, LAG-3, PD-1, PD-1, TIGIT, TIM-3, B7H4, BTLA, or Vista. In some embodiments, the kinase inhibitor may be B-RAFi, MEKi, Btk inhibitors, ibrutinib, or crizotinib. In some embodiments, the tumor microenvironment inhibitor may be an IDO inhibitor, an α-CSF1R inhibitor, an α-CCR4 inhibitor, a TGF-beta, a myeloid-derived suppressor cell, or a T-regulatory cell. In some embodiments, the agonist may be Ox40, GITR, CD137, ICOS, CD27, or HVEM.

In some embodiments, the target-binding protein may be administered during and/or after treatment in combination with one or more additional agents. In some embodiments, the target-binding protein may be formulated into a single therapeutic composition, and the target-binding protein and additional agent(s) may be administered simultaneously. Alternatively, the target-binding protein and additional agent(s) may be separate from each other, e.g., each is formulated into a separate therapeutic composition, and the target-binding protein and the additional agent are administered simultaneously, or the target-binding protein and the additional agent are administered at different times during a treatment regimen. For example, the target-binding protein may be administered prior to the administration of the additional agent, subsequent to the administration of the additional agent, or in an alternating fashion. The target-binding protein and additional agent(s) may be administered in single doses or in multiple doses.

One of more of the target-binding proteins herein may be co-formulated with, and/or co-administered with, one or more anti-inflammatory drugs, immunosuppressants, or metabolic or enzymatic inhibitors. Examples of the drugs or inhibitors that may be used include one or more of: nonsteroidal anti-inflammatory drug(s) (NSAIDs), e.g., ibuprofen, tenidap, naproxen, meloxicam, piroxicam, diclofenac, and indomethacin; sulfasalazine; corticosteroids such as prednisolone; cytokine suppressive anti-inflammatory drug(s) (CSAIDs); inhibitors of nucleotide biosynthesis, e.g., inhibitors of purine biosynthesis, folate antagonists (e.g., methotrexate (N-[4-[[(2,4-diamino-6-pteridinyl)methyl]methylamino]benzoyl]-L-glutamic acid); and inhibitors of pyrimidine biosynthesis, e.g., dihydroorotate dehydrogenase (DHODH) inhibitors. Suitable therapeutic agents for use in combination with the antibodies of the disclosure include NSAIDs, CSAIDs, (DHODH) inhibitors (e.g., leflunomide), and folate antagonists (e.g., methotrexate). Examples of additional inhibitors include one or more of: corticosteroids (oral, inhaled and local injection); immunosuppressants, e.g., cyclosporin, tacrolimus (FK-506); and mTOR inhibitors, e.g., sirolimus (rapamycin or rapamycin derivatives, e.g., soluble rapamycin derivatives (e.g., ester rapamycin derivatives, e.g., CCI-779); agents that interfere with signaling by proinflammatory cytokines such as TNFα or IL-1 (e.g. IRAK, NIK, IKK, p38 or MAP kinase inhibitors); COX2 inhibitors, e.g., celecoxib, rofecoxib, and variants thereof; phosphodiesterase inhibitors, e.g., R973401 (phosphodiesterase Type IV inhibitor); phospholipase inhibitors, e.g., inhibitors of cytosolic phospholipase 2 (cPLA2) (e.g., trifluoromethyl ketone analogs); inhibitors of vascular endothelial cell growth factor or growth factor receptor, e.g., VEGF inhibitor and/or VEGF-R inhibitor; and inhibitors of angiogenesis. Suitable therapeutic agents for use in combination with the antibodies of the disclosure are immunosuppressants, e.g., cyclosporin, tacrolimus (FK-506); mTOR inhibitors, e.g., sirolimus (rapamycin) or rapamycin derivatives, e.g., soluble rapamycin derivatives (e.g., ester rapamycin derivatives, e.g., CCI-779); COX2 inhibitors, e.g., celecoxib and variants thereof; and phospholipase inhibitors, e.g., inhibitors of cytosolic phospholipase 2 (cPLA2), e.g., trifluoromethyl ketone analogs. Additional examples of therapeutic agents that can be combined with an antibody of the disclosure include one or more of: 6-mercaptopurines (6-MP); azathioprine sulphasalazine; mesalazine; olsalazine; chloroquine/hydroxychloroquine; pencillamine; aurothiornalate (intramuscular and oral); azathioprine; colchicine; beta-2 adrenoreceptor agonists (salbutamol, terbutaline, salmeteral); xanthines (theophylline, arninophylline); cromoglycate; nedocromil; ketotifen; ipratropium and oxitropium; mycophenolate mofetil; adenosine agonists; antithrombotic agents; complement inhibitors; and adrenergic agents.

The present disclosure also provides methods of detecting presence or absence of a cleaving agent and/or the target in a subject or a sample. Such methods may comprise (i) contacting a subject or biological sample with an target-binding protein, wherein the target-binding protein includes a detectable label that is positioned on a portion of the target-binding protein that is released following cleavage of the CM substrate and (ii) measuring a level of activated target-binding protein in the subject or biological sample, wherein a detectable level of activated target-binding protein in the subject or biological sample indicates that the cleaving agent, the target or both the cleaving agent and the target are absent and/or not sufficiently present in the subject or biological sample, such that the target binding and/or protease cleavage of the target-binding protein cannot be detected in the subject or biological sample, and wherein a reduced detectable level of activated target-binding protein in the subject or biological sample indicates that the cleaving agent and the target are present in the subject or biological sample.

A reduced level of detectable label may be, for example, a reduction of at least 5%, 10%, 15%, 20%, 25%, 30%, 35%, 40%, 45%, 50%, 55%, 60%, 65%, 70%, 75%, 80%, 85%, 90%, or 95%, or a reduction of substantially 100%. In some embodiments, the detectable label may be conjugated to a component of the target-binding protein, e.g., the TB. In some embodiments, measuring the level of the target-binding protein in the subject or sample may be accomplished using a secondary reagent that specifically binds to the activated antibody, wherein the reagent comprises a detectable label. The secondary reagent may be an antibody comprising a detectable label.

In some embodiments, the target-binding proteins may also be useful in the detection of the target in patient samples and accordingly are useful as diagnostics. For example, the target-binding proteins may be used in in vitro assays, e.g., ELISA, to detect target levels in a patient sample. For example, a target-binding protein may be immobilized on a solid support (e.g., the well(s) of a microtiter plate). The immobilized target-binding protein may serve as a capture antibody for any target that may be present in a test sample. Prior to contacting the immobilized antibody with a patient sample, the solid support may be rinsed and treated with a blocking agent such as milk protein or albumin to prevent nonspecific adsorption of the analyte.

In some embodiments, based on the results obtained using the target-binding proteins in an in vitro diagnostic assay, the stage of a disease in a subject may be determined based on expression levels of the target antigen. For a given disease, samples of blood may be taken from subjects diagnosed as being at various stages in the progression of the disease, and/or at various points in the therapeutic treatment of the disease. Using a population of samples that provides statistically significant results for each stage of progression or therapy, a range of concentrations of the antigen that may be considered characteristic of each stage is designated.

The target-binding proteins herein may also be used in diagnostic and/or imaging methods. In some embodiments, such methods may be in vitro methods. In some embodiments, such methods may be in vivo methods. In some embodiments, such methods may be in situ methods. In some embodiments, such methods may be ex vivo methods. For example, target-binding proteins having a CM may be used to detect the presence or absence of an enzyme capable of cleaving the CM. Such target-binding proteins may be used in diagnostics, which can include in vivo detection (e.g., qualitative or quantitative) of enzyme activity (or, in some embodiments, an environment of increased reduction potential such as that which can provide for reduction of a disulfide bond) through measured accumulation of activated antibodies (i.e., antibodies resulting from cleavage of a target-binding protein) in a given cell or tissue of a given host organism. Such accumulation of activated antibodies indicates not only that the tissue expresses enzymatic activity (or an increased reduction potential depending on the nature of the CM) but also that the tissue expresses target to which the activated antibody binds.

For example, the CM may be selected to be a protease substrate for a protease found at the site of a tumor, at the site of a viral or bacterial infection at a biologically confined site (e.g., such as in an abscess, in an organ, and the like), and the like. The TB may be one that binds a target antigen. Using methods familiar to one skilled in the art, a detectable label (e.g., a fluorescent label or radioactive label or radiotracer) may be conjugated to a TB or other region of a target-binding protein. Suitable detectable labels may be discussed in the context of the above screening methods and additional specific examples are provided below. Using an TB specific to a protein or peptide of the disease state, along with a protease whose activity is elevated in the disease tissue of interest, target-binding proteins may exhibit an increased rate of binding to disease tissue relative to tissues where the CM specific enzyme is not present at a detectable level or is present at a lower level than in disease tissue or is inactive (e.g., in zymogen form or in complex with an inhibitor). Since small proteins and peptides are rapidly cleared from the blood by the renal filtration system, and because the enzyme specific for the CM is not present at a detectable level (or is present at lower levels in non-disease tissues or is present in inactive conformation), accumulation of activated antibodies in the disease tissue may be enhanced relative to non-disease tissues.

In some embodiments, the target-binding proteins may be useful for in vivo imaging where detection of the fluorescent signal in a subject, e.g., a mammal, including a human, indicates that the disease site contains the target and contains a protease that is specific for the CM of the target-binding protein. The in vivo imaging may be used to identify or otherwise refine a patient population suitable for treatment with a target-binding protein of the disclosure. For example, patients that test positive for both the target and a protease that cleaves the substrate in the CM of the target-binding protein being tested (e.g., accumulate activated antibodies at the disease site) are identified as suitable candidates for treatment with such a target-binding protein comprising such a CM. Likewise, patients that test negative may be identified as suitable candidates for another form of therapy (i.e., not suitable for treatment with the target-binding protein being tested). In some embodiments, such patients that test negative with respect to a first target-binding protein can be tested with other target-binding proteins comprising different CMs until a suitable target-binding protein for treatment is identified (e.g., an target-binding protein comprising a CM that is cleaved by the patient at the site of disease).

In some embodiments, in situ imaging may be useful in methods to identify which patients to treat. For example, in in situ imaging, the target-binding proteins may be used to screen patient samples to identify those patients having the appropriate protease(s) and target(s) at the appropriate location, e.g., at a tumor site. In some embodiments, in situ imaging is used to identify or otherwise refine a patient population suitable for treatment with a target-binding protein of the disclosure. For example, patients that test positive for both the target and a protease that cleaves the substrate in the CM of the target-binding protein being tested (e.g., accumulate activated antibodies at the disease site) are identified as suitable candidates for treatment with such a target-binding protein comprising such a CM. Likewise, patients that test negative for either or both of the target and the protease that cleaves the substrate in the CM substrate in the target-binding protein being tested using these methods are identified as suitable candidates for another form of therapy (i.e., not suitable for treatment with the target-binding protein being tested). In some embodiments, such patients that test negative with respect to a first target-binding protein can be tested with other target-binding proteins comprising different CMs until a suitable target-binding protein for treatment is identified (e.g., an activatable target-binding protein comprising a CM that is cleaved by the patient at the site of disease).

The present disclosure includes any combination of the following numbered items:
1. A target-binding protein comprising:
   a heavy chain variable domain comprising
      a variable heavy chain complementarity determining region 1 (VH CDR1) comprising a sequence of TYAMN (SEQ ID NO: 3),
      a variable heavy chain complementarity determining region 2 (VH CDR2) comprising a sequence of RIRSKYNNYATYYADSVKD (SEQ ID NO: 5), and
      a variable heavy chain complementarity determining region 3(VH CDR3) comprising a sequence of HGNFGNSYVSWX₁AY (SEQ ID NO: 6), wherein X₁ is W or F; and
   a light chain variable domain comprising
      a variable light chain complementarity determining region 1(VL CDR1) comprising a sequence of X₂SSTGAVTTSNYX₃N (SEQ ID NO: 10), wherein X₂ is R or G; and X₃ is P or V,
      a variable light chain complementarity determining region 2 (VL CDR2) comprising a sequence of GTNKRAP (SEQ ID NO: 14), and
      a variable light chain complementarity determining region 3 (VL CDR3) comprising a sequence of X₄LWYSNX₅WV (SEQ ID NO: 15), wherein X₄ is V or I; and X₅ is R or L,
   wherein the heavy chain variable domain and the light chain variable domain are disposed within one or more polypeptides,
   wherein the target-binding protein specifically binds to CD3 epsilon.
2. The target-binding protein of item 1, wherein:
   a. the VH CDR1 comprises a sequences of TYAMN (SEQ ID NO: 3);
   b. the VH CDR2 comprises a sequence of RIRSKYNNYATYYADSVKD (SEQ ID NO: 5);
   c. the VH CDR3 comprises a sequence of HGNFGNSYVSWWAY (SEQ ID NO: 7) or HGNFGNSYVSWFAY (SEQ ID NO: 8);
   d. the VL CDR1 comprises a sequence of RSSTGAVTTSNYPN (SEQ ID NO: 11), RSSTGAVTTSNYVN (SEQ ID NO: 12), or GSSTGAVTTSNYVN (SEQ ID NO: 13);
   e. the VL CDR2 comprises a sequence of GTNKRAP (SEQ ID NO: 14); and
   f. the VL CDR3 comprises a sequence of VLWYSNRWV (SEQ ID NO: 16), VLWYSNLWV (SEQ ID NO: 17), or ILWYSNRWV (SEQ ID NO: 18).
3. The target-binding protein of item 1, wherein
   the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWWAY (SEQ ID NO: 7), the VL CDR1 comprises RSSTGAVTTSNYPN (SEQ ID NO: 11), the VL CDR2 comprises GTNKRAP (SEQ ID NO: 14), and the VL CDR3 comprises VLWYSNRWV (SEQ ID NO: 16);
   the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWWAY (SEQ ID NO: 7), the VL CDR1 comprises GSSTGAVTTSNYVN (SEQ ID NO: 13), the VL CDR2 comprises GTNKRAP (SEQ ID NO: 14), and the VL CDR3 comprises VLWYSNRWV (SEQ ID NO: 16);
   the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWFAY (SEQ ID NO: 8), the VL CDR1 comprises GSSTGAVTTSNYVN (SEQ ID NO: 13), the VL CDR2 comprises GTNKRAP (SEQ ID NO: 14), and the VL CDR3 comprises VLWYSNRWV (SEQ ID NO: 16);
   the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWWAY (SEQ ID NO: 7), the VL CDR1 comprises RSSTGAVTTSNYVN (SEQ ID NO: 12), the VL CDR2 comprises GTNKRAP (SEQ ID NO: 14), and the VL CDR3 comprises ILWYSNRWV (SEQ ID NO: 18);
   the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWWAY (SEQ ID NO: 7), the VL CDR1 comprises GSSTGAVTTSNYVN (SEQ ID NO: 13), the VL CDR2 comprises GTNKRAP (SEQ ID NO: 14), and the VL CDR3 comprises VLWYSNLWV (SEQ ID NO: 17); or
   the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWWAY (SEQ ID NO: 7), the VL CDR1 comprises RSSTGAVTTSNYVN (SEQ ID NO: 12), the VL CDR2 comprises GTNKRAP (SEQ ID NO: 14), and the VL CDR3 comprises VLWYSNRWV (SEQ ID NO: 16).
4. The target-binding protein of item 3, wherein the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWWAY (SEQ ID NO: 7), and the light chain variable domain comprises the VL CDR1 comprising RSSTGAVTTSNYPN (SEQ ID NO: 11), the VL CDR2 comprising GTNKRAP (SEQ ID NO: 14), and the VL CDR3 comprising VLWYSNRWV (SEQ ID NO: 16).
5. The target-binding protein of item 4, wherein the heavy chain variable domain comprises a sequence that is at least 90%, or at least 91%, 92%, 93%, 94, 95%, 96%, 97%, 98%, or 99% identical to SEQ ID NO: 46; and the light chain variable domain comprises a sequence that is at least 90%, or at least 91%, 92%, 93%, 94, 95%, 96%, 97%, 98%, or 99% identical to SEQ ID NO: 49.
6. The target-binding protein of item 4, wherein the heavy chain variable domain comprises the amino acid sequence of SEQ ID NO: 46 and the light chain variable domain comprises the amino acid sequence of SEQ ID NO: 49.
7. The target-binding protein of item 3, wherein the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWWAY (SEQ ID NO: 7), and the light chain variable domain comprises the VL CDR1 comprising GSSTGAVTTSNYVN (SEQ ID NO: 13), the VL CDR2 comprising GTNKRAP (SEQ ID NO: 14), and the VL CDR3 comprising VLWYSNRWV (SEQ ID NO: 16).
8. The target-binding protein of item 7, wherein the heavy chain variable domain comprises a sequence that is at least 90%, or at least 91%, 92%, 93%, 94, 95%, 96%, 97%, 98%, or 99% identical to SEQ ID NO: 46; and the light chain variable domain comprises a sequence that is at least 90%, or at least 91%, 92%, 93%, 94, 95%, 96%, 97%, 98%, or 99% identical to SEQ ID NO: 64.
9. The target-binding protein of item 7, wherein the heavy chain variable domain comprises a sequence of SEQ ID NO: 46, and the light chain variable domain comprises a sequence of SEQ ID NO: 64.
10. The target-binding protein of item 3, wherein the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWFAY (SEQ ID NO: 8), and the light chain variable domain comprises the VL CDR1 comprising GSSTGAVTTSNYVN (SEQ ID NO: 13), the VL CDR2 comprising GTNKRAP (SEQ ID NO: 14), and the VL CDR3 comprising VLWYSNRWV (SEQ ID NO: 16).
11. The target-binding protein of item 10, wherein the heavy chain variable domain comprises a sequence that is at least 90%, or at least 91%, 92%, 93%, 94, 95%, 96%, 97%, 98%, or 99% identical to SEQ ID NO: 128; and the light chain variable domain comprises a sequence that is at least 90%, or at least 91%, 92%, 93%, 94, 95%, 96%, 97%, 98%, or 99% identical to SEQ ID NO: 122.
12. The target-binding protein of item 10, wherein the heavy chain variable domain comprises a sequence of SEQ ID NO: 128, and the light chain variable domain comprises a sequence of SEQ ID NO: 122.
13. The target-binding protein of item 3, wherein the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWWAY (SEQ ID NO: 7), and the light chain variable domain comprises the VL CDR1 comprising RSSTGAVTTSNYVN (SEQ ID NO: 12), the VL CDR2 comprising GTNKRAP (SEQ ID NO: 14), and the VL CDR3 comprising ILWYSNRWV (SEQ ID NO: 18).
14. The target-binding protein of item 13, wherein the heavy chain variable domain comprises a sequence that is at least 90%, or at least 91%, 92%, 93%, 94, 95%, 96%, 97%, 98%, or 99% identical to SEQ ID NO: 46; and the light chain variable domain comprises a sequence that is at least 90%, or at least 91%, 92%, 93%, 94, 95%, 96%, 97%, 98%, or 99% identical to SEQ ID NO: 113.
15. The target-binding protein of item 13, wherein the heavy chain variable domain comprises a sequence of SEQ ID NO: 46, and the light chain variable domain comprises a sequence of SEQ ID NO: 113.
16. The target-binding protein of item 3, wherein the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWWAY (SEQ ID NO: 7), and the light chain variable domain comprises the VL CDR1 comprising GSSTGAVTTSNYVN (SEQ ID NO: 13), the VL CDR2 comprising GTNKRAP (SEQ ID NO: 14), and the VL CDR3 comprising VLWYSNLWV (SEQ ID NO: 17).
17. The target-binding protein of item 16, wherein the heavy chain variable domain comprises a sequence that is at least 90%, or at least 91%, 92%, 93%, 94, 95%, 96%, 97%, 98%, or 99% identical to SEQ ID NO: 46; and the light chain variable domain comprises a sequence that is at least 90%, or at least 91%, 92%, 93%, 94, 95%, 96%, 97%, 98%, or 99% identical to SEQ ID NO: 98.
18. The target-binding protein of item 16, wherein the heavy chain variable domain comprises a sequence of SEQ ID NO: 46, and the light chain variable domain comprises a sequence of SEQ ID NO: 98.
19. The target-binding protein of item 3, wherein the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWWAY (SEQ ID NO: 7), and the light chain variable domain comprises the VL CDR1 comprising RSSTGAVTTSNYVN (SEQ ID NO: 12), the VL CDR2 comprising GTNKRAP (SEQ ID NO: 14), and the VL CDR3 comprising VLWYSNRWV (SEQ ID NO: 16).
20. The target-binding protein of item 19, wherein the heavy chain variable domain comprises a sequence that is at least 90%, or at least 91%, 92%, 93%, 94, 95%, 96%, 97%, 98%, or 99% identical to SEQ ID NO: 46; and the light chain variable domain comprises a sequence that is at least 90%, or at least 91%, 92%, 93%, 94, 95%, 96%, 97%, 98%, or 99% identical to SEQ ID NO: 107.
21. The target-binding protein of item 19, wherein the heavy chain variable domain comprises a sequence of SEQ ID NO: 46, and the light chain variable domain comprises a sequence of SEQ ID NO: 107.
22. A target-binding protein comprising:
   a heavy chain variable domain comprising a sequence of EVQLVESGGGLVQPGGSLKLSCAASGFTFSTYAMNWVRQASGKGLEWVGRIRS KYNNYATYYADSVKDRFTISRDDSKNTAYLQMNSLKTEDTAVYYCVRHGNFGN SYVSWX₁AYWGX₆GTLVTVSS (SEQ ID NO: 20); and
   a light chain variable domain comprising a sequence of QTVVTQEPSLTVSPGGTVTLTCX₂SSTGAVTTSNYX₃NWVQQKPGX₇APRGLIGGT NKRAPGTPARFSGSLJGGKAALTLSGX₉QPEDEAEYYCX₄LWYSNX₅WVFGGGTK LTVL (SEQ ID NO: 21),
   wherein:
   X₁ is W or F; X₂ is R or G; X₃ is P or V; X₄ is V or I; X₅ is R or L,
   X₆ is Q and X₇ is Q; or X₆ is C and X₇ is C,
   J is L when X₉ is V; or J is I when X₉ is A, and
   the target-binding protein specifically binds to CD3 epsilon.
23. The target-binding protein of item 22, wherein X₆ is Q and X₇ is Q.
24. The target-binding protein of item 22, wherein X₆ is C and X₇ is C.
25. The target-binding protein of item 22, wherein
   the heavy chain variable domain comprises a sequence of SEQ ID NO: 46 and the light chain variable domain comprises a sequence of SEQ ID NO: 49;
   the heavy chain variable domain comprises a sequence of SEQ ID NO: 46 and the light chain variable domain comprises a sequence of SEQ ID NO: 64;
   the heavy chain variable domain comprises a sequence of SEQ ID NO: 46 and the light chain variable domain comprises a sequence of SEQ ID NO: 113;
   the heavy chain variable domain comprises a sequence of SEQ ID NO: 46 and the light chain variable domain comprises a sequence of SEQ ID NO: 98;
   the heavy chain variable domain comprises a sequence of SEQ ID NO: 46 and the light chain variable domain comprises a sequence of SEQ ID NO: 107; or
   the heavy chain variable domain comprises a sequence of SEQ ID NO: 128 and the light chain variable domain comprises a sequence of SEQ ID NO: 122.
26. The target-binding protein of any one or combination of items 1-25, wherein the heavy chain variable domain and the light chain variable domain are disposed within the same polypeptide.
27. The target-binding protein of item 26, wherein the heavy chain variable domain and the light chain variable domain are coupled via a linker.
28. The target-binding protein of item 27, wherein the linker has a length of 5 to 30, 6 to 29, 7 to 28, 8 to 27, 9 to 26, 10 to 25, 11, 12, 13, 14, 15, 16, 17, 18, 19, 20, 21, 22, 23, or 24 amino acids.
29. The target-binding protein of item 26, comprising a sequence of SEQ ID NO: 50, 28, 114, 99, 108, or 32.
30. The target-binding protein of any one or combination of items 26-29, wherein the target-binding protein comprises a single chain variable fragment (scFv).
31. The target-binding protein of any one or combination of items 1-30, wherein the target-binding protein comprises a BiTE, a (scFv)2, a NANOBODY®, a nanobody-HSA VHH-scAb, a VHH-Fab, a Dual scFab, a F(ab′)2, a diabody, a CROSSMAB®, a DAF (two-in-one), a DAE (four-in-one), a DUTAMAB®, a DT-IgG, a knobs-in-holes common light chain, a knobs-in-holes assembly, a charge pair, a Fab-arm exchange, a SEEDbody, a LUZ-Y, a FcAb, a kl-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, ZYBODY™, DVI-IgG, Diabody-CH3, a triple body, a miniantibody, a minibody, a TriBi minibody, scFv-CH3 KIH, Fab-scFv, a F(ab′)2-scFv2, a scFv-KrH, a Fab-scFv-Fc, a tetravalent HCAb, a scDiabody-Fc, a Diabody-Fc, a tandem scFv-Fc, a VHH-Fc, a tandem VHH-Fc, a L′HH-Fc KiH, a Fab-VHH-Fc, an Intrabody, a dock and lock, an ImmTAC® (immune-mobilizing monoclonal TCRs (T cell receptors) against cancer), an IgG-IgG conjugate, a Cov-X-Body, a scFv1-PEG-scFv2, an Adnectin, a DARPin®, a fibronectin, an IgG, an IgM, an IgA, an IgE, an IgD, or a DEP conjugate, TMEAbody™, SAFEbody®, TRITAC®, or SHIELD antibody.
32. The target-binding protein of any one or combination of items 1-30, wherein the target-binding protein comprises an IgG, IgM, IgA, IgE, or IgD antibody or fragment thereof.
33. The target-binding protein of item 32, wherein the target-binding protein comprises an IgG1, IgG2, IgG3, or IgG4 antibody.
34. The target-binding protein of any one or combination of items 1-33, wherein the target-binding protein is humanized.
35. The target-binding protein of any one or combination of items 1-34, further comprising a masking moiety that inhibits binding of the target-binding protein to CD3 in an inactive state.
36. The target-binding protein of item 35, wherein the masking moiety is coupled to the target-binding protein via a cleavable moiety, and the cleavable moiety is a substrate for a protease.
37. The target-binding protein of any one or combination of item 36, wherein the protease is ADAMS, ADAMTS, ADAM8, ADAM9, ADAM10, ADAM12, ADAM15, ADAM17/TACE, ADAMDEC1, ADAMTS1, ADAMTS4, ADAMTS5, Aspartate proteases, BACE, Renin, Aspartic cathepsins, Cathepsin D, Cathepsin E, Caspases, Caspase 1, Caspase 2, Caspase 3, Caspase 4, Caspase 5, Caspase 6, Caspase 7, Caspase 8, Caspase 9, Caspase 10, Caspase 14, Cysteine cathepsins, Cathepsin B, Cathepsin C, Cathepsin K, Cathepsin L, Cathepsin S, Cathepsin V/L2, Cathepsin X/Z/P, Cysteine proteinases, Cruzipain, Legumain, Otubain-2, KLKs, KLK4, KLK5, KLK6, KLK7, KLK8, KLK10, KLK11, KLK13, KLK14, Metallo proteinases, Meprin, Neprilysin, PSMA, BMP-1, MMPs, MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27, Serine proteases, activated protein C, Cathepsin A, Cathepsin G, Chymase, coagulation factor proteases, FVIIa, FIXa, FXa, FXIa, FXIIa, Elastase, Granzyme B, Guanidinobenzoatase, HtrA1, Human Neutrophil Elastase, Lactoferrin, Marapsin, NS3/4A, PACE4, Plasmin, PSA, tPA, Thrombin, Tryptase, uPA, Type II Transmembrane, Serine Proteases, TTSPs, DESC1, DPP-4, FAP, Hepsin, Matriptase-2, MT-SP1/Matriptase, TMPRSS2, TMPRSS3, or TMPRSS4.
38. The target-binding protein of any one or combination of items 1-37, wherein the heavy chain variable domain and/or the light chain variable domain is conjugated to a toxin, radioisotope, small molecule, diagnostic agent, therapeutic macromolecule, targeting moiety, or detectable moiety, via a conjugating moiety.
39. The target-binding protein of item 38, wherein the conjugating moiety is cleavable by a protease.
40. The target-binding protein of item 38, wherein the conjugating moiety is non-cleavable by a protease.
41. A composition comprising the target-binding protein of any one or combination of items 1-40 and a carrier.
42. The composition of item 41, wherein the composition is a pharmaceutical composition, and wherein the carrier is a pharmaceutically acceptable carrier.
43. A container, vial, syringe, injector pen, or kit comprising at least one dose of the composition of item 41 or 42.
44. A nucleic acid comprising a sequence encoding the target-binding protein of any one or combination of items 1-40.
45. A vector comprising the nucleic acid of item 44.
46. A cell comprising the nucleic acid of item 44 or the vector of item 45.
47. A method of treating a subject in need thereof comprising administering to the subject a therapeutically effective amount of the target-binding protein of any one or combination of items 1-40 or the composition of item 41 or 42.
48. The method of item 47, wherein the subject has been identified or diagnosed as having a cancer, an inflammatory condition, disorder or disease, or an autoimmune condition, disorder or disease.
49. A method of producing a target-binding protein, comprising:
   culturing the cell of item 46 in a culture medium under a condition sufficient to produce the target-binding protein; and
   recovering the target-binding protein from the cell or the culture medium.
50. The method of item 49, further comprising isolating the target-binding protein recovered from the cell or the culture medium.
51. The method of item 50, further comprising formulating the target-binding protein into a pharmaceutical composition.

### EXAMPLES

The invention is further described in the following examples, which do not limit the scope of the invention described in the claims.

### Example 1: Production of Monovalent Bispecific Antibodies Comprising Anti-CD3 scFv

This example shows an exemplary method for producing monovalent bispecific antibodies comprising anti-CD3 scFv and a Her2-binding domain. The monovalent bispecific antibodies were prepared by recombinant methods. Proteins were prepared by transforming a host cell with expression vectors for a polynucleotide comprising a coding sequence for an anti-CD3 scFv of SEQ ID NOs: 50, 28, 114, 32, 99, 108, or 164, and other components of the monovalent bispecific antibodies, followed by cultivation of the resulting recombinant host cells under conditions suitable to produce the monovalent bispecific antibodies comprising anti-CD3 scFv. Supernatants were harvested at day 5 following transformation, titered for Protein-A binding material using an Octet assay (Table 7), and subsequently monovalent bispecific antibodies were purified using affinity chromatography (Protein A and/or CH1) and size-exclusion chromatography methods. Higher Protein A Octet titers generally correlated with desirable higher yields and several of the CD3 variants exhibited this desirable property (Table 7).

### Example 2: Characterization of Monovalent Bispecific Antibodies Comprising Anti-CD3 scFv

The CD3 binding of the monovalent bispecific antibodies produced in Example 1 was determined using Octet assay. The binding kinetics of the anti-CD3 scFv to human CD3ε were analyzed using bio-layer interferometry on an Octet system (Sartorius, Octet RED96). All proteins were diluted in kinetics buffer (PBS, 2% BSA, 0.1% Tween-20). Super Streptavidin (SSA) coated biosensors (Sartorius, 18-5057) were equilibrated in kinetics buffer for 10 min at room temperature preceding data acquisition, and experiments were performed at 30° C. with agitation at 1000 rpm. An initial baseline level was performed for 60 seconds (“s”). 10 nM of recombinant his-tagged and biotinylated CD3ε protein (AcroBiosystems, CDE-H8223) was loaded on a SSA biosensor for 120 s. Prior to analyte association, another baseline level was established for 60 s. Loaded sensor was associated with 200 nM of monovalent CD3 module in off-rate screening and into concentrations ranging from 200 nM to 3.125 nM in full kinetics assay for 150 s, and then followed by dissociation step in kinetics buffer for 300 s. A reference molecule comprising v12_LH (SEQ ID NO: 164), was included in each off-rate screening assay. A reference sensor with loaded ligand, but no analyte, was subtracted from the data before fitting. Data was fit using a mass transport model. The analysis was carried out with ForteBio Data Analysis 10.0 software. The CD3 monovalent bispecific antibodies exhibited specific binding to CD3 as shown by the K_{d} and K_{off} data in Table 7 below.

Monomer content of the monovalent bispecific antibodies was analyzed using analytical size exclusion chromatography (SEC). The analysis was performed using an Agilent 1260 Infinity HPLC system with UV detection at 280 nm absorbance. Protein aliquots (approximately g) of the affinity chromatography (Protein A or CH1) purified material were injected onto 7.8 mm×15 cm TSKgel QC-PAK GFC300 column (TOSOH Biosciences, King of Prussia, PA) that was equilibrated with 0.1 M sodium phosphate, 0.1 M sodium sulfate pH 6.8, and a flow rate of 1.0 mL/min. Low % monomer content of the affinity-purified material (prior to size-exclusion polishing step) is generally not desirable because it will negatively affect the total yield at the polishing step. The CD3 variants described in Table 7 all had an acceptable >=50% monomer content at the affinity-purification step.

Thermal denaturation was studied using Nanotemper Prometheus NT.48 (NanoTemper Technologies, Munich). The concentration of samples was between 0.8 mg/ml-1 mg/ml and heating rate was 1° C./min. Stability data was recorded using temperature-dependent change in tryptophan fluorescence at emission wavelengths of 330 nm and 350 nm. Intrinsic fluorescence of tryptophan and tyrosine (Trp/Try) was measured at both 330- and 350-nm wavelengths and plotted versus temperature from 15 to 95° C. during unfolding at heating rate of 1° C./min. Unfolding/denaturation resulted in a change in the microenvironment polarity around tryptophan residues, leading to changes in fluorescence which is reflecting in the melting curves. A plot of fluorescence ratio F350/F330 against temperature yielded clear melting transitions which were used for analyzing the melting temperature. Melting temperatures were determined by detecting the maximum of the first derivative of the fluorescence ratios (F350/F330). The melting temperature (Tm) was calculated by the first derivative of the F350/330 plots. A higher Tm indicates a more stable protein. All CD3 variants described in Table 7, except the reference v12_LH, had desirable Tm's that were above 55° C. and significantly higher than the reference v12_LH. The monovalent bispecific antibodies are identified by their CD3-binding domains in Table 7 below. Koff data are reported as relative measurements, where “+++” indicates that the Koff rate for CD3 binding is approximately the same as or better than the reference monovalent bispecific antibody comprising v12_LH (SEQ ID NO: 164) as the anti-CD3 scFv.

TABLE 7

| Monovalent | | | | | |
|---|---|---|---|---|---|
| bispecific | Octet titer | | | | |
| antibodies | (mg/mL) | % monomer | Tm | K_{D} (nM) | K_{off} |
| v619_HLc | 76/258* | 80 | 58/57 | 0.92-2.62 | +++ |
| (SEQ ID NO: 50) | | | | | |
| v619_HLh | 102/248* | 84 | 56/58 | 1.66-5.64 | +++ |
| (SEQ ID NO: 28) | | | | | |
| v619-HLh-CC4 | 140 | 62 | 59 | ~11 | +++ |
| (SEQ ID NO: 32) | | | | | |
| v619_Hh8 | 110 | 50 | 57 | | +++ |
| (SEQ ID NO: 99) | | | | | |
| v619_HLp2 | 98 | | 56 | | +++ |
| (SEQ ID NO: | | | | | |
| 114) | | | | | |
| v619_Hh13 | 64 | | 57 | | +++ |
| (SEQ ID NO: | | | | | |
| 108) | | | | | |
| v12_LH | 58 | 85 | 53 | | +++ |
| (reference, SEQ | | | | | |
| ID NO: 164) | | | | | |

*Two numbers are from two independent transformations and represent the variability in transformation/expression.

### Example 3 Cytotoxicity of Monovalent Bispecific Antibodies Comprising Anti-CD3 scFv and Her2-Binding Domain

The in vitro potency of the monovalent bispecific antibodies was determined by a cytotoxicity assay. In brief, SKOV3-luc2 target cells and human PBMC effector cells (Stemcell technologies) were plated together in a co-culture in RPMI media (Gibco cat #22400071) supplemented with 5% human serum (MP Bio cat #2930949) at 1:10 Target to Effector cell ratio. To this co-culture, titrations of the monovalent bispecific antibodies produced in Example 1 were added, and the plate was incubated for approximately 48 hours at 37° C. and 5% CO₂. Post incubation, cytotoxicity was evaluated using ONE-Glo™ Luciferase Assay System (Promega cat #E6130) and the luminescence was measured on a plate reader (TECAN). The percent cytotoxicity was calculated as follows: (1−(RLU experimental/average RLU untreated))*100. Using GraphPad PRISM, percent cytotoxicity data was plotted and EC₅₀ values were calculated. The results are shown in Table 8 below as a relative value of EC50 compared to the EC50 of an internal control monovalent bispecific antibody comprising v12-LH as the anti-CD3 scFv (SEQ ID NO: 164). Each molecule was tested at least twice, and the range of results obtained is reported in Table 8. The typical assay error is approximately 2-3 fold, so a ratio of about 0.5 to about 2 indicates that the tested monovalent bispecific antibody has similar potency to the reference monovalent bispecific antibody comprising v12-LH as the anti-CD3 binding protein. A ratio of less than about 0.5 indicates that the tested monovalent bispecific antibody is more potent than the reference monovalent bispecific antibody comprising the v12-LH anti-CD3 scFv. The monovalent bispecific antibodies are identified by their CD3-binding domains. Most of the CD3 variants (all except v619_HLp2) in Table 8 had EC50 ratios that suggested better potency than v12-LH, within limits of assay variation.

TABLE 8
Cytotoxicity of monovalent bispecific antibodies comprising anti-CD3 scFv.

| Monovalent | v619_HLc | v619_HLh | v619-HLh- | v619_Hh8 | v619_HLp2 | v619_Hh13 |
|---|---|---|---|---|---|---|
| bispecific | (SEQ ID | (SEQ ID | CC4 (SEQ | (SEQ ID | (SEQ ID | (SEQ ID |
| antibodies | NO: 50) | NO: 28) | ID NO: 32) | NO: 99) | NO: 114) | NO: 108) |
| Ratio of | 0.01-0.13 | 0.11-1.15 | 0.35-1.51 | 0.23-0.61 | 0.57-1.84 | 0.14-0.26 |
| EC50 over | | | | | | |
| EC50 of | | | | | | |
| reference | | | | | | |
| (v12-LH, | | | | | | |
| SEQ ID NO: | | | | | | |
| 164) | | | | | | |

Examples of the sequences are listed in Tables 9A and 9B below.

TABLE 9A
Exemplary sequences

| SEQ | | |
|---|---|---|
| ID | | |
| NO | Notes | Sequences |
| 1 | Wild human | DGNEEMGGITQTPYKVSISGTTVILTCPQYPGSEILWQHNDK |
| | type CD3 | NIGGDEDDKNIGSDEDHLSLKEFSELEQSGYYVCYPRGSKPE |
| | epsilon | DANFYLYLRARVCENCMEMDVMSVATIVIVDICITGGLLLLV |
| | | YYWSKNRKAKAKPVTRGAGAGGRQRGQNKERPPPVPNPDY |
| | | EPIRKGQRDLYSGLNQRRI |
| 3 | VH CDR1 | TYAMN |
| 5 | VH CDR2 | RIRSKYNNYATYYADSVKD |
| 6 | VH CDR3 | HGNFGNSYVSWX₁AY |
| | consensus | wherein X₁ is W or F |
| | sequence | |
| 7 | VH CDR3 | HGNFGNSYVSWWAY |
| 8 | VH CDR3 | HGNFGNSYVSWFAY |
| 10 | VL CDR1 | X₂SSTGAVTTSNYX₃N |
| | consensus | wherein X₂ is R or G; X₃ is P or V |
| | sequence | |
| 11 | VL CDR1 | RSSTGAVTTSNYPN |
| 12 | VL CDR1 | RSSTGAVTTSNYVN |
| 13 | VL CDR1 | GSSTGAVTTSNYVN |
| 14 | VL CDR2 | GTNKRAP |
| 15 | VL CDR3 | X₄LWYSNX₅WV |
| | consensus | wherein X₄ is V or I; X₅ is R or L |
| | sequence | |
| 16 | VL CDR3 | VLWYSNRWV |
| 17 | VL CDR3 | VLWYSNLWV |
| 18 | VL CDR3 | ILWYSNRWV |
| 20 | Heavy chain | EVQLVESGGGLVQPGGSLKLSCAASGFTESTYAMNWVRQAS |
| | variable domain | GKGLEWVGRIRSKYNNYATYYADSVKDRFTISRDDSKNTAY |
| | consensus | LQMNSLKTEDTAVYYCVRHGNFGNSYVSWX₁AYWGX&GTL |
| | sequence | VTVSS |
| | | wherein X₁ is W or F; |
| | | X₆ is the same as X₇ in SEQ ID NO: 21 |
| | | and is Q or C |
| 21 | Light chain | QTVVTQEPSLTVSPGGTVTLTCX₂SSTGAVTTSNYX₃NWVQQ |
| | variable domain | KPGX₇APRGLIGGTNKRAPGTPARFSGSLJGGKAALTLSGX₉Q |
| | consensus | PEDEAEYYCX₄LWYSNX₅WVFGGGTKLTVL |
| | sequence | wherein: |
| | | X₂ is R or G; X₃ is P or V; X₄ is V or I; X₅ is R or L, |
| | | X₇ is the same as X₆ in SEQ ID NO: 20 and is Q or C, and |
| | | J is L when X₉ is V; or J is I when X₉ is A |
| 22 | Heavy chain | EVQLVESGGGLVQPGGSLKLSCAASGFTFSTYAMNWVRQAS |
| | variable domain | GKGLEWVGRIRSKYNNYATYYADSVKDRFTISRDDSKNTAY |
| | consensus | LQMNSLKTEDTAVYYCVRHGNFGNSYVSWX₁AYWGX&GTL |
| | sequence (no | VTVSS |
| | disulfide bond | wherein X₁ is W or F; X₆ is Q |
| | mutations) | |
| 23 | Light chain | QTVVTQEPSLTVSPGGTVTLTCX₂SSTGAVTTSNYX₃NWVQQ |
| | variable domain | KPGX₇APRGLIGGTNKRAPGTPARFSGSLJGGKAALTLSGX₃Q |
| | consensus | PEDEAEYYCX₄LWYSNX₅WVFGGGTKLTVL |
| | sequence (no | wherein: |
| | disulfide | X₂ is R or G; X₃ is P or V; X₄ is V or I; X₅ is R or L, |
| | mutations) | X₇ is Q, and |
| | | J is L when X₉ is V; or J is I when X₉ is A |
| 24 | Heavy chain | EVQLVESGGGLVQPGGSLKLSCAASGFTFSTYAMNWVRQAS |
| | variable domain | GKGLEWVGRIRSKYNNYATYYADSVKDRFTISRDDSKNTAY |
| | consensus | LQMNSLKTEDTAVYYCVRHGNFGNSYVSWX₁AYWGX&GTL |
| | sequence (with | VTVSS |
| | disulfide bond | wherein X₁ is W or F; X₆ is C |
| | mutations) | |
| 25 | Light chain | QTVVTQEPSLTVSPGGTVTLTCX₂SSTGAVTTSNYX₃NWVQQ |
| | variable domain | KPGX₃APRGLIGGTNKRAPGTPARFSGSLJGGKAALTLSGX₉Q |
| | consensus | PEDEAEYYCX₄LWYSNX₅WVFGGGTKLTVL |
| | sequence (with | wherein: |
| | disulfide | X₂ is R or G; X₃ is P or V; X₄ is V or I; |
| | mutations) | X₅ is R or L, X₇ is C, and |
| | | J is L when X₉ is V; or J is I when X₉ is A |
| 26 | Fc domain hole | ESKYGPPCPPCPAPEFEGGPSVFLFPPKPKDTLMISRTPEVTCV |
| | mutant | VVDVSQEDPEVQFNWYVDGVEVHNAKTKPREEQFNSTYRV |
| | | VSVLTVLHQDWLNGKEYKCKVSNKGLPSSIEKTISKAKGQPR |
| | | EPQVCTLPPSQEEMTKNQVSLSCAVKGFYPSDIAVEWESNGQ |
| | | PENNYKTTPPVLDSDGSFFLVSRLTVDKSRWQEGNVFSCSV |
| | | MHEALHNHYTQKSLSLSLGX |
| | | X is lysine or absent. |
| 27 | Fc domain knob | GSSKYGPPCPPCPAPEFEGGPSVFLFPPKPKDTLMISRTPEV |
| | mutant | TCVVVDVSQEDPEVQFNWYVDGVEVHNAKTKPREEQFNSTYR |
| | (HC179(HC_Fc | VVSVLTVLHQDWLNGKEYKCKVSNKGLPSSIEKTISKAKGQ |
| | K_IgG4_EN)) | PREPQVYTLPPCQEEMTKNQVSLWCLVKGFYPSDIAVEWES |
| | | NGQPENNYKTTPPVLDSDGSFFLYSRLTVDKSRWQEGNVFS |
| | | CSVMHEALHNHYTQKSLSLSLGX |
| | | X is lysine or absent. |
| 28 | v619h | EVQLVESGGGLVQPGGSLKLSCAASGFTESTYAMNWVRQAS |
| | | GKGLEWVGRIRSKYNNYATYYADSVKDRFTISRDDSKNTAY |
| | | LQMNSLKTEDTAVYYCVRHGNFGNSYVSWWAYWGQGTLV |
| | | TVSSGGGGSGGGGSGGGGSQTVVTQEPSLTVSPGGTVTLTC |
| | | GSSTGAVTTSNYVNWVQQKPGQAPRGLIGGTNKRAPGTPAR |
| | | FSGSLIGGKAALTLSGAQPEDEAEYYCVLWYSNRWVFGGGT |
| | | KLTVL |
| 29 | v619h + 20gg | QGQSGSGYLWGCEWNCGGITTGSSGGSGGSGGLSGRSDDHG |
| | | GSEVQLVESGGGLVQPGGSLKLSCAASGFTFSTYAMNWVRQ |
| | | ASGKGLEWVGRIRSKYNNYATYYADSVKDRFTISRDDSKNT |
| | | AYLQMNSLKTEDTAVYYCVRHGNFGNSYVSWWAYWGQGTL |
| | | VTVSSGGGGSGGGGSGGGGSQTVVTQEPSLTVSPGGTVTLT |
| | | CGSSTGAVTTSNYVNWVQQKPGQAPRGLIGGTNKRAPGTPA |
| | | RFSGSLIGGKAALTLSGAQPEDEAEYYCVLWYSNRWVFGGG |
| | | TKLTVL |
| 30 | v619h-CC4 + | QGQSGSGYLWGCEWNCGGITTGSSGGSGGSGGLSGRSDDHG |
| | 20gg | GSEVQLVESGGGLVQPGGSLKLSCAASGFTFSTYAMNWVRQ |
| | | ASGKGLEWVGRIRSKYNNYATYYADSVKDRFTISRDDSKNT |
| | | AYLQMNSLKTEDTAVYYCVRHGNFGNSYVSWFAYWGCGTL |
| | | VTVSSGGGGSGGGGSGGGGSQTVVTQEPSLTVSPGGTVTLT |
| | | CGSSTGAVTTSNYVNWVQQKPGCAPRGLIGGTNKRAPGTPA |
| | | RFSGSLIGGKAALTLSGAQPEDEAEYYCVLWYSNRWVFGGG |
| | | TKLTVL |
| 31 | v619h-CC45, | QGQSGSGYLWGCEWNCGGITTGSSGGSGGSGGLSGRSDDHG |
| | 240 + 20gg | GSEVQLVESGGGLVQPGGSLKLSCAASGFTFSTYAMNWVRQ |
| | | ASGKGCEWVGRIRSKYNNYATYYADSVKDRFTISRDDSKNT |
| | | AYLQMNSLKTEDTAVYYCVRHGNFGNSYVSWWAYWGQGT |
| | | LVTVSSGGGGSGGGGSGGGGSQTVVTQEPSLTVSPGGTVTLT |
| | | CGSSTGAVTTSNYVNWVQQKPGQAPRGLIGGTNKRAPGTPA |
| | | RFSGSLIGGKAALTLSGAQPEDEAEYYCVLWYSNRWVCGGG |
| | | TKLTVL |
| 32 | v619h-CC4 | EVQLVESGGGLVQPGGSLKLSCAASGFTFSTYAMNWVRQAS |
| | | GKGLEWVGRIRSKYNNYATYYADSVKDRFTISRDDSKNTAY |
| | | LQMNSLKTEDTAVYYCVRHGNFGNSYVSWFAYWGCGTLVT |
| | | VSSGGGGSGGGGSGGGGSQTVVTQEPSLTVSPGGTVTLTCGS |
| | | STGAVTTSNYVNWVQQKPGCAPRGLIGGTNKRAPGTPARFS |
| | | GSLIGGKAALTLSGAQPEDEAEYYCVLWYSNRWVFGGGTKL |
| | | TVL |
| 33 | v619h- | EVQLVESGGGLVQPGGSLKLSCAASGFTFSTYAMNWVRQAS |
| | CC45, 240 | GKGCEWVGRIRSKYNNYATYYADSVKDRFTISRDDSKNTAY |
| | | LQMNSLKTEDTAVYYCVRHGNFGNSYVSWWAYWGQGTLV |
| | | TVSSGGGGSGGGGSGGGGSQTVVTQEPSLTVSPGGTVTLTC |
| | | GSSTGAVTTSNYVNWVQQKPGQAPRGLIGGTNKRAPGTPAR |
| | | FSGSLIGGKAALTLSGAQPEDEAEYYCVLWYSNRWVCGGGT |
| | | KLTVL |
| 34 | Human IgG1 | ASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKKVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSREEMTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPGX |
| | | X is lysine or absent. |
| 35 | Human IgG2 | ASTKGPSVFPLAPCSRSTSESTAALGCLVKDYFPEPVTVSWNS |
| | | GALTSGVHTFPAVLQSSGLYSLSSVVTVPSSNFGTQTYTCNV |
| | | DHKPSNTKVDKTVERKCCVECPPCPAPPVAGPSVFLFPPKPK |
| | | DTLMISRTPEVTCVVVDVSHEDPEVQFNWYVDGVEVHNAK |
| | | TKPREEQFNSTFRVVSVLTVVHQDWLNGKEYKCKVSNKGLP |
| | | APIEKTISKTKGQPREPQVYTLPPSREEMTKNQVSLTCLVKGF |
| | | YPSDIAVEWESNGQPENNYKTTPPMLDSDGSFFLYSKLTVDK |
| | | SRWQQGNVFSCSVMHEALHNHYTQKSLSLSPGX |
| | | X is lysine or absent |
| 36 | Human IgG3 | ASTKGPSVFPLAPCSRSTSGGTAALGCLVKDYFPEPVTVSWN |
| | | SGALTSGVHTFPAVLQSSGLYSLSSVVTVPSSSLGTQTYTCNV |
| | | NHKPSNTKVDKRVELKTPLGDTTHTCPRCPEPKSCDTPPPCP |
| | | RCPEPKSCDTPPPCPRCPEPKSCDTPPPCPRCPAPELLGGPSVF |
| | | LFPPKPKDTLMISRTPEVTCVVVDVSHEDPEVQFKWYVDGV |
| | | EVHNAKTKPREEQYNSTFRVVSVLTVLHQDWLNGKEYKCK |
| | | VSNKALPAPIEKTISKTKGQPREPQVYTLPPSREEMTKNQVSL |
| | | TCLVKGFYPSDIAVEWESSGQPENNYNTTPPMLDSDGSFFLY |
| | | SKLTVDKSRWQQGNIFSCSVMHEALHNRFTQKSLSLSPGX |
| | | X is lysine or absent |
| 37 | Human IgG4 | ASTKGPSVFPLAPCSRSTSESTAALGCLVKDYFPEPVTVSWNS |
| | | GALTSGVHTFPAVLQSSGLYSLSSVVTVPSSSLGTKTYTCNV |
| | | DHKPSNTKVDKRVESKYGPPCPSCPAPEFLGGPSVFLFPPKPK |
| | | DTLMISRTPEVTCVVVDVSQEDPEVQFNWYVDGVEVHNAK |
| | | TKPREEQFNSTYRVVSVLTVLHQDWLNGKEYKCKVSNKGLP |
| | | SSIEKTISKAKGQPREPQVYTLPPSQEEMTKNQVSLTCLVKGF |
| | | YPSDIAVEWESNGQPENNYKTTPPVLDSDGSFFLYSRLTVDK |
| | | SRWQEGNVFSCSVMHEALHNHYTQKSLSLSLGX |
| | | X is lysine or absent |
| 38 | hIgG4 S228P | ASTKGPSVFPLAPCSRSTSESTAALGCLVKDYFPEPVTVSWNS |
| | and L235E | GALTSGVHTFPAVLQSSGLYSLSSVVTVPSSSLGTKTYTCNV |
| | CH1-Fc | DHKPSNTKVDKRVESKYGPPCPPCPAPEFEGGPSVFLFPPKPK |
| | | DTLMISRTPEVTCVVVDVSQEDPEVQFNWYVDGVEVHNAK |
| | | TKPREEQFNSTYRVVSVLTVLHQDWLNGKEYKCKVSNKGLP |
| | | SSIEKTISKAKGQPREPQVYTLPPSQEEMTKNQVSLTCLVKGF |
| | | YPSDIAVEWESNGQPENNYKTTPPVLDSDGSFFLYSRLTVDK |
| | | SRWQEGNVFSCSVMHEALHNHYTQKSLSLSLGX |
| | | X is lysine or absent. |
| 39 | IgG4-EN KiHSS | SKYGPPCPPCPAPEFEGGPSVFLFPPKPKDTLMISRTPEVTCVV |
| | -H Chain Fc | VDVSQEDPEVQFNWYVDGVEVHNAKTKPREEQFNSTYRVV |
| | ONLY | SVLTVLHQDWLNGKEYKCKVSNKGLPSSIEKTISKAKGQPRE |
| | | PQVCTLPPSQEEMTKNQVSLSCAVKGFYPSDIAVEWESNGQP |
| | | ENNYKTTPPVLDSDGSFFLVSRLTVDKSRWQEGNVFSCSVM |
| | | HEALHNHYTQKSLSLSLGX |
| | | X is lysine or absent |
| 40 | IgG4-EN KiHSS | GPPCPPCPAPEFEGGPSVFLFPPKPKDTLMISRTPEVTCVVV |
| | -K Chain-Fc | DVSQEDPEVQFNWYVDGVEVHNAKTKPREEQFNSTYRVVSV |
| | ONLY | LTVLHQDWLNGKEYKCKVSNKGLPSSIEKTISKAKGQPREPQ |
| | | VYTLPPCQEEMTKNQVSLWCLVKGFYPSDIAVEWESNGQPE |
| | | NNYKTTPPVLDSDGSFFLYSRLTVDKSRWQEGNVFSCSVMH |
| | | EALHNHYTQKSLSLSLGX |
| | | X is lysine or absent.. |
| 41 | IgG1 KiHSS-H | DKTHTCPPCPAPELLGGPSVFLFPPKPKDTLMISRTPEVTCV |
| | Chain Fc ONLY | VVDVSHEDPEVKFNWYVDGVEVHNAKTKPREEQYNSTYRVV |
| | | SVLTVLHQDWLNGKEYKCKVSNKALPAPIEKTISKAKGQPR |
| | | EPQVCTLPPSRDELTKNQVSLSCAVKGFYPSDIAVEWESNGQ |
| | | PENNYKTTPPVLDSDGSFFLVSKLTVDKSRWQQGNVFSCSV |
| | | MHEALHNHYTQKSLSLSPGX |
| | | X is lysine or absent. |
| 42 | IgG1 KiHSS-K | DKTHTCPPCPAPELLGGPSVFLFPPKPKDTLMISRTPEVTCV |
| | Chain-Fc | VVDVSHEDPEVKFNWYVDGVEVHNAKTKPREEQYNSTYRVV |
| | ONLY | SVLTVLHQDWLNGKEYKCKVSNKALPAPIEKTISKAKGQPR |
| | | EPQVYTLPPCRDELTKNQVSLWCLVKGFYPSDIAVEWESNG |
| | | QPENNYKTTPPVLDSDGSFFLYSKLTVDKSRWQQGNVFSCS |
| | | VMHEALHNHYTQKSLSLSPGX |
| | | X is lysine or absent. |

### Table 9B Exemplary Sequences of Heavy Chain Variable Domains, Light Chain Variable Domains, and Related Molecules

CDRs in the heavy chain variable domains comprise sequences of amino acids at positions 31-35, amino acids at positions 50-68, and amino acids at positions 101-114, respectively, of the heavy chain variable domains. CDRs in the light chain variable domains comprise sequences of amino acids at positions 23-36, amino acids at positions 52-58, and amino acids at positions 91-99, respectively, of the light chain variable domains.

| Anti- | | | SEQ |
|---|---|---|---|
| CD3 | Poly- | | ID |
| module | peptides | Sequences | NO |
| v619_ | Anti-CD3 | EVQLVESGGGLVQPGGSLKLSCAASGFTFSTY | 43 |
| HL15 | Heavy | AMNWVRQASGKGLEWVGRIRSKYNNYATYYAD | |
| | chain | SVKDRFTISRDDSKNTAYLQMNSLKTEDTAVY | |
| | variable | YCTRHGNFGNSYVSWFAYWGQGTLVTVSS | |
| | domain | | |
| | Anti-CD3 | QTVVTQEPSLTVSPGGTVTLTCRSSTGAVTTSN | 44 |
| | Light chain | YANWVQQKPGQAPRGLIGGTNKRAPGTPARFSG | |
| | variable | SLIGGKAALTLSGAQPEDEAEYYCALWYSNLWV | |
| | domain | FGGGTKLTVL | |
| | Anti-CD3 | EVQLVESGGGLVQPGGSLKLSCAASGFTFSTY | 45 |
| | scFv | AMNWVRQASGKGLEWVGRIRSKYNNYATYYAD | |
| | | SVKDRFTISRDDSKNTAYLQMNSLKTEDTAVY | |
| | | YCTRHGNFGNSYVSWFAYWGQGTLVTVSSGGG | |
| | | GSGGGGSGGGGSQTVVTQEPSLTVSPGGTVTL | |
| | | TCRSSTGAVTTSNYANWVQQKPGQAPRGLIGG | |
| | | TNKRAPGTPARFSGSLIGGKAALTLSGAQPED | |
| | | EAEYYCALWYSNLWVFGGGTKLTVL | |
| v619_ | Anti-CD3 | EVQLVESGGGLVQPGGSLKLSCAASGFTFSTY | 46 |
| Hla | Heavy | AMNWVRQASGKGLEWVGRIRSKYNNYATYYAD | |
| | chain | SVKDRFTISRDDSKNTAYLQMNSLKTEDTAVY | |
| | variable | YCVRHGNFGNSYVSWWAYWGQGTLVTVSS | |
| | domain | | |
| | Anti-CD3 | QTVVTQEPSLTVSPGGTVTLTCRSSTGAVTTSN | 47 |
| | Light chain | YANWVQQKPGQAPRGLIGGTKFLAPGTPARFSG | |
| | variable | SLIGGKAALTLSGAQPEDEAEYYCALWYSNRWV | |
| | domain | FGGGTKLTVL | |
| | Anti-CD3 | EVQLVESGGGLVQPGGSLKLSCAASGFTFSTY | 48 |
| | scFv | AMNWVRQASGKGLEWVGRIRSKYNNYATYYAD | |
| | | SVKDRFTISRDDSKNTAYLQMNSLKTEDTAVY | |
| | | YCVRHGNFGNSYVSWWAYWGQGTLVTVSSGGG | |
| | | GSGGGGSGGGGSQTVVTQEPSLTVSPGGTVTL | |
| | | TCRSSTGAVTTSNYANWVQQKPGQAPRGLIGG | |
| | | TKFLAPGTPARFSGSLIGGKAALTLSGAQPED | |
| | | EAEYYCALWYSNRWVFGGGTKLTVL | |
| v619_ | Anti-CD3 | EVQLVESGGGLVQPGGSLKLSCAASGFTFSTY | 46 |
| HLc | Heavy | AMNWVRQASGKGLEWVGRIRSKYNNYATYYAD | |
| | chain | SVKDRFTISRDDSKNTAYLQMNSLKTEDTAVY | |
| | variable | YCVRHGNFGNSYVSWWAYWGQGTLVTVSS | |
| | domain | | |
| | Anti-CD3 | QTVVTQEPSLTVSPGGTVTLTCRSSTGAVTTS | 49 |
| | Light chain | NYPNWVQQKPGQAPRGLIGGTNKRAPGTPARF | |
| | variable | SGSLLGGKAALTLSGVQPEDEAEYYCVLWYSN | |
| | domain | RWVFGGGTKLTVL | |
| | Anti-CD3 | EVQLVESGGGLVQPGGSLKLSCAASGFTFSTY | 50 |
| | scFv | AMNWVRQASGKGLEWVGRIRSKYNNYATYYAD | |
| | | SVKDRFTISRDDSKNTAYLQMNSLKTEDTAVY | |
| | | YCVRHGNFGNSYVSWWAYWGQGTLVTVSSGGG | |
| | | GSGGGGSGGGGSQTVVTQEPSLTVSPGGTVTL | |
| | | TCRSSTGAVTTSNYPNWVQQKPGQAPRGLIGG | |
| | | TNKRAPGTPARFSGSLLGGKAALTLSGVQPED | |
| | | EAEYYCVLWYSNRWVFGGGTKLTVL | |
| v619_ | Anti-CD3 | EVQLVESGGGLVQPGGSLKLSCAASGFTFSTY | 51 |
| HLc1 | Heavy | AMNWVRQASGKGLEWVGRIRSKYNNYATYYAD | |
| | chain | SVKDRFTISRDDSKNTAYLQMNSLKTEDTAVY | |
| | variable | YCTVHGNFGNSYVSWWAYWGQGTLVTVSS | |
| | domain | | |
| | Anti-CD3 | SQTVVTQEPSLTVSPGGTVTLTCRSSTGAVTTS | 52 |
| | Light chain | NYPNWVQQKPGQAPRGLIGGTNKRAPGTPARFS | |
| | variable | GSLLGGKAALTLSGVQPEDEAEYYCVLWYSNRW | |
| | domain | VFGGGTKLTVL | |
| | Anti-CD3 | EVQLVESGGGLVQPGGSLKLSCAASGFTFSTY | 53 |
| | scFv | AMNWVRQASGKGLEWVGRIRSKYNNYATYYAD | |
| | | SVKDRFTISRDDSKNTAYLQMNSLKTEDTAVY | |
| | | YCTVHGNFGNSYVSWWAYWGQGTLVTVSSGGG | |
| | | GSGGGGSGGGGSQTVVTQEPSLTVSPGGTVTLT | |
| | | CRSSTGAVTTSNYPNWVQQKPGQAPRGLIGGTN | |
| | | KRAPGTPARFSGSLLGGKAALTLSGVQPEDEAE | |
| | | YYCVLWYSNRWVFGGGTKLTVL | |
| v619_ | Anti-CD3 | EVQLVESGGGLVQPGGSLKLSCAASGFTFSTY | 54 |
| HLd | Heavy | AMNWVRQASGKGLEWVGRIRSKYNNYATYYAD | |
| | chain | SVKDRFTISRDDSKNTAYLQMNSLKTEDTAVY | |
| | variable | YCVRHGNFGNSYISYWAYWGQGTLVTVSS | |
| | domain | | |
| | Anti-CD3 | QTVVTQEPSLTVSPGGTVTLTCRSSTGAVTTSN | 55 |
| | Light chain | YPNWVQQKPGQAPRGLIGGTNKRAPGTPARFSG | |
| | variable | SLIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTY | 56 |
| | scFv | AMNWVRQASGKGLEWVGRIRSKYNNYATYYAD | |
| | | SVKDRFTISRDDSKNTAYLQMNSLKTEDTAVY | |
| | | YCVRHGNFGNSYISYWAYWGQGTLVTVSSGGG | |
| | | GSGGGGSGGGGSQTVVTQEPSLTVSPGGTVTL | |
| | | TCRSSTGAVTTSNYPNWVQQKPGQAPRGLIGG | |
| | | TNKRAPGTPARFSGSLIGGKAALTLSGAQPED | |
| | | EAEYYCVLWYSNRWVFGGGTKLTVL | |
| V619_ | Anti-CD3 | EVQLVESGGGLVQPGGSLKLSCAASGFTFNKY | 57 |
| Hle | Heavy | AMNWVRQASGKGLEWVGRIRSKYNNYATYYAD | |
| | chain | SVKDRFTISRDDSKNTAYLQMNSLKTEDTAVY | |
| | variable | YCTRHGNFGNSYVSWFAYWGQGTLVTVSS | |
| | domain | | |
| | Anti-CD3 | QTVVTQEPSLTVSPGGTVTLTCRSSTGAVTSGN | 58 |
| | Light chain | YANWVQQKPGQAPRGLIGGTNKRAPGTPARFS | |
| | variable | GSLIGGKAALTLSGVQPEDEAEYYCALWYSNR | |
| | domain | WVFGGGTKLTVL | |
| | Anti-CD3 | EVQLVESGGGLVQPGGSLKLSCAASGFTFNKY | 59 |
| | scFv | AMNWVRQASGKGLEWVGRIRSKYNNYATYYAD | |
| | | SVKDRFTISRDDSKNTAYLQMNSLKTEDTAVYY | |
| | | CTRHGNFGNSYVSWFAYWGQGTLVTVSSGGGG | |
| | | SGGGGSGGGGSQTVVTQEPSLTVSPGGTVTLTC | |
| | | RSSTGAVTSGNYANWVQQKPGQAPRGLIGGTNK | |
| | | RAPGTPARFSGSLIGGKAALTLSGVQPEDEAEY | |
| | | YCALWYSNRWVFGGGTKLTVL | |
| v619_ | Anti-CD3 | EVQLVESGGGLVQPGGSLKLSCAASGFTFNKYA | 57 |
| HLf | Heavy | MNWVRQASGKGLEWVGRIRSKYNNYATYYAD | |
| | chain | SVKDRFTISRDDSKNTAYLQMNSLKTEDTAVYY | |
| | variable | CTRHGNFGNSYVSWFAYWGQGTLVTVSS | |
| | domain | | |
| | Anti-CD3 | QTVVTQEPSLTVSPGGTVTLTCGSSTGAVTTSNY | 60 |
| | Light chain | ANWVQQKPGQAPRGLIGGTNKRAPGTPARFSGS | |
| | variable | LIGGKAALTLSGAQPEDEAEYYCVLWYSNLWV | |
| | domain | FGGGTKLTVL | |
| | Anti-CD3 | EVQLVESGGGLVQPGGSLKLSCAASGFTENKY | 61 |
| | scFv | AMNWVRQASGKGLEWVGRIRSKYNNYATYYAD | |
| | | SVKDRFTISRDDSKNTAYLQMNSLKTEDTAVY | |
| | | YCTRHGNFGNSYVSWFAYWGQGTLVTVSSGGG | |
| | | GSGGGGSGGGGSQTVVTQEPSLTVSPGGTVTL | |
| | | TCGSSTGAVTTSNYANWVQQKPGQAPRGLIGG | |
| | | TNKRAPGTPARFSGSLIGGKAALTLSGAQPED | |
| | | EAEYYCVLWYSNLWVFGGGTKLTVL | |
| v619_ | Anti-CD3 | EVQLVESGGGLVQPGGSLKLSCAASGFTFSTYA | 43 |
| HLg | Heavy | MNWVRQASGKGLEWVGRIRSKYNNYATYYAD | |
| | chain | SVKDRFTISRDDSKNTAYLQMNSLKTEDTAVYY | |
| | variable | CTRHGNFGNSYVSWFAYWGQGTLVTVSS | |
| | domain | | |
| | Anti-CD3 | QTVVTQEPSLTVSPGGTVTLTCRSSTGAVTSGN | 62 |
| | Light chain | YPNWVQQKPGQAPRGLIGGTKFLAPGTPARFSG | |
| | variable | SLLGGKAALTLSGVQPEDEAEYYCALWYSNLW | |
| | domain | VFGGGTKLTVL | |
| | Anti-CD3 | EVQLVESGGGLVQPGGSLKLSCAASGFTFSTYA | 63 |
| | scFv | MNWVRQASGKGLEWVGRIRSKYNNYATYYAD | |
| | | SVKDRFTISRDDSKNTAYLQMNSLKTEDTAVYY | |
| | | CTRHGNFGNSYVSWFAYWGQGTLVTVSSGGGG | |
| | | SGGGGSGGGGSQTVVTQEPSLTVSPGGTVTLTC | |
| | | RSSTGAVTSGNYPNWVQQKPGQAPRGLIGGTKF | |
| | | LAPGTPARFSGSLLGGKAALTLSGVQPEDEAEY | |
| | | YCALWYSNLWVFGGGTKLTVL | |
| v619_ | Anti-CD3 | EVQLVESGGGLVQPGGSLKLSCAASGFTESTYA | 46 |
| HLh | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCGSSTGAVTTSNY | 64 |
| | Light chain | VNWVQQKPGQAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 28 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CGSSTGAVTTSNYVNWVQQKPGQAPRGLIGGT | |
| | | NKRAPGTPARFSGSLIGGKAALTLSGAQPEDEAE | |
| | | YYCVLWYSNRWVFGGGTKLTVL | |
| v619_ | Anti-CD3 | EVQLVESGGGLVQPGGSLKLSCAASGFTFSTYA | 51 |
| HLh1 | Heavy | MNWVRQASGKGLEWVGRIRSKYNNYATYYAD | |
| | chain | SVKDRFTISRDDSKNTAYLQMNSLKTEDTAVYY | |
| | variable | CTVHGNFGNSYVSWWAYWGQGTLVTVSS | |
| | domain | | |
| | Anti-CD3 | QTVVTQEPSLTVSPGGTVTLTCGSSTGAVTTSNY | 64 |
| | Light chain | VNWVQQKPGQAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 65 |
| | scFv | MNWVRQASGKGLEWVGRIRSKYNNYATYYAD | |
| | | SVKDRFTISRDDSKNTAYLQMNSLKTEDTAVYY | |
| | | CTVHGNFGNSYVSWWAYWGQGTLVTVSSGGG | |
| | | GSGGGGSGGGGSQTVVTQEPSLTVSPGGTVTLT | |
| | | CGSSTGAVTTSNYVNWVQQKPGQAPRGLIGGT | |
| | | NKRAPGTPARFSGSLIGGKAALTLSGAQPEDEAE | |
| | | YYCVLWYSNRWVFGGGTKLTVL | |
| v619_ | Anti-CD3 | EVQLVESGGGLVQPGGSLKLSCAASGFTFSTYA | 66 |
| Hli | Heavy | MNWVRQASGKGLEWVGRIRSKYNNYATYYAD | |
| | chain | SVKDRFTISRDDSKNTAYLQMNSLKTEDTAVYY | |
| | variable | CTRHGNFGNSYISYWAYWGQGTLVTVSS | |
| | domain | | |
| | Anti-CD3 | QTVVTQEPSLTVSPGGTVTLTCRSSTGAVTTSNY | 67 |
| | Light chain | ANWVQQKPGQAPRGLIGGTKFLAPGTPARFSGS | |
| | variable | LIGGKAALTLSGAQPEDEAEYYCALWYSNLWV | |
| | domain | FGGGTKLTVL | |
| | Anti-CD3 | EVQLVESGGGLVQPGGSLKLSCAASGFTFSTYA | 68 |
| | scFv | MNWVRQASGKGLEWVGRIRSKYNNYATYYAD | |
| | | SVKDRFTISRDDSKNTAYLQMNSLKTEDTAVYY | |
| | | CTRHGNFGNSYISYWAYWGQGTLVTVSSGGGG | |
| | | SGGGGSGGGGSQTVVTQEPSLTVSPGGTVTLTC | |
| | | RSSTGAVTTSNYANWVQQKPGQAPRGLIGGTKF | |
| | | LAPGTPARFSGSLIGGKAALTLSGAQPEDEAEYY | |
| | | CALWYSNLWVFGGGTKLTVL | |
| V619_ | Anti-CD3 | EVQLVESGGGLVQPGGSLKLSCAASGFTFSTYA | 54 |
| HLj | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYISYWAYWGQGTLVTVSS | |
| | domain | | |
| | Anti-CD3 | QTVVTQEPSLTVSPGGTVTLTCRSSTGAVTTSNY | 67 |
| | Light chain | ANWVQQKPGQAPRGLIGGTKFLAPGTPARFSGS | |
| | variable | LIGGKAALTLSGAQPEDEAEYYCALWYSNLWV | |
| | domain | FGGGTKLTVL | |
| | Anti-CD3 | EVQLVESGGGLVQPGGSLKLSCAASGFTFSTYA | 69 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYISYWAYWGQGTLVTVSSGGGG | |
| | | SGGGGSGGGGSQTVVTQEPSLTVSPGGTVTLTC | |
| | | RSSTGAVTTSNYANWVQQKPGQAPRGLIGGTKF | |
| | | LAPGTPARFSGSLIGGKAALTLSGAQPEDEAEYY | |
| | | CALWYSNLWVFGGGTKLTVL | |
| v619_ | Anti-CD3 | EVQLVESGGGLVQPGGSLKLSCAASGFTFSTYA | 70 |
| HLk | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWFAYWGQGTLVTVSS | |
| | domain | | |
| | Anti-CD3 | QTVVTQEPSLTVSPGGTVTLTCGSSTGAVTSGN | 71 |
| | Light chain | YPNWVQQKPGQAPRGLIGGTNKRAPGTPARFSG | |
| | variable | SLIGGKAALTLSGAQPEDEAEYYCALWYSNLW | |
| | domain | VFGGGTKLTVL | |
| | Anti-CD3 | EVQLVESGGGLVQPGGSLKLSCAASGFTFSTYA | 72 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWFAYWGQGTLVTVSSGGG | |
| | | GSGGGGSGGGGSQTVVTQEPSLTVSPGGTVTLT | |
| | | CGSSTGAVTSGNYPNWVQQKPGQAPRGLIGGT | |
| | | NKRAPGTPARFSGSLIGGKAALTLSGAQPEDEAE | |
| | | YYCALWYSNLWVFGGGTKLTVL | |
| v619_ | Anti-CD3 | EVQLVESGGGLVQPGGSLKLSCAASGFTFNKYA | 73 |
| HLl | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCGSSTGAVTSGN | 74 |
| | Light chain | YPNWVQQKPGQAPRGLIGGTKFLAPGTPARFSG | |
| | variable | SLIGGKAALTLSGAQPEDEAEYYCVLWYSNLW | |
| | domain | VFGGGTKLTVL | |
| | Anti-CD3 | EVQLVESGGGLVQPGGSLKLSCAASGFTFNKYA | 75 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CGSSTGAVTSGNYPNWVQQKPGQAPRGLIGGT | |
| | | KFLAPGTPARFSGSLIGGKAALTLSGAQPEDEAE | |
| | | YYCVLWYSNLWVFGGGTKLTVL | |
| v619_ | Anti-CD3 | EVQLVESGGGLVQPGGSLKLSCAASGFTFNKYA | 76 |
| HLm | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYISYWAYWGQGTLVTVSS | |
| | domain | | |
| | Anti-CD3 | QTVVTQEPSLTVSPGGTVTLTCGSSTGAVTSGN | 74 |
| | Light chain | YPNWVQQKPGQAPRGLIGGTKFLAPGTPARFSG | |
| | variable | SLIGGKAALTLSGAQPEDEAEYYCVLWYSNLW | |
| | domain | VFGGGTKLTVL | |
| | Anti-CD3 | EVQLVESGGGLVQPGGSLKLSCAASGFTFNKYA | 77 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYISYWAYWGQGTLVTVSSGGGG | |
| | | SGGGGSGGGGSQTVVTQEPSLTVSPGGTVTLTC | |
| | | GSSTGAVTSGNYPNWVQQKPGQAPRGLIGGTKF | |
| | | LAPGTPARFSGSLIGGKAALTLSGAQPEDEAEYY | |
| | | CVLWYSNLWVFGGGTKLTVL | |
| v619_ | Anti-CD3 | EVQLVESGGGLVQPGGSLKLSCAASGFTFNTYA | 78 |
| HLn | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCGSSTGAVTSGN | 74 |
| | Light chain | YPNWVQQKPGQAPRGLIGGTKFLAPGTPARFSG | |
| | variable | SLIGGKAALTLSGAQPEDEAEYYCVLWYSNLW | |
| | domain | VFGGGTKLTVL | |
| | Anti-CD3 | EVQLVESGGGLVQPGGSLKLSCAASGFTFNTYA | 79 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CGSSTGAVTSGNYPNWVQQKPGQAPRGLIGGT | |
| | | KFLAPGTPARFSGSLIGGKAALTLSGAQPEDEAE | |
| | | YYCVLWYSNLWVFGGGTKLTVL | |
| V619_ | Anti-CD3 | EVQLVESGGGLVQPGGSLKLSCAASGFTFNTYA | 78 |
| HLo | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCGSSTGVVTSSNY | 80 |
| | Light chain | PNWVQQKPGQAPRGLIGGTKFLAPGTPARFSGS | |
| | variable | LIGGKAALTLSGAQPEDEAEYYCVLWYSNLWV | |
| | domain | FGGGTKLTVL | |
| | Anti-CD3 | EVQLVESGGGLVQPGGSLKLSCAASGFTFNTYA | 81 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGSG | |
| | | GSGGGGSGGGGSQTVVTQEPSLTVSPGGTVTLT | |
| | | CGSSTGVVTSSNYPNWVQQKPGQAPRGLIGGTK | |
| | | FLAPGTPARFSGSLIGGKAALTLSGAQPEDEAEY | |
| | | YCVLWYSNLWVFGGGTKLTVL | |
| v619_ | Anti-CD3 | EVQLVESGGGLVQPGGSLKLSCAASGFTFSTYA | 82 |
| HLv2 | Heavy | MNWVRQAPGKGLEWVARIRSKYNNYATYYAD | |
| | chain | SVKDRFTISRDDSKNTAYLQMNSLKTEDTAVYY | |
| | variable | CVRHGNFGNSYVSWFAYWGQGTLVTVSS | |
| | domain | | |
| | Anti-CD3 | QTVVTQEPSLTVSPGGTVTLTCRSSTGAVTTSNY | 44 |
| | Light chain | ANWVQQKPGQAPRGLIGGTNKRAPGTPARFSGS | |
| | variable | LIGGKAALTLSGAQPEDEAEYYCALWYSNLWV | |
| | domain | FGGGTKLTVL | |
| | Anti-CD3 | EVQLVESGGGLVQPGGSLKLSCAASGFTFSTYA | 83 |
| | scFv | MNWVRQAPGKGLEWVARIRSKYNNYATYYAD | |
| | | SVKDRFTISRDDSKNTAYLQMNSLKTEDTAVYY | |
| | | CVRHGNFGNSYVSWFAYWGQGTLVTVSSGGG | |
| | | GSGGGGSGGGGSQTVVTQEPSLTVSPGGTVTLT | |
| | | CRSSTGAVTTSNYANWVQQKPGQAPRGLIGGT | |
| | | NKRAPGTPARFSGSLIGGKAALTLSGAQPEDEAE | |
| | | YYCALWYSNLWVFGGGTKLTVL | |
| v619_ | Anti-CD3 | EVQLVESGGGLVQPGGSLKLSCAASGFTFSTYA | 82 |
| HLK1 | Heavy | MNWVRQAPGKGLEWVARIRSKYNNYATYYAD | |
| | chain | SVKDRFTISRDDSKNTAYLQMNSLKTEDTAVYY | |
| | variable | CVRHGNFGNSYVSWFAYWGQGTLVTVSS | |
| | domain | | |
| | Anti-CD3 | QTVVTQEPSLTVSPGGTVTLTCGSSTGAVTSGN | 71 |
| | Light chain | YPNWVQQKPGQAPRGLIGGTNKRAPGTPARFSG | |
| | variable | SLIGGKAALTLSGAQPEDEAEYYCALWYSNLW | |
| | domain | VFGGGTKLTVL | |
| | Anti-CD3 | EVQLVESGGGLVQPGGSLKLSCAASGFTFSTYA | 84 |
| | scFv | MNWVRQAPGKGLEWVARIRSKYNNYATYYAD | |
| | | SVKDRFTISRDDSKNTAYLQMNSLKTEDTAVYY | |
| | | CVRHGNFGNSYVSWFAYWGQGTLVTVSSGGG | |
| | | GSGGGGSGGGGSQTVVTQEPSLTVSPGGTVTLT | |
| | | CGSSTGAVTSGNYPNWVQQKPGQAPRGLIGGT | |
| | | NKRAPGTPARFSGSLIGGKAALTLSGAQPEDEAE | |
| | | YYCALWYSNLWVFGGGTKLTVL | |
| v619_ | Anti-CD3 | EVQLVESGGGLVQPGGSLKLSCAASGFTFSTYA | 70 |
| Hh1 | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWFAYWGQGTLVTVSS | |
| | domain | | |
| | Anti-CD3 | QTVVTQEPSLTVSPGGTVTLTCGSSTGAVTTSNY | 64 |
| | Light chain | VNWVQQKPGQAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 85 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWFAYWGQGTLVTVSSGGG | |
| | | GSGGGGSGGGGSQTVVTQEPSLTVSPGGTVTLT | |
| | | CGSSTGAVTTSNYVNWVQQKPGQAPRGLIGGT | |
| | | NKRAPGTPARFSGSLIGGKAALTLSGAQPEDEAE | |
| | | YYCVLWYSNRWVFGGGTKLTVL | |
| v619_ | Anti-CD3 | EVQLVESGGGLVQPGGSLKLSCAASGFTFSTYA | 70 |
| Hh2 | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWFAYWGQGTLVTVSS | |
| | domain | | |
| | Anti-CD3 | QTVVTQEPSLTVSPGGTVTLTCGSSTGAVTTSNY | 86 |
| | | AVWVQQKPGQAPRGLIGGTNKRAPGTPARFSGS | |
| | Light chain | LIGGKAALTLSGAQPEDEAEYYCVLWYSNRWV | |
| | variable | FGGGTKLTVL | |
| | domain | | |
| | Anti-CD3 | EVQLVESGGGLVQPGGSLKLSCAASGFTFSTYA | 87 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWFAYWGQGTLVTVSSGGG | |
| | | GSGGGGSGGGGSQTVVTQEPSLTVSPGGTVTLT | |
| | | CGSSTGAVTTSNYAVWVQQKPGQAPRGLIGGT | |
| | | NKRAPGTPARFSGSLIGGKAALTLSGAQPEDEAE | |
| | | YYCVLWYSNRWVFGGGTKLTVL | |
| v619_ | Anti-CD3 | EVQLVESGGGLVQPGGSLKLSCAASGFTFSTYA | 70 |
| Hh3 | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWFAYWGQGTLVTVSS | |
| | domain | | |
| | Anti-CD3 | QTVVTQEPSLTVSPGGTVTLTCGSSTGAVTTSNY | 88 |
| | Light chain | AFWVQQKPGQAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 89 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWFAYWGQGTLVTVSSGGG | |
| | | GSGGGGSGGGGSQTVVTQEPSLTVSPGGTVTLT | |
| | | CGSSTGAVTTSNYAFWVQQKPGQAPRGLIGGTN | |
| | | KRAPGTPARFSGSLIGGKAALTLSGAQPEDEAEY | |
| | | YCVLWYSNRWVFGGGTKLTVL | |
| v619_ | Anti-CD3 | EVQLVESGGGLVQPGGSLKLSCAASGFTFSTYA | 90 |
| Hh4 | Heavy | MHWVRQASGKGLEWVGRIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCGSSTGAVTTSNY | 64 |
| | Light chain | VNWVQQKPGQAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 91 |
| | scFv | MHWVRQASGKGLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CGSSTGAVTTSNYVNWVQQKPGQAPRGLIGGT | |
| | | NKRAPGTPARFSGSLIGGKAALTLSGAQPEDEAE | |
| | | YYCVLWYSNRWVFGGGTKLTVL | |
| v619_ | Anti-CD3 | EVQLVESGGGLVQPGGSLKLSCAASGFTFSTYA | 92 |
| Hh5 | Heavy | MIWVRQASGKGLEWVGRIRSKYNNYATYYADS | |
| | chain | VKDRFTISRDDSKNTAYLQMNSLKTEDTAVYYC | |
| | variable | VRHGNFGNSYVSWWAYWGQGTLVTVSS | |
| | domain | | |
| | Anti-CD3 | QTVVTQEPSLTVSPGGTVTLTCGSSTGAVTTSNY | 64 |
| | Light chain | VNWVQQKPGQAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 93 |
| | scFv | MIWVRQASGKGLEWVGRIRSKYNNYATYYADS | |
| | | VKDRFTISRDDSKNTAYLQMNSLKTEDTAVYYC | |
| | | VRHGNFGNSYVSWWAYWGQGTLVTVSSGGGG | |
| | | SGGGGSGGGGSQTVVTQEPSLTVSPGGTVTLTC | |
| | | GSSTGAVTTSNYVNWVQQKPGQAPRGLIGGTN | |
| | | KRAPGTPARFSGSLIGGKAALTLSGAQPEDEAEY | |
| | | YCVLWYSNRWVFGGGTKLTVL | |
| v619_ | Anti-CD3 | EVQLVESGGGLVQPGGSLKLSCAASGFTFSTYA | 94 |
| Hh6 | Heavy | MLWVRQASGKGLEWVGRIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCGSSTGAVTTSNY | 64 |
| | Light chain | VNWVQQKPGQAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 95 |
| | scFv | MLWVRQASGKGLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CGSSTGAVTTSNYVNWVQQKPGQAPRGLIGGT | |
| | | NKRAPGTPARFSGSLIGGKAALTLSGAQPEDEAE | |
| | | YYCVLWYSNRWVFGGGTKLTVL | |
| v619_ | Anti-CD3 | EVQLVESGGGLVQPGGSLKLSCAASGFTFSTYA | 70 |
| Hh7 | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWFAYWGQGTLVTVSS | |
| | domain | | |
| | Anti-CD3 | QTVVTQEPSLTVSPGGTVTLTCGSSTGAVTTSNY | 96 |
| | Light chain | AYWVQQKPGQAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 97 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWFAYWGQGTLVTVSSGGG | |
| | | GSGGGGSGGGGSQTVVTQEPSLTVSPGGTVTLT | |
| | | CGSSTGAVTTSNYAYWVQQKPGQAPRGLIGGT | |
| | | NKRAPGTPARFSGSLIGGKAALTLSGAQPEDEAE | |
| | | YYCVLWYSNRWVFGGGTKLTVL | |
| v619_ | Anti-CD3 | EVQLVESGGGLVQPGGSLKLSCAASGFTFSTYA | 46 |
| Hh8 | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCGSSTGAVTTSNY | 98 |
| | Light chain | VNWVQQKPGQAPRGLIGGTNKRAPGTPARFSGS | |
| | variable | LIGGKAALTLSGAQPEDEAEYYCVLWYSNLWV | |
| | domain | FGGGTKLTVL | |
| | Anti-CD3 | EVQLVESGGGLVQPGGSLKLSCAASGFTFSTYA | 99 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CGSSTGAVTTSNYVNWVQQKPGQAPRGLIGGT | |
| | | NKRAPGTPARFSGSLIGGKAALTLSGAQPEDEAE | |
| | | YYCVLWYSNLWVFGGGTKLTVL | |
| v619_ | Anti-CD3 | EVQLVESGGGLVQPGGSLKLSCAASGFTFSTYA | 46 |
| Hh9 | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCRSSTGAVTTSNY | 100 |
| | Light chain | ANWVQQKPGQAPRGLIGGTNKRAPGTPARFSGS | |
| | variable | LIGGKAALTLSGAQPEDEAEYYCVLWYSNLWV | |
| | domain | FGGGTKLTVL | |
| | Anti-CD3 | EVQLVESGGGLVQPGGSLKLSCAASGFTFSTYA | 101 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CRSSTGAVTTSNYANWVQQKPGQAPRGLIGGT | |
| | | NKRAPGTPARFSGSLIGGKAALTLSGAQPEDEAE | |
| | | YYCVLWYSNLWVFGGGTKLTVL | |
| v619_ | Anti-CD3 | EVQLVESGGGLVQPGGSLKLSCAASGFTFSTYA | 102 |
| Hh10 | Heavy | MIWVRQASGKGLEWVGRIRSKYNNYATYYADS | |
| | chain | VKDRFTISRDDSKNTAYLQMNSLKTEDTAVYYC | |
| | variable | VRHGNFGNSYVSWFAYWGQGTLVTVSS | |
| | domain | | |
| | Anti-CD3 | QTVVTQEPSLTVSPGGTVTLTCRSSTGAVTTSNY | 103 |
| | Light chain | AYWVQQKPGQAPRGLIGGTNKRAPGTPARFSGS | |
| | variable | LIGGKAALTLSGAQPEDEAEYYCVLWYSNLWV | |
| | domain | FGGGTKLTVL | |
| | Anti-CD3 | EVQLVESGGGLVQPGGSLKLSCAASGFTFSTYA | 104 |
| | scFv | MIWVRQASGKGLEWVGRIRSKYNNYATYYADS | |
| | | VKDRFTISRDDSKNTAYLQMNSLKTEDTAVYYC | |
| | | VRHGNFGNSYVSWFAYWGQGTLVTVSSGGGGS | |
| | | GGGGSGGGGSQTVVTQEPSLTVSPGGTVTLTCR | |
| | | SSTGAVTTSNYAYWVQQKPGQAPRGLIGGTNK | |
| | | RAPGTPARFSGSLIGGKAALTLSGAQPEDEAEYY | |
| | | CVLWYSNLWVFGGGTKLTVL | |
| v619_ | Anti-CD3 | EVQLVESGGGLVQPGGSLKLSCAASGFTFSTYA | 46 |
| Hh12 | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCGSSTGAVTTSNY | 105 |
| | Light chain | ANWVQQKPGQAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 106 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CGSSTGAVTTSNYANWVQQKPGQAPRGLIGGT | |
| | | NKRAPGTPARFSGSLIGGKAALTLSGAQPEDEAE | |
| | | YYCVLWYSNRWVFGGGTKLTVL | |
| v619_ | Anti-CD3 | EVQLVESGGGLVQPGGSLKLSCAASGFTFSTYA | 46 |
| Hh13 | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCRSSTGAVTTSNY | 107 |
| | Light chain | VNWVQQKPGQAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 108 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CRSSTGAVTTSNYVNWVQQKPGQAPRGLIGGT | |
| | | NKRAPGTPARFSGSLIGGKAALTLSGAQPEDEAE | |
| | | YYCVLWYSNRWVFGGGTKLTVL | |
| v619_ | Anti-CD3 | EVQLVESGGGLVQPGGSLKLSCAASGFTFSTYA | 46 |
| Hh15 | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCGSSTGAVTTSNY | 109 |
| | Light chain | VNWVQQKPGQAPRGLIGGTNKRAPGTPARFSGS | |
| | variable | LIGGKAALTLSGAQPEDEAEYYCALWYSNRWV | |
| | domain | FGGGTKLTVL | |
| | Anti-CD3 | EVQLVESGGGLVQPGGSLKLSCAASGFTFSTYA | 110 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CGSSTGAVTTSNYVNWVQQKPGQAPRGLIGGT | |
| | | NKRAPGTPARFSGSLIGGKAALTLSGAQPEDEAE | |
| | | YYCALWYSNRWVFGGGTKLTVL | |
| v619_ | Anti-CD3 | EVQLVESGGGLVQPGGSLKLSCAASGFTFSTYA | 111 |
| Hh17 | Heavy | MNWVRQAPGKGLEWVARIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCGSSTGAVTTSNY | 64 |
| | Light chain | VNWVQQKPGQAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 112 |
| | scFv | MNWVRQAPGKGLEWVARIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CGSSTGAVTTSNYVNWVQQKPGQAPRGLIGGT | |
| | | NKRAPGTPARFSGSLIGGKAALTLSGAQPEDEAE | |
| | | YYCVLWYSNRWVFGGGTKLTVL | |
| v619_ | Anti-CD3 | EVQLVESGGGLVQPGGSLKLSCAASGFTFSTYA | 46 |
| HLp2 | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCRSSTGAVTTSNY | 113 |
| | Light chain | VNWVQQKPGQAPRGLIGGTNKRAPGTPARFSGS | |
| | variable | LIGGKAALTLSGAQPEDEAEYYCILWYSNRWVF | |
| | domain | GGGTKLTVL | |
| | Anti-CD3 | EVQLVESGGGLVQPGGSLKLSCAASGFTFSTYA | 114 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CRSSTGAVTTSNYVNWVQQKPGQAPRGLIGGT | |
| | | NKRAPGTPARFSGSLIGGKAALTLSGAQPEDEAE | |
| | | YYCILWYSNRWVFGGGTKLTVL | |
| v619_ | Anti-CD3 | EVQLVESGGGLVQPGGSLKLSCAASGFTFSTYA | 46 |
| HLp3 | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCRSSTGAVTTSNY | 115 |
| | Light chain | VNWVQQKPGQAPRGLIGGTNKRAPGTPARFSGS | |
| | variable | LIGGKAALTLSGAQPEDEAEYYCILWYSNLWVF | |
| | domain | GGGTKLTVL | |
| | Anti-CD3 | EVQLVESGGGLVQPGGSLKLSCAASGFTFSTYA | 116 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CRSSTGAVTTSNYVNWVQQKPGQAPRGLIGGT | |
| | | NKRAPGTPARFSGSLIGGKAALTLSGAQPEDEAE | |
| | | YYCILWYSNLWVFGGGTKLTVL | |
| v619_ | Anti-CD3 | EVQLVESGGGLVQPGGSLKLSCAASGFTFSTYA | 46 |
| HLp4 | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCPSSTGAVTTSNY | 117 |
| | Light chain | GNWVQQKPGQAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 118 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CPSSTGAVTTSNYGNWVQQKPGQAPRGLIGGTN | |
| | | KRAPGTPARFSGSLIGGKAALTLSGAQPEDEAEY | |
| | | YCVLWYSNRWVFGGGTKLTVL | |
| v619_ | Anti-CD3 | EVQLVESGGGLVQPGGSLKLSCAASGFTFSTYA | 46 |
| HLp5 | Heavy | MNWVRQASGKGLEWVGRIRSKYNNYATYYAD | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCPSSTGAVTTSNY | 119 |
| | Light chain | VNWVQQKPGQAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 120 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CPSSTGAVTTSNYVNWVQQKPGQAPRGLIGGTN | |
| | | KRAPGTPARFSGSLIGGKAALTLSGAQPEDEAEY | |
| | | YCVLWYSNRWVFGGGTKLTVL | |
| v619- | Anti-CD3 | EVQLVESGGGLVQPGGSLKLSCAASGFTFSTYA | 121 |
| HLh- | Heavy | MNWVRQASGKGLEWVGRIRSKYNNYATYYAD | |
| CC1 | chain | SVKDRFTISRDDSKNTAYLQMNSLKTEDTAVYY | |
| | variable | CVRHGNFGNSYVSWFAYWCQGTLVTVSS | |
| | domain | | |
| | Anti-CD3 | QTVVTQEPSLTVSPGGTVTLTCGSSTGAVTTSNY | 122 |
| | Light chain | VNWVQQKPGCAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 123 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWFAYWCQGTLVTVSSGGG | |
| | | GSGGGGSGGGGSQTVVTQEPSLTVSPGGTVTLT | |
| | | CGSSTGAVTTSNYVNWVQQKPGCAPRGLIGGT | |
| | | NKRAPGTPARFSGSLIGGKAALTLSGAQPEDEAE | |
| | | YYCVLWYSNRWVFGGGTKLTVL | |
| v619- | Anti-CD3 | EVQLVESGGGLVQPGGSLKLSCAASGFTFSTYA | 121 |
| HLh- | Heavy | MNWVRQASGKGLEWVGRIRSKYNNYATYYAD | |
| CC2 | chain | SVKDRFTISRDDSKNTAYLQMNSLKTEDTAVYY | |
| | variable | CVRHGNFGNSYVSWFAYWCQGTLVTVSS | |
| | domain | | |
| | Anti-CD3 | QTVVTQEPSLTVSPGGTVTLTCGSSTGAVTTSNY | 124 |
| | Light chain | VNWVQQKPGQCPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 125 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWFAYWCQGTLVTVSSGGG | |
| | | GSGGGGSGGGGSQTVVTQEPSLTVSPGGTVTLT | |
| | | CGSSTGAVTTSNYVNWVQQKPGQCPRGLIGGT | |
| | | NKRAPGTPARFSGSLIGGKAALTLSGAQPEDEAE | |
| | | YYCVLWYSNRWVFGGGTKLTVL | |
| v619- | Anti-CD3 | EVQLVESGGGLVQPGGSLKLSCAASGFTFSTYA | 121 |
| HLh- | Heavy | MNWVRQASGKGLEWVGRIRSKYNNYATYYAD | |
| CC3 | chain | SVKDRFTISRDDSKNTAYLQMNSLKTEDTAVYY | |
| | variable | CVRHGNFGNSYVSWFAYWCQGTLVTVSS | |
| | domain | | |
| | Anti-CD3 | QTVVTQEPSLTVSPGGTVTLTCGSSTGAVTTSN | 126 |
| | Light chain | YVNWVQQKPGQACRGLIGGTNKRAPGTPARFSG | |
| | variable | SLIGGKAALTLSGAQPEDEAEYYCVLWYSNRW | |
| | domain | VFGGGTKLTVL | |
| | Anti-CD3 | EVQLVESGGGLVQPGGSLKLSCAASGFTFSTY | 127 |
| | scFv | AMNWVRQASGKGLEWVGRIRSKYNNYATYYAD | |
| | | SVKDRFTISRDDSKNTAYLQMNSLKTEDTAVY | |
| | | YCVRHGNFGNSYVSWFAYWCQGTLVTVSSGGG | |
| | | GSGGGGSGGGGSQTVVTQEPSLTVSPGGTVTL | |
| | | TCGSSTGAVTTSNYVNWVQQKPGQACRGLIGG | |
| | | TNKRAPGTPARFSGSLIGGKAALTLSGAQPED | |
| | | EAEYYCVLWYSNRWVFGGGTKLTVL | |
| v619- | Anti-CD3 | EVQLVESGGGLVQPGGSLKLSCAASGFTFSTY | 128 |
| HLh- | Heavy | AMNWVRQASGKGLEWVGRIRSKYNNYATYYAD | |
| CC4 | chain | SVKDRFTISRDDSKNTAYLQMNSLKTEDTAVY | |
| | variable | YCVRHGNFGNSYVSWFAYWGCGTLVTVSS | |
| | domain | | |
| | Anti-CD3 | QTVVTQEPSLTVSPGGTVTLTCGSSTGAVTTSNY | 122 |
| | Light chain | VNWVQQKPGCAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTY | 32 |
| | scFv | AMNWVRQASGKGLEWVGRIRSKYNNYATYYAD | |
| | | SVKDRFTISRDDSKNTAYLQMNSLKTEDTAVY | |
| | | YCVRHGNFGNSYVSWFAYWGCGTLVTVSSGGG | |
| | | GSGGGGSGGGGSQTVVTQEPSLTVSPGGTVTL | |
| | | TCGSSTGAVTTSNYVNWVQQKPGCAPRGLIGG | |
| | | TNKRAPGTPARFSGSLIGGKAALTLSGAQPED | |
| | | EAEYYCVLWYSNRWVFGGGTKLTVL | |
| v619- | Anti-CD3 | EVQLVESGGGLVQPGGSLKLSCAASGFTFSTY | 128 |
| HLh- | Heavy | AMNWVRQASGKGLEWVGRIRSKYNNYATYYAD | |
| CC5 | chain | SVKDRFTISRDDSKNTAYLQMNSLKTEDTAVY | |
| | variable | YCVRHGNFGNSYVSWFAYWGCGTLVTVSS | |
| | domain | | |
| | Anti-CD3 | QTVVTQEPSLTVSPGGTVTLTCGSSTGAVTTSNY | 124 |
| | Light chain | VNWVQQKPGQCPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTY | 129 |
| | scFv | AMNWVRQASGKGLEWVGRIRSKYNNYATYYAD | |
| | | SVKDRFTISRDDSKNTAYLQMNSLKTEDTAVY | |
| | | YCVRHGNFGNSYVSWFAYWGCGTLVTVSSGGG | |
| | | GSGGGGSGGGGSQTVVTQEPSLTVSPGGTVTLT | |
| | | CGSSTGAVTTSNYVNWVQQKPGQCPRGLIGGTN | |
| | | KRAPGTPARFSGSLIGGKAALTLSGAQPEDEAE | |
| | | YYCVLWYSNRWVFGGGTKLTVL | |
| v619- | Anti-CD3 | EVQLVESGGGLVQPGGSLKLSCAASGFTFSTY | 128 |
| HLh- | Heavy | AMNWVRQASGKGLEWVGRIRSKYNNYATYYAD | |
| CC6 | chain | SVKDRFTISRDDSKNTAYLQMNSLKTEDTAVY | |
| | variable | YCVRHGNFGNSYVSWFAYWGCGTLVTVSS | |
| | domain | | |
| | Anti-CD3 | QTVVTQEPSLTVSPGGTVTLTCGSSTGAVTTSN | 126 |
| | Light chain | YVNWVQQKPGQACRGLIGGTNKRAPGTPARFSG | |
| | variable | SLIGGKAALTLSGAQPEDEAEYYCVLWYSNRW | |
| | domain | VFGGGTKLTVL | |
| | Anti-CD3 | EVQLVESGGGLVQPGGSLKLSCAASGFTFSTY | 130 |
| | scFv | AMNWVRQASGKGLEWVGRIRSKYNNYATYYAD | |
| | | SVKDRFTISRDDSKNTAYLQMNSLKTEDTAVY | |
| | | YCVRHGNFGNSYVSWFAYWGCGTLVTVSSGGG | |
| | | GSGGGGSGGGGSQTVVTQEPSLTVSPGGTVTL | |
| | | TCGSSTGAVTTSNYVNWVQQKPGQACRGLIGG | |
| | | TNKRAPGTPARFSGSLIGGKAALTLSGAQPED | |
| | | EAEYYCVLWYSNRWVFGGGTKLTVL | |
| v619- | Anti-CD3 | EVQLVESGGGLVQPGGSLKLSCAASGFTFSTYA | 131 |
| HLh- | Heavy | MNWVRQASGKGLEWVGRIRSKYNNYATYYAD | |
| CC7 | chain | SVKDRFTISRDDSKNTAYLQMNSLKTEDTAVYY | |
| | variable | CVRHGNFGNSYVSWFAYWGQCTLVTVS | |
| | domain | | |
| | Anti-CD3 | QTVVTQEPSLTVSPGGTVTLTCGSSTGAVTTSNY | 122 |
| | Light chain | VNWVQQKPGCAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTY | 132 |
| | scFv | AMNWVRQASGKGLEWVGRIRSKYNNYATYYAD | |
| | | SVKDRFTISRDDSKNTAYLQMNSLKTEDTAVY | |
| | | YCVRHGNFGNSYVSWFAYWGQCTLVTVSSGGG | |
| | | GSGGGGSGGGGSQTVVTQEPSLTVSPGGTVTL | |
| | | TCGSSTGAVTTSNYVNWVQQKPGCAPRGLIGG | |
| | | TNKRAPGTPARFSGSLIGGKAALTLSGAQPED | |
| | | EAEYYCVLWYSNRWVFGGGTKLTVL | |
| v619- | Anti-CD3 | EVQLVESGGGLVQPGGSLKLSCAASGFTFSTY | 131 |
| HLh- | Heavy | AMNWVRQASGKGLEWVGRIRSKYNNYATYYAD | |
| CC8 | chain | SVKDRFTISRDDSKNTAYLQMNSLKTEDTAVY | |
| | variable | YCVRHGNFGNSYVSWFAYWGQCTLVTVSS | |
| | domain | | |
| | Anti-CD3 | QTVVTQEPSLTVSPGGTVTLTCGSSTGAVTTSNY | 124 |
| | Light chain | VNWVQQKPGQCPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTY | 133 |
| | scFv | AMNWVRQASGKGLEWVGRIRSKYNNYATYYAD | |
| | | SVKDRFTISRDDSKNTAYLQMNSLKTEDTAVYY | |
| | | CVRHGNFGNSYVSWFAYWGQCTLVTVSSGGGG | |
| | | SGGGGSGGGGSQTVVTQEPSLTVSPGGTVTLT | |
| | | CGSSTGAVTTSNYVNWVQQKPGQCPRGLIGGT | |
| | | NKRAPGTPARFSGSLIGGKAALTLSGAQPEDE | |
| | | AEYYCVLWYSNRWVFGGGTKLTVL | |
| v619- | Anti-CD3 | EVQLVESGGGLVQPGGSLKLSCAASGFTFSTYA | 131 |
| HLh- | Heavy | MNWVRQASGKGLEWVGRIRSKYNNYATYYAD | |
| CC9 | chain | SVKDRFTISRDDSKNTAYLQMNSLKTEDTAVYY | |
| | variable | CVRHGNFGNSYVSWFAYWGQCTLVTVSS | |
| | domain | | |
| | Anti-CD3 | QTVVTQEPSLTVSPGGTVTLTCGSSTGAVTTSNY | 126 |
| | Light chain | VNWVQQKPGQACRGLIGGTNKRAPGTPARFSG | |
| | variable | SLIGGKAALTLSGAQPEDEAEYYCVLWYSNRW | |
| | domain | VFGGGTKLTVL | |
| | Anti-CD3 | EVQLVESGGGLVQPGGSLKLSCAASGFTFSTYA | 134 |
| | scFv | MNWVRQASGKGLEWVGRIRSKYNNYATYYAD | |
| | | SVKDRFTISRDDSKNTAYLQMNSLKTEDTAVYY | |
| | | CVRHGNFGNSYVSWFAYWGQCTLVTVSSGGG | |
| | | GSGGGGSGGGGSQTVVTQEPSLTVSPGGTVTLT | |
| | | CGSSTGAVTTSNYVNWVQQKPGQACRGLIGGT | |
| | | NKRAPGTPARFSGSLIGGKAALTLSGAQPEDEAE | |
| | | YYCVLWYSNRWVFGGGTKLTVL | |
| v619_ | Anti-CD3 | EVQLVESGGGLVQPGGSLKLSCAASGFTFSTYA | 135 |
| HLh- | Heavy | MNWVRQASGKCLEWVGRIRSKYNNYATYYAD | |
| CC10 | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCGSSTGAVTTSNY | 136 |
| | Light chain | VNWVQQKPGQAPRGLIGGTNKRAPGTPARFSGS | |
| | variable | LIGGKAALTLSGAQPEDEAEYYCVLWYSNRWV | |
| | domain | FGCGTKLTVL | |
| | Anti-CD3 | EVQLVESGGGLVQPGGSLKLSCAASGFTFSTYA | 137 |
| | scFv | MNWVRQASGKCLEWVGRIRSKYNNYATYYAD | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CGSSTGAVTTSNYVNWVQQKPGQAPRGLIGGT | |
| | | NKRAPGTPARFSGSLIGGKAALTLSGAQPEDEAE | |
| | | YYCVLWYSNRWVFGCGTKLTVL | |
| v619_ | Anti-CD3 | EVQLVESGGGLVQPGGSLKLSCAASGFTFSTYA | 51 |
| HLa1 | Heavy | MNWVRQASGKGLEWVGRIRSKYNNYATYYAD | |
| | chain | SVKDRFTISRDDSKNTAYLQMNSLKTEDTAVYY | |
| | variable | CTVHGNFGNSYVSWWAYWGQGTLVTVSS | |
| | domain | | |
| | Anti-CD3 | QTVVTQEPSLTVSPGGTVTLTCRSSTGAVTTSNY | 47 |
| | Light chain | ANWVQQKPGQAPRGLIGGTKFLAPGTPARFSGS | |
| | variable | LIGGKAALTLSGAQPEDEAEYYCALWYSNRWV | |
| | domain | FGGGTKLTVL | |
| | Anti-CD3 | EVQLVESGGGLVQPGGSLKLSCAASGFTFSTYA | 138 |
| | scFv | MNWVRQASGKGLEWVGRIRSKYNNYATYYAD | |
| | | SVKDRFTISRDDSKNTAYLQMNSLKTEDTAVYY | |
| | | CTVHGNFGNSYVSWWAYWGQGTLVTVSSGGG | |
| | | GSGGGGSGGGGSQTVVTQEPSLTVSPGGTVTLT | |
| | | CRSSTGAVTTSNYANWVQQKPGQAPRGLIGGT | |
| | | KFLAPGTPARFSGSLIGGKAALTLSGAQPEDEAE | |
| | | YYCALWYSNRWVFGGGTKLTVL | |
| v619_ | Anti-CD3 | EVQLVESGGGLVQPGGSLKLSCAASGFTFSTYA | 139 |
| HLd1 | Heavy | MNWVRQASGKGLEWVGRIRSKYNNYATYYAD | |
| | chain | SVKDRFTISRDDSKNTAYLQMNSLKTEDTAVYY | |
| | variable | CTVHGNFGNSYISYWAYWGQGTLVTVSS | |
| | domain | | |
| | Anti-CD3 | QTVVTQEPSLTVSPGGTVTLTCRSSTGAVTTSNY | 55 |
| | Light chain | PNWVQQKPGQAPRGLIGGTNKRAPGTPARFSGS | |
| | variable | LIGGKAALTLSGAQPEDEAEYYCVLWYSNRWV | |
| | domain | FGGGTKLTVL | |
| | Anti-CD3 | EVQLVESGGGLVQPGGSLKLSCAASGFTFSTYA | 140 |
| | scFv | MNWVRQASGKGLEWVGRIRSKYNNYATYYAD | |
| | | SVKDRFTISRDDSKNTAYLQMNSLKTEDTAVYY | |
| | | CTVHGNFGNSYISYWAYWGQGTLVTVSSGGGG | |
| | | SGGGGSGGGGSQTVVTQEPSLTVSPGGTVTLTC | |
| | | RSSTGAVTTSNYPNWVQQKPGQAPRGLIGGTNK | |
| | | RAPGTPARFSGSLIGGKAALTLSGAQPEDEAEYY | |
| | | CVLWYSNRWVFGGGTKLTVL | |
| v619- | Anti-CD3 | EVQLVESGGGLVQPGGSLKLSCAASGFTFNKYA | 57 |
| HLL1 | Heavy | MNWVRQASGKGLEWVGRIRSKYNNYATYYAD | |
| | chain | SVKDRFTISRDDSKNTAYLQMNSLKTEDTAVYY | |
| | variable | CTRHGNFGNSYVSWFAYWGQGTLVTVSS | |
| | domain | | |
| | Anti-CD3 | QTVVTQEPSLTVSPGGTVTLTCGSSTGAVTSGN | 141 |
| | Light chain | YPNWVQQKPGQAPRGLIGGTNKRAPGTPARFSG | |
| | variable | SLIGGKAALTLSGAQPEDEAEYYCVLWYSNLW | |
| | domain | VFGGGTKLTVL | |
| | Anti-CD3 | EVQLVESGGGLVQPGGSLKLSCAASGFTFNKYA | 142 |
| | scFv | MNWVRQASGKGLEWVGRIRSKYNNYATYYAD | |
| | | SVKDRFTISRDDSKNTAYLQMNSLKTEDTAVYY | |
| | | CTRHGNFGNSYVSWFAYWGQGTLVTVSSGGGG | |
| | | SGGGGSGGGGSQTVVTQEPSLTVSPGGTVTLTC | |
| | | GSSTGAVTSGNYPNWVQQKPGQAPRGLIGGTN | |
| | | KRAPGTPARFSGSLIGGKAALTLSGAQPEDEAEY | |
| | | YCVLWYSNLWVFGGGTKLTVL | |
| v619_ | Anti-CD3 | EVQLVESGGGLVQPGGSLKLSCAASGFTFSTYA | 66 |
| HLB | Heavy | MNWVRQASGKGLEWVGRIRSKYNNYATYYAD | |
| | chain | SVKDRFTISRDDSKNTAYLQMNSLKTEDTAVYY | |
| | variable | CTRHGNFGNSYISYWAYWGQGTLVTVSS | |
| | domain | | |
| | Anti-CD3 | QTVVTQEPSLTVSPGGTVTLTCRSSTGAVTTSNY | 143 |
| | Light chain | PNWVQQKPGQAPRGLIGGTKFLAPGTPARFSGS | |
| | variable | LIGGKAALTLSGAQPEDEAEYYCALWYSNLWV | |
| | domain | FGGGTKLTVL | |
| | Anti-CD3 | EVQLVESGGGLVQPGGSLKLSCAASGFTFSTY | 144 |
| | scFv | AMNWVRQASGKGLEWVGRIRSKYNNYATYYAD | |
| | | SVKDRFTISRDDSKNTAYLQMNSLKTEDTAVYY | |
| | | CTRHGNFGNSYISYWAYWGQGTLVTVSSGGGG | |
| | | SGGGGSGGGGSQTVVTQEPSLTVSPGGTVTLTC | |
| | | RSSTGAVTTSNYPNWVQQKPGQAPRGLIGGTKF | |
| | | LAPGTPARFSGSLIGGKAALTLSGAQPEDEAEY | |
| | | YCALWYSNLWVFGGGTKLTVL | |
| v619h- | Anti-CD3 | EVQLVESGGGLVQPGGSLKLSCAASGFTFSTY | 145 |
| RE | Heavy | AMNWVRQASGKGLEWVGRIRSKYNNYATYYAR | |
| | chain | SVKDRFTISRDDSKNTAYLQMNSLKTEDTAVY | |
| | variable | YCVRHGNFGNSYVSWWAYWGQGTLVTVSS | |
| | domain | | |
| | Anti-CD3 | QTVVTQEPSLTVSPGGTVTLTCGSSTGAVTTSNY | 165 |
| | Light chain | VNWVQQKPGQAPRGLIGGTNKRAPGTPARFSGS | |
| | variable | LIGGKAALTLSGAQPEDEAEYYCVLWYSNEWV | |
| | domain | FGGGTKLTVL | |
| | Anti-CD3 | EVQLVESGGGLVQPGGSLKLSCAASGFTFSTYA | 166 |
| | scFv | MNWVRQASGKGLEWVGRIRSKYNNYATYYAR | |
| | | SVKDRFTISRDDSKNTAYLQMNSLKTEDTAVYY | |
| | | CVRHGNFGNSYVSWWAYWGQGTLVTVSSGGG | |
| | | GSGGGGSGGGGSQTVVTQEPSLTVSPGGTVTLT | |
| | | CGSSTGAVTTSNYVNWVQQKPGQAPRGLIGGT | |
| | | NKRAPGTPARFSGSLIGGKAALTLSGAQPEDEAE | |
| | | YYCVLWYSNEWVFGGGTKLTVL | |
| v619h- | Anti-CD3 | EVQLVESGGGLVQPGGSLKLSCAASGFTFSTYA | 145 |
| RD | Heavy | MNWVRQASGKGLEWVGRIRSKYNNYATYYAR | |
| | chain | SVKDRFTISRDDSKNTAYLQMNSLKTEDTAVYY | |
| | variable | CVRHGNFGNSYVSWWAYWGQGTLVTVSS | |
| | domain | | |
| | Anti-CD3 | QTVVTQEPSLTVSPGGTVTLTCGSSTGAVTTSNY | 146 |
| | Light chain | VNWVQQKPGQAPRGLIGGTNKRAPGTPARFSGS | |
| | variable | LIGGKAALTLSGAQPEDEAEYYCVLWYSNDWV | |
| | domain | FGGGTKLTVL | |
| | Anti-CD3 | EVQLVESGGGLVQPGGSLKLSCAASGFTFSTY | 147 |
| | scFv | AMNWVRQASGKGLEWVGRIRSKYNNYATYYAR | |
| | | SVKDRFTISRDDSKNTAYLQMNSLKTEDTAVY | |
| | | YCVRHGNFGNSYVSWWAYWGQGTLVTVSSGGG | |
| | | GSGGGGSGGGGSQTVVTQEPSLTVSPGGTVTL | |
| | | TCGSSTGAVTTSNYVNWVQQKPGQAPRGLIGG | |
| | | TNKRAPGTPARFSGSLIGGKAALTLSGAQPED | |
| | | EAEYYCVLWYSNDWVFGGGTKLTVL | |
| v619h- | Anti-CD3 | EVQLVESGGGLVQPGGSLKLSCAASGFTFSTY | 46 |
| R237 | Heavy | AMNWVRQASGKGLEWVGRIRSKYNNYATYYAD | |
| K | chain | SVKDRFTISRDDSKNTAYLQMNSLKTEDTAVY | |
| | variable | YCVRHGNFGNSYVSWWAYWGQGTLVTVSS | |
| | domain | | |
| | Anti-CD3 | QTVVTQEPSLTVSPGGTVTLTCGSSTGAVTTSN | 148 |
| | Light chain | YVNWVQQKPGQAPRGLIGGTNKRAPGTPARFSG | |
| | variable | LSIGGKAALTLSGAQPEDEAEYYCVLWYSNKWV | |
| | domain | FGGGTKLTVL | |
| | Anti-CD3 | EVQLVESGGGLVQPGGSLKLSCAASGFTFSTY | 149 |
| | scFv | AMNWVRQASGKGLEWVGRIRSKYNNYATYYAD | |
| | | SVKDRFTISRDDSKNTAYLQMNSLKTEDTAVY | |
| | | YCVRHGNFGNSYVSWWAYWGQGTLVTVSSGGG | |
| | | GSGGGGSGGGGSQTVVTQEPSLTVSPGGTVTL | |
| | | TCGSSTGAVTTSNYVNWVQQKPGQAPRGLIGG | |
| | | TNKRAPGTPARFSGSLIGGKAALTLSGAQPED | |
| | | EAEYYCVLWYSNKWVFGGGTKLTVL | |
| v619_ | Anti-CD3 | EVQLVESGGGLVQPGGSLKLSCAASGFTFSTYA | 150 |
| hh9- | Heavy | MNWVRQASGKGLEWVGRIRSKYNNYATYYAD | |
| CC4 | chain | SVKDRFTISRDDSKNTAYLQMNSLKTEDTAVYY | |
| | variable | CVRHGNFGNSYVSWWAYWGCGTLVTVSS | |
| | domain | | |
| | Anti-CD3 | QTVVTQEPSLTVSPGGTVTLTCRSSTGAVTTSNY | 151 |
| | Light chain | ANWVQQKPGCAPRGLIGGTNKRAPGTPARFSGS | |
| | variable | LIGGKAALTLSGAQPEDEAEYYCVLWYSNLWV | |
| | domain | FGGGTKLTVL | |
| | Anti-CD3 | EVQLVESGGGLVQPGGSLKLSCAASGFTFSTY | 152 |
| | scFv | AMNWVRQASGKGLEWVGRIRSKYNNYATYYAD | |
| | | SVKDRFTISRDDSKNTAYLQMNSLKTEDTAVY | |
| | | YCVRHGNFGNSYVSWWAYWGCGTLVTVSSGGG | |
| | | GSGGGGSGGGGSQTVVTQEPSLTVSPGGTVTL | |
| | | TCRSSTGAVTTSNYANWVQQKPGCAPRGLIGG | |
| | | TNKRAPGTPARFSGSLIGGKAALTLSGAQPED | |
| | | EAEYYCVLWYSNLWVFGGGTKLTVL | |
| v619_ | Anti-CD3 | EVQLVESGGGLVQPGGSLKLSCAASGFTFSTY | 153 |
| hh9- | Heavy | AMNWVRQAPGKGLEWVARIRSKYNNYATYYAD | |
| PA- | chain | SVKDRFTISRDDSKNTAYLQMNSLKTEDTAVY | |
| CC4 | variable | YCVRHGNFGNSYVSWWAYWGCGTLVTVSS | |
| | domain | | |
| | Anti-CD3 | QTVVTQEPSLTVSPGGTVTLTCRSSTGAVTTSNY | 151 |
| | Light chain | ANWVQQKPGCAPRGLIGGTNKRAPGTPARFSGS | |
| | variable | LIGGKAALTLSGAQPEDEAEYYCVLWYSNLWV | |
| | domain | FGGGTKLTVL | |
| | Anti-CD3 | EVQLVESGGGLVQPGGSLKLSCAASGFTFSTY | 154 |
| | scFv | AMNWVRQAPGKGLEWVARIRSKYNNYATYYAD | |
| | | SVKDRFTISRDDSKNTAYLQMNSLKTEDTAVY | |
| | | YCVRHGNFGNSYVSWWAYWGCGTLVTVSSGGG | |
| | | GSGGGGSGGGGSQTVVTQEPSLTVSPGGTVTL | |
| | | TCRSSTGAVTTSNYANWVQQKPGCAPRGLIGG | |
| | | TNKRAPGTPARFSGSLIGGKAALTLSGAQPED | |
| | | EAEYYCVLWYSNLWVFGGGTKLTVL | |
| v619_ | Anti-CD3 | EVQLVESGGGLVQPGGSLKLSCAASGFTESTY | 155 |
| hh9F- | Heavy | AMNWVRQAPGKGLEWVARIRSKYNNYATYYAD | |
| CC4P | chain | SVKDRFTISRDDSKNTAYLQMNSLKTEDTAVY | |
| A | variable | YCVRHGNFGNSYVSWFAYWGCGTLVTVSS | |
| | domain | | |
| | Anti-CD3 | QTVVTQEPSLTVSPGGTVTLTCRSSTGAVTTSNY | 151 |
| | Light chain | ANWVQQKPGCAPRGLIGGTNKRAPGTPARFSGS | |
| | variable | LIGGKAALTLSGAQPEDEAEYYCVLWYSNLWV | |
| | domain | FGGGTKLTVL | |
| | Anti-CD3 | EVQLVESGGGLVQPGGSLKLSCAASGFTFSTY | 156 |
| | scFv | AMNWVRQAPGKGLEWVARIRSKYNNYATYYAD | |
| | | SVKDRFTISRDDSKNTAYLQMNSLKTEDTAVY | |
| | | YCVRHGNFGNSYVSWFAYWGCGTLVTVSSGGG | |
| | | GSGGGGSGGGGSQTVVTQEPSLTVSPGGTVTL | |
| | | TCRSSTGAVTTSNYANWVQQKPGCAPRGLIGG | |
| | | TNKRAPGTPARFSGSLIGGKAALTLSGAQPED | |
| | | EAEYYCVLWYSNLWVFGGGTKLTVL | |
| v619_ | Anti-CD3 | EVQLVESGGGLVQPGGSLKLSCAASGFTFSTY | 153 |
| Hh9- | Heavy | AMNWVRQAPGKGLEWVARIRSKYNNYATYYAD | |
| CC4- | chain | SVKDRFTISRDDSKNTAYLQMNSLKTEDTAVY | |
| PAV | variable | YCVRHGNFGNSYVSWWAYWGCGTLVTVSS | |
| | domain | | |
| | Anti-CD3 | QTVVTQEPSLTVSPGGTVTLTCRSSTGAVTTSNY | 157 |
| | Light chain | VNWVQQKPGCAPRGLIGGTNKRAPGTPARFSGS | |
| | variable | LIGGKAALTLSGAQPEDEAEYYCVLWYSNLWV | |
| | domain | FGGGTKLTVL | |
| | Anti-CD3 | EVQLVESGGGLVQPGGSLKLSCAASGFTFSTY | 158 |
| | scFv | AMNWVRQAPGKGLEWVARIRSKYNNYATYYAD | |
| | | SVKDRFTISRDDSKNTAYLQMNSLKTEDTAVY | |
| | | YCVRHGNFGNSYVSWWAYWGCGTLVTVSSGGG | |
| | | GSGGGGSGGGGSQTVVTQEPSLTVSPGGTVTL | |
| | | TCRSSTGAVTTSNYVNWVQQKPGCAPRGLIGG | |
| | | TNKRAPGTPARFSGSLIGGKAALTLSGAQPED | |
| | | EAEYYCVLWYSNLWVFGGGTKLTVL | |
| v619_ | Anti-CD3 | EVQLVESGGGLVQPGGSLKLSCAASGFTFSTY | 155 |
| Hh9F- | Heavy | AMNWVRQAPGKGLEWVARIRSKYNNYATYYAD | |
| CC4- | chain | SVKDRFTISRDDSKNTAYLQMNSLKTEDTAVY | |
| PAV | variable | YCVRHGNFGNSYVSWFAYWGCGTLVTVSS | |
| | domain | | |
| | Anti-CD3 | QTVVTQEPSLTVSPGGTVTLTCRSSTGAVTTSNY | 157 |
| | Light chain | VNWVQQKPGCAPRGLIGGTNKRAPGTPARFSGS | |
| | variable | LIGGKAALTLSGAQPEDEAEYYCVLWYSNLWV | |
| | domain | FGGGTKLTVL | |
| | Anti-CD3 | EVQLVESGGGLVQPGGSLKLSCAASGFTFSTY | 159 |
| | scFv | AMNWVRQAPGKGLEWVARIRSKYNNYATYYAD | |
| | | SVKDRFTISRDDSKNTAYLQMNSLKTEDTAVY | |
| | | YCVRHGNFGNSYVSWFAYWGCGTLVTVSSGGG | |
| | | GSGGGGSGGGGSQTVVTQEPSLTVSPGGTVTL | |
| | | TCRSSTGAVTTSNYVNWVQQKPGCAPRGLIGGT | |
| | | NKRAPGTPARFSGSLIGGKAALTLSGAQPEDEA | |
| | | EYYCVLWYSNLWVFGGGTKLTVL | |
| v619_ | Anti-CD3 | EVQLVESGGGLVQPGGSLKLSCAASGFTFSTY | 46 |
| 531V | Heavy | AMNWVRQASGKGLEWVGRIRSKYNNYATYYAD | |
| Tras | chain | SVKDRFTISRDDSKNTAYLQMNSLKTEDTAVY | |
| 1/2 | variable | YCVRHGNFGNSYVSWWAYWGQGTLVTVSS | |
| TCB | domain | | |
| | Anti-CD3 | QTVVTQEPSLTVSPGGTVTLTCRSSTGAVTTSN | 160 |
| | Light chain | YVNWVQQKPGQAPRGLIGGTNKRAPGTPARFSG | |
| | variable | SLIGGKAALTLSGAQPEDEAEYYCVLWYSNLWV | |
| | domain | FGGGTKLTVL | |
| | Anti-CD3 | EVQLVESGGGLVQPGGSLKLSCAASGFTFSTY | 161 |
| | scFv | AMNWVRQASGKGLEWVGRIRSKYNNYATYYAD | |
| | | SVKDRFTISRDDSKNTAYLQMNSLKTEDTAVY | |
| | | YCVRHGNFGNSYVSWWAYWGQGTLVTVSSGGG | |
| | | GSGGGGSGGGGSQTVVTQEPSLTVSPGGTVTL | |
| | | TCRSSTGAVTTSNYVNWVQQKPGQAPRGLIGG | |
| | | TNKRAPGTPARFSGSLIGGKAALTLSGAQPED | |
| | | EAEYYCVLWYSNLWVFGGGTKLTVL | |
| v619_ | Anti-CD3 | EVQLVESGGGLVQPGGSLKLSCAASGFTFSTY | 46 |
| c15 | Heavy | AMNWVRQASGKGLEWVGRIRSKYNNYATYYAD | |
| | chain | SVKDRFTISRDDSKNTAYLQMNSLKTEDTAVY | |
| | variable | YCVRHGNFGNSYVSWWAYWGQGTLVTVSS | |
| | domain | | |
| | Anti-CD3 | QTVVTQEPSLTVSPGGTVTLTCRSSTGAVTTSN | 49 |
| | Light chain | YPNWVQQKPGQAPRGLIGGTNKRAPGTPARFSG | |
| | variable | SLLGGKAALTLSGVQPEDEAEYYCVLWYSNRWV | |
| | domain | FGGGTKLTVL | |
| | Anti-CD3 | EVQLVESGGGLVQPGGSLKLSCAASGFTFSTY | 50 |
| | scFv | AMNWVRQASGKGLEWVGRIRSKYNNYATYYAD | |
| | | SVKDRFTISRDDSKNTAYLQMNSLKTEDTAVY | |
| | | YCVRHGNFGNSYVSWWAYWGQGTLVTVSSGGG | |
| | | GSGGGGSGGGGSQTVVTQEPSLTVSPGGTVTL | |
| | | TCRSSTGAVTTSNYPNWVQQKPGQAPRGLIGG | |
| | | TNKRAPGTPARFSGSLLGGKAALTLSGVQPED | |
| | | EAEYYCVLWYSNRWVFGGGTKLTVL | |
| v619_ | Anti-CD3 | EVQLVESGGGLVQPGGSLKLSCAASGFTFSTY | 46 |
| h15 | Heavy | AMNWVRQASGKGLEWVGRIRSKYNNYATYYAD | |
| | chain | SVKDRFTISRDDSKNTAYLQMNSLKTEDTAVY | |
| | variable | YCVRHGNFGNSYVSWWAYWGQGTLVTVSS | |
| | domain | | |
| | Anti-CD3 | QTVVTQEPSLTVSPGGTVTLTCGSSTGAVTTS | 64 |
| | Light chain | NYVNWVQQKPGQAPRGLIGGTNKRAPGTPARF | |
| | variable | SGSLIGGKAALTLSGAQPEDEAEYYCVLWYSN | |
| | domain | RWVFGGGTKLTVL | |
| | Anti-CD3 | EVQLVESGGGLVQPGGSLKLSCAASGFTFSTY | 28 |
| | scFv | AMNWVRQASGKGLEWVGRIRSKYNNYATYYAD | |
| | | SVKDRFTISRDDSKNTAYLQMNSLKTEDTAVY | |
| | | YCVRHGNFGNSYVSWWAYWGQGTLVTVSSGGG | |
| | | GSGGGGSGGGGSQTVVTQEPSLTVSPGGTVTL | |
| | | TCGSSTGAVTTSNYVNWVQQKPGQAPRGLIGG | |
| | | TNKRAPGTPARFSGSLIGGKAALTLSGAQPED | |
| | | EAEYYCVLWYSNRWVFGGGTKLTVL | |
| V12 | Anti-CD3 | EVQLVESGGGLVQPGGSLRLSCAASGFTFSTY | 162 |
| (refer- | Heavy | AMNWVRQAPGKGLEWVARIRSKYNNYATYYAD | |
| ence | chain | SVKDRFTISRDDSKNSLYLQMNSLKTEDTAVY | |
| anti- | variable | YCVRHGNFGNSYVSWFAYWGQGTLVTVSS | |
| CD3 | domain | | |
| scFv) | | | |
| | Anti-CD3 | QTVVTQEPSFSVSPGGTVTLTCRSSTGAVTTSNY | 163 |
| | Light chain | ANWVQQTPGQAPRGLIGGTNKRAPGVPDRESGS | |
| | variable | ILGNKAALTITGAQADDESDYYCALWYSNLWV | |
| | domain | FGGGTKLTVL | |
| | Anti-CD3 | QTVVTQEPSFSVSPGGTVTLTCRSSTGAVTTSNY | 164 |
| | scFv | ANWVQQTPGQAPRGLIGGTNKRAPGVPDRESGS | |
| | | ILGNKAALTITGAQADDESDYYCALWYSNLWV | |
| | | FGGGTKLTVLGGGGSGGGGSGGGGSEVQLVES | |
| | | GGGLVQPGGSLRLSCAASGFTFSTYAMNWVRQ | |
| | | APGKGLEWVARIRSKYNNYATYYADSVKDRFTI | |
| | | SRDDSKNSLYLQMNSLKTEDTAVYYCVRHGNF | |
| | | GNSYVSWFAYWGQGTLVTVSS | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition section headings, the materials, methods, and examples are illustrative only and not intended to be limiting.

What is claimed is:

1. A target-binding protein comprising:
a heavy chain variable domain comprising
a variable heavy chain complementarity determining region 1 (VH CDR1) comprising a sequence of TYAMN (SEQ ID NO: 3),
a variable heavy chain complementarity determining region 2 (VH CDR2) comprising a sequence of RIRSKYNNYATYYADSVKD (SEQ ID NO: 5), and
a variable heavy chain complementarity determining region 3(VH CDR3) comprising a sequence of HGNFGNSYVSWX₁AY (SEQ ID NO: 6), wherein X₁ is W or F; and
a light chain variable domain comprising
a variable light chain complementarity determining region 1(VL CDR1) comprising a sequence of X₂SSTGAVTTSNYX₃N (SEQ ID NO: 10), wherein X₂ is R or G; and X₃ is P or V,
a variable light chain complementarity determining region 2 (VL CDR2) comprising a sequence of GTNKRAP (SEQ ID NO: 14), and
a variable light chain complementarity determining region 3 (VL CDR3) comprising a sequence of X₄LWYSNX₅WV (SEQ ID NO: 15), wherein X₄ is V or I; and
X₅ is R or L,
wherein the heavy chain variable domain and the light chain variable domain are disposed within one or more polypeptides,
wherein the target-binding protein specifically binds to CD3 epsilon.

2. The target-binding protein of claim 1, wherein:
a. the VH CDR1 comprises a sequences of TYAMN (SEQ ID NO: 3);
b. the VH CDR2 comprises a sequence of RIRSKYNNYATYYADSVKD (SEQ ID NO: 5);
c. the VH CDR3 comprises a sequence of HGNFGNSYVSWWAY (SEQ ID NO: 7) or HGNFGNSYVSWFAY (SEQ ID NO: 8);
d. the VL CDR1 comprises a sequence of RSSTGAVTTSNYPN (SEQ ID NO: 11), RSSTGAVTTSNYVN (SEQ ID NO: 12), or GSSTGAVTTSNYVN (SEQ ID NO: 13);
e. the VL CDR2 comprises a sequence of GTNKRAP (SEQ ID NO: 14); and
f. the VL CDR3 comprises a sequence of VLWYSNRWV (SEQ ID NO: 16), VLWYSNLWV (SEQ ID NO: 17), or ILWYSNRWV (SEQ ID NO: 18).

3. The target-binding protein of claim 1, wherein
the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWWAY (SEQ ID NO: 7), the VL CDR1 comprises RSSTGAVTTSNYPN (SEQ ID NO: 11), the VL CDR2 comprises GTNKRAP (SEQ ID NO: 14), and the VL CDR3 comprises VLWYSNRWV (SEQ ID NO: 16);
the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWWAY (SEQ ID NO: 7), the VL CDR1 comprises GSSTGAVTTSNYVN (SEQ ID NO: 13), the VL CDR2 comprises GTNKRAP (SEQ ID NO: 14), and the VL CDR3 comprises VLWYSNRWV (SEQ ID NO: 16);
the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWFAY (SEQ ID NO: 8), the VL CDR1 comprises GSSTGAVTTSNYVN (SEQ ID NO: 13), the VL CDR2 comprises GTNKRAP (SEQ ID NO: 14), and the VL CDR3 comprises VLWYSNRWV (SEQ ID NO: 16);
the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWWAY (SEQ ID NO: 7), the VL CDR1 comprises RSSTGAVTTSNYVN (SEQ ID NO: 12), the VL CDR2 comprises GTNKRAP (SEQ ID NO: 14), and the VL CDR3 comprises ILWYSNRWV (SEQ ID NO: 18);
the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWWAY (SEQ ID NO: 7), the VL CDR1 comprises GSSTGAVTTSNYVN (SEQ ID NO: 13), the VL CDR2 comprises GTNKRAP (SEQ ID NO: 14), and the VL CDR3 comprises VLWYSNLWV (SEQ ID NO: 17); or
the VH CDR1 comprises TYAMN (SEQ ID NO: 3), the VH CDR2 comprises RIRSKYNNYATYYADSVKD (SEQ ID NO: 5), the VH CDR3 comprises HGNFGNSYVSWWAY (SEQ ID NO: 7), the VL CDR1 comprises RSSTGAVTTSNYVN (SEQ ID NO: 12), the VL CDR2 comprises GTNKRAP (SEQ ID NO: 14), and the VL CDR3 comprises VLWYSNRWV (SEQ ID NO: 16).

4. The target-binding protein of claim 3, wherein the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWWAY (SEQ ID NO: 7), and the light chain variable domain comprises the VL CDR1 comprising RSSTGAVTTSNYPN (SEQ ID NO: 11), the VL CDR2 comprising GTNKRAP (SEQ ID NO: 14), and the VL CDR3 comprising VLWYSNRWV (SEQ ID NO: 16).

5. The target-binding protein of claim 4, wherein the heavy chain variable domain comprises a sequence that is at least 90%, or at least 91%, 92%, 93%, 94, 95%, 96%, 97%, 98%, or 99% identical to SEQ ID NO: 46; and the light chain variable domain comprises a sequence that is at least 90%, or at least 91%, 92%, 93%, 94, 95%, 96%, 97%, 98%, or 99% identical to SEQ ID NO: 49.

6. The target-binding protein of claim 4, wherein the heavy chain variable domain comprises the amino acid sequence of SEQ ID NO: 46 and the light chain variable domain comprises the amino acid sequence of SEQ ID NO: 49.

7. The target-binding protein of claim 3, wherein the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWWAY (SEQ ID NO: 7), and the light chain variable domain comprises the VL CDR1 comprising GSSTGAVTTSNYVN (SEQ ID NO: 13), the VL CDR2 comprising GTNKRAP (SEQ ID NO: 14), and the VL CDR3 comprising VLWYSNRWV (SEQ ID NO: 16).

8. The target-binding protein of claim 7, wherein the heavy chain variable domain comprises a sequence that is at least 90%, or at least 91%, 92%, 93%, 94, 95%, 96%, 97%, 98%, or 99% identical to SEQ ID NO: 46; and the light chain variable domain comprises a sequence that is at least 90%, or at least 91%, 92%, 93%, 94, 95%, 96%, 97%, 98%, or 99% identical to SEQ ID NO: 64.

9. The target-binding protein of claim 7, wherein the heavy chain variable domain comprises a sequence of SEQ ID NO: 46, and the light chain variable domain comprises a sequence of SEQ ID NO: 64.

10. The target-binding protein of claim 3, wherein the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWFAY (SEQ ID NO: 8), and the light chain variable domain comprises the VL CDR1 comprising GSSTGAVTTSNYVN (SEQ ID NO: 13), the VL CDR2 comprising GTNKRAP (SEQ ID NO: 14), and the VL CDR3 comprising VLWYSNRWV (SEQ ID NO: 16).

11. The target-binding protein of claim 10, wherein the heavy chain variable domain comprises a sequence that is at least 90%, or at least 91%, 92%, 93%, 94, 95%, 96%, 97%, 98%, or 99% identical to SEQ ID NO: 128; and the light chain variable domain comprises a sequence that is at least 90%, or at least 91%, 92%, 93%, 94, 95%, 96%, 97%, 98%, or 99% identical to SEQ ID NO: 122.

12. The target-binding protein of claim 10, wherein the heavy chain variable domain comprises a sequence of SEQ ID NO: 128, and the light chain variable domain comprises a sequence of SEQ ID NO: 122.

13. The target-binding protein of claim 3, wherein the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWWAY (SEQ ID NO: 7), and the light chain variable domain comprises the VL CDR1 comprising RSSTGAVTTSNYVN (SEQ ID NO: 12), the VL CDR2 comprising GTNKRAP (SEQ ID NO: 14), and the VL CDR3 comprising ILWYSNRWV (SEQ ID NO: 18).

14. The target-binding protein of claim 13, wherein the heavy chain variable domain comprises a sequence that is at least 90%, or at least 91%, 92%, 93%, 94, 95%, 96%, 97%, 98%, or 99% identical to SEQ ID NO: 46; and the light chain variable domain comprises a sequence that is at least 90%, or at least 91%, 92%, 93%, 94, 95%, 96%, 97%, 98%, or 99% identical to SEQ ID NO: 113.

15. The target-binding protein of claim 13, wherein the heavy chain variable domain comprises a sequence of SEQ ID NO: 46, and the light chain variable domain comprises a sequence of SEQ ID NO: 113.

16. The target-binding protein of claim 3, wherein the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWWAY (SEQ ID NO: 7), and the light chain variable domain comprises the VL CDR1 comprising GSSTGAVTTSNYVN (SEQ ID NO: 13), the VL CDR2 comprising GTNKRAP (SEQ ID NO: 14), and the VL CDR3 comprising VLWYSNLWV (SEQ ID NO: 17).

17. The target-binding protein of claim 16, wherein the heavy chain variable domain comprises a sequence that is at least 90%, or at least 91%, 92%, 93%, 94, 95%, 96%, 97%, 98%, or 99% identical to SEQ ID NO: 46; and the light chain variable domain comprises a sequence that is at least 90%, or at least 91%, 92%, 93%, 94, 95%, 96%, 97%, 98%, or 99% identical to SEQ ID NO: 98.

18. The target-binding protein of claim 16, wherein the heavy chain variable domain comprises a sequence of SEQ ID NO: 46, and the light chain variable domain comprises a sequence of SEQ ID NO: 98.

19. The target-binding protein of claim 3, wherein the heavy chain variable domain comprises the VH CDR1 comprising TYAMN (SEQ ID NO: 3), the VH CDR2 comprising RIRSKYNNYATYYADSVKD (SEQ ID NO: 5), the VH CDR3 comprising HGNFGNSYVSWWAY (SEQ ID NO: 7), and the light chain variable domain comprises the VL CDR1 comprising RSSTGAVTTSNYVN (SEQ ID NO: 12), the VL CDR2 comprising GTNKRAP (SEQ ID NO: 14), and the VL CDR3 comprising VLWYSNRWV (SEQ ID NO: 16).

20. The target-binding protein of claim 19, wherein the heavy chain variable domain comprises a sequence that is at least 90%, or at least 91%, 92%, 93%, 94, 95%, 96%, 97%, 98%, or 99% identical to SEQ ID NO: 46; and the light chain variable domain comprises a sequence that is at least 90%, or at least 91%, 92%, 93%, 94, 95%, 96%, 97%, 98%, or 99% identical to SEQ ID NO: 107.

21. The target-binding protein of claim 19, wherein the heavy chain variable domain comprises a sequence of SEQ ID NO: 46, and the light chain variable domain comprises a sequence of SEQ ID NO: 107.

22. A target-binding protein comprising:
a heavy chain variable domain comprising a sequence of EVQLVESGGGLVQPGGSLKLSCAASGFTFSTYAMNWVRQASGKGLEWVGRIRS KYNNYATYYADSVKDRFTISRDDSKNTAYLQMNSLKTEDTAVYYCVRHGNFGN SYVSWX₁AYWGX₆GTLVTVSS (SEQ ID NO: 20); and
a light chain variable domain comprising a sequence of QTVVTQEPSLTVSPGGTVTLTCX₂SSTGAVTTSNYX₃NWVQQKPGX₇APRGLIGGT NKRAPGTPARFSGSLJGGKAALTLSGX₉QPEDEAEYYCX₄LWYSNX₅WVFGGGTK LTVL (SEQ ID NO: 21),
wherein:
X₁ is W or F; X₂ is R or G; X₃ is P or V; X₄ is V or I; X₅ is R or L,
X₆ is Q and X₇ is Q; or X₆ is C and X₇ is C,
J is L when X₉ is V; or J is I when X₉ is A, and
the target-binding protein specifically binds to CD3 epsilon.

23. The target-binding protein of claim 22, wherein X₆ is Q and X₇ is Q.

24. The target-binding protein of claim 22, wherein X₆ is C and X₇ is C.

25. The target-binding protein of claim 22, wherein
the heavy chain variable domain comprises a sequence of SEQ ID NO: 46 and the light chain variable domain comprises a sequence of SEQ ID NO: 49;
the heavy chain variable domain comprises a sequence of SEQ ID NO: 46 and the light chain variable domain comprises a sequence of SEQ ID NO: 64;
the heavy chain variable domain comprises a sequence of SEQ ID NO: 46 and the light chain variable domain comprises a sequence of SEQ ID NO: 113;
the heavy chain variable domain comprises a sequence of SEQ ID NO: 46 and the light chain variable domain comprises a sequence of SEQ ID NO: 98;
the heavy chain variable domain comprises a sequence of SEQ ID NO: 46 and the light chain variable domain comprises a sequence of SEQ ID NO: 107; or
the heavy chain variable domain comprises a sequence of SEQ ID NO: 128 and the light chain variable domain comprises a sequence of SEQ ID NO: 122.

26. The target-binding protein of any one of claims 1-25, wherein the heavy chain variable domain and the light chain variable domain are disposed within the same polypeptide.

27. The target-binding protein of claim 26, wherein the heavy chain variable domain and the light chain variable domain are coupled via a linker.

28. The target-binding protein of claim 27, wherein the linker has a length of 5 to 30, 6 to 29, 7 to 28, 8 to 27, 9 to 26, 10 to 25, 11, 12, 13, 14, 15, 16, 17, 18, 19, 20, 21, 22, 23, or 24 amino acids.

29. The target-binding protein of claim 26, comprising a sequence of SEQ ID NO: 50, 28, 114, 99, 108, or 32.

30. The target-binding protein of any one of claims 26-29, wherein the target-binding protein comprises a single chain variable fragment (scFv).

31. The target-binding protein of any one of claims 1-30, wherein the target-binding protein comprises a BiTE, a (scFv)₂, a NANOBODY®, a nanobody-HSA VHH-scAb, a VHH-Fab, a Dual scFab, a F(ab′)₂, a diabody, a CROSSMAB®, a DAF (two-in-one), a DAE (four-in-one), a DUTAMAB®, a DT-IgG, a knobs-in-holes common light chain, a knobs-in-holes assembly, a charge pair, a Fab-arm exchange, a SEEDbody, a LUZ-Y, a FcAb, a kl-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, ZYBODY™, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a LHH-Fc KiH, a Fab-VHH-Fc, an Intrabody, a dock and lock, an ImmTAC® (immune-mobilizing monoclonal TCRs (T cell receptors) against cancer), an IgG-IgG conjugate, a Cov-X-Body, a scFv1-PEG-scFv2, an Adnectin, a DARPin®, a fibronectin, an IgG, an IgM, an IgA, an IgE, an IgD, or a DEP conjugate, TMEAbody™, SAFEbody®, TRITAC®, or SHIELD antibody.

32. The target-binding protein of any one of claims 1-30, wherein the target-binding protein comprises an IgG, IgM, IgA, IgE, or IgD antibody or fragment thereof.

33. The target-binding protein of claim 32, wherein the target-binding protein comprises an IgG1, IgG2, IgG3, or IgG4 antibody.

34. The target-binding protein of any one of claims 1-33, wherein the target-binding protein is humanized.

35. The target-binding protein of any one of claims 1-34, further comprising a masking moiety that inhibits binding of the target-binding protein to CD3 in an inactive state.

36. The target-binding protein of claim 35, wherein the masking moiety is coupled to the target-binding protein via a cleavable moiety, and the cleavable moiety is a substrate for a protease.

37. The target-binding protein of any one of claim 36, wherein the protease is ADAMS, ADAMTS, ADAM8, ADAM9, ADAM10, ADAM12, ADAM15, ADAM17/TACE, ADAMDEC1, ADAMTS1, ADAMTS4, ADAMTS5, Aspartate proteases, BACE, Renin, Aspartic cathepsins, Cathepsin D, Cathepsin E, Caspases, Caspase 1, Caspase 2, Caspase 3, Caspase 4, Caspase 5, Caspase 6, Caspase 7, Caspase 8, Caspase 9, Caspase 10, Caspase 14, Cysteine cathepsins, Cathepsin B, Cathepsin C, Cathepsin K, Cathepsin L, Cathepsin S, Cathepsin V/L2, Cathepsin X/Z/P, Cysteine proteinases, Cruzipain, Legumain, Otubain-2, KLKs, KLK4, KLK5, KLK6, KLK7, KLK8, KLK10, KLK11, KLK13, KLK14, Metallo proteinases, Meprin, Neprilysin, PSMA, BMP-1, MMPs, MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27, Serine proteases, activated protein C, Cathepsin A, Cathepsin G, Chymase, coagulation factor proteases, FVIIa, FIXa, FXa, FXIa, FXIIa, Elastase, Granzyme B, Guanidinobenzoatase, HtrA1, Human Neutrophil Elastase, Lactoferrin, Marapsin, NS3/4A, PACE4, Plasmin, PSA, tPA, Thrombin, Tryptase, uPA, Type II Transmembrane, Serine Proteases, TTSPs, DESC1, DPP-4, FAP, Hepsin, Matriptase-2, MT-SP1/Matriptase, TMPRSS2, TMPRSS3, or TMPRSS4.

38. The target-binding protein of any one of claims 1-37, wherein the heavy chain variable domain and/or the light chain variable domain is conjugated to a toxin, radioisotope, small molecule, diagnostic agent, therapeutic macromolecule, targeting moiety, or detectable moiety, via a conjugating moiety.

39. The target-binding protein of claim 38, wherein the conjugating moiety is cleavable by a protease.

40. The target-binding protein of claim 38, wherein the conjugating moiety is non-cleavable by a protease.

41. A composition comprising the target-binding protein of any one of claims 1-40 and a carrier.

42. The composition of claim 41, wherein the composition is a pharmaceutical composition, and wherein the carrier is a pharmaceutically acceptable carrier.

43. A container, vial, syringe, injector pen, or kit comprising at least one dose of the composition of claim 41 or 42.

44. A nucleic acid comprising a sequence encoding the target-binding protein of any one of claims 1-40.

45. A vector comprising the nucleic acid of claim 44.

46. A cell comprising the nucleic acid of claim 44 or the vector of claim 45.

47. A method of treating a subject in need thereof comprising administering to the subject a therapeutically effective amount of the target-binding protein of any one of claims 1-40 or the composition of claim 41 or 42.

48. The method of claim 47, wherein the subject has been identified or diagnosed as having a cancer, an inflammatory condition, disorder or disease, or an autoimmune condition, disorder or disease.

49. A method of producing a target-binding protein, comprising:
culturing the cell of claim 46 in a culture medium under a condition sufficient to produce the target-binding protein; and
recovering the target-binding protein from the cell or the culture medium.

50. The method of claim 49, further comprising isolating the target-binding protein recovered from the cell or the culture medium.

51. The method of claim 50, further comprising formulating the target-binding protein into a pharmaceutical composition.
